(19) United States
(12) Patent Application Publication
(10) Pub. No.: US 2023/0174628 A1
(43) Pub. Date: Jun. 8, 2023

(54) ANTI-NOVEL CORONAVIRUS MONOCLONAL ANTIBODY AND APPLICATION THEREOF

(71) Applicant: PEKING UNIVERSITY, Beijing (CN)

(72) Inventors: Xiaoliang Sunney XIE, Beijing (CN); Yunlong CAO, Beijing (CN); Wenjie SUN, Beijing (CN); Xu ZHANG, Beijing (CN)

(21) Appl. No.: 17/921,965

(22) PCT Filed: Apr. 27, 2021
(86) PCT No.: PCT/CN2021/090146

§ 371 Date: Oct. 27, 2022

(30) Foreign Application Priority Data
Apr. 28, 2020 (CN) 202010352448.5

(51) Int. Cl. C07K 16/10; G01N 33/569; A61P 31/14

(52) U.S. Cl. CPC C07K 16/10; G01N 33/56983; A61P 31/14; C07K 2317/92; C07K 2317/76; G01N 2333/165; G01N 2469/10; A61K 2039/505

(57) ABSTRACT

The present invention relates to the fields of immunology and molecular virology, and in particular, to the field of diagnosis, prevention and treatment of a novel coronavirus. Specifically, the present invention relates to an anti-novel coronavirus monoclonal antibody and a composition (for example, a diagnostic agent and a therapeutic agent) containing same. In addition, the present invention also relates to use of the antibody. The antibody of the present invention can be used for diagnosing, preventing and/or treating novel coronavirus infections and/or diseases (for example, novel coronavirus pneumonia) caused by the infections.

### TECHNICAL FIELD

The present invention relates to the fields of immunology and molecular virology, and in particular, to the field of diagnosis, prevention and treatment of a novel coronavirus. Specifically, the present invention relates to an anti-novel coronavirus antibody and a composition (for example, a diagnostic agent and a therapeutic agent) containing same. In addition, the present invention also relates to use of the antibody. The antibody of the present invention can be used for diagnosing, preventing and/or treating novel coronavirus infections and/or diseases (for example, novel coronavirus pneumonia) caused by the infections.

### BACKGROUND ART

As a single-stranded RNA virus, the novel coronavirus (severe acute respiratory syndrome coronavirus 2, SARS-CoV-2) is the pathogen of novel coronavirus pneumonia (coronavirus disease 2019, COVID-19), and is a member of the Coronaviridae family, alongside the severe acute respiratory syndrome coronavirus (SARS-CoV) epidemic in 2002-2003 and the Middle East respiratory syndrome coronavirus (MERS-CoV) epidemic in 2012. Coronavirus is a relatively large virus with round, oval or pleomorphic particles having a diameter of 50-200 nm. Coronavirus is an enveloped virus. The capsid of the virus is enveloped with a lipid envelope, on which a wide spike protein (Spike, S protein, SEQ ID No: 1460) is arranged forming a sun halo shape. Studies have confirmed that the S protein is located on the surface of the novel coronavirus SARS-CoV-2, and can bind to a receptor, angiotensin converting enzyme 2 (ACE2) molecule of a host cell via a receptor binding domain (RBD) contained therein during the virus infection of the host, thereby initiating the fusion of the viral membrane with the host cell membrane and causing the virus to infect the host cell.

So far, a neutralizing antibody has been proved to be an effective method for treating viral diseases. In general, upon stimulated by an antigen, a B lymphocyte in a patient is activated and then transformed and differentiated into a variety of different cells, and antibodies are produced. According to existing researches and reports, there is an anti-novel coronavirus antibody in the peripheral blood of patients recovered from novel coronavirus pneumonia, which is produced and secreted by activated B cells. However, there are a variety of B cells in the plasma of the recovered patients, and the binding activities and neutralizing titers of antibodies produced by different B cells are also different. So far, there is no study reporting an anti-novel coronavirus antibody with a high binding activity and/or a high neutralizing activity.

Therefore, there is a need to develop an antibody with a high binding activity and/or a high neutralizing activity against novel coronavirus SARS-CoV-2, thereby providing effective means for diagnosing, preventing and/or treating novel coronavirus infections.

### SUMMARY OF THE INVENTION

The following technical solutions provided herein meet the above-mentioned needs and provide relevant advantages.

In one aspect, provided herein is an antigen-binding unit comprising a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3, wherein the VH CDR3 comprises a sequence selected from SEQ ID NOs: 1-360 and 2971-3005 or a sequence comprising one or more amino acid additions, deletions, or substitutions compared with SEQ ID NOs: 1-360 and 2971-3005, and/or wherein the VL CDR3 comprises a sequence selected from SEQ ID NOs: 361-720 and 3076-3110 or a sequence comprising one or more amino acid additions, deletions, or substitutions compared with SEQ ID NOs: 361-720 and 3076-3110.

In some embodiments, the antigen-binding unit binds to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

In some embodiments, the antigen-binding unit neutralizes the novel coronavirus (SARS-CoV-2) with an IC₅₀ of less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, or less than 0.001 μg/ml.

In some embodiments, the VH CDR1 of the antigen-binding unit comprises a sequence selected from SEQ ID NOs: 1461-1820 and 2901-2935 or a sequence comprising one or more amino acid additions, deletions, or substitutions compared with SEQ ID NOs: 1461-1820 and 2901-2935. In some embodiments, the VH CDR1 of the antigen-binding unit comprises a sequence selected from SEQ ID NOs: 1461-1820 and 2901-2935. In some embodiments, the VH CDR1 of the antigen-binding unit comprises a sequence comprising 5, 4, 3, 2 or 1 amino acid additions, deletions, or substitutions compared with SEQ ID NOs: 1461-1820 and 2901-2935. In some embodiments, the VH CDR1 of the antigen-binding unit comprises the same sequence as CDR1 contained in SEQ ID NOs: 721-1080 and 3111-3145.

In some embodiments, the VH CDR2 of the antigen-binding unit comprises a sequence selected from SEQ ID NOs: 1821-2180 and 2936-2970 or a sequence comprising one or more amino acid additions, deletions, or substitutions compared with SEQ ID NOs: 1821-2180 and 2936-2970. In some embodiments, the VH CDR2 of the antigen-binding unit comprises a sequence selected from SEQ ID NOs: 1821-2180 and 2936-2970. In some embodiments, the VH CDR2 of the antigen-binding unit comprises a sequence comprising 5, 4, 3, 2 or 1 amino acid additions, deletions, or substitutions compared with SEQ ID NOs: 1821-2180 and 2936-2970. In some embodiments, the VH CDR2 of the antigen-binding unit comprises the same sequence as CDR2 contained in SEQ ID NOs: 721-1080 and 3111-3145.

In some embodiments, the VL CDR1 of the antigen-binding unit comprises a sequence selected from SEQ ID NOs: 2181-2540 and 3006-3040 or a sequence comprising one or more amino acid additions, deletions, or substitutions compared with SEQ ID NOs: 2181-2540 and 3006-3040. In some embodiments, the VL CDR1 of the antigen-binding unit comprises a sequence selected from SEQ ID NOs: 2181-2540 and 3006-3040. In some embodiments, the VL CDR1 of the antigen-binding unit comprises a sequence comprising 5, 4, 3, 2 or 1 amino acid additions, deletions, or substitutions compared with SEQ ID NOs: 2181-2540 and 3006-3040. In some embodiments, the VL CDR1 of the antigen-binding unit comprises the same sequence as CDR1 contained in SEQ ID NOs: 1081-1440 and 3146-3180.

In some embodiments, the VL CDR2 of the antigen-binding unit comprises a sequence selected from SEQ ID NOs: 2541-2900 and 3041-3075 or a sequence comprising one or more amino acid additions, deletions, or substitutions compared with SEQ ID NOs: 2541-2900 and 3041-3075. In some embodiments, the VL CDR2 of the antigen-binding unit comprises a sequence selected from SEQ ID NOs: 2541-2900 and 3041-3075. In some embodiments, the VL CDR2 of the antigen-binding unit comprises a sequence comprising 5, 4, 3, 2 or 1 amino acid additions, deletions, or substitutions compared with SEQ ID NOs: 2541-2900 and 3041-3075. In some embodiments, the VL CDR2 of the antigen-binding unit comprises the same sequence as CDR2 contained in SEQ ID NOs: 1081-1440 and 3146-3180.

In some embodiments, the VH of the antigen-binding unit comprises a sequence selected from SEQ ID NOs: 721-1080 and 3111-3145 or a sequence comprising one or more amino acid additions, deletions, or substitutions compared with SEQ ID NOs: 721-1080 and 3111-3145. In some embodiments, the VH of the antigen-binding unit comprises a sequence selected from SEQ ID NOs: 721-1080 and 3111-3145. In some embodiments, the VH of the antigen-binding unit comprises a sequence comprising 10, 9, 8, 7, 6, 5, 4, 3, 2 or 1 amino acid additions, deletions, or substitutions compared with SEQ ID NOs: 721-1080 and 3111-3145. In some embodiments, the VH of the antigen-binding unit comprises a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145.

In some embodiments, the VL of the antigen-binding unit comprises a sequence selected from SEQ ID NOs: 1081-1440 and 3146-3180 or a sequence comprising one or more amino acid additions, deletions, or substitutions compared with SEQ ID NOs: 1081-1440 and 3146-3180. In some embodiments, the VL of the antigen-binding unit comprises a sequence selected from SEQ ID NOs: 1081-1440 and 3146-3180. In some embodiments, the VL of the antigen-binding unit comprises a sequence comprising 10, 9, 8, 7, 6, 5, 4, 3, 2 or 1 amino acid additions, deletions, or substitutions compared with SEQ ID NOs: 1081-1440 and 3146-3180. In some embodiments, the VL of the antigen-binding unit comprises a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180.

In another aspect, provided herein is an antigen-binding unit comprising a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3, wherein the VH CDR1 comprises a sequence selected from SEQ ID NOs: 1461-1820 and 2901-2935, a sequence comprising one or more amino acid additions, deletions, or substitutions compared with SEQ ID NOs: 1461-1820 and 2901-2935, or the same sequence as CDR1 contained in SEQ ID NOs: 721-1080 and 3111-3145, wherein the VH CDR2 comprises a sequence selected from SEQ ID NOs: 1821-2180 and 2936-2970, a sequence comprising one or more amino acid additions, deletions, or substitutions compared with SEQ ID NOs: 1821-2180 and 2936-2970, or the same sequence as CDR2 contained in SEQ ID NOs: 721-1080 and 3111-3145, and wherein the VH CDR3 comprises a sequence selected from SEQ ID NOs: 1-360 and 2971-3005, a sequence comprising one or more amino acid additions, deletions, or substitutions compared with SEQ ID NOs: 1-360 and 2971-3005, or the same sequence as CDR3 contained in SEQ ID NOs: 721-1080 and 3111-3145, and/or wherein the VL CDR1 comprises a sequence selected from SEQ ID NOs: 2181-2540 and 3006-3040, a sequence comprising one or more amino acid additions, deletions, or substitutions compared with SEQ ID NOs: 2181-2540 and 3006-3040, or the same sequence as CDR1 contained in SEQ ID NOs: 1081-1440 and 3146-3180, the VL CDR2 comprises a sequence selected from SEQ ID NOs: 2541-2900 and 3041-3075, a sequence comprising one or more amino acid additions, deletions, or substitutions compared with SEQ ID NOs: 2541-2900 and 3041-3075, or the same sequence as CDR2 contained in SEQ ID NOs: 1081-1440 and 3146-3180, and the VL CDR3 comprises a sequence selected from SEQ ID NOs: 361-720 and 3076-3110, a sequence comprising one or more amino acid additions, deletions, or substitutions compared with SEQ ID NOs: 361-720 and 3076-3110, or the same sequence as CDR3 contained in SEQ ID NOs: 1081-1440 and 3146-3180.

In another aspect, provided herein is an antigen-binding unit comprising a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3, wherein the VH CDR1 comprises a sequence selected from SEQ ID NOs: 1461-1820 and 2901-2935 or a sequence comprising one or more amino acid additions, deletions, or substitutions compared with SEQ ID NOs: 1461-1820 and 2901-2935, wherein the VH CDR2 comprises a sequence selected from SEQ ID NOs: 1821-2180 and 2936-2970 or a sequence comprising one or more amino acid additions, deletions, or substitutions compared with SEQ ID NOs: 1821-2180 and 2936-2970, and wherein the VH CDR3 comprises a sequence selected from SEQ ID NOs: 1-360 and 2971-3005 or a sequence comprising one or more amino acid additions, deletions, or substitutions compared with SEQ ID NOs: 1-360 and 2971-3005, and/or wherein the VL CDR1 comprises a sequence selected from SEQ ID NOs: 2181-2540 and 3006-3040 or a sequence comprising one or more amino acid additions, deletions, or substitutions compared with SEQ ID NOs: 2181-2540 and 3006-3040, the VL CDR2 comprises a sequence selected from SEQ ID NOs: 2541-2900 and 3041-3075 or a sequence comprising one or more amino acid additions, deletions, or substitutions compared with SEQ ID NOs: 2541-2900 and 3041-3075, and the VL CDR3 comprises a sequence selected from SEQ ID NOs: 361-720 and 3076-3110 or a sequence comprising one or more amino acid additions, deletions, or substitutions compared with SEQ ID NOs: 361-720 and 3076-3110.

In some embodiments, the VH of the antigen-binding unit comprises a sequence selected from SEQ ID NOs: 721-1080 and 3111-3145 or a sequence comprising one or more amino acid additions, deletions, or substitutions compared with SEQ ID NOs: 721-1080 and 3111-3145. In some embodiments, the VH of the antigen-binding unit comprises a sequence selected from SEQ ID NOs: 721-1080 and 3111-3145. In some embodiments, the VH of the antigen-binding unit comprises a sequence comprising 10, 9, 8, 7, 6, 5, 4, 3, 2 or 1 amino acid additions, deletions, or substitutions compared with SEQ ID NOs: 721-1080 and 3111-3145. In some embodiments, the VH of the antigen-binding unit comprises a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145.

In some embodiments, the VL of the antigen-binding unit comprises a sequence selected from SEQ ID NOs: 1081-1440 and 3146-3180 or a sequence comprising one or more amino acid additions, deletions, or substitutions compared with SEQ ID NOs: 1081-1440 and 3146-3180. In some embodiments, the VL of the antigen-binding unit comprises a sequence selected from SEQ ID NOs: 1081-1440 and 3146-3180. In some embodiments, the VL of the antigen-binding unit comprises a sequence comprising 10, 9, 8, 7, 6, 5, 4, 3, 2 or 1 amino acid additions, deletions, or substitutions compared with SEQ ID NOs: 1081-1440 and 3146-3180. In some embodiments, the VL of the antigen-binding unit comprises a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180.

In some embodiments, the antigen-binding unit binds to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

In some embodiments, the antigen-binding unit neutralizes the novel coronavirus (SARS-CoV-2) with an IC50 of less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, or less than 0.001 μg/ml.

In another aspect, provided herein is an antigen-binding unit comprising a heavy chain variable region (VH) and a light chain variable region (VL), wherein the VH comprises a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145, and/or wherein the VL comprises a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180.

In some embodiments, the antigen-binding unit binds to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

In some embodiments, the antigen-binding unit neutralizes the novel coronavirus (SARS-CoV-2) with an IC50 of less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, or less than 0.001 μg/ml.

In some embodiments, the antigen-binding unit further comprises a heavy chain constant region (CH). In some embodiments, the CH of the antigen-binding unit comprises a sequence of SEQ ID NO: 1457 or a sequence comprising one or more amino acid additions, deletions, or substitutions compared with SEQ ID NO: 1457. In some embodiments, the CH of the antigen-binding unit comprises a sequence selected from SEQ ID NO: 1457. In some embodiments, the CH of the antigen-binding unit comprises a sequence comprising 10, 9, 8, 7, 6, 5, 4, 3, 2 or 1 amino acid additions, deletions, or substitutions compared with SEQ ID NO: 1457. In some embodiments, the CH of the antigen-binding unit comprises a sequence having at least 80%, 85%, 90%, 91%, 92%, 93%, 94%, 95%, 96%, 97%, 98%, 99% or 99% identity to a sequence selected from SEQ ID NO: 1457.

In some embodiments, the antigen-binding unit further comprises a light chain constant region (CL). In some embodiments, the CL of the antigen-binding unit comprises a sequence of SEQ ID NO: 1458 or a sequence comprising one or more amino acid additions, deletions, or substitutions compared with SEQ ID NO: 1458. In some embodiments, the CL of the antigen-binding unit comprises a sequence selected from SEQ ID NO: 1458. In some embodiments, the CL of the antigen-binding unit comprises a sequence comprising 10, 9, 8, 7, 6, 5, 4, 3, 2 or 1 amino acid additions, deletions, or substitutions compared with SEQ ID NO: 1458. In some embodiments, the CL of the antigen-binding unit comprises a sequence having at least 80%, 85%, 90%, 91%, 92%, 93%, 94%, 95%, 96%, 97%, 98%, 99% or 99% identity to a sequence selected from SEQ ID NO: 1458.

In another aspect, provided herein is an isolated nucleic acid molecule encoding the antigen-binding unit of the present invention as defined above.

In another aspect, provided herein is a vector, comprising the isolated nucleic acid molecule as defined above. The vector of the present invention can be a cloning vector and can also be an expression vector. In some embodiments, the vector of the present invention is for example, a plasmid, a cosmid, a phage or the like.

In another aspect, further provided is a host cell comprising the isolated nucleic acid molecule or the vector of the present invention. Such host cells include, but are not limited to, a prokaryotic cell, for example an Escherichia coli cell, and a eukaryotic cell such as a yeast cell, an insect cell, a plant cell, and an animal cell (such as, a mammal cell, e.g., a mouse cell, a human cell, etc.). The cell of the present invention can also be a cell line, for example, an HEK293 cell.

In another aspect, further provided is a method for preparing the antigen-binding unit of the present invention, comprising culturing the host cell of the present invention under suitable conditions, and recovering the antigen-binding unit of the present invention from a cell culture.

In another aspect, provided herein is a composition, comprising the antigen-binding unit, the isolated nucleic acid molecule, the vector or the host cell as described above.

In another aspect, provided herein is a kit comprising the antigen-binding unit of the present invention. In some embodiments, the antigen-binding unit of the present invention further comprises a detectable label. In some embodiments, the kit further comprises a second antibody, which specifically recognizes the antigen-binding unit of the present invention. Preferably, the second antibody further comprises a detectable label. Such detectable labels are well known to a person skilled in the art and include, but are not limited to, a radioisotope, a fluorescent material, a luminescent material, a colored material, an enzyme (e.g., horseradish peroxidase), etc.

In another aspect, provided herein is a method for detecting presence of a novel coronavirus, an S protein thereof or a RBD of the S protein, or a level thereof in a sample, comprising using the antigen-binding unit of the present invention. In some embodiments, the antigen-binding unit of the present invention further comprises a detectable label. In another preferred embodiment, the method further comprises detecting the antigen-binding unit of the present invention by using a second antibody carrying a detectable label. The method can be used for a diagnostic purpose (for example, the sample is a sample from a patient), or for a non-diagnostic purpose (for example, the sample is a cell sample rather than a sample from a patient).

In another aspect, provided herein is a method for diagnosing whether a subject is infected with a novel coronavirus, comprising: using the antigen-binding unit of the present invention to detect presence of a novel coronavirus, or an S protein thereof or a RBD of the S protein in a sample from the subject. In some embodiments, the antigen-binding unit of the present invention further comprises a detectable label. In another preferred embodiment, the method further comprises detecting the antigen-binding unit of the present invention by using a second antibody carrying a detectable label.

In another aspect, provided is the use of the antigen-binding unit of the present invention in the preparation of a kit, wherein the kit is used for detecting presence of a novel coronavirus, an S protein thereof or a RBD of the S protein, or a level thereof in a sample, or for diagnosing whether a subject is infected with the novel coronavirus.

In another aspect, provided herein is a pharmaceutical composition, comprising the antigen-binding unit of the present invention, and a pharmaceutically acceptable carrier and/or excipient.

In another aspect, provided herein is a method for neutralizing virulence of a novel coronavirus in a sample, comprising contacting the sample comprising the novel coronavirus with the antigen-binding unit of the present invention. Such methods can be used for therapeutic purposes, or for non-therapeutic purposes (for example, the sample is a cell sample, rather than a sample of or from a patient).

In another aspect, provided is the use of the antigen-binding unit of the present invention for preparing a drug, wherein the drug is used for neutralizing virulence of a novel coronavirus in a sample. In another aspect, provided herein is the antigen-binding unit as described above for neutralizing virulence of a novel coronavirus in a sample.

In another aspect, provided is the use of the antigen-binding unit of the present invention in the preparation of a pharmaceutical composition, wherein the pharmaceutical composition is used for preventing or treating novel coronavirus infections or diseases related to the novel coronavirus infections (e.g., novel coronavirus pneumonia) of a subject. In another aspect, provided herein is the antigen-binding unit as described above, for preventing and treating novel coronavirus infections or diseases related to the novel coronavirus infections (e.g., novel coronavirus pneumonia) of a subject.

In another aspect, provided herein is a method for preventing and treating novel coronavirus infections or diseases related to the novel coronavirus infections (e.g., novel coronavirus pneumonia) of a subject, comprising administering to a subject in need thereof a prophylactically or therapeutically effective amount of the antigen-binding unit of the present invention, or the pharmaceutical composition of the present invention.

In some embodiments, the subject is a mammal, for example human.

The antigen-binding unit of the present invention, or the pharmaceutical composition of the present invention can be administered to a subject by any suitable route of administration. Such routes of administration include, but are not limited to, oral, buccal, sublingual, topical, parenteral, rectal, intravaginal, or nasal routes.

The drug and pharmaceutical composition provided in the present invention can be used alone or in combination, or can be used in combination with other pharmacologically active agents (e.g., an antiviral drug, such as favipiravir, remdesivir and interferon). In some embodiments, the pharmaceutical composition also contains a pharmaceutically acceptable carrier and/or excipient.

In another aspect, provided herein is a conjugate comprising the antigen-binding unit as described above, wherein the antigen-binding unit is conjugated to a chemically functional moiety. In some embodiments, the chemically functional moiety is selected from a radioisotope, an enzyme, a fluorescent compound, a chemiluminescent compound, a bioluminescent compound, a substrate, a cofactor and an inhibitor.

### BRIEF DESCRIPTION OF THE DRAWINGS

FIGS. 1A-1C exemplarily show SDS-PAGE detection results of antigen-binding units ABU-174, ABU-175 and ABU190.

FIGS. 2A-2E exemplarily show measurement results regarding the affinity of antigen-binding units ABU-174 (A), ABU-175 (B), ABU190 (C), ABU297 (D) and ABU367 (E) for the S protein by using SPR technology.

FIGS. 3A-3C exemplarily show measurement results regarding the neutralizing inhibitory activity of antigen-binding units ABU-174 (A), ABU-175 (B) and ABU190 (C) against SARS-CoV-2 pseudovirus.

FIG. 4 exemplarily shows CPE measurement results regarding the neutralizing inhibitory activity of ABU-175 antibody against SARS-CoV-2 euvirus.

FIG. 5 exemplarily shows PRNT measurement results of the neutralizing inhibitory activity of antigen-binding units ABU-174, ABU-175 and ABU190 against SARS-CoV-2 euvirus.

### DETAILED DESCRIPTION OF EMBODIMENTS

While preferred embodiments of the present invention have been shown and described herein, it would have been obvious to a person skilled in the art that such embodiments are provided by way of example only. Numerous variations, changes, and substitutions will now occur to a person skilled in the art without departing from the present invention. It should be understood that various alternatives to the embodiments of the present invention described herein may be employed during practicing the processes described herein. It is intended that the following claims define the scope of the present invention so as to encompass methods and structures within the scope of these claims, and equivalents thereof.

When a numerical range is provided, it should be understood that each intervening value between the upper and lower limits of that range (accurate to the tenth of the unit of the lower limit, unless the context clearly dictates otherwise) and any other stated or intervening values within the stated range are encompassed within the present invention. The upper and lower limits of these smaller ranges may be independently included in the smaller ranges, and are also encompassed within the present invention, except for any specifically excluded limit within the stated range. Where the stated range encompasses one or both limits, ranges excluding either or both of those limits included therein are also encompassed within the present invention.

As used herein, the terms “polypeptide”, “peptide” and “protein” are used interchangeably herein to refer to polymers of amino acids of any length. The polymers can be linear, cyclic or branched, can comprise modified amino acids, and can be interrupted by non-amino acids. The terms also include an amino acid polymer that has been modified; for example, by sulfation, glycosylation, lipidation, acetylation, phosphorylation, iodination, methylation, oxidation, proteolytic processing, phosphorylation, prenylation, racemization, selenylation, transfer RNA-mediated addition of an amino acid to a protein (e.g., arginylation), ubiquitination, or any other manipulation, such as conjugation to a labeled component. As used herein, the term “amino acid” refers to natural and/or non-natural or synthetic amino acids, including glycine and a D or L optical isomer, as well as an amino acid analog and a peptidomimetic. A polypeptide or amino acid sequence “derived from” an specified protein refers to the origin of the polypeptide. Preferably, the polypeptide has an amino acid sequence that is substantially identical to the amino acid sequence of the polypeptide encoded in a sequence, or a portion thereof, wherein the portion consists of at least 10-20 amino acids or at least 20-30 amino acids or at least 30-50 amino acids, or can be identified immunologically with the polypeptide encoded in the sequence. The term also includes a polypeptide expressed by a specified nucleic acid sequence. As used herein, the term “domain” refers to a portion of a protein that is physically or functionally distinct from other portions of the protein or peptide. A physically defined domain includes an amino acid sequence which is extremely hydrophobic or hydrophilic, such as those membrane or cytoplasm-bound sequences. A domain can also be defined by internal homology that results, for example, from gene duplication. Functionally defined domains have distinct biological functions. For example, an antigen-binding domain refers to the portion of an antigen-binding unit or antibody that binds to an antigen. A functionally defined domain does not need to be encoded by a contiguous amino acid sequence, and a functionally defined domain can contain one or more physically defined domains.

As used herein, the term “amino acid” refers to natural and/or non-natural or synthetic amino acids, including but not limited to a D or L optical isomer, as well as an amino acid analog and a peptidomimetic. Standard one-letter or three-letter code is used to designate an amino acid. In the present invention, an amino acid is generally represented by one-letter and three-letter abbreviations well known in the art. For example, alanine can be represented by A or Ala.

As used herein, the term “antibody” refers to an immunoglobulin molecule generally consisting of two pairs of polypeptide chains, wherein each pair has one “light” (L) chain and one “heavy” (H) chain. Light chains of an antibody can be classified as a κ light chain and a λ light chain. Heavy chains can be classified as μ, δ, γ, α, and ε, and the isotypes of an antibody are defined as IgM, IgD, IgG, IgA, and IgE, respectively. In light and heavy chains, variable regions and constant regions are connected by a “J” region having about 12 or more amino acids, and a heavy chain also contains a “D” region having about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain CL. The constant region of the antibody can mediate the binding of the immunoglobulin to a host tissue or factor, comprising various cells (e.g., effector cells) of the immune system and the first component of the classical complement system (C1q). VH and VL regions can also be subdivided into regions with high variability (called complementarity determining regions (CDRs)), which are interspersed with more conserved regions called framework regions (FRs). Each VH and VL consists of three CDRs and four FRs arranged in the following order: FR1, CDR1, FR2, CDR2, FR3, CDR3, and FR4 from amino terminus to carboxy terminus. The variable regions of each heavy/light chain pair (VH and VL) form an antibody binding site, respectively. Distribution of amino acids in various regions or domains follows the definitions in: Kabat Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883, or IMGT (ImMunoGenTics) (Lefranc, M.-P., The Immunologist, 7, 132-136 (1999); Lefranc, M.-P. et al., Dev. Comp. Immunol., 27, 55-77 (2003)). Unless indicated otherwise, the CDRs in the VH and VL of the antibody in the present application are defined on the basis of the IMGT numbering system. According to the Kabat numbering system, the CDR amino acid residues in VH are numbered 31-35 (CDR1), 50-65 (CDR2) and 95-102 (CDR3); and the CDR amino acid residues in VL are numbered 24-34 (CDR1), 50-56 (CDR2) and 89-97 (CDR3). According to Chothia, the CDR amino acids in VH are numbered 26-32 (CDR1), 52-56 (CDR2) and 95-102 (CDR3); and the amino acid residues in VL are numbered 24-34 (CDR1), 50-56 (CDR2) and 89-97 (CDR3). According to the IMGT numbering system, the CDR amino acid residues in VH are numbered approximately 26-33 (CDR1), 51-56 (CDR2) and 93-102 (CDR3); and the CDR amino acid residues in VL are numbered approximately 27-32 (CDR1), 50-51 (CDR2) and 89-97 (CDR3) (as disclosed in https://www.novoprolabs.com/tools/cdr).

The term “antibody” is not limited by any particular method for producing an antibody. For example, the antibody comprises a recombinant antibody, a monoclonal antibody and a polyclonal antibody. The antibody can be antibodies of different isotypes, for example, an IgG (e.g., an IgG1, IgG2, IgG3 or IgG4 subtype), IgA1, IgA2, IgD, IgE or IgM antibody.

As used herein, the term “antigen-binding fragment” of an antibody refers to a polypeptide comprising a fragment of a full-length antibody that retains the ability to specifically bind to the same antigen to which the full-length antibody binds and/or competes with the full-length antibody for specific binding to the antigen, which is also referred to as an “antigen-binding moiety”. See generally, Fundamental Immunology, Ch. 7 Paul, W., ed., 2nd Edition, Raven Press, N.Y. (1989), which is incorporated herein by reference in its entirety for all purposes. An antigen-binding fragment of an antibody can be generated by recombinant DNA techniques or by enzymatic or chemical cleavage of an intact antibody. In some cases, an antigen-binding fragment comprises Fab, Fab′, F(ab′)₂, Fd, Fv, dAb and a complementarity determining region (CDR) fragment, a single chain antibody (e.g., scFv), a chimeric antibody, a diabody and a polypeptide comprising at least a portion of an antibody sufficient to confer a specific antigen-binding ability to the polypeptide. In some cases, an antigen-binding fragment of an antibody is a single chain antibody (e.g., scFv), wherein VL and VH domains are paired by a linker which enables them to be produced as a single polypeptide chain, thereby forming a monovalent molecule (see, e.g., Bird et al., Science 242:423 426 (1988) and Huston et al., Proc. Natl. Acad. Sci. USA 85:5879 5883 (1988)). Such scFv molecules can have a general structure of NH2-VL-linker-VH—COOH or NH2-VH-linker-VL-COOH. Suitable linkers in the prior art consist of a repeated GGGGS amino acid sequence or a variant thereof. For example, a linker having an amino acid sequence (GGGGS)₄ can be used, and a variant thereof can also be used (Holliger et al. (1993), Proc. Natl. Acad. Sci. USA 90: 6444-6448). Other linkers which can be used in the present invention are described in Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol.

In some cases, an antigen-binding fragment of an antibody is a diabody, i.e., a bivalent antibody, wherein VH and VL domains are expressed on a single polypeptide chain; however, the linker used is too short to allow pairing between the two domains of the same chain, thereby forcing the domain to pair with the complementary domains of another chain and producing two antigen-binding sites (see, e.g., Holliger P. et al., Proc. Natl. Acad. Sci. USA 90:6444 6448 (1993), and Poljak R. J. et al., Structure 2:1121 1123 (1994)).

An antigen-binding fragment of an antibody (e.g., the above-mentioned antibody fragment) can be obtained from a given antibody (e.g., the antibody provided in the present invention) by using conventional techniques known to a person skilled in the art (e.g., recombinant DNA techniques or enzymatic or chemical cleavage) and the antigen-binding fragment of the antibody can be screened for specificity in the same manner as for an intact antibody.

Unless the context clearly dictates, the term “antibody” when referred to herein comprises not only an intact antibody but also an antigen-binding fragment of an antibody.

Unless the context clearly dictates, the term “antigen-binding unit” herein includes the antibody and the antigen-binding fragment thereof as defined above.

As used herein, the term “monoclonal antibody” refers to an antibody or a fragment of an antibody from a population of highly homologous antibody molecules, i.e., a population of identical antibody molecules, except for possible naturally occurring mutations. The monoclonal antibody is highly specific for a single epitope on an antigen. Relative to a monoclonal antibody, a polyclonal antibody generally comprises at least 2 or more different antibodies, and these different antibodies generally recognize different epitopes on an antigen. A monoclonal antibody can usually be obtained by using the hybridoma technique first reported by Kohler et al. (Nature, 256:495, 1975), and can also be obtained by using recombinant DNA techniques (for example, see Journal of virological methods, 2009, 158(1-2): 171-179).

As used herein, a “neutralizing antibody” refers to an antibody or antibody fragment that can clear or significantly reduce virulence (e.g., ability to infect cells) of a target virus.

As used herein, in the case of a polypeptide, a “sequence” is the order of amino acids in the polypeptide that are arranged in the direction from the amino terminus to the carboxy terminus, wherein residues adjacent to each other in the sequence are contiguous in the primary structure of the polypeptide. The sequence can also be a linear sequence of a portion of a polypeptide known to contain additional residues in one or both directions.

As used herein, “identity”, “homology” or “sequence identity” refers to the sequence similarity or interchangeability between two or more polynucleotide sequences or between two or more polypeptide sequences. When a program, such as Emboss Needle or BestFit is used to determine sequence identity, similarity or homology between two different amino acid sequences, a default setting can be used, or an appropriate scoring matrix, such as blosum45 or blosum80, can be selected to optimize the score of identity, similarity or homology. Preferably, homologous polynucleotides are those polynucleotides that hybridize under stringent conditions as defined herein and have at least 70%, preferably at least 80%, more preferably at least 90%, more preferably 95%, more preferably 97%, more preferably 98% and even more preferably 99% sequence identity to these sequences. When sequences of comparable lengths are optimally aligned, the homologous polypeptide preferably has at least 80%, or at least 90%, or at least 95%, or at least 97%, or at least 98% sequence identity, or at least 99% sequence identity.

With respect to the antigen-binding units determined herein, “percent sequence identity (%)” is defined as the percentage of amino acid residues in the query sequence that are identical to amino acid residues of the second, reference polypeptide sequence or a portion thereof, after aligning the sequences and introducing gaps, if necessary, to achieve maximum percentage of sequence identity, and not considering any conservative replacements as a part of sequence identity. The alignment aimed at determining the percent amino acid sequence identity can be achieved in various ways within the skill in the art, for example, by using a publicly available computer software, such as BLAST, BLAST-2, ALIGN, NEEDLE or Megalign (DNASTAR) software. A person skilled in the art can determine appropriate parameters for measuring the alignment, including any algorithm needed to achieve the maximal alignment over the full length of the sequences being compared. The percent identity may be measured over the length of the entire defined polypeptide sequence, or may be measured over a shorter length, for example, over the length of a fragment taken from a larger, defined polypeptide sequence, such as a fragment of at least 5, at least 10, at least 15, at least 20, at least 50, at least 100 or at least 200 contiguous residues. These lengths are exemplary only, and it should be understood that any fragment length supported by the sequences shown in the Tables, Figures or Sequence Listing of the present invention can be used to describe the length over which percent identity can be measured.

The antigen-binding unit described herein may have one or more modifications relative to a reference sequence. The modifications may be deletions, insertions or additions, or substitutions or replacements of amino acid residues. “Deletion” refers to a change in an amino acid sequence due to the lack of one or more amino acid residues. “Insertion” or “addition” refers to a change in an amino acid sequence due to the addition of one or more amino acid residues compared with a reference sequence. “Substitution” or “replacement” refers to that one or more amino acids are substituted with different amino acids. In the present invention, mutations of the antigen-binding unit relative to the reference sequence can be determined by comparing the antigen-binding unit with the reference sequence. Optimal alignment of sequences for comparison can be performed according to any method known in the art.

As used herein, the term “antigen” refers to a substance that is recognized and specifically bound by an antigen-binding unit. An antigen can include a peptide, a protein, a glycoprotein, a polysaccharide, and a lipid; a portion thereof, and a combination thereof. Non-limiting exemplary antigens include a protein from a coronavirus such as SARS-CoV-2, and other homologs thereof.

As used herein, the term “isolated” refers to being isolated from cellular and other ingredients with which polynucleotides, peptides, polypeptides, proteins, antibodies or fragments thereof are associated under normal circumstances in nature. It is known to a person skilled in the art that a non-naturally occurring polynucleotide, peptide, polypeptide, protein, antibody or a fragment thereof does not need to be “isolated” to distinguish same from a naturally occurring counterpart thereof. In addition, the “concentrated”, “isolated” or “diluted” polynucleotide, peptide, polypeptide, protein, antibody, or the fragment thereof is distinguishable from the naturally occurring counterpart thereof, because the concentration or number of molecules per unit volume is greater than (“concentrated”) or less than the naturally occurring counterpart thereof (“isolated”). Enrichment may be measured on the basis of an absolute amount, such as the weight of a solution per unit volume, or same can be measured relative to a second, potentially interfering substance present in the source mixture.

The terms “polynucleotides”, “nucleic acids”, “nucleotides” and “oligonucleotides” are used interchangeably. They refer to polymerized nucleotides (deoxyribonucleotides or ribonucleotides) or analogs thereof of any length. A polynucleotide can have any three-dimensional structure and can perform any known or unknown function. The following are non-limiting examples of a polynucleotide: a coding region or a non-coding region of a gene or a gene fragment, a locus determined by linkage analysis, an exon, an intron, messenger RNA (mRNA), transfer RNA, ribosomal RNA, a ribozyme, cDNA, a recombinant polynucleotide, a branched polynucleotide, a plasmid, a vector, an isolated DNA of any sequence, an isolated RNA of any sequence, a nucleic acid probe, a primer, an oligonucleotide, or a synthetic DNA. A polynucleotide may contain a modified nucleotide, such as a methylated nucleotide, and a nucleotide analog. If present, a modification to a nucleotide structure can be implemented before or after the assembly of a polymer. The sequence of a nucleotide can be interrupted by non-nucleotide components. A polynucleotide can be further modified after polymerization, for example, by conjugation with a labeled component.

When used for a polynucleotide, “recombinant” means that the polynucleotide is a product of various combinations of cloning, restriction digestion and/or ligation steps, and other procedures that produce a construct different from the polynucleotide found in nature.

The term “gene” or “gene fragment” can be used interchangeably herein. They refer to polynucleotides containing at least one open reading frame capable of encoding a specific protein following transcription and translation. The gene or gene fragment may be genomic, cDNA, or synthetic, as long as the polynucleotide contains at least one open reading frame, which may cover the entire coding region or a segment thereof.

The term “operably linked” or “effectively linked” refers to the state of being juxtaposed in which the components so described are allowed to function in a intended manner. For example, if a promoter sequence promotes the transcription of a coding sequence, the promoter sequence is operably linked to the coding sequence.

As used herein, “expression” refers to the process by which polynucleotides are transcribed into mRNA, and/or the process by which the transcribed mRNA (also called “transcript”) is subsequently translated into peptides, polypeptides or proteins. The transcript and the encoded polypeptide are collectively referred to as the gene product. If the polynucleotide is derived from genomic DNA, the expression can include splicing of mRNA in an eukaryotic cell.

As used herein, the term “vector” refers to a nucleic acid delivery vehicle into which a polynucleotide can be inserted. When the vector allows for the expression of the protein encoded by the inserted polynucleotide, the vector is called an expression vector. A vector can be introduced into a host cell by transformation, transduction or transfection, and the genetic substance elements carried thereby can be expressed in the host cell. The vector is well known to a person skilled in the art, and includes but is not limited to: a plasmid; a phagemid; an artificial chromosome such as a yeast artificial chromosome (YAC), a bacterial artificial chromosome (BAC) or a P1-derived artificial chromosome (PAC); a phage such as a λ phage or an M13 phage, and an animal virus. The animal virus that can be used as a vector includes but is not limited to a retrovirus (comprising a lentivirus), an adenovirus, an adeno-associated virus, a herpes virus (e.g., a herpes simplex virus), a poxvirus, a baculovirus, a papilloma virus and a papovavirus (such as SV40). A vector can contain a variety of elements that control expression, including, but not limited to: a promoter sequence, a transcription initiation sequence, an enhancer sequence, a selection element, and a reporter gene. In addition, the vector also can contain a replication initiation site.

As used herein, the term “host cell” refers to a cell that can be used to introduce a vector, including but not limited to a prokaryotic cell such as Escherichia coli or Bacillus subtilis, a fungal cell such as a yeast cell or Aspergillus, an insect cell such as Drosophila S2 cell or Sf9, and an animal cell such as a fibroblast, a CHO cell, a COS cell, a NSO cell, an HeLa cell, a BHK cell, an HEK293 cell or a human cell.

As used herein, the term “biological sample” includes various types of samples obtained from an organism and can be used in a diagnostic or monitoring experiment. The term includes blood and other liquid samples derived from an organism, a solid tissue sample such as a biopsy specimen or tissue culture, or a cell derived therefrom and a progeny thereof. The term includes a sample that has been treated in any way following acquisition, such as by treatment with a reagent, dissolution, or enrichment of certain components. The term includes a clinical sample, and further includes cells in a cell culture, a cell supernatant, a cell lysate, serum, plasma, a biological fluid, and a tissue sample.

As used herein, the terms “recipient”, “individual”, “subject”, “host” and “patient” are used interchangeably herein and refer to any mammalian subject, particularly human, for whom diagnosis, treatment or treating is desired.

As used herein, the terms “treating”, “treatment”, etc. are used herein to generally refer to a process of obtaining a desired pharmacological and/or physiological effect. The effect may be prophylactic in terms of completely or partially preventing a disease or a symptom thereof, and/or may be therapeutic in terms of partially or completely stabilizing or curing a disease and/or adverse effects attributable to the disease. “Treating” as used herein encompasses any treatment of a disease in a mammal, such as a mouse, a rat, a rabbit, a pig, and a primate including human and other apes, particularly human, and the term includes: (a) preventing the occurrence of a disease or symptom in a subject who may be susceptible to the disease or symptom but has not yet been diagnosed; (b) inhibiting the symptom of the disease; (c) preventing the progression of the disease; (d) alleviating the symptom of the disease; (e) causing regression of the diseases or symptom; or any combination thereof. As used herein, the term “specifically binding” refers to a non-random binding reaction between two molecules, such as a reaction between an antibody and its corresponding antigen. In certain embodiments, an antibody specifically binding to an antigen (or an antibody specific for an antigen) refers to an antibody that binds to the antigen with an affinity (KD) less than about 10⁻⁵ M, for example less than about 10⁻⁶ M, 10⁻⁷ M, 10⁻⁸ M, 10⁻⁹ M or 10⁻¹⁰ M or less.

As used herein, the term “KD” refers to the dissociation equilibrium constant of a particular antibody-antigen interaction, which is used to describe the binding affinity between an antibody and an antigen. In the present invention, KD is defined as the ratio of two kinetic rate constants Ka/Kd, wherein “Ka” refers to the rate constant for the binding of an antibody to an antigen and “Kd” refers to the rate constant for the dissociation of the antibody from the antibody/antigen complex. The smaller the equilibrium dissociation constant KD, the tighter the antibody-antigen-binding and the higher the affinity between the antibody and the antigen. Generally, an antibody binds to an antigen with a dissociation equilibrium constant (KD) less than about 10⁻⁵ M. The property of the specific binding between two molecules can be determined using a method well known in the art, e.g. determined by surface plasmon resonance (SPR) in a BIACORE instrument.

As used herein, the term “neutralizing activity” refers to the functional activity of an antibody or antibody fragment binding to an antigen protein on a virus, thereby preventing viral infection of cells and/or maturation of viral progeny and/or release of viral progeny. The antibody or antibody fragment with a neutralizing activity can prevent the amplification of the virus, thereby inhibiting or eliminating virus infection. In some embodiments, the neutralizing activity is represented by the IC₅₀ of an antibody or an antibody fragment in term of viral inhibition. The “half-maximal inhibitory concentration” (IC₅₀) is a measure of a drug, such as an antibody, in terms of inhibiting biological or biochemical functions, etc., such as viral potency. The IC₅₀ herein is calculated by a Reed-Muench method according to the neutralization inhibition rate of the antigen-binding fragment against viral (e.g., pseudoviral or euviral) infection in a cell. Provided herein is an antigen-binding unit which can specifically recognize and target an S protein of a novel coronavirus, particularly a receptor binding domain (RBD) of the S protein, and shows an efficient ability to neutralize the virus. Therefore, the antigen-binding unit of the present invention is particularly suitable for diagnosing, preventing and treating novel coronavirus infections or diseases related to the novel coronavirus infections (e.g., novel coronavirus pneumonia).

### Antigen-Binding Unit

In one aspect, the antigen-binding unit of the present invention comprises a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3.

The VH of the antigen-binding unit of the present invention can comprise a sequence selected from SEQ ID NOs: 721-1080 and 3111-3145, a sequence comprising one or more amino acid additions, deletions, or substitutions compared with a sequence selected from SEQ ID NOs: 721-1080 and 3111-3145, or a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145. When the VH of the antigen-binding unit of the present invention has amino acid additions, deletions, or substitutions compared with the reference polypeptide sequence, the VH of the antigen-binding unit of the present invention can have 1, 2, 3, 4, 5, 6, 7, 8, 9, 10, 11, 12, 13, 14, 15, 16, 17, 18, 19 or 20 additions, deletions, or substitutions compared with the reference polypeptide. When the VH of the antigen-binding unit of the present invention has amino acid additions, deletions, or substitutions compared with the reference polypeptide sequence, the VH of the antigen-binding unit of the present invention can have more than 1, 2, 3, 4, 5, 6, 7, 8, 9, 10, 11, 12, 13, 14, 15, 16, 17, 18, 19 or 20 additions, deletions, or substitutions compared with the reference polypeptide. When the VH of the antigen-binding unit of the present invention has amino acid additions, deletions, or substitutions compared with the reference polypeptide sequence, the VH of the antigen-binding unit of the present invention can have less than 2, 3, 4, 5, 6, 7, 8, 9, 10, 11, 12, 13, 14, 15, 16, 17, 18, 19 or 20 additions, deletions, or substitutions compared with the reference polypeptide.

The VH CDR1 of the antigen-binding unit of the present invention can comprise a sequence selected from SEQ ID NOs: 1461-1820 and 2901-2935, a sequence comprising one or more amino acid additions, deletions, or substitutions compared with a sequence selected from SEQ ID NOs: 1461-1820 and 2901-2935, or a sequence having at least 80%, 85%, 90%, 91%, 92%, 93%, 94%, 95%, 96%, 97%, 98%, 99% or 99% identity to a sequence selected from SEQ ID NOs: 1461-1820 and 2901-2935. When the VH CDR1 of the antigen-binding unit of the present invention has amino acid additions, deletions, or substitutions compared with the reference polypeptide sequence, the VH CDR1 of the antigen-binding unit of the present invention can have 1, 2, 3, 4 or 5 additions, deletions, or substitutions compared with the reference polypeptide. When the VH CDR1 of the antigen-binding unit of the present invention has amino acid additions, deletions, or substitutions compared with the reference polypeptide sequence, the VH CDR1 of the antigen-binding unit of the present invention can have more than 1, 2, 3, 4, or 5 additions, deletions, or substitutions compared with the reference polypeptide. When the VH CDR1 of the antigen-binding unit of the present invention has amino acid additions, deletions, or substitutions compared with the reference polypeptide sequence, the VH CDR1 of the antigen-binding unit of the present invention can have less than 2, 3, 4, or 5 additions, deletions, or substitutions compared with the reference polypeptide.

The VH CDR2 of the antigen-binding unit of the present invention can comprise a sequence selected from SEQ ID NOs: 1821-2180 and 2936-2970, a sequence comprising one or more amino acid additions, deletions, or substitutions compared with a sequence selected from SEQ ID NOs: 1821-2180 and 2936-2970, or a sequence having at least 80%, 85%, 90%, 91%, 92%, 93%, 94%, 95%, 96%, 97%, 98%, 99% or 99% identity to a sequence selected from SEQ ID NOs: 1821-2180 and 2936-2970. When the VH CDR2 of the antigen-binding unit of the present invention has amino acid additions, deletions, or substitutions compared with the reference polypeptide sequence, the VH CDR2 of the antigen-binding unit of the present invention can have 1, 2, 3, 4 or 5 additions, deletions, or substitutions compared with the reference polypeptide. When the VH CDR2 of the antigen-binding unit of the present invention has amino acid additions, deletions, or substitutions compared with the reference polypeptide sequence, the VH CDR2 of the antigen-binding unit of the present invention can have more than 1, 2, 3, 4, or 5 additions, deletions, or substitutions compared with the reference polypeptide. When the VH CDR2 of the antigen-binding unit of the present invention has amino acid additions, deletions, or substitutions compared with the reference polypeptide sequence, the VH CDR2 of the antigen-binding unit of the present invention can have less than 2, 3, 4, or 5 additions, deletions, or substitutions compared with the reference polypeptide.

The VH CDR3 of the antigen-binding unit of the present invention can comprise a sequence selected from SEQ ID NOs: 1-360 and 2971-3005, a sequence comprising one or more amino acid additions, deletions, or substitutions compared with a sequence selected from SEQ ID NOs: 1-360 and 2971-3005, or a sequence having at least 80%, 85%, 90%, 91%, 92%, 93%, 94%, 95%, 96%, 97%, 98%, 99% or 99% identity to a sequence selected from SEQ ID NOs: 1-360 and 2971-3005. When the VH CDR3 of the antigen-binding unit of the present invention has amino acid additions, deletions, or substitutions compared with the reference polypeptide sequence, the VH CDR3 of the antigen-binding unit of the present invention can have 1, 2, 3, 4 or 5 additions, deletions, or substitutions compared with the reference polypeptide. When the VH CDR3 of the antigen-binding unit of the present invention has amino acid additions, deletions, or substitutions compared with the reference polypeptide sequence, the VH CDR3 of the antigen-binding unit of the present invention can have more than 1, 2, 3, 4, or 5 additions, deletions, or substitutions compared with the reference polypeptide. When the VH CDR2 of the antigen-binding unit of the present invention has amino acid additions, deletions, or substitutions compared with the reference polypeptide sequence, the VH CDR3 of the antigen-binding unit of the present invention can have less than 2, 3, 4, or 5 additions, deletions, or substitutions compared with the reference polypeptide.

The VL of the antigen-binding unit of the present invention can comprise a sequence selected from SEQ ID NOs: 1081-1440 and 3146-3180, a sequence comprising one or more amino acid additions, deletions, or substitutions compared with a sequence selected from SEQ ID NOs: 1081-1440 and 3146-3180, or a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180. When the VL of the antigen-binding unit of the present invention has amino acid additions, deletions, or substitutions compared with the reference polypeptide sequence, the VL of the antigen-binding unit of the present invention can have 1, 2, 3, 4, 5, 6, 7, 8, 9, 10, 11, 12, 13, 14, 15, 16, 17, 18, 19 or 20 additions, deletions, or substitutions compared with the reference polypeptide. When the VL of the antigen-binding unit of the present invention has amino acid additions, deletions, or substitutions compared with the reference polypeptide sequence, the VL of the antigen-binding unit of the present invention can have more than 1, 2, 3, 4, 5, 6, 7, 8, 9, 10, 11, 12, 13, 14, 15, 16, 17, 18, 19 or 20 amino acid additions, deletions, or substitutions compared with the reference polypeptide. When the VL of the antigen-binding unit of the present invention has amino acid additions, deletions, or substitutions compared with the reference polypeptide sequence, the VL of the antigen-binding unit of the present invention can have less than 2, 3, 4, 5, 6, 7, 8, 9, 10, 11, 12, 13, 14, 15, 16, 17, 18, 19 or 20 additions, deletions, or substitutions compared with the reference polypeptide.

The VL CDR1 of the antigen-binding unit of the present invention can comprise a sequence selected from SEQ ID NOs: 2181-2540 and 3006-3040, a sequence comprising one or more amino acid additions, deletions, or substitutions compared with a sequence selected from SEQ ID NOs: 2181-2540 and 3006-3040, or a sequence having at least 80%, 85%, 90%, 91%, 92%, 93%, 94%, 95%, 96%, 97%, 98%, 99% or 99% identity to a sequence selected from SEQ ID NOs: 2181-2540 and 3006-3040. When the VL CDR1 of the antigen-binding unit of the present invention has amino acid additions, deletions, or substitutions compared with the reference polypeptide sequence, the VL CDR1 of the antigen-binding unit of the present invention can have 1, 2, 3, 4 or 5 additions, deletions, or substitutions compared with the reference polypeptide. When the VL CDR1 of the antigen-binding unit of the present invention has amino acid additions, deletions, or substitutions compared with the reference polypeptide sequence, the VL CDR1 of the antigen-binding unit of the present invention can have more than 1, 2, 3, 4, or 5 additions, deletions, or substitutions compared with the reference polypeptide. When the VL CDR1 of the antigen-binding unit of the present invention has amino acid additions, deletions, or substitutions compared with the reference polypeptide sequence, the VL CDR1 of the antigen-binding unit of the present invention can have less than 2, 3, 4, or 5 additions, deletions, or substitutions compared with the reference polypeptide.

The VL CDR2 of the antigen-binding unit of the present invention can comprise a sequence selected from SEQ ID NOs: 2541-2900 and 3041-3075, a sequence comprising one or more amino acid additions, deletions, or substitutions compared with a sequence selected from SEQ ID NOs: 2541-2900 and 3041-3075, or a sequence having at least 80%, 85%, 90%, 91%, 92%, 93%, 94%, 95%, 96%, 97%, 98%, 99% or 99% identity to a sequence selected from SEQ ID NOs: 2541-2900 and 3041-3075. When the VL CDR2 of the antigen-binding unit of the present invention has amino acid additions, deletions, or substitutions compared with the reference polypeptide sequence, the VL CDR2 of the antigen-binding unit of the present invention can have 1, 2, 3, 4 or 5 additions, deletions, or substitutions compared with the reference polypeptide. When the VL CDR2 of the antigen-binding unit of the present invention has amino acid additions, deletions, or substitutions compared with the reference polypeptide sequence, the VL CDR2 of the antigen-binding unit of the present invention can have more than 1, 2, 3, 4, or 5 additions, deletions, or substitutions compared with the reference polypeptide. When the VL CDR2 of the antigen-binding unit of the present invention has amino acid additions, deletions, or substitutions compared with the reference polypeptide sequence, the VL CDR2 of the antigen-binding unit of the present invention can have less than 2, 3, 4, or 5 additions, deletions, or substitutions compared with the reference polypeptide.

The VL CDR3 of the antigen-binding unit of the present invention can comprise a sequence selected from SEQ ID NOs: 361-720 and 3076-3110, a sequence comprising one or more amino acid additions, deletions, or substitutions compared with a sequence selected from SEQ ID NOs: 361-720 and 3076-3110, or a sequence having at least 80%, 85%, 90%, 91%, 92%, 93%, 94%, 95%, 96%, 97%, 98%, 99% or 99% identity to a sequence selected from SEQ ID NOs: 361-720 and 3076-3110. When the VL CDR3 of the antigen-binding unit of the present invention has amino acid additions, deletions, or substitutions compared with the reference polypeptide sequence, the VL CDR3 of the antigen-binding unit of the present invention can have 1, 2, 3, 4 or 5 additions, deletions, or substitutions compared with the reference polypeptide. When the VL CDR3 of the antigen-binding unit of the present invention has amino acid additions, deletions, or substitutions compared with the reference polypeptide sequence, the VL CDR3 of the antigen-binding unit of the present invention can have more than 1, 2, 3, 4, or 5 additions, deletions, or substitutions compared with the reference polypeptide. When the VL CDR3 of the antigen-binding unit of the present invention has amino acid additions, deletions, or substitutions compared with the reference polypeptide sequence, the VL CDR3 of the antigen-binding unit of the present invention can have less than 2, 3, 4, or 5 additions, deletions, or substitutions compared with the reference polypeptide.

The VH CDR1 of the antigen-binding unit of the present invention can comprise a sequence selected from SEQ ID NOs: 1461-1820 and 2901-2935, a sequence comprising one or more amino acid additions, deletions, or substitutions compared with a sequence selected from SEQ ID NOs: 1461-1820 and 2901-2935, or a sequence having at least 80%, 85%, 90%, 91%, 92%, 93%, 94%, 95%, 96%, 97%, 98%, 99% or 99% identity to a sequence selected from SEQ ID NOs: 1461-1820 and 2901-2935; and the VL CDR1 of the antigen-binding unit of the present invention can comprise a sequence selected from SEQ ID NOs: 2181-2540 and 3006-3040, a sequence comprising one or more amino acid additions, deletions, or substitutions compared with a sequence selected from SEQ ID NOs: 2181-2540 and 3006-3040, or a sequence having at least 80%, 85%, 90%, 91%, 92%, 93%, 94%, 95%, 96%, 97%, 98%, 99% or 99% identity to a sequence selected from SEQ ID NOs: 2181-2540 and 3006-3040.

The VH CDR2 of the antigen-binding unit of the present invention can comprise a sequence selected from SEQ ID NOs: 1821-2180 and 2936-2970, a sequence comprising one or more amino acid additions, deletions, or substitutions compared with a sequence selected from SEQ ID NOs: 1821-2180 and 2936-2970, or a sequence having at least 80%, 85%, 90%, 91%, 92%, 93%, 94%, 95%, 96%, 97%, 98%, 99% or 99% identity to a sequence selected from SEQ ID NOs: 1821-2180 and 2936-2970; and the VL CDR2 of the antigen-binding unit of the present invention can comprise a sequence selected from SEQ ID NOs: 2541-2900 and 3041-3075, a sequence comprising one or more amino acid additions, deletions, or substitutions compared with a sequence selected from SEQ ID NOs: 2541-2900 and 3041-3075, or a sequence having at least 80%, 85%, 90%, 91%, 92%, 93%, 94%, 95%, 96%, 97%, 98%, 99% or 99% identity to a sequence selected from SEQ ID NOs: 2541-2900 and 3041-3075.

The VH CDR3 of the antigen-binding unit of the present invention can comprise a sequence selected from SEQ ID NOs: 1-360 and 2971-3005, a sequence comprising one or more amino acid additions, deletions, or substitutions compared with a sequence selected from SEQ ID NOs: 1-360 and 2971-3005, or a sequence having at least 80%, 85%, 90%, 91%, 92%, 93%, 94%, 95%, 96%, 97%, 98%, 99% or 99% identity to a sequence selected from SEQ ID NOs: 1-360 and 2971-3005; and the VL CDR3 of the antigen-binding unit of the present invention can comprise a sequence selected from SEQ ID NOs: 361-720 and 3076-3110, a sequence comprising one or more amino acid additions, deletions, or substitutions compared with a sequence selected from SEQ ID NOs: 361-720 and 3076-3110, or a sequence having at least 80%, 85%, 90%, 91%, 92%, 93%, 94%, 95%, 96%, 97%, 98%, 99% or 99% identity to a sequence selected from SEQ ID NOs: 361-720 and 3076-3110.

The VH of the antigen-binding unit of the present invention can comprise VH CDR1, VH CDR2 and VH CDR3, wherein the VH CDR1 is a sequence selected from SEQ ID NOs: 1461-1820 and 2901-2935, a sequence comprising one or more amino acid additions, deletions, or substitutions compared with a sequence selected from SEQ ID NOs: 1461-1820 and 2901-2935, or a sequence having at least 80%, 85%, 90%, 91%, 92%, 93%, 94%, 95%, 96%, 97%, 98%, 99% or 99% identity to a sequence selected from SEQ ID NOs: 1461-1820 and 2901-2935; wherein the VH CDR2 is a sequence selected from SEQ ID NOs: 1821-2180 and 2936-2970, a sequence comprising one or more amino acid additions, deletions, or substitutions compared with a sequence selected from SEQ ID NOs: 1821-2180 and 2936-2970, or a sequence having at least 80%, 85%, 90%, 91%, 92%, 93%, 94%, 95%, 96%, 97%, 98%, 99% or 99% identity to a sequence selected from SEQ ID NOs: 1821-2180 and 2936-2970; and wherein the VH CDR3 is a sequence selected from SEQ ID NOs: 1-360 and 2971-3005, a sequence comprising one or more amino acid additions, deletions, or substitutions compared with a sequence selected from SEQ ID NOs: 1-360 and 2971-3005, or a sequence having at least 80%, 85%, 90%, 91%, 92%, 93%, 94%, 95%, 96%, 97%, 98%, 99% or 99% identity to a sequence selected from SEQ ID NOs: 1-360 and 2971-3005.

The VL of the antigen-binding unit of the present invention can comprise VL CDR1, VL CDR2 and VL CDR3, wherein the VL CDR1 is a sequence selected from SEQ ID NOs: 2181-2540 and 3006-3040, a sequence comprising one or more amino acid additions, deletions, or substitutions compared with a sequence selected from SEQ ID NOs: 2181-2540 and 3006-3040, or a sequence having at least 80%, 85%, 90%, 91%, 92%, 93%, 94%, 95%, 96%, 97%, 98%, 99% or 99% identity to a sequence selected from SEQ ID NOs: 2181-2540 and 3006-3040; wherein the VL CDR2 is a sequence selected from SEQ ID NOs: 2541-2900 and 3041-3075, a sequence comprising one or more amino acid additions, deletions, or substitutions compared with a sequence selected from SEQ ID NOs: 2541-2900 and 3041-3075, or a sequence having at least 80%, 85%, 90%, 91%, 92%, 93%, 94%, 95%, 96%, 97%, 98%, 99% or 99% identity to a sequence selected from SEQ ID NOs: 2541-2900 and 3041-3075; and wherein the VL CDR3 is a sequence selected from SEQ ID NOs: 361-720 and 3076-3110, a sequence comprising one or more amino acid additions, deletions, or substitutions compared with a sequence selected from SEQ ID NOs: 361-720 and 3076-3110, or a sequence having at least 80%, 85%, 90%, 91%, 92%, 93%, 94%, 95%, 96%, 97%, 98%, 99% or 99% identity to a sequence selected from SEQ ID NOs: 361-720 and 3076-3110.

The VH of the antigen-binding unit described herein can comprise a sequence selected from combinations of CDR1, CDR2, and CDR3 as following:

| HCDR1 | HCDR2 | HCDR3 | ABU No. | HCDR1 | HCDR2 | HCDR3 | ABU No. | HCDR1 | HCDR2 | HCDR3 | ABU No. |
|---|---|---|---|---|---|---|---|---|---|---|---|
| 1461 | 1821 | 1 | ABU-1 | 1581 | 1941 | 121 | ABU-121 | 1701 | 2061 | 241 | ABU-241 |
| 1462 | 1822 | 2 | ABU-2 | 1582 | 1942 | 122 | ABU-122 | 1702 | 2062 | 242 | ABU-242 |
| 1463 | 1823 | 3 | ABU-3 | 1583 | 1943 | 123 | ABU-123 | 1703 | 2063 | 243 | ABU-243 |
| 1464 | 1824 | 4 | ABU-4 | 1584 | 1944 | 124 | ABU-124 | 1704 | 2064 | 244 | ABU-244 |
| 1465 | 1825 | 5 | ABU-5 | 1585 | 1945 | 125 | ABU-125 | 1705 | 2065 | 245 | ABU-245 |
| 1466 | 1826 | 6 | ABU-6 | 1586 | 1946 | 126 | ABU-126 | 1706 | 2066 | 246 | ABU-246 |
| 1467 | 1827 | 7 | ABU-7 | 1587 | 1947 | 127 | ABU-127 | 1707 | 2067 | 247 | ABU-247 |
| 1468 | 1828 | 8 | ABU-8 | 1588 | 1948 | 128 | ABU-128 | 1708 | 2068 | 248 | ABU-248 |
| 1469 | 1829 | 9 | ABU-9 | 1589 | 1949 | 129 | ABU-129 | 1709 | 2069 | 249 | ABU-249 |
| 1470 | 1830 | 10 | ABU-10 | 1590 | 1950 | 130 | ABU-130 | 1710 | 2070 | 250 | ABU-250 |
| 1471 | 1831 | 11 | ABU-11 | 1591 | 1951 | 131 | ABU-131 | 1711 | 2071 | 251 | ABU-251 |
| 1472 | 1832 | 12 | ABU-12 | 1592 | 1952 | 132 | ABU-132 | 1712 | 2072 | 252 | ABU-252 |
| 1473 | 1833 | 13 | ABU-13 | 1593 | 1953 | 133 | ABU-133 | 1713 | 2073 | 253 | ABU-253 |
| 1474 | 1834 | 14 | ABU-14 | 1594 | 1954 | 134 | ABU-134 | 1714 | 2074 | 254 | ABU-254 |
| 1475 | 1835 | 15 | ABU-15 | 1595 | 1955 | 135 | ABU-135 | 1715 | 2075 | 255 | ABU-255 |
| 1476 | 1836 | 16 | ABU-16 | 1596 | 1956 | 136 | ABU-136 | 1716 | 2076 | 256 | ABU-256 |
| 1477 | 1837 | 17 | ABU-17 | 1597 | 1957 | 137 | ABU-137 | 1717 | 2077 | 257 | ABU-257 |
| 1478 | 1838 | 18 | ABU-18 | 1598 | 1958 | 138 | ABU-138 | 1718 | 2078 | 258 | ABU-258 |
| 1479 | 1839 | 19 | ABU-19 | 1599 | 1959 | 139 | ABU-139 | 1719 | 2079 | 259 | ABU-259 |
| 1480 | 1840 | 20 | ABU-20 | 1600 | 1960 | 140 | ABU-140 | 1720 | 2080 | 260 | ABU-260 |
| 1481 | 1841 | 21 | ABU-21 | 1601 | 1961 | 141 | ABU-141 | 1721 | 2081 | 261 | ABU-261 |
| 1482 | 1842 | 22 | ABU-22 | 1602 | 1962 | 142 | ABU-142 | 1722 | 2082 | 262 | ABU-262 |
| 1483 | 1843 | 23 | ABU-23 | 1603 | 1963 | 143 | ABU-143 | 1723 | 2083 | 263 | ABU-263 |
| 1484 | 1844 | 24 | ABU-24 | 1604 | 1964 | 144 | ABU-144 | 1724 | 2084 | 264 | ABU-264 |
| 1485 | 1845 | 25 | ABU-25 | 1605 | 1965 | 145 | ABU-145 | 1725 | 2085 | 265 | ABU-265 |
| 1486 | 1846 | 26 | ABU-26 | 1606 | 1966 | 146 | ABU-146 | 1726 | 2086 | 266 | ABU-266 |
| 1487 | 1847 | 27 | ABU-27 | 1607 | 1967 | 147 | ABU-147 | 1727 | 2087 | 267 | ABU-267 |
| 1488 | 1848 | 28 | ABU-28 | 1608 | 1968 | 148 | ABU-148 | 1728 | 2088 | 268 | ABU-268 |
| 1489 | 1849 | 29 | ABU-29 | 1609 | 1969 | 149 | ABU-149 | 1729 | 2089 | 269 | ABU-269 |
| 1490 | 1850 | 30 | ABU-30 | 1610 | 1970 | 150 | ABU-150 | 1730 | 2090 | 270 | ABU-270 |
| 1491 | 1851 | 31 | ABU-31 | 1611 | 1971 | 151 | ABU-151 | 1731 | 2091 | 271 | ABU-271 |
| 1492 | 1852 | 32 | ABU-32 | 1612 | 1972 | 152 | ABU-152 | 1732 | 2092 | 272 | ABU-272 |
| 1493 | 1853 | 33 | ABU-33 | 1613 | 1973 | 153 | ABU-153 | 1733 | 2093 | 273 | ABU-273 |
| 1494 | 1854 | 34 | ABU-34 | 1614 | 1974 | 154 | ABU-154 | 1734 | 2094 | 274 | ABU-274 |
| 1495 | 1855 | 35 | ABU-35 | 1615 | 1975 | 155 | ABU-155 | 1735 | 2095 | 275 | ABU-275 |
| 1496 | 1856 | 36 | ABU-36 | 1616 | 1976 | 156 | ABU-156 | 1736 | 2096 | 276 | ABU-276 |
| 1497 | 1857 | 37 | ABU-37 | 1617 | 1977 | 157 | ABU-157 | 1737 | 2097 | 277 | ABU-277 |
| 1498 | 1858 | 38 | ABU-38 | 1618 | 1978 | 158 | ABU-158 | 1738 | 2098 | 278 | ABU-278 |
| 1499 | 1859 | 39 | ABU-39 | 1619 | 1979 | 159 | ABU-159 | 1739 | 2099 | 279 | ABU-279 |
| 1500 | 1860 | 40 | ABU-40 | 1620 | 1980 | 160 | ABU-160 | 1740 | 2100 | 280 | ABU-280 |
| 1501 | 1861 | 41 | ABU-41 | 1621 | 1981 | 161 | ABU-161 | 1741 | 2101 | 281 | ABU-281 |
| 1502 | 1862 | 42 | ABU-42 | 1622 | 1982 | 162 | ABU-162 | 1742 | 2102 | 282 | ABU-282 |
| 1503 | 1863 | 43 | ABU-43 | 1623 | 1983 | 163 | ABU-163 | 1743 | 2103 | 283 | ABU-283 |
| 1504 | 1864 | 44 | ABU-44 | 1624 | 1984 | 164 | ABU-164 | 1744 | 2104 | 284 | ABU-284 |
| 1505 | 1865 | 45 | ABU-45 | 1625 | 1985 | 165 | ABU-165 | 1745 | 2105 | 285 | ABU-285 |
| 1506 | 1866 | 46 | ABU-46 | 1626 | 1986 | 166 | ABU-166 | 1746 | 2106 | 286 | ABU-286 |
| 1507 | 1867 | 47 | ABU-47 | 1627 | 1987 | 167 | ABU-167 | 1747 | 2107 | 287 | ABU-287 |
| 1508 | 1868 | 48 | ABU-48 | 1628 | 1988 | 168 | ABU-168 | 1748 | 2108 | 288 | ABU-288 |
| 1509 | 1869 | 49 | ABU-49 | 1629 | 1989 | 169 | ABU-169 | 1749 | 2109 | 289 | ABU-289 |
| 1510 | 1870 | 50 | ABU-50 | 1630 | 1990 | 170 | ABU-170 | 1750 | 2110 | 290 | ABU-290 |
| 1511 | 1871 | 51 | ABU-51 | 1631 | 1991 | 171 | ABU-171 | 1751 | 2111 | 291 | ABU-291 |
| 1512 | 1872 | 52 | ABU-52 | 1632 | 1992 | 172 | ABU-172 | 1752 | 2112 | 292 | ABU-292 |
| 1513 | 1873 | 53 | ABU-53 | 1633 | 1993 | 173 | ABU-173 | 1753 | 2113 | 293 | ABU-293 |
| 1514 | 1874 | 54 | ABU-54 | 1634 | 1994 | 174 | ABU-174 | 1754 | 2114 | 294 | ABU-294 |
| 1515 | 1875 | 55 | ABU-55 | 1635 | 1995 | 175 | ABU-175 | 1755 | 2115 | 295 | ABU-295 |
| 1516 | 1876 | 56 | ABU-56 | 1636 | 1996 | 176 | ABU-176 | 1756 | 2116 | 296 | ABU-296 |
| 1517 | 1877 | 57 | ABU-57 | 1637 | 1997 | 177 | ABU-177 | 1757 | 2117 | 297 | ABU-297 |
| 1518 | 1878 | 58 | ABU-58 | 1638 | 1998 | 178 | ABU-178 | 1758 | 2118 | 298 | ABU-298 |
| 1519 | 1879 | 59 | ABU-59 | 1639 | 1999 | 179 | ABU-179 | 1759 | 2119 | 299 | ABU-299 |
| 1520 | 1880 | 60 | ABU-60 | 1640 | 2000 | 180 | ABU-180 | 1760 | 2120 | 300 | ABU-300 |
| 1521 | 1881 | 61 | ABU-61 | 1641 | 2001 | 181 | ABU-181 | 1761 | 2121 | 301 | ABU-301 |
| 1522 | 1882 | 62 | ABU-62 | 1642 | 2002 | 182 | ABU-182 | 1762 | 2122 | 302 | ABU-302 |
| 1523 | 1883 | 63 | ABU-63 | 1643 | 2003 | 183 | ABU-183 | 1763 | 2123 | 303 | ABU-303 |
| 1524 | 1884 | 64 | ABU-64 | 1644 | 2004 | 184 | ABU-184 | 1764 | 2124 | 304 | ABU-304 |
| 1525 | 1885 | 65 | ABU-65 | 1645 | 2005 | 185 | ABU-185 | 1765 | 2125 | 305 | ABU-305 |
| 1526 | 1886 | 66 | ABU-66 | 1646 | 2006 | 186 | ABU-186 | 1766 | 2126 | 306 | ABU-306 |
| 1527 | 1887 | 67 | ABU-67 | 1647 | 2007 | 187 | ABU-187 | 1767 | 2127 | 307 | ABU-307 |
| 1528 | 1888 | 68 | ABU-68 | 1648 | 2008 | 188 | ABU-188 | 1768 | 2128 | 308 | ABU-308 |
| 1529 | 1889 | 69 | ABU-69 | 1649 | 2009 | 189 | ABU-189 | 1769 | 2129 | 309 | ABU-309 |
| 1530 | 1890 | 70 | ABU-70 | 1650 | 2010 | 190 | ABU-190 | 1770 | 2130 | 310 | ABU-310 |
| 1531 | 1891 | 71 | ABU-71 | 1651 | 2011 | 191 | ABU-191 | 1771 | 2131 | 311 | ABU-311 |
| 1532 | 1892 | 72 | ABU-72 | 1652 | 2012 | 192 | ABU-192 | 1772 | 2132 | 312 | ABU-312 |
| 1533 | 1893 | 73 | ABU-73 | 1653 | 2013 | 193 | ABU-193 | 1773 | 2133 | 313 | ABU-313 |
| 1534 | 1894 | 74 | ABU-74 | 1654 | 2014 | 194 | ABU-194 | 1774 | 2134 | 314 | ABU-314 |
| 1535 | 1895 | 75 | ABU-75 | 1655 | 2015 | 195 | ABU-195 | 1775 | 2135 | 315 | ABU-315 |
| 1536 | 1896 | 76 | ABU-76 | 1656 | 2016 | 196 | ABU-196 | 1776 | 2136 | 316 | ABU-316 |
| 1537 | 1897 | 77 | ABU-77 | 1657 | 2017 | 197 | ABU-197 | 1777 | 2137 | 317 | ABU-317 |
| 1538 | 1898 | 78 | ABU-78 | 1658 | 2018 | 198 | ABU-198 | 1778 | 2138 | 318 | ABU-318 |
| 1539 | 1899 | 79 | ABU-79 | 1659 | 2019 | 199 | ABU-199 | 1779 | 2139 | 319 | ABU-319 |
| 1540 | 1900 | 80 | ABU-80 | 1660 | 2020 | 200 | ABU-200 | 1780 | 2140 | 320 | ABU-320 |
| 1541 | 1901 | 81 | ABU-81 | 1661 | 2021 | 201 | ABU-201 | 1781 | 2141 | 321 | ABU-321 |
| 1542 | 1902 | 82 | ABU-82 | 1662 | 2022 | 202 | ABU-202 | 1782 | 2142 | 322 | ABU-322 |
| 1543 | 1903 | 83 | ABU-83 | 1663 | 2023 | 203 | ABU-203 | 1783 | 2143 | 323 | ABU-323 |
| 1544 | 1904 | 84 | ABU-84 | 1664 | 2024 | 204 | ABU-204 | 1784 | 2144 | 324 | ABU-324 |
| 1545 | 1905 | 85 | ABU-85 | 1665 | 2025 | 205 | ABU-205 | 1785 | 2145 | 325 | ABU-325 |
| 1546 | 1906 | 86 | ABU-86 | 1666 | 2026 | 206 | ABU-206 | 1786 | 2146 | 326 | ABU-326 |
| 1547 | 1907 | 87 | ABU-87 | 1667 | 2027 | 207 | ABU-207 | 1787 | 2147 | 327 | ABU-327 |
| 1548 | 1908 | 88 | ABU-88 | 1668 | 2028 | 208 | ABU-208 | 1788 | 2148 | 328 | ABU-328 |
| 1549 | 1909 | 89 | ABU-89 | 1669 | 2029 | 209 | ABU-209 | 1789 | 2149 | 329 | ABU-329 |
| 1550 | 1910 | 90 | ABU-90 | 1670 | 2030 | 210 | ABU-210 | 1790 | 2150 | 330 | ABU-330 |
| 1551 | 1911 | 91 | ABU-91 | 1671 | 2031 | 211 | ABU-211 | 1791 | 2151 | 331 | ABU-331 |
| 1552 | 1912 | 92 | ABU-92 | 1672 | 2032 | 212 | ABU-212 | 1792 | 2152 | 332 | ABU-332 |
| 1553 | 1913 | 93 | ABU-93 | 1673 | 2033 | 213 | ABU-213 | 1793 | 2153 | 333 | ABU-333 |
| 1554 | 1914 | 94 | ABU-94 | 1674 | 2034 | 214 | ABU-214 | 1794 | 2154 | 334 | ABU-334 |
| 1555 | 1915 | 95 | ABU-95 | 1675 | 2035 | 215 | ABU-215 | 1795 | 2155 | 335 | ABU-335 |
| 1556 | 1916 | 96 | ABU-96 | 1676 | 2036 | 216 | ABU-216 | 1796 | 2156 | 336 | ABU-336 |
| 1557 | 1917 | 97 | ABU-97 | 1677 | 2037 | 217 | ABU-217 | 1797 | 2157 | 337 | ABU-337 |
| 1558 | 1918 | 98 | ABU-98 | 1678 | 2038 | 218 | ABU-218 | 1798 | 2158 | 338 | ABU-338 |
| 1559 | 1919 | 99 | ABU-99 | 1679 | 2039 | 219 | ABU-219 | 1799 | 2159 | 339 | ABU-339 |
| 1560 | 1920 | 100 | ABU-100 | 1680 | 2040 | 220 | ABU-220 | 1800 | 2160 | 340 | ABU-340 |
| 1561 | 1921 | 101 | ABU-101 | 1681 | 2041 | 221 | ABU-221 | 1801 | 2161 | 341 | ABU-341 |
| 1562 | 1922 | 102 | ABU-102 | 1682 | 2042 | 222 | ABU-222 | 1802 | 2162 | 342 | ABU-342 |
| 1563 | 1923 | 103 | ABU-103 | 1683 | 2043 | 223 | ABU-223 | 1803 | 2163 | 343 | ABU-343 |
| 1564 | 1924 | 104 | ABU-104 | 1684 | 2044 | 224 | ABU-224 | 1804 | 2164 | 344 | ABU-344 |
| 1565 | 1925 | 105 | ABU-105 | 1685 | 2045 | 225 | ABU-225 | 1805 | 2165 | 345 | ABU-345 |
| 1566 | 1926 | 106 | ABU-106 | 1686 | 2046 | 226 | ABU-226 | 1806 | 2166 | 346 | ABU-346 |
| 1567 | 1927 | 107 | ABU-107 | 1687 | 2047 | 227 | ABU-227 | 1807 | 2167 | 347 | ABU-347 |
| 1568 | 1928 | 108 | ABU-108 | 1688 | 2048 | 228 | ABU-228 | 1808 | 2168 | 348 | ABU-348 |
| 1569 | 1929 | 109 | ABU-109 | 1689 | 2049 | 229 | ABU-229 | 1809 | 2169 | 349 | ABU-349 |
| 1570 | 1930 | 110 | ABU-110 | 1690 | 2050 | 230 | ABU-230 | 1810 | 2170 | 350 | ABU-350 |
| 1571 | 1931 | 111 | ABU-111 | 1691 | 2051 | 231 | ABU-231 | 1811 | 2171 | 351 | ABU-351 |
| 1572 | 1932 | 112 | ABU-112 | 1692 | 2052 | 232 | ABU-232 | 1812 | 2172 | 352 | ABU-352 |
| 1573 | 1933 | 113 | ABU-113 | 1693 | 2053 | 233 | ABU-233 | 1813 | 2173 | 353 | ABU-353 |
| 1574 | 1934 | 114 | ABU-114 | 1694 | 2054 | 234 | ABU-234 | 1814 | 2174 | 354 | ABU-354 |
| 1575 | 1935 | 115 | ABU-115 | 1695 | 2055 | 235 | ABU-235 | 1815 | 2175 | 355 | ABU-355 |
| 1576 | 1936 | 116 | ABU-116 | 1696 | 2056 | 236 | ABU-236 | 1816 | 2176 | 356 | ABU-356 |
| 1577 | 1937 | 117 | ABU-117 | 1697 | 2057 | 237 | ABU-237 | 1817 | 2177 | 357 | ABU-357 |
| 1578 | 1938 | 118 | ABU-118 | 1698 | 2058 | 238 | ABU-238 | 1818 | 2178 | 358 | ABU-358 |
| 1579 | 1939 | 119 | ABU-119 | 1699 | 2059 | 239 | ABU-239 | 1819 | 2179 | 359 | ABU-359 |
| 1580 | 1940 | 120 | ABU-120 | 1700 | 2060 | 240 | ABU-240 | 1820 | 2180 | 360 | ABU-360 |
| 2901 | 2936 | 2971 | ABU-361 | 2913 | 2948 | 2983 | ABU-373 | 2925 | 2960 | 2995 | ABU-385 |
| 2902 | 2937 | 2972 | ABU-362 | 2914 | 2949 | 2984 | ABU-374 | 2926 | 2961 | 2996 | ABU-386 |
| 2903 | 2938 | 2973 | ABU-363 | 2915 | 2950 | 2985 | ABU-375 | 2927 | 2962 | 2997 | ABU-387 |
| 2904 | 2939 | 2974 | ABU-364 | 2916 | 2951 | 2986 | ABU-376 | 2928 | 2963 | 2998 | ABU-388 |
| 2905 | 2940 | 2975 | ABU-365 | 2917 | 2952 | 2987 | ABU-377 | 2929 | 2964 | 2999 | ABU-389 |
| 2906 | 2941 | 2976 | ABU-366 | 2918 | 2953 | 2988 | ABU-378 | 2930 | 2965 | 3000 | ABU-390 |
| 2907 | 2942 | 2977 | ABU-367 | 2919 | 2954 | 2989 | ABU-379 | 2931 | 2966 | 3001 | ABU-391 |
| 2908 | 2943 | 2978 | ABU-368 | 2920 | 2955 | 2990 | ABU-380 | 2932 | 2967 | 3002 | ABU-392 |
| 2909 | 2944 | 2979 | ABU-369 | 2921 | 2956 | 2991 | ABU-381 | 2933 | 2968 | 3003 | ABU-393 |
| 2910 | 2945 | 2980 | ABU-370 | 2922 | 2957 | 2992 | ABU-382 | 2934 | 2969 | 3004 | ABU-394 |
| 2911 | 2946 | 2981 | ABU-371 | 2923 | 2958 | 2993 | ABU-383 | 2935 | 2970 | 3005 | ABU-395 |
| 2912 | 2947 | 2982 | ABU-372 | 2924 | 2959 | 2994 | ABU-384 | | | | |

The VL of the antigen binding unit of the present invention can comprise a sequence selected from combinations of CDR1, CDR2, and CDR3 as following:

| LCDR1 | LCDR2 | LCDR3 | ABU No. | LCDR1 | LCDR2 | LCDR3 | ABU No. | LCDR1 | LCDR2 | LCUR3 | ABU No. |
|---|---|---|---|---|---|---|---|---|---|---|---|
| 2181 | 2541 | 361 | ABU-1 | 2301 | 2661 | 481 | ABU-121 | 2421 | 2781 | 601 | ABU-241 |
| 2182 | 2542 | 362 | ABU-2 | 2302 | 2662 | 482 | ABU-122 | 2422 | 2782 | 602 | ABU-242 |
| 2183 | 2543 | 363 | ABU-3 | 2303 | 2663 | 483 | ABU-123 | 2423 | 2783 | 603 | ABU-243 |
| 2184 | 2544 | 364 | ABU-4 | 2304 | 2664 | 484 | ABU-124 | 2424 | 2784 | 604 | ABU-244 |
| 2185 | 2545 | 365 | ABU-5 | 2305 | 2665 | 485 | ABU-125 | 2425 | 2785 | 605 | ABU-245 |
| 2186 | 2546 | 366 | ABU-6 | 2306 | 2666 | 486 | ABU-126 | 2426 | 2786 | 606 | ABU-246 |
| 2187 | 2547 | 367 | ABU-7 | 2307 | 2667 | 487 | ABU-127 | 2427 | 2787 | 607 | ABU-247 |
| 2188 | 2548 | 368 | ABU-8 | 2308 | 2668 | 488 | ABU-128 | 2428 | 2788 | 608 | ABU-248 |
| 2189 | 2549 | 369 | ABU-9 | 2309 | 2669 | 489 | ABU-129 | 2429 | 2789 | 609 | ABU-249 |
| 2190 | 2550 | 370 | ABU-10 | 2310 | 2670 | 490 | ABU-130 | 2430 | 2790 | 610 | ABU-250 |
| 2191 | 2551 | 371 | ABU-11 | 2311 | 2671 | 491 | ABU-131 | 2431 | 2791 | 611 | ABU-251 |
| 2192 | 2552 | 372 | ABU-12 | 2312 | 2672 | 492 | ABU-132 | 2432 | 2792 | 612 | ABU-252 |
| 2193 | 2553 | 373 | ABU-13 | 2313 | 2673 | 493 | ABU-133 | 2433 | 2793 | 613 | ABU-253 |
| 2194 | 2554 | 374 | ABU-14 | 2314 | 2674 | 494 | ABU-134 | 2434 | 2794 | 614 | ABU-254 |
| 2195 | 2555 | 375 | ABU-15 | 2315 | 2675 | 495 | ABU-135 | 2435 | 2795 | 615 | ABU-255 |
| 2196 | 2556 | 376 | ABU-16 | 2316 | 2676 | 496 | ABU-136 | 2436 | 2796 | 616 | ABU-256 |
| 2197 | 2557 | 377 | ABU-17 | 2317 | 2677 | 497 | ABU-137 | 2437 | 2797 | 617 | ABU-257 |
| 2198 | 2558 | 378 | ABU-18 | 2318 | 2678 | 498 | ABU-138 | 2438 | 2798 | 618 | ABU-258 |
| 2199 | 2559 | 379 | ABU-19 | 2319 | 2679 | 499 | ABU-139 | 2439 | 2799 | 619 | ABU-259 |
| 2200 | 2560 | 380 | ABU-20 | 2320 | 2680 | 500 | ABU-140 | 2440 | 2800 | 620 | ABU-260 |
| 2201 | 2561 | 381 | ABU-21 | 2321 | 2681 | 501 | ABU-141 | 2441 | 2801 | 621 | ABU-261 |
| 2202 | 2562 | 382 | ABU-22 | 2322 | 2682 | 502 | ABU-142 | 2442 | 2802 | 622 | ABU-262 |
| 2203 | 2563 | 383 | ABU-23 | 2323 | 2683 | 503 | ABU-143 | 2443 | 2803 | 623 | ABU-263 |
| 2204 | 2564 | 384 | ABU-24 | 2324 | 2684 | 504 | ABU-144 | 2444 | 2804 | 624 | ABU-264 |
| 2205 | 2565 | 385 | ABU-25 | 2325 | 2685 | 505 | ABU-145 | 2445 | 2805 | 625 | ABU-265 |
| 2206 | 2566 | 386 | ABU-26 | 2326 | 2686 | 506 | ABU-146 | 2446 | 2806 | 626 | ABU-266 |
| 2207 | 2567 | 387 | ABU-27 | 2327 | 2687 | 507 | ABU-147 | 2447 | 2807 | 627 | ABU-267 |
| 2208 | 2568 | 388 | ABU-28 | 2328 | 2688 | 508 | ABU-148 | 2448 | 2808 | 628 | ABU-268 |
| 2209 | 2569 | 389 | ABU-29 | 2329 | 2689 | 509 | ABU-149 | 2449 | 2809 | 629 | ABU-269 |
| 2210 | 2570 | 390 | ABU-30 | 2330 | 2690 | 510 | ABU-150 | 2450 | 2810 | 630 | ABU-270 |
| 2211 | 2571 | 391 | ABU-31 | 2331 | 2691 | 511 | ABU-151 | 2451 | 2811 | 631 | ABU-271 |
| 2212 | 2572 | 392 | ABU-32 | 2332 | 2692 | 512 | ABU-152 | 2452 | 2812 | 632 | ABU-272 |
| 2213 | 2573 | 393 | ABU-33 | 2333 | 2693 | 513 | ABU-153 | 2453 | 2813 | 633 | ABU-273 |
| 2214 | 2574 | 394 | ABU-34 | 2334 | 2694 | 514 | ABU-154 | 2454 | 2814 | 634 | ABU-274 |
| 2215 | 2575 | 395 | ABU-35 | 2335 | 2695 | 515 | ABU-155 | 2455 | 2815 | 635 | ABU-275 |
| 2216 | 2576 | 396 | ABU-36 | 2336 | 2696 | 516 | ABU-156 | 2456 | 2816 | 636 | ABU-276 |
| 2217 | 2577 | 397 | ABU-37 | 2337 | 2697 | 517 | ABU-157 | 2457 | 2817 | 637 | ABU-277 |
| 2218 | 2578 | 398 | ABU-38 | 2338 | 2698 | 518 | ABU-158 | 2458 | 2818 | 638 | ABU-278 |
| 2219 | 2579 | 399 | ABU-39 | 2339 | 2699 | 519 | ABU-159 | 2459 | 2819 | 639 | ABU-279 |
| 2220 | 2580 | 400 | ABU-40 | 2340 | 2700 | 520 | ABU-160 | 2460 | 2820 | 640 | ABU-280 |
| 2221 | 2581 | 401 | ABU-41 | 2341 | 2701 | 521 | ABU-161 | 2461 | 2821 | 641 | ABU-281 |
| 2222 | 2582 | 402 | ABU-42 | 2342 | 2702 | 522 | ABU-162 | 2462 | 2822 | 642 | ABU-282 |
| 2223 | 2583 | 403 | ABU-43 | 2343 | 2703 | 523 | ABU-163 | 2463 | 2823 | 643 | ABU-283 |
| 2224 | 2584 | 404 | ABU-44 | 2344 | 2704 | 524 | ABU-164 | 2464 | 2824 | 644 | ABU-284 |
| 2225 | 2585 | 405 | ABU-45 | 2345 | 2705 | 525 | ABU-165 | 2465 | 2825 | 645 | ABU-285 |
| 2226 | 2586 | 406 | ABU-46 | 2346 | 2706 | 526 | ABU-166 | 2466 | 2826 | 646 | ABU-286 |
| 2227 | 2587 | 407 | ABU-47 | 2347 | 2707 | 527 | ABU-167 | 2467 | 2827 | 647 | ABU-287 |
| 2228 | 2588 | 408 | ABU-48 | 2348 | 2708 | 528 | ABU-168 | 2468 | 2828 | 648 | ABU-288 |
| 2229 | 2589 | 409 | ABU-49 | 2349 | 2709 | 529 | ABU-169 | 2469 | 2829 | 649 | ABU-289 |
| 2230 | 2590 | 410 | ABU-50 | 2350 | 2710 | 530 | ABU-170 | 2470 | 2830 | 650 | ABU-290 |
| 2231 | 2591 | 411 | ABU-51 | 2351 | 2711 | 531 | ABU-171 | 2471 | 2831 | 651 | ABU-291 |
| 2232 | 2592 | 412 | ABU-52 | 2352 | 2712 | 532 | ABU-172 | 2472 | 2832 | 652 | ABU-292 |
| 2233 | 2593 | 413 | ABU-53 | 2353 | 2713 | 533 | ABU-173 | 2473 | 2833 | 653 | ABU-293 |
| 2234 | 2594 | 414 | ABU-54 | 2354 | 2714 | 534 | ABU-174 | 2474 | 2834 | 654 | ABU-294 |
| 2235 | 2595 | 415 | ABU-55 | 2355 | 2715 | 535 | ABU-175 | 2475 | 2835 | 655 | ABU-295 |
| 2236 | 2596 | 416 | ABU-56 | 2356 | 2716 | 536 | ABU-176 | 2476 | 2836 | 656 | ABU-296 |
| 2237 | 2597 | 417 | ABU-57 | 2357 | 2717 | 537 | ABU-177 | 2477 | 2837 | 657 | ABU-297 |
| 2238 | 2598 | 418 | ABU-58 | 2358 | 2718 | 538 | ABU-178 | 2478 | 2838 | 658 | ABU-298 |
| 2239 | 2599 | 419 | ABU-59 | 2359 | 2719 | 539 | ABU-179 | 2479 | 2839 | 659 | ABU-299 |
| 2240 | 2600 | 420 | ABU-60 | 2360 | 2720 | 540 | ABU-180 | 2480 | 2840 | 660 | ABU-300 |
| 2241 | 2601 | 421 | ABU-61 | 2361 | 2721 | 541 | ABU-181 | 2481 | 2841 | 661 | ABU-301 |
| 2242 | 2602 | 422 | ABU-62 | 2362 | 2722 | 542 | ABU-182 | 2482 | 2842 | 662 | ABU-302 |
| 2243 | 2603 | 423 | ABU-63 | 2363 | 2723 | 543 | ABU-183 | 2483 | 2843 | 663 | ABU-303 |
| 2244 | 2604 | 424 | ABU-64 | 2364 | 2724 | 544 | ABU-184 | 2484 | 2844 | 664 | ABU-304 |
| 2245 | 2605 | 425 | ABU-65 | 2365 | 2725 | 545 | ABU-185 | 2485 | 2845 | 665 | ABU-305 |
| 2246 | 2606 | 426 | ABU-66 | 2366 | 2726 | 546 | ABU-186 | 2486 | 2846 | 666 | ABU-306 |
| 2247 | 2607 | 427 | ABU-67 | 2367 | 2727 | 547 | ABU-187 | 2487 | 2847 | 667 | ABU-307 |
| 2248 | 2608 | 428 | ABU-68 | 2368 | 2728 | 548 | ABU-188 | 2488 | 2848 | 668 | ABU-308 |
| 2249 | 2609 | 429 | ABU-69 | 2369 | 2729 | 549 | ABU-189 | 2489 | 2849 | 669 | ABU-309 |
| 2250 | 2610 | 430 | ABU-70 | 2370 | 2730 | 550 | ABU-190 | 2490 | 2850 | 670 | ABU-310 |
| 2251 | 2611 | 431 | ABU-71 | 2371 | 2731 | 551 | ABU-191 | 2491 | 2851 | 671 | ABU-311 |
| 2252 | 2612 | 432 | ABU-72 | 2372 | 2732 | 552 | ABU-192 | 2492 | 2852 | 672 | ABU-312 |
| 2253 | 2613 | 433 | ABU-73 | 2373 | 2733 | 553 | ABU-193 | 2493 | 2853 | 673 | ABU-313 |
| 2254 | 2614 | 434 | ABU-74 | 2374 | 2734 | 554 | ABU-194 | 2494 | 2854 | 674 | ABU-314 |
| 2255 | 2615 | 435 | ABU-75 | 2375 | 2735 | 555 | ABU-195 | 2495 | 2855 | 675 | ABU-315 |
| 2256 | 2616 | 436 | ABU-76 | 2376 | 2736 | 556 | ABU-196 | 2496 | 2856 | 676 | ABU-316 |
| 2257 | 2617 | 437 | ABU-77 | 2377 | 2737 | 557 | ABU-197 | 2497 | 2857 | 677 | ABU-317 |
| 2258 | 2618 | 438 | ABU-78 | 2378 | 2738 | 558 | ABU-198 | 2498 | 2858 | 678 | ABU-318 |
| 2259 | 2619 | 439 | ABU-79 | 2379 | 2739 | 559 | ABU-199 | 2499 | 2859 | 679 | ABU-319 |
| 2260 | 2620 | 440 | ABU-80 | 2380 | 2740 | 560 | ABU-200 | 2500 | 2860 | 680 | ABU-320 |
| 2261 | 2621 | 441 | ABU-81 | 2381 | 2741 | 561 | ABU-201 | 2501 | 2861 | 681 | ABU-321 |
| 2262 | 2622 | 442 | ABU-82 | 2382 | 2742 | 562 | ABU-202 | 2502 | 2862 | 682 | ABU-322 |
| 2263 | 2623 | 443 | ABU-83 | 2383 | 2743 | 563 | ABU-203 | 2503 | 2863 | 683 | ABU-323 |
| 2264 | 2624 | 444 | ABU-84 | 2384 | 2744 | 564 | ABU-204 | 2504 | 2864 | 684 | ABU-324 |
| 2265 | 2625 | 445 | ABU-85 | 2385 | 2745 | 565 | ABU-205 | 2505 | 2865 | 685 | ABU-325 |
| 2266 | 2626 | 446 | ABU-86 | 2386 | 2746 | 566 | ABU-206 | 2506 | 2866 | 686 | ABU-326 |
| 2267 | 2627 | 447 | ABU-87 | 2387 | 2747 | 567 | ABU-207 | 2507 | 2867 | 687 | ABU-327 |
| 2268 | 2628 | 448 | ABU-88 | 2388 | 2748 | 568 | ABU-208 | 2508 | 2868 | 688 | ABU-328 |
| 2269 | 2629 | 449 | ABU-89 | 2389 | 2749 | 569 | ABU-209 | 2509 | 2869 | 689 | ABU-329 |
| 2270 | 2630 | 450 | ABU-90 | 2390 | 2750 | 570 | ABU-210 | 2510 | 2870 | 690 | ABU-330 |
| 2271 | 2631 | 451 | ABU-91 | 2391 | 2751 | 571 | ABU-211 | 2511 | 2871 | 691 | ABU-331 |
| 2272 | 2632 | 452 | ABU-92 | 2392 | 2752 | 572 | ABU-212 | 2512 | 2872 | 692 | ABU-332 |
| 2273 | 2633 | 453 | ABU-93 | 2393 | 2753 | 573 | ABU-213 | 2513 | 2873 | 693 | ABU-333 |
| 2274 | 2634 | 454 | ABU-94 | 2394 | 2754 | 574 | ABU-214 | 2514 | 2874 | 694 | ABU-334 |
| 2275 | 2635 | 455 | ABU-95 | 2395 | 2755 | 575 | ABU-215 | 2515 | 2875 | 695 | ABU-335 |
| 2276 | 2636 | 456 | ABU-96 | 2396 | 2756 | 576 | ABU-216 | 2516 | 2876 | 696 | ABU-336 |
| 2277 | 2637 | 457 | ABU-97 | 2397 | 2757 | 577 | ABU-217 | 2517 | 2877 | 697 | ABU-337 |
| 2278 | 2638 | 458 | ABU-98 | 2398 | 2758 | 578 | ABU-218 | 2518 | 2878 | 698 | ABU-338 |
| 2279 | 2639 | 459 | ABU-99 | 2399 | 2759 | 579 | ABU-219 | 2519 | 2879 | 699 | ABU-339 |
| 2280 | 2640 | 460 | ABU-100 | 2400 | 2760 | 580 | ABU-220 | 2520 | 2880 | 700 | ABU-340 |
| 2281 | 2641 | 461 | ABU-101 | 2401 | 2761 | 581 | ABU-221 | 2521 | 2881 | 701 | ABU-341 |
| 2282 | 2642 | 462 | ABU-102 | 2402 | 2762 | 582 | ABU-222 | 2522 | 2882 | 702 | ABU-342 |
| 2283 | 2643 | 463 | ABU-103 | 2403 | 2763 | 583 | ABU-223 | 2523 | 2883 | 703 | ABU-343 |
| 2284 | 2644 | 464 | ABU-104 | 2404 | 2764 | 584 | ABU-224 | 2524 | 2884 | 704 | ABU-344 |
| 2285 | 2645 | 465 | ABU-105 | 2405 | 2765 | 585 | ABU-225 | 2525 | 2885 | 705 | ABU-345 |
| 2286 | 2646 | 466 | ABU-106 | 2406 | 2766 | 586 | ABU-226 | 2526 | 2886 | 706 | ABU-346 |
| 2287 | 2647 | 467 | ABU-107 | 2407 | 2767 | 587 | ABU-227 | 2527 | 2887 | 707 | ABU-347 |
| 2288 | 2648 | 468 | ABU-108 | 2408 | 2768 | 588 | ABU-228 | 2528 | 2888 | 708 | ABU-348 |
| 2289 | 2649 | 469 | ABU-109 | 2409 | 2769 | 589 | ABU-229 | 2529 | 2889 | 709 | ABU-349 |
| 2290 | 2650 | 470 | ABU-110 | 2410 | 2770 | 590 | ABU-230 | 2530 | 2890 | 710 | ABU-350 |
| 2291 | 2651 | 471 | ABU-111 | 2411 | 2771 | 591 | ABU-231 | 2531 | 2891 | 711 | ABU-351 |
| 2292 | 2652 | 472 | ABU-112 | 2412 | 2772 | 592 | ABU-232 | 2532 | 2892 | 712 | ABU-352 |
| 2293 | 2653 | 473 | ABU-113 | 2413 | 2773 | 593 | ABU-233 | 2533 | 2893 | 713 | ABU-353 |
| 2294 | 2654 | 474 | ABU-114 | 2414 | 2774 | 594 | ABU-234 | 2534 | 2894 | 714 | ABU-354 |
| 2295 | 2655 | 475 | ABU-115 | 2415 | 2775 | 595 | ABU-235 | 2535 | 2895 | 715 | ABU-355 |
| 2296 | 2656 | 476 | ABU-116 | 2416 | 2776 | 596 | ABU-236 | 2536 | 2896 | 716 | ABU-356 |
| 2297 | 2657 | 477 | ABU-117 | 2417 | 2777 | 597 | ABU-237 | 2537 | 2897 | 717 | ABU-357 |
| 2298 | 2658 | 478 | ABU-118 | 2418 | 2778 | 598 | ABU-238 | 2538 | 2898 | 718 | ABU-358 |
| 2299 | 2659 | 479 | ABU-119 | 2419 | 2779 | 599 | ABU-239 | 2539 | 2899 | 719 | ABU-359 |
| 2300 | 2660 | 480 | ABU-120 | 2420 | 2780 | 600 | ABU-240 | 2540 | 2900 | 720 | ABU-360 |
| 3006 | 3041 | 3076 | ABU-361 | 3018 | 3053 | 3088 | ABU-373 | 3030 | 3065 | 3100 | ABU-385 |
| 3007 | 3042 | 3077 | ABU-362 | 3019 | 3054 | 3089 | ABU-374 | 3031 | 3066 | 3101 | ABU-386 |
| 3008 | 3043 | 3078 | ABU-363 | 3020 | 3055 | 3090 | ABU-375 | 3032 | 3067 | 3102 | ABU-387 |
| 3009 | 3044 | 3079 | ABU-364 | 3021 | 3056 | 3091 | ABU-376 | 3033 | 3068 | 3103 | ABU-388 |
| 3010 | 3045 | 3080 | ABU-365 | 3022 | 3057 | 3092 | ABU-377 | 3034 | 3069 | 3104 | ABU-389 |
| 3011 | 3046 | 3081 | ABU-366 | 3023 | 3058 | 3093 | ABU-378 | 3035 | 3070 | 3105 | ABU-390 |
| 3012 | 3047 | 3082 | ABU-367 | 3024 | 3059 | 3094 | ABU-379 | 3036 | 3071 | 3106 | ABU-391 |
| 3013 | 3048 | 3083 | ABU-368 | 3025 | 3060 | 3095 | ABU-380 | 3037 | 3072 | 3107 | ABU-392 |
| 3014 | 3049 | 3084 | ABU-369 | 3026 | 3061 | 3096 | ABU-381 | 3038 | 3073 | 3108 | ABU-393 |
| 3015 | 3050 | 3085 | ABU-370 | 3027 | 3062 | 3097 | ABU-382 | 3039 | 3074 | 3109 | ABU-394 |
| 3016 | 3051 | 3086 | ABU-371 | 3028 | 3063 | 3098 | ABU-383 | 3040 | 3075 | 3110 | ABU-395 |
| 3017 | 3052 | 3087 | ABU-372 | 3029 | 3064 | 3099 | ABU-384 | | | | |

In the antigen binding unit of the present invention, the VH can comprise a sequence selected from combinations of CDR1, CDR2, and CDR3 as following:

| HCDR1 | HCDR2 | HCDR3 | ABU No. | HCDR1 | HCDR2 | HCDR3 | ABU No. | HCDR1 | HCDR2 | HCDR3 | ABU No. |
|---|---|---|---|---|---|---|---|---|---|---|---|
| 1461 | 1821 | 1 | ABU-1 | 1581 | 1941 | 121 | ABU-121 | 1701 | 2061 | 241 | ABU-241 |
| 1462 | 1822 | 2 | ABU-2 | 1582 | 1942 | 122 | ABU-122 | 1702 | 2062 | 242 | ABU-242 |
| 1463 | 1823 | 3 | ABU-3 | 1583 | 1943 | 123 | ABU-123 | 1703 | 2063 | 243 | ABU-243 |
| 1464 | 1824 | 4 | ABU-4 | 1584 | 1944 | 124 | ABU-124 | 1704 | 2064 | 244 | ABU-244 |
| 1465 | 1825 | 5 | ABU-5 | 1585 | 1945 | 125 | ABU-125 | 1705 | 2065 | 245 | ABU-245 |
| 1466 | 1826 | 6 | ABU-6 | 1586 | 1946 | 126 | ABU-126 | 1706 | 2066 | 246 | ABU-246 |
| 1467 | 1827 | 7 | ABU-7 | 1587 | 1947 | 127 | ABU-127 | 1707 | 2067 | 247 | ABU-247 |
| 1468 | 1828 | 8 | ABU-8 | 1588 | 1948 | 128 | ABU-128 | 1708 | 2068 | 248 | ABU-248 |
| 1469 | 1829 | 9 | ABU-9 | 1589 | 1949 | 129 | ABU-129 | 1709 | 2069 | 249 | ABU-249 |
| 1470 | 1830 | 10 | ABU-10 | 1590 | 1950 | 130 | ABU-130 | 1710 | 2070 | 250 | ABU-250 |
| 1471 | 1831 | 11 | ABU-11 | 1591 | 1951 | 131 | ABU-131 | 1711 | 2071 | 251 | ABU-251 |
| 1472 | 1832 | 12 | ABU-12 | 1592 | 1952 | 132 | ABU-132 | 1712 | 2072 | 252 | ABU-252 |
| 1473 | 1833 | 13 | ABU-13 | 1593 | 1953 | 133 | ABU-133 | 1713 | 2073 | 253 | ABU-253 |
| 1474 | 1834 | 14 | ABU-14 | 1594 | 1954 | 134 | ABU-134 | 1714 | 2074 | 254 | ABU-254 |
| 1475 | 1835 | 15 | ABU-15 | 1595 | 1955 | 135 | ABU-135 | 1715 | 2075 | 255 | ABU-255 |
| 1476 | 1836 | 16 | ABU-16 | 1596 | 1956 | 136 | ABU-136 | 1716 | 2076 | 256 | ABU-256 |
| 1477 | 1837 | 17 | ABU-17 | 1597 | 1957 | 137 | ABU-137 | 1717 | 2077 | 257 | ABU-257 |
| 1478 | 1838 | 18 | ABU-18 | 1598 | 1958 | 138 | ABU-138 | 1718 | 2078 | 258 | ABU-258 |
| 1479 | 1839 | 19 | ABU-19 | 1599 | 1959 | 139 | ABU-139 | 1719 | 2079 | 259 | ABU-259 |
| 1480 | 1840 | 20 | ABU-20 | 1600 | 1960 | 140 | ABU-140 | 1720 | 2080 | 260 | ABU-260 |
| 1481 | 1841 | 21 | ABU-21 | 1601 | 1961 | 141 | ABU-141 | 1721 | 2081 | 261 | ABU-261 |
| 1482 | 1842 | 22 | ABU-22 | 1602 | 1962 | 142 | ABU-142 | 1722 | 2082 | 262 | ABU-262 |
| 1483 | 1843 | 23 | ABU-23 | 1603 | 1963 | 143 | ABU-143 | 1723 | 2083 | 263 | ABU-263 |
| 1484 | 1844 | 24 | ABU-24 | 1604 | 1964 | 144 | ABU-144 | 1724 | 2084 | 264 | ABU-264 |
| 1485 | 1845 | 25 | ABU-25 | 1605 | 1965 | 145 | ABU-145 | 1725 | 2085 | 265 | ABU-265 |
| 1486 | 1846 | 26 | ABU-26 | 1606 | 1966 | 146 | ABU-146 | 1726 | 2086 | 266 | ABU-266 |
| 1487 | 1847 | 27 | ABU-27 | 1607 | 1967 | 147 | ABU-147 | 1727 | 2087 | 267 | ABU-267 |
| 1488 | 1848 | 28 | ABU-28 | 1608 | 1968 | 148 | ABU-148 | 1728 | 2088 | 268 | ABU-268 |
| 1489 | 1849 | 29 | ABU-29 | 1609 | 1969 | 149 | ABU-149 | 1729 | 2089 | 269 | ABU-269 |
| 1490 | 1850 | 30 | ABU-30 | 1610 | 1970 | 150 | ABU-150 | 1730 | 2090 | 270 | ABU-270 |
| 1491 | 1851 | 31 | ABU-31 | 1611 | 1971 | 151 | ABU-151 | 1731 | 2091 | 271 | ABU-271 |
| 1492 | 1852 | 32 | ABU-32 | 1612 | 1972 | 152 | ABU-152 | 1732 | 2092 | 272 | ABU-272 |
| 1493 | 1853 | 33 | ABU-33 | 1613 | 1973 | 153 | ABU-153 | 1733 | 2093 | 273 | ABU-273 |
| 1494 | 1854 | 34 | ABU-34 | 1614 | 1974 | 154 | ABU-154 | 1734 | 2094 | 274 | ABU-274 |
| 1495 | 1855 | 35 | ABU-35 | 1615 | 1975 | 155 | ABU-155 | 1735 | 2095 | 275 | ABU-275 |
| 1496 | 1856 | 36 | ABU-36 | 1616 | 1976 | 156 | ABU-156 | 1736 | 2096 | 276 | ABU-276 |
| 1497 | 1857 | 37 | ABU-37 | 1617 | 1977 | 157 | ABU-157 | 1737 | 2097 | 277 | ABU-277 |
| 1498 | 1858 | 38 | ABU-38 | 1618 | 1978 | 158 | ABU-158 | 1738 | 2098 | 278 | ABU-278 |
| 1499 | 1859 | 39 | ABU-39 | 1619 | 1979 | 159 | ABU-159 | 1739 | 2099 | 279 | ABU-279 |
| 1500 | 1860 | 40 | ABU-40 | 1620 | 1980 | 160 | ABU-160 | 1740 | 2100 | 280 | ABU-280 |
| 1501 | 1861 | 41 | ABU-41 | 1621 | 1981 | 161 | ABU-161 | 1741 | 2101 | 281 | ABU-281 |
| 1502 | 1862 | 42 | ABU-42 | 1622 | 1982 | 162 | ABU-162 | 1742 | 2102 | 282 | ABU-282 |
| 1503 | 1863 | 43 | ABU-43 | 1623 | 1983 | 163 | ABU-163 | 1743 | 2103 | 283 | ABU-283 |
| 1504 | 1864 | 44 | ABU-44 | 1624 | 1984 | 164 | ABU-164 | 1744 | 2104 | 284 | ABU-284 |
| 1505 | 1865 | 45 | ABU-45 | 1625 | 1985 | 165 | ABU-165 | 1745 | 2105 | 285 | ABU-285 |
| 1506 | 1866 | 46 | ABU-46 | 1626 | 1986 | 166 | ABU-166 | 1746 | 2106 | 286 | ABU-286 |
| 1507 | 1867 | 47 | ABU-47 | 1627 | 1987 | 167 | ABU-167 | 1747 | 2107 | 287 | ABU-287 |
| 1508 | 1868 | 48 | ABU-48 | 1628 | 1988 | 168 | ABU-168 | 1748 | 2108 | 288 | ABU-288 |
| 1509 | 1869 | 49 | ABU-49 | 1629 | 1989 | 169 | ABU-169 | 1749 | 2109 | 289 | ABU-289 |
| 1510 | 1870 | 50 | ABU-50 | 1630 | 1990 | 170 | ABU-170 | 1750 | 2110 | 290 | ABU-290 |
| 1511 | 1871 | 51 | ABU-51 | 1631 | 1991 | 171 | ABU-171 | 1751 | 2111 | 291 | ABU-291 |
| 1512 | 1872 | 52 | ABU-52 | 1632 | 1992 | 172 | ABU-172 | 1752 | 2112 | 292 | ABU-292 |
| 1513 | 1873 | 53 | ABU-53 | 1633 | 1993 | 173 | ABU-173 | 1753 | 2113 | 293 | ABU-293 |
| 1514 | 1874 | 54 | ABU-54 | 1634 | 1994 | 174 | ABU-174 | 1754 | 2114 | 294 | ABU-294 |
| 1515 | 1875 | 55 | ABU-55 | 1635 | 1995 | 175 | ABU-175 | 1755 | 2115 | 295 | ABU-295 |
| 1516 | 1876 | 56 | ABU-56 | 1636 | 1996 | 176 | ABU-176 | 1756 | 2116 | 296 | ABU-296 |
| 1517 | 1877 | 57 | ABU-57 | 1637 | 1997 | 177 | ABU-177 | 1757 | 2117 | 297 | ABU-297 |
| 1518 | 1878 | 58 | ABU-58 | 1638 | 1998 | 178 | ABU-178 | 1758 | 2118 | 298 | ABU-298 |
| 1519 | 1879 | 59 | ABU-59 | 1639 | 1999 | 179 | ABU-179 | 1759 | 2119 | 299 | ABU-299 |
| 1520 | 1880 | 60 | ABU-60 | 1640 | 2000 | 180 | ABU-180 | 1760 | 2120 | 300 | ABU-300 |
| 1521 | 1881 | 61 | ABU-61 | 1641 | 2001 | 181 | ABU-181 | 1761 | 2121 | 301 | ABU-301 |
| 1522 | 1882 | 62 | ABU-62 | 1642 | 2002 | 182 | ABU-182 | 1762 | 2122 | 302 | ABU-302 |
| 1523 | 1883 | 63 | ABU-63 | 1643 | 2003 | 183 | ABU-183 | 1763 | 2123 | 303 | ABU-303 |
| 1524 | 1884 | 64 | ABU-64 | 1644 | 2004 | 184 | ABU-184 | 1764 | 2124 | 304 | ABU-304 |
| 1525 | 1885 | 65 | ABU-65 | 1645 | 2005 | 185 | ABU-185 | 1765 | 2125 | 305 | ABU-305 |
| 1526 | 1886 | 66 | ABU-66 | 1646 | 2006 | 186 | ABU-186 | 1766 | 2126 | 306 | ABU-306 |
| 1527 | 1887 | 67 | ABU-67 | 1647 | 2007 | 187 | ABU-187 | 1767 | 2127 | 307 | ABU-307 |
| 1528 | 1888 | 68 | ABU-68 | 1648 | 2008 | 188 | ABU-188 | 1768 | 2128 | 308 | ABU-308 |
| 1529 | 1889 | 69 | ABU-69 | 1649 | 2009 | 189 | ABU-189 | 1769 | 2129 | 309 | ABU-309 |
| 1530 | 1890 | 70 | ABU-70 | 1650 | 2010 | 190 | ABU-190 | 1770 | 2130 | 310 | ABU-310 |
| 1531 | 1891 | 71 | ABU-71 | 1651 | 2011 | 191 | ABU-191 | 1771 | 2131 | 311 | ABU-311 |
| 1532 | 1892 | 72 | ABU-72 | 1652 | 2012 | 192 | ABU-192 | 1772 | 2132 | 312 | ABU-312 |
| 1533 | 1893 | 73 | ABU-73 | 1653 | 2013 | 193 | ABU-193 | 1773 | 2133 | 313 | ABU-313 |
| 1534 | 1894 | 74 | ABU-74 | 1654 | 2014 | 194 | ABU-194 | 1774 | 2134 | 314 | ABU-314 |
| 1535 | 1895 | 75 | ABU-75 | 1655 | 2015 | 195 | ABU-195 | 1775 | 2135 | 315 | ABU-315 |
| 1536 | 1896 | 76 | ABU-76 | 1656 | 2016 | 196 | ABU-196 | 1776 | 2136 | 316 | ABU-316 |
| 1537 | 1897 | 77 | ABU-77 | 1657 | 2017 | 197 | ABU-197 | 1777 | 2137 | 317 | ABU-317 |
| 1538 | 1898 | 78 | ABU-78 | 1658 | 2018 | 198 | ABU-198 | 1778 | 2138 | 318 | ABU-318 |
| 1539 | 1899 | 79 | ABU-79 | 1659 | 2019 | 199 | ABU-199 | 1779 | 2139 | 319 | ABU-319 |
| 1540 | 1900 | 80 | ABU-80 | 1660 | 2020 | 200 | ABU-200 | 1780 | 2140 | 320 | ABU-320 |
| 1541 | 1901 | 81 | ABU-81 | 1661 | 2021 | 201 | ABU-201 | 1781 | 2141 | 321 | ABU-321 |
| 1542 | 1902 | 82 | ABU-82 | 1662 | 2022 | 202 | ABU-202 | 1782 | 2142 | 322 | ABU-322 |
| 1543 | 1903 | 83 | ABU-83 | 1663 | 2023 | 203 | ABU-203 | 1783 | 2143 | 323 | ABU-323 |
| 1544 | 1904 | 84 | ABU-84 | 1664 | 2024 | 204 | ABU-204 | 1784 | 2144 | 324 | ABU-324 |
| 1545 | 1905 | 85 | ABU-85 | 1665 | 2025 | 205 | ABU-205 | 1785 | 2145 | 325 | ABU-325 |
| 1546 | 1906 | 86 | ABU-86 | 1666 | 2026 | 206 | ABU-206 | 1786 | 2146 | 326 | ABU-326 |
| 1547 | 1907 | 87 | ABU-87 | 1667 | 2027 | 207 | ABU-207 | 1787 | 2147 | 327 | ABU-327 |
| 1548 | 1908 | 88 | ABU-88 | 1668 | 2028 | 208 | ABU-208 | 1788 | 2148 | 328 | ABU-328 |
| 1549 | 1909 | 89 | ABU-89 | 1669 | 2029 | 209 | ABU-209 | 1789 | 2149 | 329 | ABU-329 |
| 1550 | 1910 | 90 | ABU-90 | 1670 | 2030 | 210 | ABU-210 | 1790 | 2150 | 330 | ABU-330 |
| 1551 | 1911 | 91 | ABU-91 | 1671 | 2031 | 211 | ABU-211 | 1791 | 2151 | 331 | ABU-331 |
| 1552 | 1912 | 92 | ABU-92 | 1672 | 2032 | 212 | ABU-212 | 1792 | 2152 | 332 | ABU-332 |
| 1553 | 1913 | 93 | ABU-93 | 1673 | 2033 | 213 | ABU-213 | 1793 | 2153 | 333 | ABU-333 |
| 1554 | 1914 | 94 | ABU-94 | 1674 | 2034 | 214 | ABU-214 | 1794 | 2154 | 334 | ABU-334 |
| 1555 | 1915 | 95 | ABU-95 | 1675 | 2035 | 215 | ABU-215 | 1795 | 2155 | 335 | ABU-335 |
| 1556 | 1916 | 96 | ABU-96 | 1676 | 2036 | 216 | ABU-216 | 1796 | 2156 | 336 | ABU-336 |
| 1557 | 1917 | 97 | ABU-97 | 1677 | 2037 | 217 | ABU-217 | 1797 | 2157 | 337 | ABU-337 |
| 1558 | 1918 | 98 | ABU-98 | 1678 | 2038 | 218 | ABU-218 | 1798 | 2158 | 338 | ABU-338 |
| 1559 | 1919 | 99 | ABU-99 | 1679 | 2039 | 219 | ABU-219 | 1799 | 2159 | 339 | ABU-339 |
| 1560 | 1920 | 100 | ABU-100 | 1680 | 2040 | 220 | ABU-220 | 1800 | 2160 | 340 | ABU-340 |
| 1561 | 1921 | 101 | ABU-101 | 1681 | 2041 | 221 | ABU-221 | 1801 | 2161 | 341 | ABU-341 |
| 1562 | 1922 | 102 | ABU-102 | 1682 | 2042 | 222 | ABU-222 | 1802 | 2162 | 342 | ABU-342 |
| 1563 | 1923 | 103 | ABU-103 | 1683 | 2043 | 223 | ABU-223 | 1803 | 2163 | 343 | ABU-343 |
| 1564 | 1924 | 104 | ABU-104 | 1684 | 2044 | 224 | ABU-224 | 1804 | 2164 | 344 | ABU-344 |
| 1565 | 1925 | 105 | ABU-105 | 1685 | 2045 | 225 | ABU-225 | 1805 | 2165 | 345 | ABU-345 |
| 1566 | 1926 | 106 | ABU-106 | 1686 | 2046 | 226 | ABU-226 | 1806 | 2166 | 346 | ABU-346 |
| 1567 | 1927 | 107 | ABU-107 | 1687 | 2047 | 227 | ABU-227 | 1807 | 2167 | 347 | ABU-347 |
| 1568 | 1928 | 108 | ABU-108 | 1688 | 2048 | 228 | ABU-228 | 1808 | 2168 | 348 | ABU-348 |
| 1569 | 1929 | 109 | ABU-109 | 1689 | 2049 | 229 | ABU-229 | 1809 | 2169 | 349 | ABU-349 |
| 1570 | 1930 | 110 | ABU-110 | 1690 | 2050 | 230 | ABU-230 | 1810 | 2170 | 350 | ABU-350 |
| 1571 | 1931 | 111 | ABU-111 | 1691 | 2051 | 231 | ABU-231 | 1811 | 2171 | 351 | ABU-351 |
| 1572 | 1932 | 112 | ABU-112 | 1692 | 2052 | 232 | ABU-232 | 1812 | 2172 | 352 | ABU-352 |
| 1573 | 1933 | 113 | ABU-113 | 1693 | 2053 | 233 | ABU-233 | 1813 | 2173 | 353 | ABU-353 |
| 1574 | 1934 | 114 | ABU-114 | 1694 | 2054 | 234 | ABU-234 | 1814 | 2174 | 354 | ABU-354 |
| 1575 | 1935 | 115 | ABU-115 | 1695 | 2055 | 235 | ABU-235 | 1815 | 2175 | 355 | ABU-355 |
| 1576 | 1936 | 116 | ABU-116 | 1696 | 2056 | 236 | ABU-236 | 1816 | 2176 | 356 | ABU-356 |
| 1577 | 1937 | 117 | ABU-117 | 1697 | 2057 | 237 | ABU-237 | 1817 | 2177 | 357 | ABU-357 |
| 1578 | 1938 | 118 | ABU-118 | 1698 | 2058 | 238 | ABU-238 | 1818 | 2178 | 358 | ABU-358 |
| 1579 | 1939 | 119 | ABU-119 | 1699 | 2059 | 239 | ABU-239 | 1819 | 2179 | 359 | ABU-359 |
| 1580 | 1940 | 120 | ABU-120 | 1700 | 2060 | 240 | ABU-240 | 1820 | 2180 | 360 | ABU-360 |
| 2901 | 2936 | 2971 | ABU-361 | 2913 | 2948 | 2983 | ABU-373 | 2925 | 2960 | 2995 | ABU-385 |
| 2902 | 2937 | 2972 | ABU-362 | 2914 | 2949 | 2984 | ABU-374 | 2926 | 2961 | 2996 | ABU-386 |
| 2903 | 2938 | 2973 | ABU-363 | 2915 | 2950 | 2985 | ABU-375 | 2927 | 2962 | 2997 | ABU-387 |
| 2904 | 2939 | 2974 | ABU-364 | 2916 | 2951 | 2986 | ABU-376 | 2928 | 2963 | 2998 | ABU-388 |
| 2905 | 2940 | 2975 | ABU-365 | 2917 | 2952 | 2987 | ABU-377 | 2929 | 2964 | 2999 | ABU-389 |
| 2906 | 2941 | 2976 | ABU-366 | 2918 | 2953 | 2988 | ABU-378 | 2930 | 2965 | 3000 | ABU-390 |
| 2907 | 2942 | 2977 | ABU-367 | 2919 | 2954 | 2989 | ABU-379 | 2931 | 2966 | 3001 | ABU-391 |
| 2908 | 2943 | 2978 | ABU-368 | 2920 | 2955 | 2990 | ABU-380 | 2932 | 2967 | 3002 | ABU-392 |
| 2909 | 2944 | 2979 | ABU-369 | 2921 | 2956 | 2991 | ABU-381 | 2933 | 2968 | 3003 | ABU-393 |
| 2910 | 2945 | 2980 | ABU-370 | 2922 | 2957 | 2992 | ABU-382 | 2934 | 2969 | 3004 | ABU-394 |
| 2911 | 2946 | 2981 | ABU-371 | 2923 | 2958 | 2993 | ABU-383 | 2935 | 2970 | 3005 | ABU-395 |
| 2912 | 2947 | 2982 | ABU-372 | 2924 | 2959 | 2994 | ABU-384 | | | | |

and the VL can comprise a sequence selected from combinations of CDR1, CDR2, and CDR3 as following:

| LCDR1 | LCDR2 | LCDR3 | ABU No. | LCDR1 | LCDR2 | LCDR3 | ABU No. | LCDR1 | LCDR2 | LCDR3 | ABU No. |
|---|---|---|---|---|---|---|---|---|---|---|---|
| 2181 | 2541 | 361 | ABU-1 | 2301 | 2661 | 481 | ABU-121 | 2421 | 2781 | 601 | ABU-241 |
| 2182 | 2542 | 362 | ABU-2 | 2302 | 2662 | 482 | ABU-122 | 2422 | 2782 | 602 | ABU-242 |
| 2183 | 2543 | 363 | ABU-3 | 2303 | 2663 | 483 | ABU-123 | 2423 | 2783 | 603 | ABU-243 |
| 2184 | 2544 | 364 | ABU-4 | 2304 | 2664 | 484 | ABU-124 | 2424 | 2784 | 604 | ABU-244 |
| 2185 | 2545 | 365 | ABU-5 | 2305 | 2665 | 485 | ABU-125 | 2425 | 2785 | 605 | ABU-245 |
| 2186 | 2546 | 366 | ABU-6 | 2306 | 2666 | 486 | ABU-126 | 2426 | 2786 | 606 | ABU-246 |
| 2187 | 2547 | 367 | ABU-7 | 2307 | 2667 | 487 | ABU-127 | 2427 | 2787 | 607 | ABU-247 |
| 2188 | 2548 | 368 | ABU-8 | 2308 | 2668 | 488 | ABU-128 | 2428 | 2788 | 608 | ABU-248 |
| 2189 | 2549 | 369 | ABU-9 | 2309 | 2669 | 489 | ABU-129 | 2429 | 2789 | 609 | ABU-249 |
| 2190 | 2550 | 370 | ABU-10 | 2310 | 2670 | 490 | ABU-130 | 2430 | 2790 | 610 | ABU-250 |
| 2191 | 2551 | 371 | ABU-11 | 2311 | 2671 | 491 | ABU-131 | 2431 | 2791 | 611 | ABU-251 |
| 2192 | 2552 | 372 | ABU-12 | 2312 | 2672 | 492 | ABU-132 | 2432 | 2792 | 612 | ABU-252 |
| 2193 | 2553 | 373 | ABU-13 | 2313 | 2673 | 493 | ABU-133 | 2433 | 2793 | 613 | ABU-253 |
| 2194 | 2554 | 374 | ABU-14 | 2314 | 2674 | 494 | ABU-134 | 2434 | 2794 | 614 | ABU-254 |
| 2195 | 2555 | 375 | ABU-15 | 2315 | 2675 | 495 | ABU-135 | 2435 | 2795 | 615 | ABU-255 |
| 2196 | 2556 | 376 | ABU-16 | 2316 | 2676 | 496 | ABU-136 | 2436 | 2796 | 616 | ABU-256 |
| 2197 | 2557 | 377 | ABU-17 | 2317 | 2677 | 497 | ABU-137 | 2437 | 2797 | 617 | ABU-257 |
| 2198 | 2558 | 378 | ABU-18 | 2318 | 2678 | 498 | ABU-138 | 2438 | 2798 | 618 | ABU-258 |
| 2199 | 2559 | 379 | ABU-19 | 2319 | 2679 | 499 | ABU-139 | 2439 | 2799 | 619 | ABU-259 |
| 2200 | 2560 | 380 | ABU-20 | 2320 | 2680 | 500 | ABU-140 | 2440 | 2800 | 620 | ABU-260 |
| 2201 | 2561 | 381 | ABU-21 | 2321 | 2681 | 501 | ABU-141 | 2441 | 2801 | 621 | ABU-261 |
| 2202 | 2562 | 382 | ABU-22 | 2322 | 2682 | 502 | ABU-142 | 2442 | 2802 | 622 | ABU-262 |
| 2203 | 2563 | 383 | ABU-23 | 2323 | 2683 | 503 | ABU-143 | 2443 | 2803 | 623 | ABU-263 |
| 2204 | 2564 | 384 | ABU-24 | 2324 | 2684 | 504 | ABU-144 | 2444 | 2804 | 624 | ABU-264 |
| 2205 | 2565 | 385 | ABU-25 | 2325 | 2685 | 505 | ABU-145 | 2445 | 2805 | 625 | ABU-265 |
| 2206 | 2566 | 386 | ABU-26 | 2326 | 2686 | 506 | ABU-146 | 2446 | 2806 | 626 | ABU-266 |
| 2207 | 2567 | 387 | ABU-27 | 2327 | 2687 | 507 | ABU-147 | 2447 | 2807 | 627 | ABU-267 |
| 2208 | 2568 | 388 | ABU-28 | 2328 | 2688 | 508 | ABU-148 | 2448 | 2808 | 628 | ABU-268 |
| 2209 | 2569 | 389 | ABU-29 | 2329 | 2689 | 509 | ABU-149 | 2449 | 2809 | 629 | ABU-269 |
| 2210 | 2570 | 390 | ABU-30 | 2330 | 2690 | 510 | ABU-150 | 2450 | 2810 | 630 | ABU-270 |
| 2211 | 2571 | 391 | ABU-31 | 2331 | 2691 | 511 | ABU-151 | 2451 | 2811 | 631 | ABU-271 |
| 2212 | 2572 | 392 | ABU-32 | 2332 | 2692 | 512 | ABU-152 | 2452 | 2812 | 632 | ABU-272 |
| 2213 | 2573 | 393 | ABU-33 | 2333 | 2693 | 513 | ABU-153 | 2453 | 2813 | 633 | ABU-273 |
| 2214 | 2574 | 394 | ABU-34 | 2334 | 2694 | 514 | ABU-154 | 2454 | 2814 | 634 | ABU-274 |
| 2215 | 2575 | 395 | ABU-35 | 2335 | 2695 | 515 | ABU-155 | 2455 | 2815 | 635 | ABU-275 |
| 2216 | 2576 | 396 | ABU-36 | 2336 | 2696 | 516 | ABU-156 | 2456 | 2816 | 636 | ABU-276 |
| 2217 | 2577 | 397 | ABU-37 | 2337 | 2697 | 517 | ABU-157 | 2457 | 2817 | 637 | ABU-277 |
| 2218 | 2578 | 398 | ABU-38 | 2338 | 2698 | 518 | ABU-158 | 2458 | 2818 | 638 | ABU-278 |
| 2219 | 2579 | 399 | ABU-39 | 2339 | 2699 | 519 | ABU-159 | 2459 | 2819 | 639 | ABU-279 |
| 2220 | 2580 | 400 | ABU-40 | 2340 | 2700 | 520 | ABU-160 | 2460 | 2820 | 640 | ABU-280 |
| 2221 | 2581 | 401 | ABU-41 | 2341 | 2701 | 521 | ABU-161 | 2461 | 2821 | 641 | ABU-281 |
| 2222 | 2582 | 402 | ABU-42 | 2342 | 2702 | 522 | ABU-162 | 2462 | 2822 | 642 | ABU-282 |
| 2223 | 2583 | 403 | ABU-43 | 2343 | 2703 | 523 | ABU-163 | 2463 | 2823 | 643 | ABU-283 |
| 2224 | 2584 | 404 | ABU-44 | 2344 | 2704 | 524 | ABU-164 | 2464 | 2824 | 644 | ABU-284 |
| 2225 | 2585 | 405 | ABU-45 | 2345 | 2705 | 525 | ABU-165 | 2465 | 2825 | 645 | ABU-285 |
| 2226 | 2586 | 406 | ABU-46 | 2346 | 2706 | 526 | ABU-166 | 2466 | 2826 | 646 | ABU-286 |
| 2227 | 2587 | 407 | ABU-47 | 2347 | 2707 | 527 | ABU-167 | 2467 | 2827 | 647 | ABU-287 |
| 2228 | 2588 | 408 | ABU-48 | 2348 | 2708 | 528 | ABU-168 | 2468 | 2828 | 648 | ABU-288 |
| 2229 | 2589 | 409 | ABU-49 | 2349 | 2709 | 529 | ABU-169 | 2469 | 2829 | 649 | ABU-289 |
| 2230 | 2590 | 410 | ABU-50 | 2350 | 2710 | 530 | ABU-170 | 2470 | 2830 | 650 | ABU-290 |
| 2231 | 2591 | 411 | ABU-51 | 2351 | 2711 | 531 | ABU-171 | 2471 | 2831 | 651 | ABU-291 |
| 2232 | 2592 | 412 | ABU-52 | 2352 | 2712 | 532 | ABU-172 | 2472 | 2832 | 652 | ABU-292 |
| 2233 | 2593 | 413 | ABU-53 | 2353 | 2713 | 533 | ABU-173 | 2473 | 2833 | 653 | ABU-293 |
| 2234 | 2594 | 414 | ABU-54 | 2354 | 2714 | 534 | ABU-174 | 2474 | 2834 | 654 | ABU-294 |
| 2235 | 2595 | 415 | ABU-55 | 2355 | 2715 | 535 | ABU-175 | 2475 | 2835 | 655 | ABU-295 |
| 2236 | 2596 | 416 | ABU-56 | 2356 | 2716 | 536 | ABU-176 | 2476 | 2836 | 656 | ABU-296 |
| 2237 | 2597 | 417 | ABU-57 | 2357 | 2717 | 537 | ABU-177 | 2477 | 2837 | 657 | ABU-297 |
| 2238 | 2598 | 418 | ABU-58 | 2358 | 2718 | 538 | ABU-178 | 2478 | 2838 | 658 | ABU-298 |
| 2239 | 2599 | 419 | ABU-59 | 2359 | 2719 | 539 | ABU-179 | 2479 | 2839 | 659 | ABU-299 |
| 2240 | 2600 | 420 | ABU-60 | 2360 | 2720 | 540 | ABU-180 | 2480 | 2840 | 660 | ABU-300 |
| 2241 | 2601 | 421 | ABU-61 | 2361 | 2721 | 541 | ABU-181 | 2481 | 2841 | 661 | ABU-301 |
| 2242 | 2602 | 422 | ABU-62 | 2362 | 2722 | 542 | ABU-182 | 2482 | 2842 | 662 | ABU-302 |
| 2243 | 2603 | 423 | ABU-63 | 2363 | 2723 | 543 | ABU-183 | 2483 | 2843 | 663 | ABU-303 |
| 2244 | 2604 | 424 | ABU-64 | 2364 | 2724 | 544 | ABU-184 | 2484 | 2844 | 664 | ABU-304 |
| 2245 | 2605 | 425 | ABU-65 | 2365 | 2725 | 545 | ABU-185 | 2485 | 2845 | 665 | ABU-305 |
| 2246 | 2606 | 426 | ABU-66 | 2366 | 2726 | 546 | ABU-186 | 2486 | 2846 | 666 | ABU-306 |
| 2247 | 2607 | 427 | ABU-67 | 2367 | 2727 | 547 | ABU-187 | 2487 | 2847 | 667 | ABU-307 |
| 2248 | 2608 | 428 | ABU-68 | 2368 | 2728 | 548 | ABU-188 | 2488 | 2848 | 668 | ABU-308 |
| 2249 | 2609 | 429 | ABU-69 | 2369 | 2729 | 549 | ABU-189 | 2489 | 2849 | 669 | ABU-309 |
| 2250 | 2610 | 430 | ABU-70 | 2370 | 2730 | 550 | ABU-190 | 2490 | 2850 | 670 | ABU-310 |
| 2251 | 2611 | 431 | ABU-71 | 2371 | 2731 | 551 | ABU-191 | 2491 | 2851 | 671 | ABU-311 |
| 2252 | 2612 | 432 | ABU-72 | 2372 | 2732 | 552 | ABU-192 | 2492 | 2852 | 672 | ABU-312 |
| 2253 | 2613 | 433 | ABU-73 | 2373 | 2733 | 553 | ABU-193 | 2493 | 2853 | 673 | ABU-313 |
| 2254 | 2614 | 434 | ABU-74 | 2374 | 2734 | 554 | ABU-194 | 2494 | 2854 | 674 | ABU-314 |
| 2255 | 2615 | 435 | ABU-75 | 2375 | 2735 | 555 | ABU-195 | 2495 | 2855 | 675 | ABU-315 |
| 2256 | 2616 | 436 | ABU-76 | 2376 | 2736 | 556 | ABU-196 | 2496 | 2856 | 676 | ABU-316 |
| 2257 | 2617 | 437 | ABU-77 | 2377 | 2737 | 557 | ABU-197 | 2497 | 2857 | 677 | ABU-317 |
| 2258 | 2618 | 438 | ABU-78 | 2378 | 2738 | 558 | ABU-198 | 2498 | 2858 | 678 | ABU-318 |
| 2259 | 2619 | 439 | ABU-79 | 2379 | 2739 | 559 | ABU-199 | 2499 | 2859 | 679 | ABU-319 |
| 2260 | 2620 | 440 | ABU-80 | 2380 | 2740 | 560 | ABU-200 | 2500 | 2860 | 680 | ABU-320 |
| 2261 | 2621 | 441 | ABU-81 | 2381 | 2741 | 561 | ABU-201 | 2501 | 2861 | 681 | ABU-321 |
| 2262 | 2622 | 442 | ABU-82 | 2382 | 2742 | 562 | ABU-202 | 2502 | 2862 | 682 | ABU-322 |
| 2263 | 2623 | 443 | ABU-83 | 2383 | 2743 | 563 | ABU-203 | 2503 | 2863 | 683 | ABU-323 |
| 2264 | 2624 | 444 | ABU-84 | 2384 | 2744 | 564 | ABU-204 | 2504 | 2864 | 684 | ABU-324 |
| 2265 | 2625 | 445 | ABU-85 | 2385 | 2745 | 565 | ABU-205 | 2505 | 2865 | 685 | ABU-325 |
| 2266 | 2626 | 446 | ABU-86 | 2386 | 2746 | 566 | ABU-206 | 2506 | 2866 | 686 | ABU-326 |
| 2267 | 2627 | 447 | ABU-87 | 2387 | 2747 | 567 | ABU-207 | 2507 | 2867 | 687 | ABU-327 |
| 2268 | 2628 | 448 | ABU-88 | 2388 | 2748 | 568 | ABU-208 | 2508 | 2868 | 688 | ABU-328 |
| 2269 | 2629 | 449 | ABU-89 | 2389 | 2749 | 569 | ABU-209 | 2509 | 2869 | 689 | ABU-329 |
| 2270 | 2630 | 450 | ABU-90 | 2390 | 2750 | 570 | ABU-210 | 2510 | 2870 | 690 | ABU-330 |
| 2271 | 2631 | 451 | ABU-91 | 2391 | 2751 | 571 | ABU-211 | 2511 | 2871 | 691 | ABU-331 |
| 2272 | 2632 | 452 | ABU-92 | 2392 | 2752 | 572 | ABU-212 | 2512 | 2872 | 692 | ABU-332 |
| 2273 | 2633 | 453 | ABU-93 | 2393 | 2753 | 573 | ABU-213 | 2513 | 2873 | 693 | ABU-333 |
| 2274 | 2634 | 454 | ABU-94 | 2394 | 2754 | 574 | ABU-214 | 2514 | 2874 | 694 | ABU-334 |
| 2275 | 2635 | 455 | ABU-95 | 2395 | 2755 | 575 | ABU-215 | 2515 | 2875 | 695 | ABU-335 |
| 2276 | 2636 | 456 | ABU-96 | 2396 | 2756 | 576 | ABU-216 | 2516 | 2876 | 696 | ABU-336 |
| 2277 | 2637 | 457 | ABU-97 | 2397 | 2757 | 577 | ABU-217 | 2517 | 2877 | 697 | ABU-337 |
| 2278 | 2638 | 458 | ABU-98 | 2398 | 2758 | 578 | ABU-218 | 2518 | 2878 | 698 | ABU-338 |
| 2279 | 2639 | 459 | ABU-99 | 2399 | 2759 | 579 | ABU-219 | 2519 | 2879 | 699 | ABU-339 |
| 2280 | 2640 | 460 | ABU-100 | 2400 | 2760 | 580 | ABU-220 | 2520 | 2880 | 700 | ABU-340 |
| 2281 | 2641 | 461 | ABU-101 | 2401 | 2761 | 581 | ABU-221 | 2521 | 2881 | 701 | ABU-341 |
| 2282 | 2642 | 462 | ABU-102 | 2402 | 2762 | 582 | ABU-222 | 2522 | 2882 | 702 | ABU-342 |
| 2283 | 2643 | 463 | ABU-103 | 2403 | 2763 | 583 | ABU-223 | 2523 | 2883 | 703 | ABU-343 |
| 2284 | 2644 | 464 | ABU-104 | 2404 | 2764 | 584 | ABU-224 | 2524 | 2884 | 704 | ABU-344 |
| 2285 | 2645 | 465 | ABU-105 | 2405 | 2765 | 585 | ABU-225 | 2525 | 2885 | 705 | ABU-345 |
| 2286 | 2646 | 466 | ABU-106 | 2406 | 2766 | 586 | ABU-226 | 2526 | 2886 | 706 | ABU-346 |
| 2287 | 2647 | 467 | ABU-107 | 2407 | 2767 | 587 | ABU-227 | 2527 | 2887 | 707 | ABU-347 |
| 2288 | 2648 | 468 | ABU-108 | 2408 | 2768 | 588 | ABU-228 | 2528 | 2888 | 708 | ABU-348 |
| 2289 | 2649 | 469 | ABU-109 | 2409 | 2769 | 589 | ABU-229 | 2529 | 2889 | 709 | ABU-349 |
| 2290 | 2650 | 470 | ABU-110 | 2410 | 2770 | 590 | ABU-230 | 2530 | 2890 | 710 | ABU-350 |
| 2291 | 2651 | 471 | ABU-111 | 2411 | 2771 | 591 | ABU-231 | 2531 | 2891 | 711 | ABU-351 |
| 2292 | 2652 | 472 | ABU-112 | 2412 | 2772 | 592 | ABU-232 | 2532 | 2892 | 712 | ABU-352 |
| 2293 | 2653 | 473 | ABU-113 | 2413 | 2773 | 593 | ABU-233 | 2533 | 2893 | 713 | ABU-353 |
| 2294 | 2654 | 474 | ABU-114 | 2414 | 2774 | 594 | ABU-234 | 2534 | 2894 | 714 | ABU-354 |
| 2295 | 2655 | 475 | ABU-115 | 2415 | 2775 | 595 | ABU-235 | 2535 | 2895 | 715 | ABU-355 |
| 2296 | 2656 | 476 | ABU-116 | 2416 | 2776 | 596 | ABU-236 | 2536 | 2896 | 716 | ABU-356 |
| 2297 | 2657 | 477 | ABU-117 | 2417 | 2777 | 597 | ABU-237 | 2537 | 2897 | 717 | ABU-357 |
| 2298 | 2658 | 478 | ABU-118 | 2418 | 2778 | 598 | ABU-238 | 2538 | 2898 | 718 | ABU-358 |
| 2299 | 2659 | 479 | ABU-119 | 2419 | 2779 | 599 | ABU-239 | 2539 | 2899 | 719 | ABU-359 |
| 2300 | 2660 | 480 | ABU-120 | 2420 | 2780 | 600 | ABU-240 | 2540 | 2900 | 720 | ABU-360 |
| 3006 | 3041 | 3076 | ABU-361 | 3018 | 3053 | 3088 | ABU-373 | 3030 | 3065 | 3100 | ABU-385 |
| 3007 | 3042 | 3077 | ABU-362 | 3019 | 3054 | 3089 | ABU-374 | 3031 | 3066 | 3101 | ABU-386 |
| 3008 | 3043 | 3078 | ABU-363 | 3020 | 3055 | 3090 | ABU-375 | 3032 | 3067 | 3102 | ABU-387 |
| 3009 | 3044 | 3079 | ABU-364 | 3021 | 3056 | 3091 | ABU-376 | 3033 | 3068 | 3103 | ABU-388 |
| 3010 | 3045 | 3080 | ABU-365 | 3022 | 3057 | 3092 | ABU-377 | 3034 | 3069 | 3104 | ABU-389 |
| 3011 | 3046 | 3081 | ABU-366 | 3023 | 3058 | 3093 | ABU-378 | 3035 | 3070 | 3105 | ABU-390 |
| 3012 | 3047 | 3082 | ABU-367 | 3024 | 3059 | 3094 | ABU-379 | 3036 | 3071 | 3106 | ABU-391 |
| 3013 | 3048 | 3083 | ABU-368 | 3025 | 3060 | 3095 | ABU-380 | 3037 | 3072 | 3107 | ABU-392 |
| 3014 | 3049 | 3084 | ABU-369 | 3026 | 3061 | 3096 | ABU-381 | 3038 | 3073 | 3108 | ABU-393 |
| 3015 | 3050 | 3085 | ABU-370 | 3027 | 3062 | 3097 | ABU-382 | 3039 | 3074 | 3109 | ABU-394 |
| 3016 | 3051 | 3086 | ABU-371 | 3028 | 3063 | 3098 | ABU-383 | 3040 | 3075 | 3110 | ABU-395 |
| 3017 | 3052 | 3087 | ABU-372 | 3029 | 3064 | 3099 | ABU-384 | | | | |

The VH CDR1 of the antigen-binding unit of the present invention can comprise the same sequence as CDR1 contained in SEQ ID NOs: 721-1080 and 3111-3145; the VH CDR2 of the antigen-binding unit of the present invention can comprise the same sequence as CDR2 contained in SEQ ID NOs: 721-1080 and 3111-3145; the VH CDR3 of the antigen-binding unit of the present invention can comprise the same sequence as CDR3 contained in SEQ ID NOs: 721-1080 and 3111-3145; the VL CDR1 of the antigen-binding unit can comprise the same sequence as CDR1 contained in SEQ ID NOs: 1081-1440 and 3146-3180; the VL CDR2 of the antigen-binding unit can comprise the same sequence as CDR2 contained in SEQ ID NOs: 1081-1440 and 3146-3180; and/or the VL CDR3 of the antigen-binding unit can comprise the same sequence as CDR3 contained in SEQ ID NOs: 1081-1440 and 3146-3180.

In one embodiment, the antibody provided in the present invention comprises one, two, three, four, five or six amino acid sequences, wherein each amino acid sequence is independently selected from the amino acid sequences listed below:

a. amino acid sequences of light chain variable region CDR1: SEQ ID NO: 2354, SEQ ID NO: 2355, SEQ ID NO: 2370, SEQ ID NO: 2477, and SEQ ID NO: 3012;

b. amino acid sequences of light chain variable region CDR2: SEQ ID NO: 2714, SEQ ID NO: 2715, SEQ ID NO: 2730, SEQ ID NO: 2837, and SEQ ID NO: 3047;

c. amino acid sequences of light chain variable region CDR3: SEQ ID NO: 534, SEQ ID NO: 535, SEQ ID NO: 550, SEQ ID NO: 657, and SEQ ID NO: 3082;

d. amino acid sequences of heavy chain variable region CDR1: SEQ ID NO: 1634, SEQ ID NO: 1635, SEQ ID NO: 1650, SEQ ID NO: 1757, and SEQ ID NO: 2907;

e. amino acid sequences of heavy chain variable region CDR2: SEQ ID NO: 1994, SEQ ID NO: 1995, SEQ ID NO: 2010, SEQ ID NO: 2117, and SEQ ID NO: 2942; and

f. amino acid sequences of heavy chain variable region CDR3: SEQ ID NO: 174, SEQ ID NO: 175, SEQ ID NO: 190, SEQ ID NO: 297, and SEQ ID NO: 2977.

In one embodiment, the antibody provided in the present invention comprises one, two, three, four, five or six amino acid sequences, wherein each amino acid sequence is independently selected from the amino acid sequences listed below:

a. amino acid sequences of light chain variable region CDR1: SEQ ID NO: 2354;

b. amino acid sequences of light chain variable region CDR2: SEQ ID NO: 2714;

c. amino acid sequences of light chain variable region CDR3: SEQ ID NO: 534;

d. amino acid sequences of heavy chain variable region CDR1: SEQ ID NO: 1634;

e. amino acid sequences of heavy chain variable region CDR2: SEQ ID NO: 1994; and

f. amino acid sequences of heavy chain variable region CDR3: SEQ ID NO: 174.

In one embodiment, the antibody provided in the present invention comprises one, two, three, four, five or six amino acid sequences, wherein each amino acid sequence is independently selected from the amino acid sequences listed below:

a. amino acid sequences of light chain variable region CDR1: SEQ ID NO: 2355;

b. amino acid sequences of light chain variable region CDR2: SEQ ID NO: 2715;

c. amino acid sequences of light chain variable region CDR3: SEQ ID NO: 535;

d. amino acid sequences of heavy chain variable region CDR1: SEQ ID NO: 1635;

e. amino acid sequences of heavy chain variable region CDR2: SEQ ID NO: 1995; and

f. amino acid sequences of heavy chain variable region CDR3: SEQ ID NO: 175.

In one embodiment, the antibody provided in the present invention comprises one, two, three, four, five or six amino acid sequences, wherein each amino acid sequence is independently selected from the amino acid sequences listed below:

a. amino acid sequences of light chain variable region CDR1: SEQ ID NO: 2370;

b. amino acid sequences of light chain variable region CDR2: SEQ ID NO: 2730;

c. amino acid sequences of light chain variable region CDR3: SEQ ID NO: 550;

d. amino acid sequences of heavy chain variable region CDR1: SEQ ID NO: 1650;

e. amino acid sequences of heavy chain variable region CDR2: SEQ ID NO: 2010; and

f. amino acid sequences of heavy chain variable region CDR3: SEQ ID NO: 190.

In one embodiment, the antibody provided in the present invention comprises one, two, three, four, five or six amino acid sequences, wherein each amino acid sequence is independently selected from the amino acid sequences listed below:

a. amino acid sequences of light chain variable region CDR1: SEQ ID NO: 2477;

b. amino acid sequences of light chain variable region CDR2: SEQ ID NO: 2837;

c. amino acid sequences of light chain variable region CDR3: SEQ ID NO: 657;

d. amino acid sequences of heavy chain variable region CDR1: SEQ ID NO: 1757;

e. amino acid sequences of heavy chain variable region CDR2: SEQ ID NO: 2117; and

f. amino acid sequences of heavy chain variable region CDR3: SEQ ID NO: 297.

In one embodiment, the antibody provided in the present invention comprises one, two, three, four, five or six amino acid sequences, wherein each amino acid sequence is independently selected from the amino acid sequences listed below:

a. amino acid sequences of light chain variable region CDR1: SEQ ID NO: 3012;

b. amino acid sequences of light chain variable region CDR2: SEQ ID NO: 3047;

c. amino acid sequences of light chain variable region CDR3: SEQ ID NO: 3082;

d. amino acid sequences of heavy chain variable region CDR1: SEQ ID NO: 2907;

e. amino acid sequences of heavy chain variable region CDR2: SEQ ID NO: 2942; and

f. amino acid sequences of heavy chain variable region CDR3: SEQ ID NO: 2977.

In one embodiment, the antibody provided in the present invention comprises one or two amino acid sequences, wherein each amino acid sequence is independently selected from the amino acid sequences listed below:

a. amino acid sequences of a light chain variable region: SEQ ID NO: 1377 and SEQ ID NO: 3152; and

b. amino acid sequences of a heavy chain variable region: SEQ ID NO: 1017 and SEQ ID NO: 3117.

In one embodiment, the antibody provided in the present invention comprises one or two amino acid sequences, wherein each amino acid sequence is independently selected from the amino acid sequences listed below:

a. amino acid sequences of a light chain variable region: SEQ ID NO: 1254; and

b. amino acid sequences of a heavy chain variable region: SEQ ID NO: 894.

In one embodiment, the antibody provided in the present invention comprises one or two amino acid sequences, wherein each amino acid sequence is independently selected from the amino acid sequences listed below:

a. amino acid sequences of a light chain variable region: SEQ ID NO: 1255; and

b. amino acid sequences of a heavy chain variable region: SEQ ID NO: 895.

In one embodiment, the antibody provided in the present invention comprises one or two amino acid sequences, wherein each amino acid sequence is independently selected from the amino acid sequences listed below:

a. amino acid sequences of a light chain variable region: SEQ ID NO: 1270; and

b. amino acid sequences of a heavy chain variable region: SEQ ID NO: 910.

In one embodiment, the antibody provided in the present invention comprises one or two amino acid sequences, wherein each amino acid sequence is independently selected from the amino acid sequences listed below:

a. amino acid sequences of a light chain variable region: SEQ ID NO: 1377; and

b. amino acid sequences of a heavy chain variable region: SEQ ID NO: 1017.

In one embodiment, the antibody provided in the present invention comprises one or two amino acid sequences, wherein each amino acid sequence is independently selected from the amino acid sequences listed below:

a. amino acid sequences of a light chain variable region: SEQ ID NO: 3152; and

b. amino acid sequences of a heavy chain variable region: SEQ ID NO: 3117.

The antigen-binding unit of the present invention can bind to the S protein of a novel coronavirus (SARS-CoV-2). The antigen-binding unit of the present invention can bind to a receptor binding domain (RBD) of an S protein of a novel coronavirus (SARS-CoV-2). Binding of the antigen-binding unit to the RBD can be characterized or represented by any method known in the art. For example, binding can be characterized by binding affinity, which can be the strength of the interaction between the antigen-binding unit and the antigen. Binding affinity can be determined by any method known in the art, such as in vitro binding experiment. The binding affinity of the antigen-binding unit of the present invention can be represented by KD, which is defined as the ratio of two kinetic rate constants Ka/Kd, wherein “Ka” refers to the rate constant for the binding of an antibody to an antigen and “Kd” refers to the rate constant for the dissociation of the antibody from the antibody/antigen complex. The antigen-binding unit as disclosed herein specifically binds to a receptor binding domain (RBD) of an S protein of a novel coronavirus (SARS-CoV-2) with a KD in the range of about 10 μM to about 1 fM. For example, the antigen-binding unit can specifically bind to a receptor binding domain (RBD) of an S protein of a novel coronavirus (SARS-CoV-2) with a KD of less than about 10 μM, 1 μM, 0.1 μM, 50 nM, 20 nM, 15 nM, 10 nM, 5 nM, 4 nM, 3 nM, 2 nM, 1 nM, 0.5 nM, 0.1 nM, 50 pM, 10 pM, 1 pM, 0.1 pM, 10 fM, 1 fM, 0.1 fM or less than 0.1 fM. The antigen-binding unit disclosed herein can bind to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

The antigen-binding unit of the present invention has a neutralizing activity against a novel coronavirus (SARS-CoV-2). The neutralizing activity of the antigen-binding unit of the present invention against the novel coronavirus (SARS-CoV-2) can be analyzed using pseudovirus. The pseudovirus has similar cell infection characteristics to the euvirus, can be used to simulate the early process of euvirus infection in a cell, and can be safely and quickly detected and analyzed. The neutralizing activity of the antigen-binding unit of the present invention against the novel coronavirus (SARS-CoV-2) can be detected by a method known in the art, such as using cell microneutralization assay, which is performed with reference to the description of Temperton N J et al., Emerg Infect Dis, 2005, 11(3), 411-416.

The neutralizing activity of the antigen-binding unit of the present invention against the novel coronavirus (SARS-CoV-2) can be detected by using an experimental cell, such as Huh-7 cell and pseudovirus SARS-CoV-2. The antigen-binding unit of the present invention can neutralize the novel coronavirus (SARS-CoV-2) pseudovirus with an IC₅₀ of less than 100 μg/ml, less than 50 μg/ml, less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, less than 1 ng/ml, less than 0.5 ng/ml, less than 0.25 ng/ml, less than 0.2 ng/ml, less than 0.1 ng/ml, less than 50 pg/ml, less than 25 pg/ml, less than 20 pg/ml, less than 10 pg/ml, less than 5 pg/ml, less than 2.5 pg/ml, less than 2 pg/ml, or less than 1 pg/ml.

The neutralizing activity of the antigen-binding unit of the present invention against the novel coronavirus (SARS-CoV-2) can be detected by Plaque Reduction Neutralization Test (PRNT) using a SARS-CoV-2 euvirus, wherein the IC₅₀ of the antigen-binding unit of the present invention for neutralization of the SARS-CoV-2 euvirus is calculated according to the reduction of plaques after incubation. The antigen-binding unit of the present invention can neutralize the novel coronavirus (SARS-CoV-2) euvirus with an IC₅₀ of less than 100 μg/ml, less than 50 μg/ml, less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, less than 1 ng/ml, less than 0.5 ng/ml, less than 0.25 ng/ml, less than 0.2 ng/ml, less than 0.1 ng/ml, less than 50 pg/ml, less than 25 pg/ml, less than 20 pg/ml, less than 10 pg/ml, less than 5 pg/ml, less than 2.5 pg/ml, less than 2 pg/ml, or less than 1 pg/ml.

### Preparation of Antigen-Binding Unit

Provided herein is a method for producing any of the antigen-binding units disclosed herein, wherein the method comprises culturing a host cell expressing the antigen-binding unit under conditions suitable for the expression of the antigen-binding unit and isolating the antigen-binding unit expressed by the host cell.

The expressed antigen-binding unit can be isolated using various protein purification techniques known in the art. Generally, the antigen-binding units are isolated from media as secreted polypeptides, although they can also be recovered from a host cell lysate or bacterial periplasm when produced directly in the absence of a signal peptide. If the antigen-binding units are membrane-bound, they can be dissolved in a suitable detergent solution commonly used by a person skilled in the art. The recovered antigen-binding units can be further purified by salt precipitation (e.g., with ammonium sulfate), ion exchange chromatography (e.g., running on a cation or anion exchange column at neutral pH and eluting with a step gradient of increasing ionic strength), gel filtration chromatography (including gel filtration HPLC) and tag affinity column chromatography, or affinity resin, such as protein A, protein G, hydroxyapatite and anti-immunoglobulins.

The derived immunoglobulins to which the following moieties are added can be used in the methods and compositions of the present invention: a chemical linker, a detectable moiety such as a fluorescent dye, an enzyme, a substrate, a chemiluminescent moiety, a specific binding moiety such as streptavidin, avidin or biotin, or a drug conjugate.

The present invention further provides an antigen-binding unit conjugated to a chemically functional moiety. Generally, the moiety is a label capable of producing a detectable signal. These conjugated antigen-binding units can be used, for example, in a detection system, such as for detecting the severity of viral infection, imaging of infection focus, etc. Such labels are known in the art and include but are not limited to a radioisotope, an enzyme, a fluorescent compound, a chemiluminescent compound, a bioluminescent compound, a substrate, a cofactor and an inhibitor. See U.S. Pat. Nos. 3,817,837; 3,850,752; 3,939,350; 3,996,345; 4,277,437; 4,275,149; and 4,366,241 for examples of patents teaching the use of such labels. The moiety can be covalently linked or recombinantly linked to the antigen-binding unit, or conjugated to the antigen-binding unit via a second reagent such as a second antibody, protein A or a biotin-avidin complex.

Other functional moieties include a signal peptide, a reagent enhancing immunoreactivity, a reagent facilitating coupling to a solid support, a vaccine carrier, a biological response modifier, a paramagnetic label, and a drug. The signal peptide is a short amino acid sequence that guides a newly synthesized protein through the cell membrane (usually the endoplasmic reticulum in an eukaryotic cell) and the inner membrane or both inner and outer membranes of a bacterium. The signal peptide can be located at the N-terminal portion of a polypeptide or the C-terminal portion of a polypeptide, and can be enzymatically removed from the cell between the biosynthesis and secretion of the polypeptide. Such peptides can be introduced into the antigen-binding unit to allow secretion of a synthetic molecule.

The reagent enhancing immunoreactivity includes but is not limited to a bacterial superantigen. The reagent facilitating coupling to a solid support includes but is not limited to biotin or avidin. The immunogen carrier includes but is not limited to, any physiologically acceptable buffers. The biological response modifier includes a cytokine, particularly tumor necrosis factor (TNF), interleukin-2, interleukin-4, granulocyte macrophage colony stimulating factor and y-interferon.

The chemically functional moiety can be prepared recombinantly, for example by generating a fusion gene encoding the antigen-binding unit and the functional moiety. Alternatively, the antigen-binding unit can be chemically bonded to the moiety by any of various well-known chemical procedures. For example, when the moiety is a protein, the linkage can be achieved by a heterobifunctional crosslinking agent, e.g., SPDP, carbodiimide glutaraldehyde, etc. The moiety can be covalently linked or conjugated via a second reagent, such as a second antibody, protein A or a biotin-avidin complex. The paramagnetic moiety and the conjugation thereof to an antibody are well known in the art. See, for example, Miltenyi et al. (1990) Cytometry 11:231-238.

### Nucleic Acids

In one aspect, provided herein is an isolated polynucleotide encoding the antigen-binding unit of the present invention. Nucleotide sequences corresponding to various regions of the L or H chain of an existing antibody can be readily obtained and sequenced using conventional techniques including, but not limited to, hybridization, PCR, and DNA sequencing. The hybridoma cell producing a monoclonal antibody is used as a preferred source of an antibody nucleotide sequence. Large numbers of hybridoma cells producing a series of monoclonal antibodies may be obtained from a public or private repositories. The largest storage institution is the American Type Culture Collection, which provides a variety of well-characterized hybridoma cell lines. Alternatively, the antibody nucleotide can be obtained from an immunized or non-immunized rodent or human, and from an organ such as spleen and peripheral blood lymphocyte. Specific techniques suitable for extraction and synthesis of antibody nucleotides are described in Orlandi et al. (1989) Proc. Natl. Acad. Sci. U.S.A 86: 3833-3837; Larrick et al. (1989) biochem. Biophys. Res. Commun. 160: 1250-1255; Sastry et al. (1989) Proc. Natl. Acad. Sci., U.S.A. 86: 5728-5732; and U.S. Pat. No. 5,969,108.

The antibody nucleotide sequence can also be modified, for example, by substituting human heavy and light chain constant regions with coding sequences, to replace homologous non-human sequences. The chimeric antibody prepared in this manner retains the binding specificity of the original antibody.

In addition, the polynucleotide encoding the heavy chain and/or light chain of the antigen-binding unit can be subjected to codon optimization to achieve optimized expression of the antigen-binding unit of the subject in a desired host cell. For example, in one codon optimization method, a natural codon is substituted by the most common codon from the reference genome, wherein the translation rate of the codon for each amino acid is designed to be relatively high. Additional exemplary methods for generating a codon-optimized polynucleotide for expressing the desired protein are described in Kanaya et al., Gene, 238:143-155 (1999), Wang et al., Mol. Biol. Evol., 18(5):792-800 (2001), U.S. Pat. No. 5,795,737, US Publication No. 2008/0076161 and WO 2008/000632, and the methods can be applied to the heavy chain and/or light chain of the antigen-binding unit.

The polynucleotides of the present invention includes polynucleotides encoding a functional equivalent of the exemplary polypeptide and a fragment thereof.

Due to the degeneracy of the genetic code, there can be considerable variation in the nucleotides of the L and H sequences and a heterodimerization sequence suitable for construction of the polynucleotide and vector of the present invention. These variations are included in the present invention.

### Method of Treatment

Provided herein is a method for preventing or treating a novel coronavirus (SARS-CoV-2) infection in a subject by using the antigen-binding unit of the present invention, comprising administering to the subject the antigen-binding unit of the present invention.

Provided herein is a method for treating a disease, condition or disorder in a mammal using the antigen-binding unit of the present invention in combination with a second agent. The second agent can be administered with, before or after an antibody. The second agent may be an antiviral agent. The antiviral agent includes but is not limited to telaprevir, boceprevir, semiprevir, sofosbuvir, daclastavir, asunaprevir, lamivudine, adefovir, entecavir, tenofovir, telbivudine, interferon α and PEGylated interferon α. The second agent can be selected from hydroxychloroquine, chloroquine, favipiravir, Gimsilumab, AdCOVID (University of Alabama at Birmingham), AT-100 (Airway Therapeutics), TZLS-501 (Tiziana Life Sciences), OYA1 (OyaGen), BPI-002 (BeyondSpring), INO-4800 (Inovio Pharmaceutical), NP-120 (ifenprodil), remdesivir (GS-5734), Actemra (Roche), Galidesivir (BCX4430), SNG001 (Synairgen Research), or a combination thereof.

The second agent may be an agent for alleviating symptoms of a concurrent inflammatory condition in a subject. The anti-inflammatory agent includes non-steroidal anti-inflammatory drugs (NSAIDs) and corticosteroids. NSAID includes but is not limited to salicylate, such as acetylsalicylic acid; diflunisal, salicylic acid and salsalate; propionic acid derivative, such as ibuprofen; naproxen; dexibuprofen, dexketoprofen, flurbiprofen, oxaprozin, fenoprofen, loxoprofen, and ketoprofen; acetic acid derivative such as indomethacin, diclofenac, tolmetin, aceclofenac, sulindac, nabumetone, etodolac and ketorolac; enolic acid derivative such as piroxicam, lornoxicam, meloxicam, isoxicam, tenoxicam, phenylbutazone and droxicam; anthranilic acid derivative such as mefenamic acid, flufenamic acid, meclofenamic acid and tolfenamic acid; selective COX-2 inhibitor, such as celecoxib, lumiracoxib, rofecoxib, etoricoxib, valdecoxib, firocoxib, and parecoxib; sulfonanilide, such as nimesulide; and other non-steroidal anti-inflammatory drugs such as clonixin and licofelone. The corticosteroids include but are not limited to cortisone, dexamethasone, hydrocortisone, methylprednisolone, prednisone, and prednisolone.

The second agent may be an immunosuppressive agent. The immunosuppressive agent that can be used in combination with the antigen-binding unit includes but is not limited to hydroxychloroquine, sulfasalazine, leflunomide, etanercept, infliximab, adalimumab, D-penicillamine, oral gold compound, injectable gold compound (by intramuscular injection), minocycline, gold sodium thiomalate, auranofin, D-penicillamine, lobenzarit, bucillamine, actarit, cyclophosphamide, azathioprine, methotrexate, mizoribine, cyclosporin and tacrolimus.

The specific dose will vary depending on the specific antigen-binding unit selected, the dosing regimen to be followed, whether it is administered in combination with other agents, the time of administration, the tissue to which it is administered, and the physical delivery system carrying the specific antigen-binding unit. In some embodiments, during the treatment cycle, the antigen-binding unit is administered to the subject at a dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 mg per week on average. For example, the antigen-binding unit is administered to the subject at a dose of about 35, 36, 37, 38, 39, 40, 41, 42, 43, 44, 45, 46, 47, 48, 49, 50, 51, 52, 53, 54 or 55 mg per week. In some embodiments, the antigen-binding unit is administered to the subject at a dose of about 40, 41, 42, 43, 44, 45, 46, 47, 48, 49, 50, 51, 52, 53, 54 or 55 mg per week.

During the treatment cycle, the antigen-binding unit can be administered to the subject at a dose of greater than 1, 1.5, 2, 2.5, 3, 3.5, 4, 4.5, 5, 5.5, 6, 6.5, 7, 7.5, 8, 8.5, 9, 9.5 or 10 mg per day on average. For example, during the treatment cycle, the antigen-binding unit is administered to the subject at a dose of about 6 to 10 mg, about 6.5 to 9.5 mg, about 6.5 to 8.5 mg, about 6.5 to 8 mg, or about 7 to 9 mg per day on average.

The dose of the antigen-binding unit can be about, at least about, or at most about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 mg or mg/kg, or any range derived therefrom. It is contemplated that the dose in mg/kg refers to the amount of the antigen-binding unit in mg per kilogram of the total body weight of the subject. It is contemplated that when multiple doses are administered to a patient, the doses can vary in amount or can be the same.

### Pharmaceutical Composition

Provided herein is a pharmaceutical composition comprising a subject antibody or a functional fragment thereof and a pharmaceutically acceptable carrier, excipient or stabilizer, including, but not limited to, an inert solid diluent and a filler, a diluent, a sterile aqueous solution and various organic solvents, a penetration enhancer, a solubilizer and an adjuvant. (Remington's Pharmaceutical Sciences 16th edition, Osol, A. Ed. (1980)).

The pharmaceutical composition can be in a unit dosage form suitable for single administration at a precise dose. The pharmaceutical composition can further comprise an antigen-binding unit as an active ingredient, and may include a conventional pharmaceutical carrier or excipient. In addition, it may include other drugs or agents, carriers, adjuvants, etc. An exemplary parenteral administration form includes a solution or suspension of an active polypeptide and/or PEG-modified polypeptide in a sterile aqueous solution, such as aqueous propylene glycol or dextrose solution. If desired, such dosage forms can be suitably buffered with a salt such as histidine and/or phosphate.

The composition can further include one or more pharmaceutically acceptable additives and excipients. These additives and excipients include but are not limited to an anti-adhesive agent, an anti-foaming agent, a buffer, a polymer, an antioxidant, a preservative, a chelating agent, a viscomodulator, a tension regulator, a flavoring agent, a colorant, a flavor enhancer, an opacifier, a suspending agent, a binder, a filler, a plasticizer, a lubricant and a mixture thereof.

### Kit

The kit of the present invention comprises the antigen-binding unit of the present invention or a conjugate thereof of the present invention. Further provided is the use of the antigen-binding unit of the present invention in the preparation of a kit, wherein the kit is used for detecting presence of a novel coronavirus, an S protein thereof or a RBD of the S protein, or a level thereof in a sample, or for diagnosing whether a subject is infected with the novel coronavirus.

In some embodiments, the sample includes, but is not limited to, an excrement, an oral or nasal secretion, an alveolar lavage fluid, etc. from a subject (e.g., mammal, preferably human).

General methods for detecting presence of a target virus or antigen (e.g., a novel coronavirus, or an S protein thereof or a RBD of the S protein) or a level thereof in a sample by using an antibody or an antigen-binding fragment thereof is well known to a person skilled in the art. In some embodiments, the detection method may involve enzyme linked immunosorbent assay (ELISA), enzyme immunodetection, chemiluminescence immunodetection, radioimmunodetection, fluorescence immunodetection, immunochromatography, a competition method, and a similar detection method.

### EXAMPLES

The present invention is described with reference to the following examples, which are meant to illustrate the present invention (but not limit the present invention).

Unless specifically stated, the molecular biology experimental methods and immunodetection methods used in the present invention were basically carried out with reference to J. Sambrook et al., Molecular Cloning: A Laboratory Manual, 2nd Edition, Cold Spring Harbor Laboratory Press, 1989 and F. M. Ausubel et al., Short Protocols in Molecular Biology, 3rd Edition, John Wiley & Sons, Inc., 1995; and restriction enzymes were used under the conditions as recommended by the product manufacturer. If no specific conditions are indicated in the examples, conventional conditions or the conditions suggested by the manufacturer shall be followed. The reagents or instruments used without indicating the manufacturers are commercially available conventional products. It is known to a person skilled in the art that the examples illustrate the present invention by way of example and are not intended to limit the claimed scope of the present invention.

### Example 1: Isolation of Memory B Cell

Blood was collected from people once infected with SARS-CoV-2 virus but recovered and discharged (provided by Beijing Youan Hospital), and PBMCs were extracted using STEMCELL SepMate™-15 (Stemcell Technologies, Cat #86415) in a Biosafety Physical Containment Level-2+ Laboratory. Then, memory B cells were enriched from the extracted PBMCs using STEMCELL EasySep Human Memory B Cell Isolation Kit (Stemcell Technologies, Cat #17864) according to the manufacturer's instructions.

### Example 2: Acquisition and Identification of Sequence of Antigen-Binding Unit

Single-cell transcriptome VDJ sequencing of the above-mentioned enriched memory B cells was performed using Chromium Single Cell V(D)J Reagent Kits (purchased from 10× genomics, Cat #100006) according to the manufacturer's instructions. The sequencing results were analyzed, and 360 antigen-binding units were obtained and named as ABU 1-395. The sequence information of the obtained antigen-binding units is as shown in Table 1 below.

TABLE 1
Exemplary antigen-binding units obtained herein

| ABU No. | VH SEQ ID No. | VL SEQ ID NO. |
|---|---|---|
| ABU-1 | 721 | 1081 |
| ABU-2 | 722 | 1082 |
| ABU-3 | 723 | 1083 |
| ABU-4 | 724 | 1084 |
| ABU-5 | 725 | 1085 |
| ABU-6 | 726 | 1086 |
| ABU-7 | 727 | 1087 |
| ABU-8 | 728 | 1088 |
| ABU-9 | 729 | 1089 |
| ABU-10 | 730 | 1090 |
| ABU-11 | 731 | 1091 |
| ABU-12 | 732 | 1092 |
| ABU-13 | 733 | 1093 |
| ABU-14 | 734 | 1094 |
| ABU-15 | 735 | 1095 |
| ABU-16 | 736 | 1096 |
| ABU-17 | 737 | 1097 |
| ABU-18 | 738 | 1098 |
| ABU-19 | 739 | 1099 |
| ABU-20 | 740 | 1100 |
| ABU-21 | 741 | 1101 |
| ABU-22 | 742 | 1102 |
| ABU-23 | 743 | 1103 |
| ABU-24 | 744 | 1104 |
| ABU-25 | 745 | 1105 |
| ABU-26 | 746 | 1106 |
| ABU-27 | 747 | 1107 |
| ABU-28 | 748 | 1108 |
| ABU-29 | 749 | 1109 |
| ABU-30 | 750 | 1110 |
| ABU-31 | 751 | 1111 |
| ABU-32 | 752 | 1112 |
| ABU-33 | 753 | 1113 |
| ABU-34 | 754 | 1114 |
| ABU-35 | 755 | 1115 |
| ABU-36 | 756 | 1116 |
| ABU-37 | 757 | 1117 |
| ABU-38 | 758 | 1118 |
| ABU-39 | 759 | 1119 |
| ABU-40 | 760 | 1120 |
| ABU-41 | 761 | 1121 |
| ABU-42 | 762 | 1122 |
| ABU-43 | 763 | 1123 |
| ABU-44 | 764 | 1124 |
| ABU-45 | 765 | 1125 |
| ABU-46 | 766 | 1126 |
| ABU-47 | 767 | 1127 |
| ABU-48 | 768 | 1128 |
| ABU-49 | 769 | 1129 |
| ABU-50 | 770 | 1130 |
| ABU-51 | 771 | 1131 |
| ABU-52 | 772 | 1132 |
| ABU-53 | 773 | 1133 |
| ABU-54 | 774 | 1134 |
| ABU-55 | 775 | 1135 |
| ABU-56 | 776 | 1136 |
| ABU-57 | 777 | 1137 |
| ABU-58 | 778 | 1138 |
| ABU-59 | 779 | 1139 |
| ABU-60 | 780 | 1140 |
| ABU-61 | 781 | 1141 |
| ABU-62 | 782 | 1142 |
| ABU-63 | 783 | 1143 |
| ABU-64 | 784 | 1144 |
| ABU-65 | 785 | 1145 |
| ABU-66 | 786 | 1146 |
| ABU-67 | 787 | 1147 |
| ABU-68 | 788 | 1148 |
| ABU-69 | 789 | 1149 |
| ABU-70 | 790 | 1150 |
| ABU-71 | 791 | 1151 |
| ABU-72 | 792 | 1152 |
| ABU-73 | 793 | 1153 |
| ABU-74 | 794 | 1154 |
| ABU-75 | 795 | 1155 |
| ABU-76 | 796 | 1156 |
| ABU-77 | 797 | 1157 |
| ABU-78 | 798 | 1158 |
| ABU-79 | 799 | 1159 |
| ABU-80 | 800 | 1160 |
| ABU-81 | 801 | 1161 |
| ABU-82 | 802 | 1162 |
| ABU-83 | 803 | 1163 |
| ABU-84 | 804 | 1164 |
| ABU-85 | 805 | 1165 |
| ABU-86 | 806 | 1166 |
| ABU-87 | 807 | 1167 |
| ABU-88 | 808 | 1168 |
| ABU-89 | 809 | 1169 |
| ABU-90 | 810 | 1170 |
| ABU-91 | 811 | 1171 |
| ABU-92 | 812 | 1172 |
| ABU-93 | 813 | 1173 |
| ABU-94 | 814 | 1174 |
| ABU-95 | 815 | 1175 |
| ABU-96 | 816 | 1176 |
| ABU-97 | 817 | 1177 |
| ABU-98 | 818 | 1178 |
| ABU-99 | 819 | 1179 |
| ABU-100 | 820 | 1180 |
| ABU-101 | 821 | 1181 |
| ABU-102 | 822 | 1182 |
| ABU-103 | 823 | 1183 |
| ABU-104 | 824 | 1184 |
| ABU-105 | 825 | 1185 |
| ABU-106 | 826 | 1186 |
| ABU-107 | 827 | 1187 |
| ABU-108 | 828 | 1188 |
| ABU-109 | 829 | 1189 |
| ABU-110 | 830 | 1190 |
| ABU-111 | 831 | 1191 |
| ABU-112 | 832 | 1192 |
| ABU-113 | 833 | 1193 |
| ABU-114 | 834 | 1194 |
| ABU-115 | 835 | 1195 |
| ABU-116 | 836 | 1196 |
| ABU-117 | 837 | 1197 |
| ABU-118 | 838 | 1198 |
| ABU-119 | 839 | 1199 |
| ABU-120 | 840 | 1200 |
| ABU-121 | 841 | 1201 |
| ABU-122 | 842 | 1202 |
| ABU-123 | 843 | 1203 |
| ABU-124 | 844 | 1204 |
| ABU-125 | 845 | 1205 |
| ABU-126 | 846 | 1206 |
| ABU-127 | 847 | 1207 |
| ABU-128 | 848 | 1208 |
| ABU-129 | 849 | 1209 |
| ABU-130 | 850 | 1210 |
| ABU-131 | 851 | 1211 |
| ABU-132 | 852 | 1212 |
| ABU-133 | 853 | 1213 |
| ABU-134 | 854 | 1214 |
| ABU-135 | 855 | 1215 |
| ABU-136 | 856 | 1216 |
| ABU-137 | 857 | 1217 |
| ABU-138 | 858 | 1218 |
| ABU-139 | 859 | 1219 |
| ABU-140 | 860 | 1220 |
| ABU-141 | 861 | 1221 |
| ABU-142 | 862 | 1222 |
| ABU-143 | 863 | 1223 |
| ABU-144 | 864 | 1224 |
| ABU-145 | 865 | 1225 |
| ABU-146 | 866 | 1226 |
| ABU-147 | 867 | 1227 |
| ABU-148 | 868 | 1228 |
| ABU-149 | 869 | 1229 |
| ABU-150 | 870 | 1230 |
| ABU-151 | 871 | 1231 |
| ABU-152 | 872 | 1232 |
| ABU-153 | 873 | 1233 |
| ABU-154 | 874 | 1234 |
| ABU-155 | 875 | 1235 |
| ABU-156 | 876 | 1236 |
| ABU-157 | 877 | 1237 |
| ABU-158 | 878 | 1238 |
| ABU-159 | 879 | 1239 |
| ABU-160 | 880 | 1240 |
| ABU-161 | 881 | 1241 |
| ABU-162 | 882 | 1242 |
| ABU-163 | 883 | 1243 |
| ABU-164 | 884 | 1244 |
| ABU-165 | 885 | 1245 |
| ABU-166 | 886 | 1246 |
| ABU-167 | 887 | 1247 |
| ABU-168 | 888 | 1248 |
| ABU-169 | 889 | 1249 |
| ABU-170 | 890 | 1250 |
| ABU-171 | 891 | 1251 |
| ABU-172 | 892 | 1252 |
| ABU-173 | 893 | 1253 |
| ABU-174 | 894 | 1254 |
| ABU-175 | 895 | 1255 |
| ABU-176 | 896 | 1256 |
| ABU-177 | 897 | 1257 |
| ABU-178 | 898 | 1258 |
| ABU-179 | 899 | 1259 |
| ABU-180 | 900 | 1260 |
| ABU-181 | 901 | 1261 |
| ABU-182 | 902 | 1262 |
| ABU-183 | 903 | 1263 |
| ABU-184 | 904 | 1264 |
| ABU-185 | 905 | 1265 |
| ABU-186 | 906 | 1266 |
| ABU-187 | 907 | 1267 |
| ABU-188 | 908 | 1268 |
| ABU-189 | 909 | 1269 |
| ABU-190 | 910 | 1270 |
| ABU-191 | 911 | 1271 |
| ABU-192 | 912 | 1272 |
| ABU-193 | 913 | 1273 |
| ABU-194 | 914 | 1274 |
| ABU-195 | 915 | 1275 |
| ABU-196 | 916 | 1276 |
| ABU-197 | 917 | 1277 |
| ABU-198 | 918 | 1278 |
| ABU-199 | 919 | 1279 |
| ABU-200 | 920 | 1280 |
| ABU-201 | 921 | 1281 |
| ABU-202 | 922 | 1282 |
| ABU-203 | 923 | 1283 |
| ABU-204 | 924 | 1284 |
| ABU-205 | 925 | 1285 |
| ABU-206 | 926 | 1286 |
| ABU-207 | 927 | 1287 |
| ABU-208 | 928 | 1288 |
| ABU-209 | 929 | 1289 |
| ABU-210 | 930 | 1290 |
| ABU-211 | 931 | 1291 |
| ABU-212 | 932 | 1292 |
| ABU-213 | 933 | 1293 |
| ABU-214 | 934 | 1294 |
| ABU-215 | 935 | 1295 |
| ABU-216 | 936 | 1296 |
| ABU-217 | 937 | 1297 |
| ABU-218 | 938 | 1298 |
| ABU-219 | 939 | 1299 |
| ABU-220 | 940 | 1300 |
| ABU-221 | 941 | 1301 |
| ABU-222 | 942 | 1302 |
| ABU-223 | 943 | 1303 |
| ABU-224 | 944 | 1304 |
| ABU-225 | 945 | 1305 |
| ABU-226 | 946 | 1306 |
| ABU-227 | 947 | 1307 |
| ABU-228 | 948 | 1308 |
| ABU-229 | 949 | 1309 |
| ABU-230 | 950 | 1310 |
| ABU-231 | 951 | 1311 |
| ABU-232 | 952 | 1312 |
| ABU-233 | 953 | 1313 |
| ABU-234 | 954 | 1314 |
| ABU-235 | 955 | 1315 |
| ABU-236 | 956 | 1316 |
| ABU-237 | 957 | 1317 |
| ABU-238 | 958 | 1318 |
| ABU-239 | 959 | 1319 |
| ABU-240 | 960 | 1320 |
| ABU-241 | 961 | 1321 |
| ABU-242 | 962 | 1322 |
| ABU-243 | 963 | 1323 |
| ABU-244 | 964 | 1324 |
| ABU-245 | 965 | 1325 |
| ABU-246 | 966 | 1326 |
| ABU-247 | 967 | 1327 |
| ABU-248 | 968 | 1328 |
| ABU-249 | 969 | 1329 |
| ABU-250 | 970 | 1330 |
| ABU-251 | 971 | 1331 |
| ABU-252 | 972 | 1332 |
| ABU-253 | 973 | 1333 |
| ABU-254 | 974 | 1334 |
| ABU-255 | 975 | 1335 |
| ABU-256 | 976 | 1336 |
| ABU-257 | 977 | 1337 |
| ABU-258 | 978 | 1338 |
| ABU-259 | 979 | 1339 |
| ABU-260 | 980 | 1340 |
| ABU-261 | 981 | 1341 |
| ABU-262 | 982 | 1342 |
| ABU-263 | 983 | 1343 |
| ABU-264 | 984 | 1344 |
| ABU-265 | 985 | 1345 |
| ABU-266 | 986 | 1346 |
| ABU-267 | 987 | 1347 |
| ABU-268 | 988 | 1348 |
| ABU-269 | 989 | 1349 |
| ABU-270 | 990 | 1350 |
| ABU-271 | 991 | 1351 |
| ABU-272 | 992 | 1352 |
| ABU-273 | 993 | 1353 |
| ABU-274 | 994 | 1354 |
| ABU-275 | 995 | 1355 |
| ABU-276 | 996 | 1356 |
| ABU-277 | 997 | 1357 |
| ABU-278 | 998 | 1358 |
| ABU-279 | 999 | 1359 |
| ABU-280 | 1000 | 1360 |
| ABU-281 | 1001 | 1361 |
| ABU-282 | 1002 | 1362 |
| ABU-283 | 1003 | 1363 |
| ABU-284 | 1004 | 1364 |
| ABU-285 | 1005 | 1365 |
| ABU-286 | 1006 | 1366 |
| ABU-287 | 1007 | 1367 |
| ABU-288 | 1008 | 1368 |
| ABU-289 | 1009 | 1369 |
| ABU-290 | 1010 | 1370 |
| ABU-291 | 1011 | 1371 |
| ABU-292 | 1012 | 1372 |
| ABU-293 | 1013 | 1373 |
| ABU-294 | 1014 | 1374 |
| ABU-295 | 1015 | 1375 |
| ABU-296 | 1016 | 1376 |
| ABU-297 | 1017 | 1377 |
| ABU-298 | 1018 | 1378 |
| ABU-299 | 1019 | 1379 |
| ABU-300 | 1020 | 1380 |
| ABU-301 | 1021 | 1381 |
| ABU-302 | 1022 | 1382 |
| ABU-303 | 1023 | 1383 |
| ABU-304 | 1024 | 1384 |
| ABU-305 | 1025 | 1385 |
| ABU-306 | 1026 | 1386 |
| ABU-307 | 1027 | 1387 |
| ABU-308 | 1028 | 1388 |
| ABU-309 | 1029 | 1389 |
| ABU-310 | 1030 | 1390 |
| ABU-311 | 1031 | 1391 |
| ABU-312 | 1032 | 1392 |
| ABU-313 | 1033 | 1393 |
| ABU-314 | 1034 | 1394 |
| ABU-315 | 1035 | 1395 |
| ABU-316 | 1036 | 1396 |
| ABU-317 | 1037 | 1397 |
| ABU-318 | 1038 | 1398 |
| ABU-319 | 1039 | 1399 |
| ABU-320 | 1040 | 1400 |
| ABU-321 | 1041 | 1401 |
| ABU-322 | 1042 | 1402 |
| ABU-323 | 1043 | 1403 |
| ABU-324 | 1044 | 1404 |
| ABU-325 | 1045 | 1405 |
| ABU-326 | 1046 | 1406 |
| ABU-327 | 1047 | 1407 |
| ABU-328 | 1048 | 1408 |
| ABU-329 | 1049 | 1409 |
| ABU-330 | 1050 | 1410 |
| ABU-331 | 1051 | 1411 |
| ABU-332 | 1052 | 1412 |
| ABU-333 | 1053 | 1413 |
| ABU-334 | 1054 | 1414 |
| ABU-335 | 1055 | 1415 |
| ABU-336 | 1056 | 1416 |
| ABU-337 | 1057 | 1417 |
| ABU-338 | 1058 | 1418 |
| ABU-339 | 1059 | 1419 |
| ABU-340 | 1060 | 1420 |
| ABU-341 | 1061 | 1421 |
| ABU-342 | 1062 | 1422 |
| ABU-343 | 1063 | 1423 |
| ABU-344 | 1064 | 1424 |
| ABU-345 | 1065 | 1425 |
| ABU-346 | 1066 | 1426 |
| ABU-347 | 1067 | 1427 |
| ABU-348 | 1068 | 1428 |
| ABU-349 | 1069 | 1429 |
| ABU-350 | 1070 | 1430 |
| ABU-351 | 1071 | 1431 |
| ABU-352 | 1072 | 1432 |
| ABU-353 | 1073 | 1433 |
| ABU-354 | 1074 | 1434 |
| ABU-355 | 1075 | 1435 |
| ABU-356 | 1076 | 1436 |
| ABU-357 | 1077 | 1437 |
| ABU-358 | 1078 | 1438 |
| ABU-359 | 1079 | 1439 |
| ABU-360 | 1080 | 1440 |
| ABU-361 | 3111 | 3146 |
| ABU-362 | 3112 | 3147 |
| ABU-363 | 3113 | 3148 |
| ABU-364 | 3114 | 3149 |
| ABU-365 | 3115 | 3150 |
| ABU-366 | 3116 | 3151 |
| ABU-367 | 3117 | 3152 |
| ABU-368 | 3118 | 3153 |
| ABU-369 | 3119 | 3154 |
| ABU-370 | 3120 | 3155 |
| ABU-371 | 3121 | 3156 |
| ABU-372 | 3122 | 3157 |
| ABU-373 | 3123 | 3158 |
| ABU-374 | 3124 | 3159 |
| ABU-375 | 3125 | 3160 |
| ABU-376 | 3126 | 3161 |
| ABU-377 | 3127 | 3162 |
| ABU-378 | 3128 | 3163 |
| ABU-379 | 3129 | 3164 |
| ABU-380 | 3130 | 3165 |
| ABU-381 | 3131 | 3166 |
| ABU-382 | 3132 | 3167 |
| ABU-383 | 3133 | 3168 |
| ABU-384 | 3134 | 3169 |
| ABU-385 | 3135 | 3170 |
| ABU-386 | 3136 | 3171 |
| ABU-387 | 3137 | 3172 |
| ABU-388 | 3138 | 3173 |
| ABU-389 | 3139 | 3174 |
| ABU-390 | 3140 | 3175 |
| ABU-391 | 3141 | 3176 |
| ABU-392 | 3142 | 3177 |
| ABU-393 | 3143 | 3178 |
| ABU-394 | 3144 | 3179 |
| ABU-395 | 3145 | 3180 |

### Example 3: Preparation and Purification of Antigen-Binding Unit of the Present Invention

According to the sequence information of the antigen-binding units obtained in example 2, Sino Biological Inc. was entrusted to express and purify the obtained antigen-binding units, and the antigenic reactivity thereof was detected.

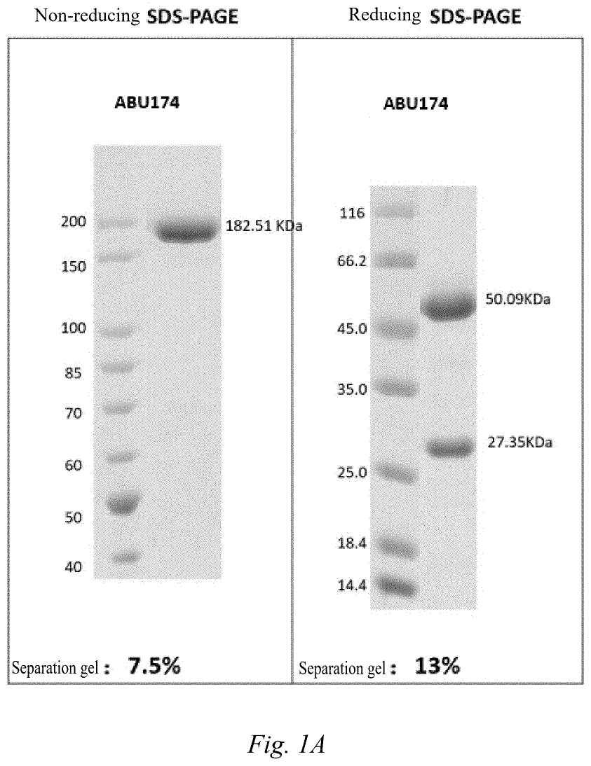

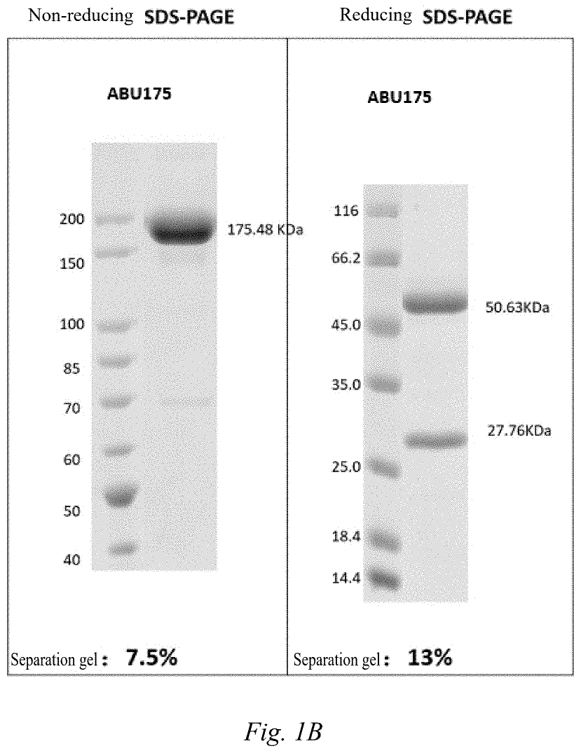

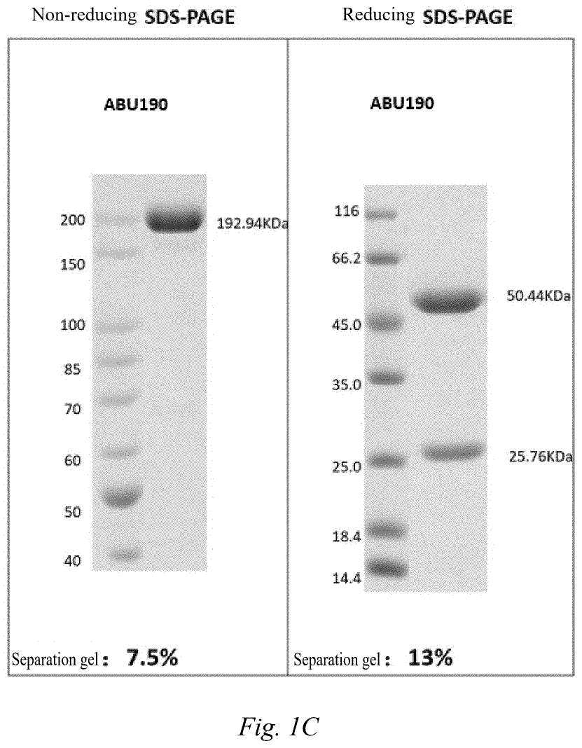

In short, nucleic acid molecules encoding the heavy and light chains of the antibody were synthesized in vitro and then cloned into expression vectors, respectively, thereby obtaining recombinant expression vectors encoding the heavy and light chains of the antibody, respectively. HEK293 cells were co-transfected with the above-mentioned recombinant expression vectors encoding the heavy and light chains of the antibody, respectively. 4-6 hours after the transfection, the cell culture solution was changed to a serum-free medium, which was cultured at 37° C. for another 6 days. After cultivation, the antibody protein expressed by the cells was purified from the culture by an affinity purification column. Then, the purified protein of interest was detected by reducing and non-reducing SDS-PAGE. By taking ABU-174, ABU-175 and ABU190 as examples, the electrophoresis results thereof after preparation are shown in FIGS. 1A-1C, respectively. The results show that the purities of purified ABU-174, ABU-175 and ABU190 are 95.9%, 96.4% and 98.2%, respectively.

Then, the antigenic reactivity of the purified antibody to be detected was detected by ELISA experiments using the RBD of the recombinantly expressed S protein as a coating antigen and using Goat anti-human IgG Fc labeled with horseradish peroxidase (HRP) as a secondary antibody. In short, a 96-well plate was coated with the RBD of the recombinantly expressed S protein (with an amino acid sequence as shown in SEQ ID NO: 1459 and at a concentration of 0.01 μg/ml or 1 μg/ml), and then the 96-well plate was blocked with a blocking solution. Then, the monoclonal antibodies to be detected (a control antibody, ABU-174, ABU-175 and ABU190; each at a concentration of 0.1 μg/ml) were added and incubated, respectively. After the plate was washed with an ELISA washing liquid, Goat anti-human IgG Fc labeled with horseradish peroxidase (HRP) was added as a secondary antibody (diluted at 1:500); and the plate was again incubated. Then, the ELISA plate was washed with PBST, and a color developing agent was added to develop the color. Then, the absorbance at OD450 nm was read on a microplate reader. The results are as shown in Table 2. It can be seen from Table 2 that ABU-174, ABU-175 and ABU190 can specifically recognize and bind to RBD of S protein.

TABLE 2
Reactivity of antigen-binding units of ABU-174, ABU-175 and ABU190
with RBD of S protein detected by ELISA (OD450 reading)

Concentration of RBD protein

| Sample to be detected | 0.01 μg/ml | 1 μg/ml |
|---|---|---|
| Irrelevant antibody (1 ug/ml) | 0.006 | 0.025 |
| ABU-174 (1 ug/ml) | 1.261 | 2.909 |
| ABU-175 (1 ug/ml) | 2.274 | 2.963 |
| ABU190 (1 ug/ml) | 0.288 | 3.057 |

### Example 4: Evaluation of Binding Ability of Antigen-Binding Unit of the Present Invention to S Protein

In the example, surface plasmon resonance (SPR) was used to detect the affinity of the antibody to the RBD region of the Spike protein. Biacore T200 was used for measurement. The biotin-labeled SARS-COV-2 RBD domain was first coupled to the SA chip (GE), and the RU value of the signal resonance unit was increased by 100 units. The running buffer was PBS at PH 7.4 plus 0.005% P20, ensuring that the buffer in the analyte (such as antibody) was the same as the running buffer. The purified antibody was subjected to 3-fold gradient dilution to a concentration between 50-0.78125 nM. The measurement results were analyzed using Biacore Evaluation software, all the curves were fitted to a 1:1 model to obtain the rate constant Ka for the binding of the antibody to the antigen and the rate constant Kd for the dissociation of the antibody from the antibody/antigen complex, and the dissociation equilibrium constant KD was calculated, wherein KD=Kd/Ka. The results are shown in Table 3 below.

The binding affinity of the exemplary antigen-binding unit of the present invention for the RBD region of the Spike protein is listed in Table 3, wherein the KD value of each antigen-binding unit is less than 20 nM.

TABLE 3
KD value of the binding affinity of the exemplary antigen-binding
unit of the present invention for the RBD region of Spike protein

| AUB No. | KD (Kd/Ka, nM) |
|---|---|
| ABU-145 | <10 |
| ABU-149 | <10 |
| ABU-174 | <1 |
| ABU-175 | <1 |
| ABU-181 | <10 |
| ABU-190 | <10 |
| ABU-205 | <10 |
| ABU-207 | <10 |
| ABU-208 | <1 |
| ABU-210 | <10 |
| ABU-211 | <20 |
| ABU-254 | <10 |
| ABU-257 | <10 |
| ABU-258 | <1 |
| ABU-288 | <1 |
| ABU-289 | <10 |
| ABU-290 | <1 |
| ABU-291 | <1 |
| ABU-296 | <1 |
| ABU-297 | <1 |
| ABU-298 | <20 |
| ABU-305 | <20 |
| ABU-308 | <10 |
| ABU-312 | <20 |
| ABU-316 | <10 |
| ABU-317 | <20 |
| ABU-319 | <10 |
| ABU-320 | <10 |
| ABU-322 | <1 |
| ABU-323 | <20 |
| ABU-325 | <10 |
| ABU-327 | <20 |
| ABU-328 | <10 |
| ABU-329 | <10 |
| ABU-330 | <10 |
| ABU-337 | <20 |
| ABU-339 | <20 |
| ABU-340 | <10 |
| ABU-341 | <10 |
| ABU-343 | <20 |
| ABU-344 | <1 |
| ABU-346 | <10 |
| ABU-348 | <10 |
| ABU-349 | <1 |
| ABU-351 | <10 |
| ABU-352 | <10 |
| ABU-354 | <1 |
| ABU-355 | <1 |
| ABU-356 | <10 |
| ABU-357 | <10 |
| ABU-358 | <10 |
| ABU-359 | <10 |
| ABU-360 | <1 |
| ABU-361 | <20 |
| ABU-362 | <20 |
| ABU-365 | <10 |
| ABU-367 | <1 |
| ABU-368 | <20 |
| ABU-369 | <10 |
| ABU-371 | <20 |
| ABU-372 | <20 |
| ABU-373 | <10 |
| ABU-375 | <10 |
| ABU-376 | <10 |
| ABU-377 | <10 |
| ABU-379 | <10 |
| ABU-380 | <1 |
| ABU-381 | <1 |
| ABU-382 | <10 |
| ABU-383 | <20 |
| ABU-384 | <20 |
| ABU-385 | <20 |
| ABU-386 | <10 |
| ABU-390 | <10 |
| ABU-391 | <20 |
| ABU-392 | <10 |
| ABU-393 | <20 |
| ABU-394 | <20 |
| ABU-395 | <10 |

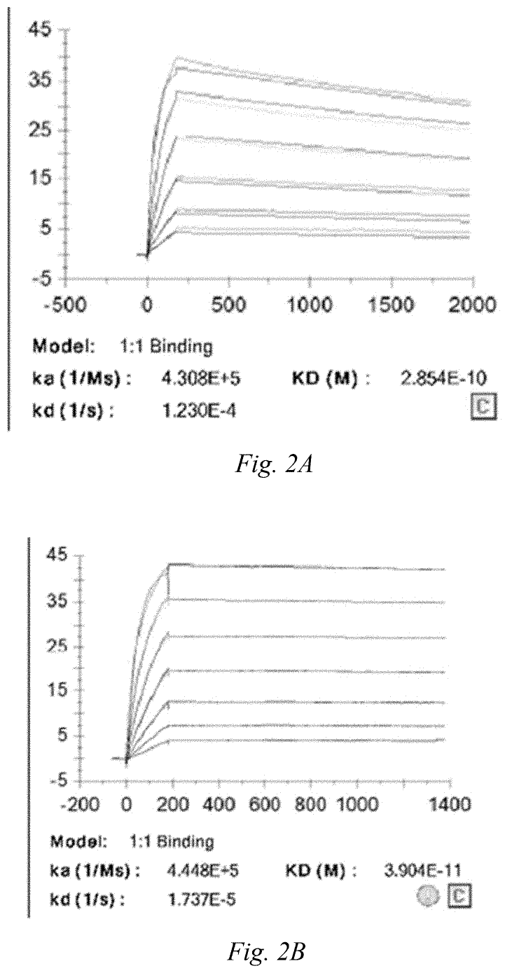

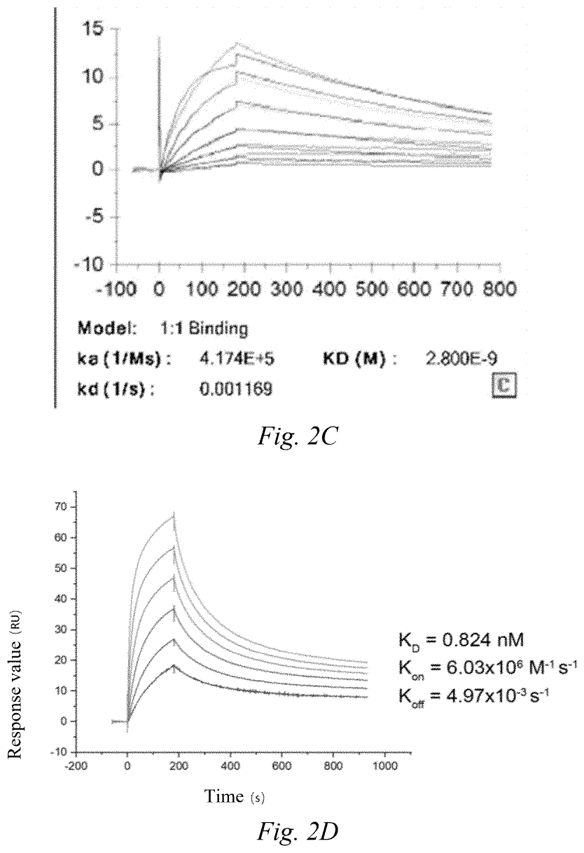

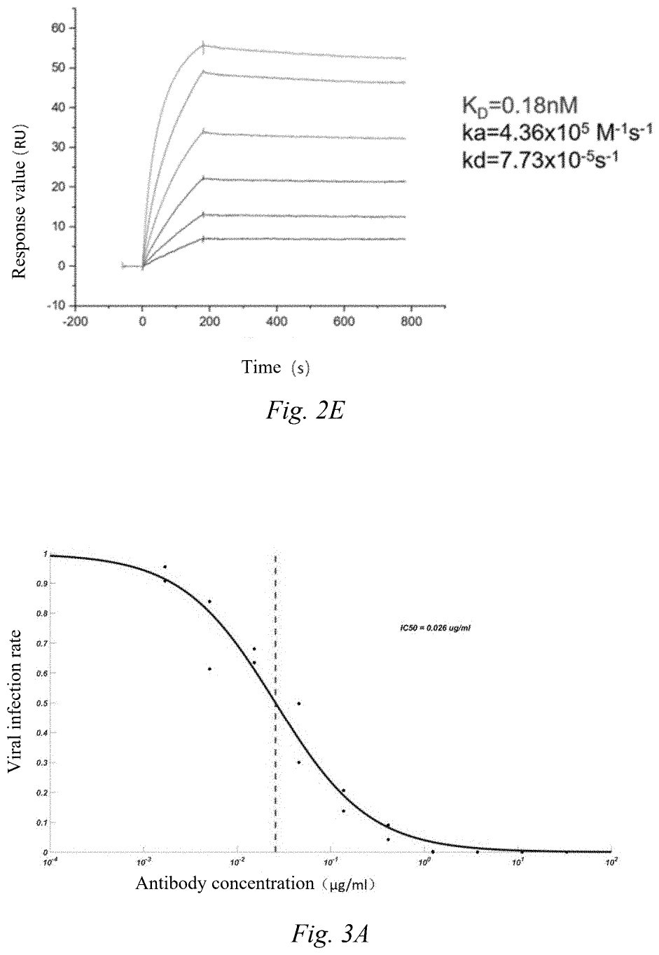

FIGS. 2A-2E further exemplarily show the binding affinity of ABU-174, ABU-175, ABU190, ABU297 and ABU367 for the RBD region of the Spike protein. It can be seen from FIGS. 2A-2E that ABU-174 has a KD value of 0.29 nM, ABU-175 has a KD value of 0.039 nM, ABU190 has a KD value of 2.8 nM, ABU297 has a KD value of 0.824 nM, and ABU has a KD value of 0.18 nM. FIGS. 2A-2E show that ABU-174, ABU-175, ABU190, ABU297 and ABU367 all have good affinity for the S protein of the novel coronavirus.

### Example 5: Evaluation of Ability of Antigen-Binding Unit of the Present Invention to Neutralize SARS-CoV-2 Pseudovirus

In this example, the cell microneutralization assay was used to detect the neutralizing activity of the antigen-binding unit of the present invention against SARS-CoV-2 pseudovirus with reference to the description of Temperton N J et al., Emerg Infect Dis, 2005, 11(3), 411-416. The SARS-CoV-2 pseudovirus used in this example was provided by China National Institutes for Food and Drug Control, has similar cell infection characteristics to the euvirus, can be used to simulate the early process of euvirus infection of a cell, and carries reporter gene luciferase, which can be quickly and easily detected and analyzed. The safety for operating the pseudovirus is high, and the neutralization experiment can be completed in Biosafety Physical Containment Level-2 Laboratory to detect the neutralization activity (Neutralization titer) of the antibody. The specific steps of the experiment method are as follows:

### 1. Reagent for Equilibration

The reagent (0.25% trypsin-EDTA, DMEM complete medium) stored at 2° C.-8° C. was taken out and equilibrated at room temperature for more than 30 minutes.

### 2. Experimental Operation

(1) A 96-well plate was taken, and the arrangement of the samples was set up as shown in Table 4; A2-H2 wells were set as cell control wells (CC), which only contain experimental cells; A3-H3 wells were set as virus control wells (VV), which contain experimental cells and pseudovirus; A4-A11, B4-B11, C4-C11, D4-D11, E4-E11, F4-F11, G4-G11 and H4-H11 wells were set as experimental wells, which contain experimental cells, pseudovirus and different concentrations of antibody to be detected; and other wells were set as blank. The experimental cells and pseudovirus used in this example were Huh-7 cells and SARS-CoV-2 virus (both provided by China National Institutes for Food and Drug Control), respectively.

TABLE 4
Arrangement of samples in 96-well plate

| | 1 | 2 | 3 | 4 | 5-10 | 11 | 12 |
|---|---|---|---|---|---|---|---|
| A | — | CC | VV | Dilution 1 | Dilution 1 | Dilution 1 | — |
| B | — | CC | VV | Dilution 2 | Dilution 2 | Dilution 2 | — |
| C | — | CC | VV | Dilution 3 | Dilution 3 | Dilution 3 | — |
| D | — | CC | VV | Dilution 4 | Dilution 4 | Dilution 4 | — |
| E | — | CC | VV | Dilution 5 | Dilution 5 | Dilution 5 | — |
| F | — | CC | VV | Dilution 6 | Dilution 6 | Dilution 6 | — |
| G | — | CC | VV | Dilution 7 | Dilution 7 | Dilution 7 | — |
| H | — | CC | VV | Dilution 8 | Dilution 8 | Dilution 8 | — |

(2) DMEM complete mediums (containing 1% antibiotic, 25 mM HEPES, 10% FBS) were added at 100 μl/well to the cell control wells; DMEM complete mediums were added at 100 μl/well to the virus control wells; and the indicated concentration of the antibody to be detected diluted in DMEM complete mediums was added to the experimental wells at 50 μl/well. The antibody concentrations of dilutions 1-8 used in Table 4 were 1/30 μg/μl, 1/90 μg/μl, 1/270 μg/μl, 1/810 μg/μl, 1/2430 μg/μl, 1/7290 μg/μl, 1/21870 μg/μl, and 1/65610 μg/μl, respectively.

(3) The SARS-CoV-2 pseudovirus was diluted to about 1.3×10⁴/ml (TCID50) with DMEM complete mediums; and then, the SARS-CoV-2 pseudovirus was added at 50 μl/well to the virus control wells and the experimental wells.

(4) The 96-well plate was placed in a cell incubator (37° C., 5% CO₂) and incubated for 1 hour.

(5) The pre-cultured Huh-7 cells were diluted to 2×10⁵ cells/ml with DMEM complete mediums. After the incubation in the previous step, cells were added at 100 μl/well to the cell control wells, virus control wells and experimental wells.

(6) The 96-well plate was placed in a cell incubator (37° C., 5% CO₂) and cultured for 20-28 hours.

(7) The 96-well plate was taken out from the cell incubator; 150 μl of the supernatant was aspirated from each well and discarded; and then 100 μl of luciferase detection reagents were added, and reacted at room temperature for 2 minutes in the dark.

(8) After the reaction was completed, the liquid in each well was pipetted 6 to 8 times repeatedly using a pipette until the cells were fully lysed. Then, 150 μl of liquid was aspirated from each well and transferred to the corresponding 96-well chemiluminescence detection plate, and the luminescence value was read with a chemiluminescence detector (Perkinelmer EnSight multimode microplate reader).

(9) Calculation of neutralization inhibition rate:

Inhibition rate=[1−(mean luminescence intensity of experimental wells−mean luminescence intensity of CC wells)/(mean luminescence intensity of VV wells−mean luminescence intensity of CC wells)]×100%.

(10) IC₅₀ of the antibody to be detected was calculated by Reed-Muench method according to the result of the neutralization inhibition rate.

Table 5 lists IC₅₀ of the exemplary antigen-binding unit of the present invention for neutralizing SARS-CoV-2 pseudovirus, wherein the IC₅₀ value of each antigen-binding unit is less than 1 μg/ml.

TABLE 5
IC50 of exemplary antigen-binding unit of the present
invention for neutralizing SARS-CoV-2 pseudovirus

| ABU No. | IC50 (μg/ml) |
|---|---|
| ABU-174 | <0.1 |
| ABU-175 | <0.1 |
| ABU-190 | <0.1 |
| ABU-207 | <0.5 |
| ABU-208 | <0.5 |
| ABU-257 | <0.5 |
| ABU-290 | <0.1 |
| ABU-291 | <0.5 |
| ABU-296 | <0.1 |
| ABU-297 | <0.1 |
| ABU-308 | <0.5 |
| ABU-322 | <0.1 |
| ABU-340 | <0.5 |
| ABU-341 | <0.1 |
| ABU-344 | <1 |
| ABU-349 | <0.1 |
| ABU-351 | <0.1 |
| ABU-352 | <0.1 |
| ABU-354 | <0.1 |
| ABU-355 | <0.1 |
| ABU-356 | <0.1 |
| ABU-357 | <1 |
| ABU-358 | <0.1 |
| ABU-359 | <0.1 |
| ABU-360 | <0.1 |
| ABU-361 | <0.5 |
| ABU-362 | <0.5 |
| ABU-365 | <0.1 |
| ABU-367 | <0.1 |
| ABU-368 | <0.5 |
| ABU-369 | <0.1 |
| ABU-371 | <1 |
| ABU-372 | <0.5 |
| ABU-373 | <0.5 |
| ABU-375 | <0.1 |
| ABU-376 | <0.1 |
| ABU-377 | <0.5 |
| ABU-379 | <0.5 |
| ABU-380 | <0.1 |
| ABU-381 | <0.1 |
| ABU-382 | <0.1 |
| ABU-386 | <0.1 |
| ABU-391 | <1 |
| ABU-392 | <0.1 |
| ABU-395 | <0.1 |

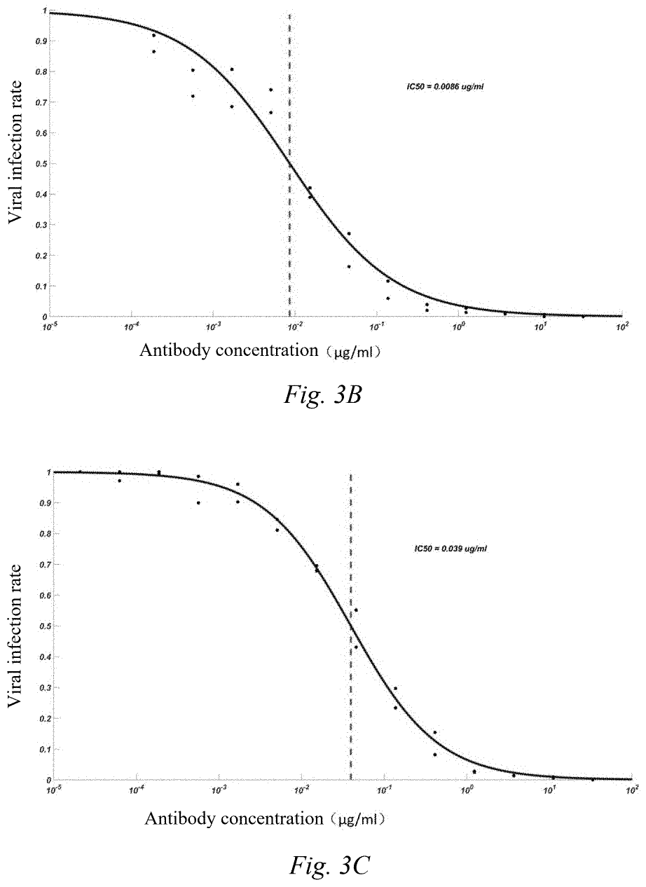

FIGS. 3A-3C further exemplarily show the neutralizing activity of ABU-174, ABU-175 and ABU190 against the SARS-CoV-2 pseudovirus. It can be seen from FIGS. 3A-3C that ABU-174, ABU-175 and ABU190 all have a good neutralizing activity, and the IC₅₀ thereof are 0.026 μg/ml (ABU-174), 0.0086 μg/ml (ABU-175), and 0.039 μg/ml (ABU190), respectively.

### Example 6: Evaluation of Ability of Antigen-Binding Unit of the Present Invention to Neutralize SARS-CoV-2 Euvirus

In this example, neutralizing activities of the antibodies to be detected were evaluated by cytopathic effect (CPE) assay and Plaque Reduction Neutralization Test (PRNT), respectively. The SARS-CoV-2 virus used was provided by Academy of Military Medical Sciences, the titer thereof (TCID50) was 10⁵/ml, and all experimental operations were completed in a BSL-3 laboratory.

### 6.1 Cytopathic Effect (CPE) Assay

(1) 100 μl of Vero E6 cells were added to each well of a 96-well culture plate at a concentration of 5×10⁴/ml, and cultured at 37° C., 5% CO₂ for 24 hours.

(2) The antibody to be detected was diluted to 10 concentrations: 1/10 μg/μl, 1/30 μg/μl, 1/90 μg/μl, 1/270 μg/μl, 1/810 μg/μl, 1/2430 μg/μl, 1/7290 μg/μl, 1/21870 μg/μl 1/65610 μg/μl, and 1/196830 μg/μl. 100 μl of the antibody to be detected at a specified concentration was taken out; an equal volume of SARS-CoV-2 euvirus (100 TCID50) was added; and the mixture was incubated at 37° C., 5% CO₂ for 1 h.

(3) After cultivation in step (1), the cell culture solution in the 96-well culture plate was discarded, and the mixture solution (200 μl) containing the antibody to be detected and the euvirus prepared in step (2) was added as an experimental group. After the mixture was incubated for 1 h, the supernatant was aspirated from the wells, and 200 μl of DMEM mediums (containing 2% antibiotic and 16 μg/ml of trypsin) were added to each well.

During the experiment, the cell control group and the virus control group were set in parallel. In the cell control group (4 replicate wells), after the cell culture solution in the wells was discarded; 200 μl of DMEM mediums (containing 2% antibiotic and 16 μg/ml of trypsin) were added to each well. In the virus control group (3 replicate wells), after the cell culture solution in the wells was discarded; 100 TCID50 of euvirus (100 μl) was added to each well, and the mixture was incubated at 37° C. for 1 h; after the incubation, the supernatant was aspirated from the wells, and 200 μl of DMEM mediums (containing 2% antibiotic and 16 μg/ml of trypsin) were added to each well.

(4) The cells were cultured for 4-5 days at 37° C., 5% CO₂.

(5) The cytopathic effect (CPE) was observed under the optical microscope, and the inhibitory activities of different concentrations of a monoclonal antibody against CPE were evaluated according to conditions of the cytopathic effect.

The detection results of the antigen-binding unit ABU-174 are shown in Table 6 below. The results show that the antigen-binding unit ABU-174 has an inhibitory effect on the virus at a cellular level, and the neutralizing antibody titer is 1.6 ng/μl.

TABLE 6
Neutralizing activity effect of antigen-
binding unit ABU-174 on SARS-CoV-2

| Antibody to be | | | | |
|---|---|---|---|---|
| detected | Dilution | Results (3 replicate wells) | | |
| Antigen-binding | 1:10 | − | − | − |
| unit ABU-174 | 1:30 | − | − | − |
| | 1:90 | − | − | − |
| | 1:270 | − | − | − |
| | 1:810 | − | + | + |
| | 1:2430 | + | + | + |
| | 1:7290 | + | + | + |
| | 1:21870 | + | + | + |
| | 1:65610 | + | + | + |
| | 1:196830 | + | + | + |
| Cell control | 200 μl DMEM | − | − | − |
| Negative control | 100TCID50 | + | + | + |

“+” means that the cell has CPE change, and means “−” that the cell does not have CPE change or has a normal cell morphology

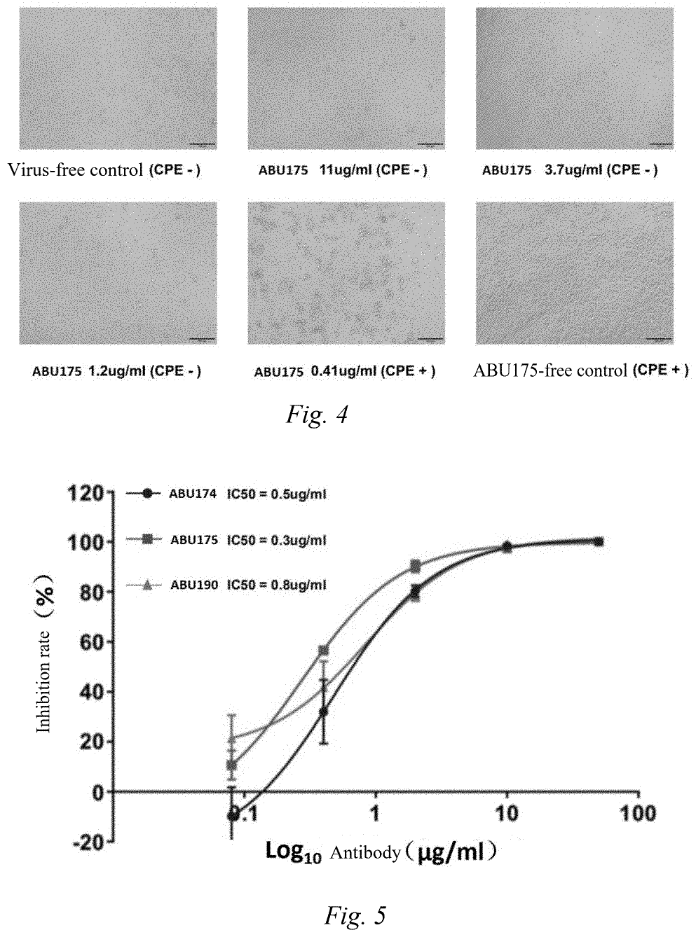

The detection results of the antigen-binding unit ABU-175 are shown in Table 7 and FIG. 4 below. The results show that the antigen-binding unit ABU-175 has an inhibitory effect on the virus at a cellular level, and the neutralizing antibody titer is 0.7 ng/μl.

TABLE 7
Neutralizing activity effect of antigen-
binding unit ABU-175 on SARS-CoV-2

| Antibody to be | | | | |
|---|---|---|---|---|
| detected | Dilution | Results (3 replicate wells) | | |
| Antigen-binding | 1:10 | − | − | − |
| unit ABU-175 | 1:30 | − | − | − |
| | 1:90 | − | − | − |
| | 1:270 | − | − | − |
| | 1:810 | − | − | − |
| | 1:2430 | + | + | + |
| | 1:7290 | + | + | + |
| | 1:21870 | + | + | + |
| | 1:65610 | + | + | + |
| | 1:196830 | + | + | + |
| Cell control | 200 μl DMEM | − | − | − |
| Negative control | 100TCID50 | + | + | + |

“+” means that the cell has CPE change, and “−” means that the cell does not have CPE change or has a normal cell morphology

### 6.2 Plaque Reduction Neutralization Test (PRNT):

(1) 100 μl of Vero E6 cells were added to each well of a 96-well culture plate at a concentration of 5×10⁴/ml, and cultured at 37° C., 5% CO₂ for 24 hours.

(2) The antibody to be detected was diluted to 5 concentrations: 50 μg/ml, 10 μg/ml, 2 μg/ml, 0.4 μg/ml, and 0.08 μg/ml.

(3) After cultivation in step (1), the cell culture solution in the 96-well culture plate was discarded, and the mixture solution (200 μl) containing the antibody to be detected and the euvirus prepared in step (2) was added as an experimental group. After the mixture was incubated for 1 h, the supernatant was aspirated from the wells, and 200 μl of DMEM mediums (containing 2% antibiotic and 16 μg/ml of trypsin) were added to each well.

During the experiment, the cell control group and the virus control group were set in parallel. In the cell control group, after the cell culture solution in the wells was discarded; 200 μl of DMEM mediums (containing 2% antibiotic and 16 μg/ml of trypsin) were added to each well. In the virus control group (4 replicate wells), after the cell culture solution in the wells was discarded; 100 TCID50 of euvirus (100 μl) was added to each well, and the mixture was incubated at 37° C. for 1 h; after the incubation, the supernatant was aspirated from the wells, and 200 μl of DMEM mediums (containing 2% antibiotic and 16 μg/ml of trypsin) were added to each well.

(4) The cells were cultured for 4 days at 37° C., 5% CO₂.

(5) After fixed with formaldehyde, the cells were labeled with rabbit anti-SARS-COV serum (Sino Biological) and peroxidase-labeled goat anti-rabbit IgG (Dako). The plaques were observed after the cells were developed with TMB (True Blue, KPL), the inhibition rate was calculated and the dose-response curve was drawn.

FIG. 5 shows dose-response curves for the exemplary antigen-binding units ABU-174, ABU-175 and ABU190 of the present invention. It can be seen from FIG. 5 that the antigen-binding units ABU-174, ABU-175 and ABU190 all have good neutralizing activities against SARS-CoV-2 euvirus, and can effectively inhibit virus infection and cell invasion, and the IC50 are 0.5 μg/ml (ABU-174), 0.3 μg/ml (ABU-175) and 0.8 μg/ml (ABU-190), respectively.

### Example 7. In Vivo Potency of the Antigen-Binding Unit of the Present Invention

SARS-CoV-2 infects a cell by interaction with the hACE2 receptor. The neutralizing potency of the antigen-binding unit of the present invention against SARS-CoV-2 in vivo was evaluated in two different animal models.

7.1 Potency of the Antigen-Binding Unit in hACE2 Transgenic Mice

In the first model, hACE2 transgenic mice were used as a animal model and treated with 2 different modes, i.e., pre-exposure prophylaxis and post-exposure prophylaxis. Specifically, hACE2 transgenic mice were intranasally infected with SARS-CoV-2 viruses (2019-nCoV Beta CoV/Wuhan/AMMS01/2020) at a dose of 105 TCID50.

In the pre-exposure prophylaxis treatment mode, the antigen-binding unit of the present invention was injected intraperitoneally at a dose of 20 mg/kg into hACE2 transgenic mice 24 hours prior to viral infection and the potency of the antigen-binding unit as a pre-exposure prophylactic intervention was detected.

In the post-exposure prophylaxis mode, 2 hours after viral infection, mice were injected with the antigen-binding unit at a dose of 20 mg/kg. HG1K (IgG1 antibody against H7N9 virus) was used as a negative control, and 2 hours after virus infection, same was injected at 20 mg/kg. Body weights that reflect the health condition of the infected mice were recorded daily for 5 consecutive days.

### 7.2 In Vivo Potency of Antigen-Binding Unit in Hamster

In the second model, hamsters (Mesocricetus auratus) were used as a animal model and treated with 2 different modes, i.e., pre-exposure prophylaxis and post-exposure prophylaxis. Specifically, hamsters were intranasally infected with SARS-CoV-2 proviruses (SARS-COV-2/WH-09/human/020/CHN) at a dose of 105 TCID50, which is similar to hACE2 transgenic mice.

In the pre-exposure prophylaxis treatment mode of hamsters, the antigen-binding units of the present invention were injected at a dose of 20 mg/kg into hamsters 1 day prior to viral infection. In the control group, 2 hours after infection, animals were injected with PBS.

In the post-exposure prophylaxis treatment mode of hamsters, 2 hours after infection, the antigen-binding units of the present invention were injected intraperitoneally into hamsters at different doses (including 20, 10, 5 and 2 mg/kg) according to body weights. In addition, the hamster injected with phosphate buffered saline (PBS) was used as a control. Body weights of the infected hamsters were recorded daily for 7 consecutive days. Hamsters were sacrificed 7 days after infection and lungs were collected for viral load analysis.

### Sequence Information

The information of partial sequences involved herein is as shown in Table 8 below.

TABLE 8
Sequence Listing

| SEQ ID | Sequence |
|---|---|
| 1 | ARDVTLVRGTASPRFDY |
| 2 | ARDVTLVRGTASPRFDY |
| 3 | ARSTRRWLQFVFPFDY |
| 4 | ARSTRRWLQFVFPFDY |
| 5 | ARSTRRWLQFVFPFDY |
| 6 | ARSTRRWLQFVFPFDY |
| 7 | ARSTRRWLQFVFPFDY |
| 8 | ARQAPGGGLLGYYHGLDV |
| 9 | ARQAPGGGLLGYYHGLDV |
| 10 | ARDRYCGGDCSGPHYYYYGMDV |
| 11 | ARWDCSGGSCNYYYYYNMDV |
| 12 | ARWDCSGGSCNYYYYYNMDV |
| 13 | AREDILLVPAASNFYYFGMDV |
| 14 | ARGDYYDPDDRYNAYYSLGA |
| 15 | TKGSMLLEVY |
| 16 | ARAPSDSSGINGAFDI |
| 17 | ARPKAPGYSYLSLDY |
| 18 | CGFGVVTTDAYGMDV |
| 19 | VKDKACTTTSCYEGTFFDY |
| 20 | VRGDDSILTPTFDH |
| 21 | ARAGKGFMVITHFDY |
| 22 | ARPHTNSWDQFDY |
| 23 | ARPQGGSSWYRDYYYGMDV |
| 24 | ATSTAVLRYFAPTGGWFDP |
| 25 | AKDNGHSYGYSWFDP |
| 26 | ATDGATIPINYYGMDV |
| 27 | ARSPITMIVVVNAFDI |
| 28 | ARARITMIVVVNHFDY |
| 29 | ARVQSTGYKYWYFDI |
| 30 | ARGFDY |
| 31 | ARARDYGSGSPMDV |
| 32 | ARDGVYYGSVIYHHYDLHV |
| 33 | ARGGGELLRYPFDY |
| 34 | AKAGLGLETSGGNYFES |
| 35 | AKDRVTMNYFDY |
| 36 | ARVREGYTSGWYADY |
| 37 | ARDRSYYHSSGYHYYFDY |
| 38 | VRDRIVGGYSYGGDY |
| 39 | AKGRLSPRL |
| 40 | ARVKVDNVVFDL |
| 41 | ARDRGLAARPAGWVDL |
| 42 | ARENFHFSGTPPLY |
| 43 | ARKYTYDTSGFFLSSSRNAFDV |
| 44 | ARLGSNGYGL |
| 45 | ARTYSYDSSGFFLTSSREAFDI |
| 46 | VRKYSFDVSGFFLSSSRHAFDV |
| 47 | ARKYSYDTSGFFLTSSRDAFDV |
| 48 | VRKFSYDISGFFLTSSRDAFDV |
| 49 | ATEGV |
| 50 | LLIEGMGATSGD |
| 51 | ATTNDGYYYGMDV |
| 52 | ATNPHNTAMVLDYYGMDV |
| 53 | AGAYIAAAGWGWELFQYYFDY |
| 54 | AHQAPFEWFGVDY |
| 55 | TTDGLYCSGGSCYYHSYYYYYGMDV |
| 56 | ARDGLGNYDILTGYTERAFDI |
| 57 | ARVKPILRVVVVAATPCDY |
| 58 | ARHARGYQLLSPRLGELSLYRSFDY |
| 59 | ARATTTKMIVVVINAFDI |
| 60 | ARHWITMIVVVIKGGWFDP |
| 61 | ARIRGQWLVGKYYYGMDV |
| 62 | AHRGWGFSSSFFDY |
| 63 | ARMSSSLQHYYGMDV |
| 64 | ARMSSSLQHYYGMDV |
| 65 | ARDVTLVRGTASPRFDY |
| 66 | ARDVTLVRGTASPRFDY |
| 67 | AQEGRNYDRNWFDP |
| 68 | ARLIPIDGRDV |
| 69 | TTYWDQYTSTWT |
| 70 | ASIVKYDSSGYNFDY |
| 71 | TRDPWHESEHRFDP |
| 72 | AKDNKVSSWYSFDI |
| 73 | ARGLGYYVAL |
| 74 | VRGGQEVSLRRLDWFVGY |
| 75 | AKERGGSGKMYDY |
| 76 | ARRGAAVAGTTGGSAFDI |
| 77 | TKTSDLLYYGSGSYLPY |
| 78 | TRDGGAWD |
| 79 | ARGIPREYTTRWENAFDI |
| 80 | ARDRGADKDSNSGDVFDI |
| 81 | VGPQGAY |
| 82 | ARDPRGSSTSCSYDY |
| 83 | TGQERITIFGVVIISSDY |
| 84 | ARRLNDGANHS |
| 85 | SWDATVYYDMAV |
| 86 | ARPSSGSYADPFDI |
| 87 | VASRSSSLDY |
| 88 | ARSRGYGGLAGVDY |
| 89 | ARAYFDDSSGGFDY |
| 90 | AGSTYGDYVPHFYF |
| 91 | ARGLSSFTTIVVVFVGASFYFDS |
| 92 | ARGTTSTTMIVIVITAVSTWFDP |
| 93 | ARHPLKVDTIFGVVIIDPAPFDY |
| 94 | ARIASYYYDSSGYYQTRPIGHAFDI |
| 95 | AKDRAQLLWFGQSRGMDV |
| 96 | TSTSDW |
| 97 | TRLRSGLVGFDWLPLYGMDV |
| 98 | ARRGVGILKDLPVYAMDV |
| 99 | AREARQIFITMMTTKTSWFDP |
| 100 | ARVSSTAVVTGLDYYYGMDV |
| 101 | TTISVGLLWFGLAVRDHYYFDY |
| 102 | ARSYYDSSTGYYPDALDL |
| 103 | AKSGSVWGSYHKTYYFDY |
| 104 | AKEILKGYSSGWKYYYYGMDV |
| 105 | ARATTTMVRGVIYHYYYYGMDV |
| 106 | ARERLGRMVRGVNWFDP |
| 107 | ASWTMVRGVIRWFDP |
| 108 | ARQFHYVGIVVVVAPHYYYGMDV |
| 109 | ASPRGYSYGPFDY |
| 110 | ARVLYYDILTGYWWYYYGMDV |
| 11 | ARGAPITIFGVVISTWFDP |
| 112 | ARAHTDSLELGI |
| 113 | VRKYTYDTSGFFLTSTRSAFDV |
| 114 | ARKHVYDTSGFFLSSSRNAFDV |
| 115 | ARKYSFDISGFFLSSSRYALDV |
| 116 | ARDEGVTFHDHWANEIRYGMDV |
| 117 | ARARTTMIVVVSQFDY |
| 118 | ARDRGGWLLGSYYYYGMDV |
| 119 | ARGQISHYGFGESH |
| 120 | AHSGIAVVGNQLFHYYAMDV |
| 121 | AKERSSGSQWGWTYYYYGMDV |
| 122 | ARDPYGGNRRFHGWVYYYYGMDV |
| 123 | ARESTPDVRGVMNY |
| 124 | AKDAVASAGSPDY |
| 125 | ARDKLLWFGEPVVGYYYYYYMDV |
| 126 | ARDGGGDYAQIYFDY |
| 127 | ARDRLMTTYNYYSSMDV |
| 128 | AREPGDCSGGSCYYYGMDV |
| 129 | ARATRGYSYDDAFDI |
| 130 | ASPSYTDLLTGYYVPVDY |
| 131 | AKDPRVNELLWFGSLTQFYFDD |
| 132 | AKSGGPFHLSLYYYMDV |
| 133 | ARAFYGHAFDF |
| 134 | AKGLTIPFDK |
| 135 | AKGLTIPFDK |
| 136 | ARRGKYCSGGRCYSWWFDP |
| 137 | ARVASLIGDDY |
| 138 | ARVASLIGDDY |
| 139 | AHKPSGWSLRFDS |
| 140 | ARESLFNWFDS |
| 141 | AKGLTIPFDN |
| 142 | ARVDYDSSRNY |
| 143 | ARVERWLVLGYYYYGMDV |
| 144 | GSIDY |
| 145 | AKMYSDYDDNYYGLDV |
| 146 | ARDRYCSSTSCGGYYYYMDV |
| 147 | ARAPNDFWSGYPYYFDY |
| 148 | TRDGSTAAIFGNIDY |
| 149 | ARGVVRNDYGDPGFDY |
| 150 | ATAPAYCSGGSCPENNWFDP |
| 151 | AILWFGEFYFYDLFYNAVDV |
| 152 | AILWFGEFYFYDLFYNAVDV |
| 153 | ASRREQWLGDLGYYYYGMDV |
| 154 | ARGGAHSEDY |
| 155 | ARHQDPLDIVATVDWGGLDY |
| 156 | ARVASLIGDDY |
| 157 | ATTGTDNYYYYMDV |
| 158 | ARKNCSGGICYFHDY |
| 159 | AHKPSGWSLRFDS |
| 160 | AKGQTIQLWLFGAL |
| 161 | ALTVSSWYPGIFEN |
| 162 | AKAFSGSYWDAFDI |
| 163 | AKAASGARGYYGMDV |
| 164 | ARSSSGHYVSDLGY |
| 165 | ARALNGYRYNDY |
| 166 | AREEGGGSSTHFDC |
| 167 | ARTREGSYYYGMDV |
| 168 | VRGGLQFVVAVGPYGVDV |
| 169 | VRGGLQFVVAVGPYGVDV |
| 170 | ARDIGGGAPDY |
| 171 | AIKPSIPGYFDP |
| 172 | ARVGGWQRSPRPN |
| 173 | ARVGGWQRSPRPN |
| 174 | ARGQGYGRVLLWFGE |
| 175 | ARGQGYGRVLLWFGE |
| 176 | ARPSSGSRFDY |
| 177 | ARGFDY |
| 178 | AKARGVVLFDY |
| 179 | ARHSYGSGTYLDPFDY |
| 180 | ARQPHLAYYYDSSGYNDAFDI |
| 181 | ARGAVVTPFGLDS |
| 182 | ASEDYYDSSGYYWY |
| 183 | ARLSAIAVVGYYYYAMDV |
| 184 | ARDFIAASPFYYYYYMDV |
| 185 | ATSPGGYGVRRTVLEDFRH |
| 186 | WTMEYDDYSFVYDY |
| 187 | ARGGKQQLVRNYYLDS |
| 188 | ATGFGGVIVRGFDY |
| 189 | ARVYGDYSYYMDV |
| 190 | ARDLGEAGGMDV |
| 191 | VREIESGVDFWSGHYY |
| 192 | ARDSAYYDTIGYYSGDY |
| 193 | GRSFRGSCFDYL |
| 194 | ALGTGSYYGVNY |
| 195 | AKDMGGRYSSGLYYYYYGMDV |
| 196 | ARELRGYFDY |
| 197 | ARDPNDFWSGFPRGAFDI |
| 198 | ASHARYEEETFDY |
| 199 | VRDSYTSAWTPAGYFDL |
| 200 | AKDHYGSIDY |
| 201 | ARPYTSRWFWSN |
| 202 | ALLPPNAYDYGDGLLDH |
| 203 | ARHRAAGGNYYYGMDV |
| 204 | ARERVGPAAGYMDV |
| 205 | ARAAYYYDSSGYGWFDP |
| 206 | ARGDYTEYSYYYMDV |
| 207 | ALPTGASSSYSGPNY |
| 208 | ARDEVIAVATGEGMDV |
| 209 | AKDMGYDILTGSGLGDY |
| 210 | AKEPLFGETYGMDV |
| 211 | ARDKGSGSYYSGAYYYYMDV |
| 212 | ATFNSGNDNAYEY |
| 213 | AREYPDFWSGHYYYYMDV |
| 214 | ARLPYGMDV |
| 215 | ARGLYDKSGYRSDGFDS |
| 216 | ARGFEGYCSGGRCYSYFDY |
| 217 | ARVKNWDYGLY |
| 218 | ARDGQSDWHFDL |
| 219 | ARVYGDYLDH |
| 220 | AHRSFLYNIFNGYSYAPFDY |
| 221 | AKDLFSGDRDF |
| 222 | AKDSGAVLLWFGADF |
| 223 | AREGAYDIWRGSYMRAYDH |
| 224 | ARYIEMFDP |
| 225 | ARQAYGDYGWDYYYGMDV |
| 226 | LKDWDWEYEDSRPTLRGSVY |
| 227 | ARGSVFWFGEGKNWFDP |
| 228 | ARGSVFWFGEGKNWFDP |
| 229 | AREDSSGWSRGDY |
| 230 | ARRFVVREVEYNWFDP |
| 231 | ARDGYCNSMRCYRYYHGMDV |
| 232 | ATGPTAKPNKQWGYWFDP |
| 233 | ASPVSVEQDFDI |
| 234 | TTPVGDF |
| 235 | STSHPPFFDY |
| 236 | ARGLWQLVSPVFDY |
| 237 | AKVTNRGVRGLYFDY |
| 238 | ASPVSVEQDFDI |
| 239 | AINTLLVTA |
| 240 | VHRSFLYDIFSGYSYAPFDY |
| 241 | AHRSFLYNIFDGYSYAPFDY |
| 242 | AGGADCRRTSCHYLVSNREEYMGV |
| 243 | ARGLVLSGTRYSYFYGMDV |
| 244 | VKDWDWEYEDNRPTLRGSVY |
| 245 | VKDWDWEYEESRPTLRGSVY |
| 246 | AKGGPIFWLGEGKNWFDA |
| 247 | ARDKGGILMLRGADF |
| 248 | ARTLIAAAGSAFDI |
| 249 | ARGPTSITMIVVVDDAFDI |
| 250 | ARVMNSSWYTRYYYNYMDV |
| 251 | ARRGGGCSEGVCYNFDR |
| 252 | ARGDPRDY |
| 253 | ARGSYYYDSSGYYLDY |
| 254 | ARAAYYYDSSGYGWFDP |
| 255 | TTDLGATGIYYYYYMDV |
| 256 | ARFPRDYYDSSGYLIQEGNFDY |
| 257 | ARVTRAGAAGDGGAFDI |
| 258 | ARSVVPVAGTDY |
| 259 | ARDQHPGYPALVYYYYYMDV |
| 260 | ARDNIQTFDY |
| 261 | ATSSPVAGYNSWFDP |
| 262 | ATGPAVIPLRWFDP |
| 263 | ATAPAAAGPTDWFDP |
| 264 | AISPSVHSLWWFDP |
| 265 | ARDEIHYDILTGYYNRFWFHP |
| 266 | ARDAETGYYDSSGYPINWFDP |
| 267 | ARHYYDTGAYYVPFDH |
| 268 | AHFQGFGESEYFQH |
| 269 | AHRHPLTGFDS |
| 270 | ATPRGYSYGPLDY |
| 271 | ASPRGYSYGPFDY |
| 272 | ARDRVDKGYDFWSSWYFDL |
| 273 | ASGGGSYFDAFDI |
| 274 | ARDRSGSYYGGFDY |
| 275 | AKAVYGGNSVYFDY |
| 276 | ARIYGGNYENYFDY |
| 277 | ARESEAGTTPSFDY |
| 278 | ARSLVRGVITYFDY |
| 279 | ARGLSMEV |
| 280 | ARGGYSSSWYGTKYYFDY |
| 281 | ARGPTVTTFFRRNAWFDP |
| 282 | ARGRYSSGWYGSRNWFDP |
| 283 | ARLSMGAARQSGFDP |
| 284 | ARDGGRDGYNELGARVYYYYGMDV |
| 285 | ARIGSYGI |
| 286 | AKLGCSGGSCYYYYGMDV |
| 287 | ARGDHYYDRSGPHKFDY |
| 288 | ARDSPLKFDSFGYPLYGMDV |
| 289 | ARGIVGATPGYFDY |
| 290 | AKAVSGWPIYFDA |
| 291 | AKAVSGWPIYFDA |
| 292 | AHTIHSGYDRTFDS |
| 293 | AREESYSSSSPLDY |
| 294 | AAGSDFWSGYYVNYYMDV |
| 295 | ARLTAAGVYFDY |
| 296 | AKTRGRGLYDYVWGSKDY |
| 297 | AKTRGRGLYDYVWGSKDY |
| 298 | ARDESGSYYGDQAFDI |
| 299 | ARDRRARAYEIPFGSDHYYFGMDV |
| 300 | ARDYYGSGSYPIGYMDV |
| 301 | TTSYCSTKVCFDYWFDP |
| 302 | ASNLYATSPYGGVKN |
| 303 | AKDIGSGSPDAFDI |
| 304 | VKDLEFRGGTGGFDL |
| 305 | ARDGHSAWGAFDI |
| 306 | ARDHPTLRRAFDY |
| 307 | ARDRGSSSWWGWLDP |
| 308 | ATRRGYSGYGAAYYFDY |
| 309 | AREVYVGGEDDYSYYYGLDV |
| 310 | TTDLGEAGPTEWLRSSLFDY |
| 311 | TTSYCNPKVCFDYWFDP |
| 312 | AKEYYYDSSGYYYREDAFDI |
| 313 | AKDGGLTAYLEY |
| 314 | ATEKWEVVDVCFDY |
| 315 | AKDIGWDVVVVAATHGVFDY |
| 316 | AKDPYYYGSGSSNFFDY |
| 317 | ARGPDYYDTGGYFDL |
| 318 | ARDGYKQIYWYLDL |
| 319 | AKGEGVYGSGSRYFLDY |
| 320 | AREWSRGAVAGTGYFDY |
| 321 | AKVAKLPGDYYGMDV |
| 322 | ARELRGAFDI |
| 323 | ARDWGEYYFDY |
| 324 | ARDYGDLYFDY |
| 325 | ARDRRVGSPYYYYYMDV |
| 326 | ARDLGDNAFDI |
| 327 | ARDRYSGYDF |
| 328 | ARLSGTGYGGDGGWFDP |
| 329 | AGKKIYYGSSFDP |
| 330 | ARGGSGSGWYGGRFDY |
| 331 | ARVWRETYYYDSSGDSFDY |
| 332 | ARGRSITGIRDVDF |
| 333 | ARGRGNYMFRWFDP |
| 334 | ARGGLWYDSINYYGMDV |
| 335 | ARLILRWPTTWDYFDY |
| 336 | ARVDGPFDY |
| 337 | ARCPFWNYGHCYLDN |
| 338 | ARPSVRWYYHAMDV |
| 339 | AKERRPVLRYFDWLPIEAPDY |
| 340 | ARGQYDILTGYQYGAFDI |
| 341 | AAHYYSRTDAFHI |
| 342 | ARDSVSGSGSYYKGLWFDP |
| 343 | VVGIGYCSSPSCPPLRWFDY |
| 344 | ARERGYSGSGSLYYFDY |
| 345 | AHYSSSRPPLFDY |
| 346 | AKGHWST |
| 347 | ANGAYYYGSGSYYNGAAY |
| 348 | AKGGYYDILTGYFPFDY |
| 349 | ARDLVVYGMDV |
| 350 | ARDPIRNGMDV |
| 351 | ARDLVVYGMDV |
| 352 | ARDAMSYGMDV |
| 353 | ARDRVVYGMDV |
| 354 | ARDAAVYGIDV |
| 355 | ARDLISRGMDV |
| 356 | ARDRVVYGMDV |
| 357 | ARDLVSYGMDV |
| 358 | ARDLVVYGMDV |
| 359 | ARDAQNYGMDV |
| 360 | ARDRGLVSDY |
| 361 | QQTYIIPYS |
| 362 | QQYYSYPYT |
| 363 | SSYAGSNNLV |
| 364 | QRYDSYRT |
| 365 | QQSYSTPYT |
| 366 | QQYDNLPLT |
| 367 | QQYATSPWT |
| 368 | AAWDDSLSSWG |
| 369 | QTWGTGTVV |
| 370 | QSADSSGTWV |
| 371 | QQRSDWTPT |
| 372 | QQFNSYPRT |
| 373 | CSYAGNTTF |
| 374 | STWDASLKEVL |
| 375 | MQGTHWPLT |
| 376 | QQYDSYPWT |
| 377 | QQLTTYPRT |
| 378 | QSADSSGTWV |
| 379 | QQFYSTPVT |
| 380 | QSYDGSNVV |
| 381 | QQYYSTPLT |
| 382 | QQYYDTPMYT |
| 383 | QQYNSYPYT |
| 384 | SSYTSSSTFV |
| 385 | QSADSSGTYSNWV |
| 386 | SSYTSSSTW |
| 387 | QQYGSSPLT |
| 388 | QQYGSSPLT |
| 389 | QQYGA |
| 390 | QQYGSSPWT |
| 391 | AVWDDSLNGVV |
| 392 | SSFAGSNNPYV |
| 393 | QQYYSTPYT |
| 394 | HQYDSWPPT |
| 395 | QNRDDWPPLFT |
| 396 | QQYYSTPRT |
| 397 | QQAHSFLSLT |
| 398 | QSADTSGTYLWV |
| 399 | QQYDSLPIT |
| 400 | QQYYGIPT |
| 401 | QKCDNFPWT |
| 402 | AAWDDSLSVVV |
| 403 | QQSYSSPPT |
| 404 | QSYDDTLTI |
| 405 | QQSYGAPPT |
| 406 | QQSYSTPPT |
| 407 | QQSFSTPPT |
| 408 | QQSYSSPPT |
| 409 | YSTDSSGNHWV |
| 410 | LLSYSGVRI |
| 411 | QSYDSSLSKV |
| 412 | QAWDSSTFYV |
| 413 | GTWDSSLSAVV |
| 414 | QQYNNWPWT |
| 415 | LLSYSGARPV |
| 416 | QQSYSTPPYT |
| 417 | SSYTSSSTRVV |
| 418 | QQYYSTPIT |
| 419 | QQYGSSPLT |
| 420 | GTWDSSLSVVV |
| 421 | SSYTSSSTFAV |
| 422 | MQALQTPLT |
| 423 | MQALQTVFT |
| 424 | MQALQTVFT |
| 425 | QQTYIIPYS |
| 426 | QQYYSYPYT |
| 427 | QVWDSSSDHVV |
| 428 | QAWDSSTSYW |
| 429 | GTWDSSLSVGV |
| 430 | NSYTSNSTAV |
| 431 | QQSYNWPRT |
| 432 | LQHNSYPYT |
| 433 | QQYNGYPHT |
| 434 | QQYSYYSA |
| 435 | QQYGT |
| 436 | SAWDSSLSAWV |
| 437 | QQYYSTPIT |
| 438 | QSFDDNDQV |
| 439 | LLYVGGGIWV |
| 440 | QQYNIWLT |
| 441 | MQGTLLLT |
| 442 | ETWDSSLDAVI |
| 443 | AAWDDSLSGRV |
| 444 | MQGTHWPHPT |
| 445 | MQGTPWPT |
| 446 | QQSGSSYT |
| 447 | MQSLPSGFT |
| 448 | MQSLDLPPT |
| 449 | QQGSSFPLT |
| 450 | QQYDSSPIT |
| 451 | NSRDSSGQLHVVV |
| 452 | NSRDNNDDLPL |
| 453 | SSYAGSNNLGV |
| 454 | QSYDSSLSGVV |
| 455 | QQYYSTPFT |
| 456 | MQGTHWPIT |
| 457 | SSYTSSSTLVV |
| 458 | QQSYSTPYT |
| 459 | CSYAGSYVV |
| 460 | QQSYSTLHT |
| 461 | NSRDSSGNHLV |
| 462 | QAWDTITHEEV |
| 463 | QQYNYYPVA |
| 464 | TQATQFPLT |
| 465 | QQSYSTPPYT |
| 466 | QSYDSSLSSPVV |
| 467 | AAWDDSLSGPV |
| 468 | NSRDSSGNHLV |
| 469 | QQYDNLPYT |
| 470 | GTWDSSLSAGV |
| 471 | QQYNNWPPWT |
| 472 | QAWDSSTYW |
| 473 | QQSYSSPPT |
| 474 | QQSYSSPPT |
| 475 | QQSYSSPPT |
| 476 | HHYGTSPPFT |
| 477 | QQYGSSPLT |
| 478 | QSADSSGTYYV |
| 479 | QQSYSTPRT |
| 480 | QAWDSSTVV |
| 481 | MQSIQLPLT |
| 482 | MQSIQLPFT |
| 483 | MQALQTYT |
| 484 | YSTDSSGNHRRV |
| 485 | SSYTSSSTLV |
| 486 | YSTDSSGNHRGV |
| 487 | QQYNSFPYT |
| 488 | QQRSNWPVT |
| 489 | LQHNSYPLT |
| 490 | LQHNSYPFT |
| 491 | QQYGTSAGT |
| 492 | QQYGNLPPFT |
| 493 | QQYYSTPLT |
| 494 | MQNRHLYT |
| 495 | MQNRHLYT |
| 496 | MQTLQTSIT |
| 497 | QQYGSSQYS |
| 498 | QQYGSSQYT |
| 499 | QHYDTLLT |
| 500 | QQYFDTPWT |
| 501 | MQNRQLYT |
| 502 | QQFDNLPPFT |
| 503 | QQSYSARMST |
| 504 | MQGTQWPWT |
| 505 | QQFDNSPPWT |
| 506 | QSADSSGTYVV |
| 507 | CSYAGSYTLV |
| 508 | QQSYSTPFT |
| 509 | MQGTHSYT |
| 510 | QAWDSSTASYV |
| 511 | SSYTSASTW |
| 512 | SSYTSASTW |
| 513 | MQGTHSPWT |
| 514 | GTWDSSLSAWV |
| 515 | QSADGRGDWV |
| 516 | QQYGSSQYS |
| 517 | QQYDSYSGT |
| 518 | ETWDSPYW |
| 519 | QHYDSLLT |
| 520 | SSYTSSSTW |
| 521 | MQALQTLT |
| 522 | QQYNSYPLFT |
| 523 | MQGTHWPMT |
| 524 | QQYGSSPMYT |
| 525 | QQANSFPA |
| 526 | QAWDSHTVV |
| 527 | QQYNSYSWT |
| 528 | QQYTSWPLT |
| 529 | QQYTSWPLT |
| 530 | YSPKV |
| 531 | QQYNILPHT |
| 532 | QQYYNAPLS |
| 533 | QQYYNAPLS |
| 534 | QQRSNWIT |
| 535 | QQRSNWIT |
| 536 | AAWDDSLNGPV |
| 537 | QQYGSSPQT |
| 538 | QQYNNWPPLT |
| 539 | QQYYSYSLT |
| 540 | CSYAGSSTFYV |
| 541 | QSADSSGTWV |
| 542 | QQYGSSPEMYT |
| 543 | HQYGSGLGT |
| 544 | MQSIQLRT |
| 545 | QQCSSWPLSLT |
| 546 | QQYNNWPPIT |
| 547 | QQSNSFPPT |
| 548 | QSYDISLSAYV |
| 549 | QQYNTYSLT |
| 550 | QQLNSYPPA |
| 551 | QQYYRTPLT |
| 552 | LQHHTYPLT |
| 553 | MQSIQLWS |
| 554 | LLSYSGPWV |
| 555 | SSYAGSNNYV |
| 556 | QQYDNLPSFT |
| 557 | CSYAGSYTLV |
| 558 | CSYAGSSTVV |
| 559 | QQSYNVPPWT |
| 560 | MQGTHWPWT |
| 561 | QSYDINLSAV |
| 562 | HQYHNSPWT |
| 563 | MQALQTPYT |
| 564 | QVWDSSSDHYV |
| 565 | QQYGSSPRT |
| 566 | QQYDNWLPYT |
| 567 | LLSYSGAYVL |
| 568 | QQYSNWPLYT |
| 569 | AAWDDSLNGPYV |
| 570 | SSYTSISTVL |
| 571 | QVWDGGSDDRGYV |
| 572 | SSFTSNGAWV |
| 573 | QQNYIRPYT |
| 574 | QQYDNLPIT |
| 575 | ATWDDSLNGV |
| 576 | QQYNNWPYT |
| 577 | GADHGSGSNFVYV |
| 578 | CSYAGSSTLV |
| 579 | QQHDSAPYT |
| 580 | QQYNSYVT |
| 581 | MQGKHLRWT |
| 582 | YSTDYSGNHGV |
| 583 | QQCSNWPNT |
| 584 | QSADSNDSWV |
| 585 | GTWDSSLSAGV |
| 586 | QQGHNFPWT |
| 587 | QQYGSLPLT |
| 588 | QQYGSLPLT |
| 589 | QSYDSSLSGWV |
| 590 | QQRRNWPLT |
| 591 | QHRSNWPYT |
| 592 | AAWDDSLNGW |
| 593 | MQTTQFPRT |
| 594 | QSQDSSATYVV |
| 595 | QAWDSSIEV |
| 596 | QQYGSSPPWT |
| 597 | QSGDSSGTYVV |
| 598 | MQTTQFPRT |
| 599 | LQYNTYSYS |
| 600 | QQYNSYIT |
| 601 | QQYNSYVT |
| 602 | YSTDSSDNQRV |
| 603 | QHLKSYPLT |
| 604 | QQGHNFPWT |
| 605 | QQGHNFPWT |
| 606 | QQYHNFP |
| 607 | QSYDSSLSVV |
| 608 | QAWDSNTGV |
| 609 | QSYDSSLSGSV |
| 610 | QSVDNTGASPHVV |
| 611 | QQYHTYWT |
| 612 | QAWDSGT |
| 613 | QQYGSSPRT |
| 614 | QQYGSSPRT |
| 615 | QHYGTSPYT |
| 616 | QQYGSSTLVT |
| 617 | CSYAGSSLWV |
| 618 | QSYDSSFWV |
| 619 | GTWDSSLSAVV |
| 620 | YSTDRSGNHRGV |
| 621 | NSRDSSGNHLYWV |
| 622 | QSYDSSLSGHVV |
| 623 | GTWDSSLSAGGV |
| 624 | CSYAGSSTFVV |
| 625 | GTWDSSLSAVV |
| 626 | QQLNSYPPT |
| 627 | QQSYSTLWT |
| 628 | QQYGDSPET |
| 629 | QAWDSSTVV |
| 630 | QQYDNLPYT |
| 631 | QQYDNLPYT |
| 632 | QQRSNWPSIT |
| 633 | QQANSFPLA |
| 634 | QQSYSTPFG |
| 635 | LSYDSSLSGSV |
| 636 | QQFNNYPLT |
| 637 | QQYDNLPFT |
| 638 | SSYTSSSAYV |
| 639 | NSRDSSGNHW |
| 640 | CSYAGSYPVV |
| 641 | SSYAGSNKV |
| 642 | QQYGSSGGYT |
| 643 | QQSYSTPYT |
| 644 | QQYGSSSWT |
| 645 | QQSYSTPYT |
| 646 | QAWDSSTANWV |
| 647 | SSFTDSSTLVV |
| 648 | QQSYSVPHT |
| 649 | QQYNNWLT |
| 650 | QQYNNWPPIT |
| 651 | QQYNNWPPIT |
| 652 | SSYAGTNKIL |
| 653 | QQSYSTPLT |
| 654 | SSYTSSSTWV |
| 655 | QQSYSTPYT |
| 656 | MQALQTPGT |
| 657 | MQALQTPGT |
| 658 | QQYNSYSA |
| 659 | QAWDRTTAT |
| 660 | QSYDSSLSGWV |
| 661 | SSYTSLNTLEVV |
| 662 | MQALQTPYS |
| 663 | QVWDSSSDRTVV |
| 664 | ASWDDKVRGWV |
| 665 | QQYGSSPWT |
| 666 | QQYNSYSRT |
| 667 | QQYNTSPLT |
| 668 | QSYDSSLSGSL |
| 669 | QSADSSGTYRV |
| 670 | QQYGRT |
| 671 | SSYTNIDTLEIV |
| 672 | LQHNSYPRT |
| 673 | QVWHSSFDPWV |
| 674 | QQSYSTPPTT |
| 675 | QQYNSYFPT |
| 676 | QVWDSSSDHYWV |
| 677 | GTWDSSLSAGV |
| 678 | QTWGTGPQVL |
| 679 | QQYDNLLT |
| 680 | QVWDSSGDHWV |
| 681 | QQRSNWLT |
| 682 | QQHDNLPSFT |
| 683 | QQYGSSPRT |
| 684 | QQYGSSPRT |
| 685 | LLYYGGAPV |
| 686 | QQLNSYPPA |
| 687 | QQYDNLPQT |
| 688 | CSYAGSSLWV |
| 689 | QSYDSSNQV |
| 690 | QQRSNWLFT |
| 691 | GTWDSSLSAGV |
| 692 | MQASQFPLT |
| 693 | CSFAGSNRE |
| 694 | QQYGSSPWT |
| 695 | GTWDSSLSAWV |
| 696 | QHYSSSAPIT |
| 697 | QQRNKWPGT |
| 698 | QQYGDSPYT |
| 699 | QQLNSYPLT |
| 700 | CTYAGSSTWV |
| 701 | QQSYSSPYT |
| 702 | QQANSFPRT |
| 703 | QQFNDYPLT |
| 704 | QSYDSSLSGSV |
| 705 | QQYSTYYT |
| 706 | MQGSHWPWT |
| 707 | AAWDDSLNGPWV |
| 708 | CSYAGSYTWV |
| 709 | QQLNSYPFT |
| 710 | QQYDNLPRT |
| 711 | QQLNSYPLT |
| 712 | QQSYSTPPDT |
| 713 | QQYDNLPPT |
| 714 | QQSYTTPLFT |
| 715 | QQLNGYPHSA |
| 716 | HQYDNLPPT |
| 717 | QQLNSYPLT |
| 718 | QQLNSNPPIT |
| 719 | QQSYSTPPYT |
| 720 | HQYDNLPRT |
| 721 | QVQLVESGGGVVQPGRSLRLSCAASGFTFSSYGMH |
| | WVRQAPGKGLEWVAVIWYDGSNKYYADSVKGRFTI |
| | SRDNSKNTLYLQMNSLRA |
| | EDTAVYYCAREGVDTAMVGFDYWGQGTLVTVSS |
| 722 | QVQLVESGGGVVQPGRSLRLSCAASGFTFSSYGMH |
| | WVRQAPGKGLEWVAVIWYDGSNKYYADSVKGRFTI |
| | SRDNSKNTLYLQMNSLRAEDTAVYYCAREGVDTAM |
| | VGFDYWGQGTLVTVSS |
| 723 | EVQLVQSGAEVKKPGESLRISCKGSGYSFTSYWIS |
| | WVRQMPGKGLEWMGRIDPSDSYTNYSPSFQGHVTI |
| | SADKSISTAYLQWSSLKASDTAMYYCARSTRRWLQ |
| | FVFPFDYWGQGTLVTVSS |
| 724 | EVQLVQSGAEVKKPGESLRISCKGSGYSFTSYWIS |
| | WVRQMPGKGLEWMGRIDPSDSYTNYSPSFQGHVTI |
| | SADKSISTAYLQWSSLKASDTAMYYCARSTRRWLQ |
| | FVFPFDYWGQGTLVTVSS |
| 725 | EVQLVQSGAEVKKPGESLRISCKGSGYSFTSYWIS |
| | WVRQMPGKGLEWMGRIDPSDSYTNYSPSFQGHVTI |
| | SADKSISTAYLQWSSLKASDTAMYYCARSTRRWLQ |
| | FVFPFDYWGQGTLVTVSS |
| 726 | EVQLVQSGAEVKKPGESLRISCKGSGYSFTSYWIS |
| | WVRQMPGKGLEWMGRIDPSDSYTNYSPSFQGHVTI |
| | SADKSISTAYLQWSSLKASDTAMYYCARSTRRWLQ |
| | FVFPFDYWGQGTLVTVSS |
| 727 | EVQLVQSGAEVKKPGESLRISCKGSGYSFTSYWIS |
| | WVRQMPGKGLEWMGRIDPSDSYTNYSPSFQGHVTI |
| | SADKSISTAYLQWSSLKASDTAMYYCARSTRRWLQ |
| | FVFPFDYWGQGTLVTVSS |
| 728 | QMQLQESGPGLVEPSETLALTCTVSGGSINRNHFW |
| | AWLRRPPGKGLEWIGSASYTGTTHDNPSLRSRLTI |
| | SVDTSKNQFSLKMTSVTVADTAVYFCARQAPGGGL |
| | LGYYHGLDVWGQGTTVTVSP |
| 729 | QMQLQESGPGLVEPSETLALTCTVSGGSINRNHFW |
| | AWLRRPPGKGLEWIGSASYTGTTHDNPSLRSRLTI |
| | SVDTSKNQFSLKMTSVTVADTAVYFCARQAPGGGL |
| | LGYYHGLDVWGQGTTVTVSP |
| 730 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARDRYCGGD |
| | CSGPHYYYYGMDVWGQGTTVTVSS |
| 731 | EVQLVESGGGLVQPGGSLRLSCAASGFTFSYFEMN |
| | WVRQAPGKGLEWISYISSSGTNIYYADSVKGRFTI |
| | SRDNAENSLYLQMNSLRVEDTAVYYCARWDCSGGS |
| | CNYYYYYNMDVWGQGTRVTVSS |
| 732 | EVQLVESGGGLVQPGGSLRLSCAASGFTFSYFEMN |
| | WVRQAPGKGLEWISYISSSGTNIYYADSVKGRFTI |
| | SRDNAENSLYLQMNSLRVEDTAVYYCARWDCSGGS |
| | CNYYYYYNMDVWGQGTRVTVSS |
| 733 | QVQLVQSGAEVKKPGASVKVSCKASGYKFSNYYIH |
| | WVRQAPGQGLEWMGWINPYSGETNYAQKFQGRVTM |
| | TRDTSTSTAYMELSRLRADDTAVFFCAREDILLVP |
| | AASNFYYFGMDVWGQGTTVAVSS |
| 734 | QVQLVQSGAEVRKPGASVKISCKSSGYIFTNFYVD |
| | WVRQAPGRGLEWMGRVNPNDGSSIYAQKFRDRFSL |
| | TSDTSTSTVFLNLRGLTSEDTALYFCARGDYYDPD |
| | DRYNAYYSLGAWGQGTTVIVSS |
| 735 | EVQLLESGGGLQQRGGSLRLSCAASGFNFSSYAMS |
| | WVRQAPGKGLEWVSSISATGGTTFYADSEKGRFTI |
| | SRDNSKNILYLQMNSLRAEDTAVYYCTKGSMLLEV |
| | YWGQGTLVTVSS |
| 736 | EVQLVESGGGLVQPGGSLRLSCGVSGIIVSRNEMS |
| | WVRQAPGKGLEWVSYISSSGTGVHYADSVKGRFTS |
| | SRDSAKNSVYLQMHSLRAEDTAVYYCARAPSDSSG |
| | INGAFDIWGQGTMVTVSS |
| 737 | QVQLVQSGAEVKKPGSSVKVSCKASGGTFSTYAIS |
| | WVRQAPGQGLEWMGGIIPIFGTPTYAQRFQGRVTI |
| | TADESTSTAYMELTSLRSDDTAVFYCARPKAPGYS |
| | YLSLDYWGQGTLVTVSS |
| 738 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCCGFGVVTTD |
| | AYGMDVWGQGTTVTVSS |
| 739 | EVQLVESGGGLVQPGGSLRLSCSASGFTFNNYAMH |
| | WVRQAPGKGLEHVSVISSYGDNTFYADSVKGRFTI |
| | SRDNSKNTLYLQMSSLRAEDTAVYYCVKDKACTTT |
| | SCYEGTFFDYWGQGTLVTVSS |
| 740 | EVQLVESGGGLVQPGGSLRLSCAASGFVFSNYWMT |
| | WVRQAPGKGLEWVANIKQDESEEYYRDSLKGRFTI |
| | SRDNAKNSVFLQMDSLRVEDSAVYYCVRGDDSILT |
| | PTFDHWGQGTLVTVSS |
| 741 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARAGKGFM |
| | VITHFDYWGQGTLVTVSS |
| 742 | EVELVQSGAEMKEPGESLKISCKGFGYNFNNYWVA |
| | WVRQTPGKGLEWMGIIYGGDSDTRYNPSMQGQVTI |
| | SADKSINTIYLEWDVLRASDSGIYYCARPHTNSWD |
| | QFDYWGQGTLVTVSS |
| 743 | QVQLVQSGSELKKPGASVKVSCKASGYTFTSYAMN |
| | WVRQAPGQGLEWMGWINTNTGNPTYAQGFTGRFVF |
| | SLDTSVSTAYLQISSLKAEDTAVYYCARPQGGSSW |
| | YRDYYYGMDVWGQGTTVTVSS |
| 744 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATSTAVLRY |
| | FAPTGGWFDPWGQGTLVTVSS |
| 745 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCAKDNGHSYG |
| | YSWFDPWGQGTLVTVSS |
| 746 | QVQLVESGGGVVQPGRSLRLSCAASGFTFSSYPMH |
| | WVRQAPGKGLEWVAVISYDGSNKYYADSVKGRFTI |
| | SRDNSKNTLYLQMNSLRAEDTAVYYCATDGATIPI |
| | NYYGMDVWGQGTTVTVSS |
| 747 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARSPITMI |
| | VVVNAFDIWGQGTMVTVSS |
| 748 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARARITMI |
| | VVVNHFDYWGQGTLVTVSS |
| 749 | EVQLVESGGRSVQPGGSLRLSCEASGFTVSSNYMN |
| | WVRQAPGKGLEWLSVLYSGGNEYYADSVRGRFTIS |
| | RHSSKNTLFLQMNRLRPEDTAVYYCARVQSTGYKY |
| | WYFDIWGRGTLVIVSS |
| 750 | QVQLQESGPGLVKPSETLSLTCTVSGGSISSYYWS |
| | WIRQPPGKGLEWIGYIYYSGSTNYNPSLKSRVTIS |
| | VDTSKNQFSLKLSSVTAADTAVYYCARGFDYWGQG |
| | TLVTVSS |
| 751 | QVQLVQSGAEVKKPGASVKVSCKASGYTFTGYFIY |
| | WVRQAPGQGLEWMGRINPSSGVANYAQKFQGRVTM |
| | TRDTSITTAYMELSRLTSDDTVVYYCARARDYGSG |
| | SPMDVWGQGTTVTVSS |
| 752 | EVQLVESGGGLVQPGGSLRLSCVASGFTASSNYMN |
| | WVRQAPGKGLEWVSVIYAGGGTHYADSVKGRFTIS |
| | RDNFKNTVYLQMNSLRSEDTAVYYCARDGVYYGSV |
| | IYHHYDLHVWGQGTTVTVSS |
| 753 | QVQLVQSGPEVKKPGSSVKVSCKVSGGTFSSYGIS |
| | WVRLAPGRGLEWMGRILPVLDTTTYAPKFEGRVTI |
| | TADESTTTAYMELTSLKSDDTAVYYCARGGGELLR |
| | YPFDYWGQGTPVTVSS |
| 754 | QVHLVQSGPEVKKPGSSVKVSCKASGGRFGSFAFS |
| | WLRQAPGQGLEWMGKVTPIVGVPVYAEKFQGTVTI |
| | SADESTNTAYMEVSSLRSEDTALYYCAKAGLGLET |
| | SGGNYFESWGQGTLVTVSS |
| 755 | QVRLVESGGGLVQPGRSLRLSCAASGFTFTDYAIH |
| | WVRQAPGKGLEWMATISYDGNDKYFAASVRGRFSI |
| | SRDNSNNTLFLQMNNLRAEDTAVYYCAKDRVTMNY |
| | FDYWGQGTLVSVSS |
| 756 | QVQLQESGPGLVKPSETLSLTCTVSGGSISSYYWS |
| | WIRQPPGKGLEWIGYIYYSGSTNYNPSLKSRVTIS |
| | VDTSKNQFSLKLSSVTAADTAVYYCARVREGYTSG |
| | WYADYWGQGTLVTVSS |
| 757 | QVQLVQSGAEVQKPGASVRVSCKASGYTFTDYYIH |
| | WVRQAPGQGLEWMGWVNPNRGGTNNAQKFQGRVTM |
| | TRDTSITTAYMELHSLRSDDTAVYYCARDRSYYHS |
| | SGYHYYFDYWGQGSLVTVSS |
| 758 | QVQLVQSGAEVKKPGASVKVSCKASGYSFTGHYIH |
| | WVRQAPGQGLEWMGWINPDSGGTNNAQKFQGRVTM |
| | ARDTSISTAYMDLSTLTNDDTAVYYCVRDRIVGGY |
| | SYGGDYWGQGTLVTVSS |
| 759 | EVQLLESGGGLVQPGGSLRLSCAASGFTFSNYGMS |
| | WVRQAPGKGLEWVSAITGSGGSTHYADSVKGRFTI |
| | SRDNSNNTLSLQMNSLRAEDTAVYYCAKGRLSPRL |
| | GQGTLVTVSS |
| 760 | QLQLKESGSGLVKSSQTLSLTCAVSGGSISSDVYS |
| | WSWIRQAPGKGLEYIGYVFHTGSAYYNPSLKSRVI |
| | ISVDRSKNQVSLNVTSVTAADTAIYYCARVKVDNV |
| | VFDLWGQGTMVTVSS |
| 761 | QVQLVQSGTEVKKPGSSVKVSCKASGDTFNSYAIS |
| | WVRQAPGQGLEWMGRIIPILRLATYAQEFQGRVTI |
| | TADKSTTTTYMEVTSLKSEDTAIYYCARDRGLAAR |
| | PAGWVDLWGQGTLVTVSS |
| 762 | QTQLVESGGGVVQPGRSLRLSCAASGFTFSHYGMH |
| | WVRQAPGKGLEWVALIWYDGSKKYYADSVKGRFTI |
| | SRDISENTLYLQMNSLRAEDTAVYYCARENFHFSG |
| | TPPLYWGQGTLVTVSS |
| 763 | EVQLVQSAAEQKKPGESLKLSCKGSGYSFPAHWID |
| | WVRQMPGGGLEWVGSIFPGDSDTKYSPSFEGQVNI |
| | SADRSINTAYLQWSSLKASDTAIYYCARKYTYDTS |
| | GFFLSSSRNAFDVWGQGSMVFVSS |
| 764 | EVQLVQSGAEVKKPGESLKISCKGSGYNFDTYWIA |
| | WVRQTPGKGLEWMGDIYPGDSDSRYSPSFQGRVTF |
| | SADKSISVAYLQWSTLKASDTAMYFCARLGSNGYG |
| | LWGQGTLITVSS |
| 765 | EVQLVQSGAEVKEPGESLKISCKGSGYSFSGYWIA |
| | WVRQRPGKGLEWMGTIFPSDSDTRYSPSFEGQVTI |
| | STDKSISTAYLQWSSLKASDTAMYYCARTYSYDSS |
| | GFFLTSSREAFDIWGQGTMVIVSS |
| 766 | EVQLVQSGAEVKKPGESLKISCKASGYYFAAHWID |
| | WVRQMPGRGLEWMGSIFPSDSDTEYGPSFQGQVNI |
| | SADKSITTAYLQLKNLKASDTALYYCVRKYSFDVS |
| | GFFLSSSRHAFDVWGQGTMVTVSS |
| 767 | EVHLVQSGPEQKKPGESLRISCKGSGYSFPAFWIV |
| | WVRQMPGEGLEWMGSVFPGDSDTEYSPSFQGQVTI |
| | SADKSISTAYLQWSSLKASDTAMYYCARKYSYDTS |
| | GFFLTSSRDAFDVWGQGTMIAVSS |
| 768 | DVQLVQSGAEEKKPGEFLKISCKGSGYSFPAYWIG |
| | WVRQMPGKGLEWMGSIFPGDSDTEYSPSFQGHVTI |
| | SADKSISTAYLQWSSLKASDTAMYYCVRKFSYDIS |
| | GFFLTSSRDAFDVWGQGTKVTISS |
| 769 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATEGVWGQG |
| | TTVTVSS |
| 770 | QVQLVQSGAEAKKPGASVKVSCKASGYTFTRYWMH |
| | WVRQGPGQGLEWMGLMKPGDGKTIYAQKFQYRVTL |
| | TRDTSTSTVYMELRSLTSADTAMYYCLLIEGMGAT |
| | SGDWGQGTLVTVSS |
| 771 | EVQLVESGGGLVKPGGSLRLSCAASGFTFSSYSMN |
| | WVRQAPGKGLEWVSSISSSSSYIYYADSVKGRFTI |
| | SRDNAKNSLYLQMNSLRAEDTAVYYCATTNDGYYY |
| | GMDVWGQGTTVTVSS |
| 772 | EVQLVESGGGLVQPGGSLRLSCAASGFTFSSYSMN |
| | WVRQAPGKGLEWVSYISSSSSTIYYADSVKGRFTI |
| | SRDNAKNSLYLQMNSLRAEDTAVYYCATNPHNTAM |
| | VLDYYGMDVWGQGTTVTVSS |
| 773 | QLQLQESGPGLVKPSETLSLTCTVSGGSISSSSYY |
| | WGWIRQPPGKGLEWIGSIYYSGSTYYNPSLKSRVT |
| | ISVDTSKNQFSLKLSSVTAADTAVYYCAGAYIAAA |
| | GWGWELFQYYFDYWGQGTLVTVSS |
| 774 | QITLKESGPTLVKPTQTLTLTCTFSGFSLSTSGVG |
| | VGWIRQPPGKALEWLALIYWDDDKRYSPSLKSRLT |
| | ITKDTSKNQVVLTMTNMDPVDTATYYCAHQAPFEW |
| | FGVDYWGQGTLVTVSS |
| 775 | EVQLVESGGGLVKPGGSLRLSCAASGFTFSNAWMS |
| | WVRQAPGKGLEWVGRIKSKTDGGTTDYAAPVKGRF |
| | TISRDDSKNTLYLQMNSLKTEDTAVYYCTTDGLYC |
| | SGGSCYYHSYYYYYGMDVWGQGTTVTVSS |
| 776 | EVQLVESGGGLVQPGGSLRLSCAASGFTFSSYEMN |
| | WVRQAPGKGLEWVSYISSSGSTIYYADSVKGRFTI |
| | SRDNAKNSLYLQMNSLRAEDTAVYYCARDGLGNYD |
| | ILTGYTERAFDIWGQGTMVTVSS |
| 777 | EVQLVESGGGLVQPGGSLRLSCAASGFTFSSYEMN |
| | WVRQAPGKGLEWVSYISSSGSTIYYADSVKGRFTI |
| | SRDNAKNSLYLQMNSLRAEDTAVYYCARVKPILRV |
| | VVVAATPCDYWGQGTLVTVSS |
| 778 | QLQLQESGPGLVKPSETLSLTCTVSGGSISSSSYY |
| | WGWIRQPPGKGLEWIGSIYYSGSTYYNPSLKSRVT |
| | ISVDTSKNQFSLKLSSVTAADTAVYYCARHARGYQ |
| | LLSPRLGELSLYRSFDYWGQGTLVTVSS |
| 779 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARATTTKM |
| | IVVVINAFDIWGQGTMVTVSS |
| 780 | QLQLQESGPGLVKPSETLSLTCTVSGGSISSRSYY |
| | WGWIRQPPGKGLEWIGSIYYSGSTYYNPSLKSRVT |
| | ISVDTSKNQFSLKLSSVTAADTAVYYCARHWITMI |
| | VVVIKGGWFDPWGQGTLVTVSS |
| 781 | QVTLKESGPVLVKPTETLTLTCTVSGFSLSNARMG |
| | VSWIRQPPGKALEWLAHIFSNDEKSYSTSLKSRLT |
| | ISKDTSKSQVVLTMTNMDPVDTATYYCARIRGQWL |
| | VGKYYYGMDVWGQGTTVTVSS |
| 782 | QITLKESGPTLVKPTQTLTLTCTFSGFSLSTSGVG |
| | VGWIRQPPGKALEWLALIYWDDDKRYSPSLKSRLT |
| | ITKDTSKNQVVLTMTNMDPVDTATYYCAHRGWGFS |
| | SSFFDYWGQGTLVTVSS |
| 783 | QVQLVESGGGVVQPGRSLRLSCAASGFTISPYGMH |
| | WVRQAPGKGLECVAIIWYDGSNKYYADSVKGRFTI |
| | SRDSSKNTLYLQMDRLRAEDTAVYYCARMSSSLQH |
| | YYGMDVWGQGTTVTVSS |
| 784 | QVQLVESGGGVVQPGRSLRLSCAASGFTISPYGMH |
| | WVRQAPGKGLECVAIIWYDGSNKYYADSVKGRFTI |
| | SRDSSKNTLYLQMDRLRAEDTAVYFCARMSSSLQH |
| | YYGMDVWGQGTTVTVSS |
| 785 | QVQVVQSEGEVKKPGASVKVSCMASGYTFGDYGIS |
| | WVRQAPGQGLEWMGWISGYNGDPKYAQKFQGRITL |
| | TTDAATSSAYMELRSLRSDDTAVYFCARDVTLVRG |
| | TASPRFDYWGQGTLITVSS |
| 786 | QVQVVQSEGEVKKPGASVKVSCMASGYTFGDYGIS |
| | WVRQAPGQGLEWMGWISGYNGDPKYAQKFQGRITL |
| | TTDAATSSAYMELRSLRSDDTAVYFCARDVTLVRG |
| | TASPRFDYWGQGTLITVSS |
| 787 | QITLKESGPTLVKPTQTLTLTCTFSGFSLSTSGVG |
| | VGWIRQPPGKALEWLALIYWDDDKRYSPSLKSRLT |
| | ITKDTSKNQVVLTMTNMDPVDTATYYCAQEGRNYD |
| | RNWFDPWGQGTLVTVSS |
| 788 | QVRLQESGPGLVKPSETLSLTCTVSGGSISTYRWS |
| | WIRQPPGKGLEWIGYIYYSGRTNYHPSLKSRVTMS |
| | VDTSKNQFSLKLTFVSAADTAVYYCARLIPIDGRD |
| | VWGRGTTVTVSS |
| 789 | EVQLVESGGGLVEPGGSLRLSCAASGFTFSNAWMC |
| | WVRQAPGKGLEWVGRIKRIIDGGTINYAAPVKGRF |
| | TISRDDSTNTVYLQMNSLRSEDTAVYYCTTYWDQY |
| | TSTWTWGQGTLVTVSS |
| 790 | QVQLVQSGSELKKPGASVKVSCKASGYIFTNYAIN |
| | WVRQAPGQGLEWMGWTNTNTGNPTYAQGFTGRFVF |
| | SLDTSVSTAYLQISSLKAEDTAVYYCASIVKYDSS |
| | GYNFDYWGQGTLVTVSS |
| 791 | QVQLVQSGAEVKKPGASVKLSCKTSGYAFTSYQVH |
| | WVRQAPGQGLEWMGMINPSGSATHYAQKWQGRVSM |
| | TADTSTTTVYMELSGLRSEDTAVYYCTRDPWHESE |
| | HRFDPWGQGTLVTVSS |
| 792 | EVQLVESGGGLVQPGRSLRLSCAASGFTFGDYAMH |
| | WVRQVPGKGLEWVSSITWNSGNIGYADSVKGRFTI |
| | SRDNAKNSLYLQMNSLRIEDTALYYCAKDNKVSSW |
| | YSFDIWGQGTMVTVSS |
| 793 | QVQLQQWGAGLLKPSETLSLTCAVSGASFSSYYWT |
| | WIRQPPGKGLEWIGDISQSASTNYSPSLKSRVTIS |
| | ADASRTQFSLNLISVTAADTAVYYCARGLGYYVAL |
| | GQGTLVTVSS |
| 794 | EVQLVQSGVEVKEPGESLKISCKSSGYSFTKYWIG |
| | WVRQMPGKGLEWLGIIYPDDSETRYSPSFRGQVTI |
| | SADKSISTAYLAWDRLKASDTAIYYCVRGGQEVSL |
| | RRLDWFVGYWGQGTLVTVSS |
| 795 | EVQLLESGGGLVQPGGSLRLSCAASGFTFSSYAMS |
| | WVRQAPGKGLEWVSSISGSGDKTYYADSVKGRFTI |
| | SRDNSKNTLYLQMNSLRAEDTALYYCAKERGGSGK |
| | MYDYWGQGNLVTVSS |
| 796 | QVQLQQSGPGLLKPSQTLSLTCAISGDSVSSNTVA |
| | WSWIRQSPSRGLEWLGRTYYRSNWYNDYAVSVKGR |
| | ITLNSDTSKNQLSLQLNSVTPEDTAVYYCARRGAA |
| | VAGTTGGSAFDIWGQGTMVTVSS |
| 797 | EVQLVESGGGLVQPGRSLRLSCAASGFTFDDYGMN |
| | WVRQAPGKGLEWVSGISWNSNSVAYADSVNGRFTI |
| | SRDNAKNSLYLQMNSLRIEDTAFYYCTKTSDLLYY |
| | GSGSYLPYWGQGTLVVVSS |
| 798 | AVQLVESGGGFVQPGRSLRLSCAGSGFAFDDFAMH |
| | WVRQAPGKGLEWVSGINWNSDNIAYAASVKGRFIV |
| | SRDNGKNSLYLQMNSLRPEDTALYYCTRDGGAWDW |
| | GRGTLVTVSS |
| 799 | EVQVVESGGGLVQPGGSLRLSCAASGFTVSSTFMS |
| | WVRQAPGKGLEWVSVIYTVGDTFYADSVKGRFTIS |
| | RHTSNNALYFQMNSLRTEDTAVYYCARGIPREYTT |
| | RWENAFDIWGQGTMVTVSS |
| 800 | QVQLQESGSGLVKPSQTLSLTCSVSGGSIKRRGYY |
| | WSWIRQHPGKGLEWIGYIYYSGTTYYNPSLQSRVN |
| | ISVDTSKNQFSLNLRSVTAADTAVYYCARDRGADK |
| | DSNSGDVFDIWGQGTMVTVSS |
| 801 | QVQLQQWGAGLLKPSETLSLTCAVYGGSFSAYYWS |
| | WIRQPPGKGLEWIGEINRRGNTNYNPSLKGRVTIS |
| | IHTSKNQFSLNLSSMTAADTAVYYCVGPQGAYWGQ |
| | GTLVTVSS |
| 802 | QLQLQESGPGLVKPSETLSLTCVVSGGSISSSDYY |
| | WGWIRQPPGKGLEWIGTIYYSGNTFYNPSLKSRVT |
| | MSVDPSKNQFSLKLSSVTAADTAVYYCARDPRGSS |
| | TSCSYDYWGQGTLVTVSS |
| 803 | EVQLVESGGGLVKPGGSLRLSCAASGFTFSNAWMS |
| | WVRQAPGKGLEWVGRIKSKTDGGTTDYAAPVKGRF |
| | TISRDDSKNTLYLQMNSLKTEDTAVYYCTGQERIT |
| | IFGVVIISSDYWGQGTLVTVSS |
| 804 | QVHLVQSGAEVKKPGSSVKVSCKASGGTFSTYAIS |
| | WVRQAPGQGLEYMGGIIPSLRTANYAQRFQDRVSI |
| | TADESTTTAYMELSSLRSDDTAVYYCARRLNDGAN |
| | HSWGQGTRVTVSS |
| 805 | EVQLVQSGGGLVKPGESLRLSCAVSGLRFTDAWLN |
| | WVRQAPGKGLEWVGRIKSRGSGGTIELAAPVKGRF |
| | TISRDDSKSTLFLQMNSLRTEDTAIYYCSWDATVY |
| | YDMAVWGQGTTVTVSS |
| 806 | QVQLVESGGGVVQPGGSLRLSCAASGFSFSSYALH |
| | WVRQAPGKGLEWVALISYDGRNKYYADSVKGRFTI |
| | SRDNSKKTLYLQMSTLTAEDTAVFYCARPSSGSYA |
| | DPFDIWGQGTMVTVSS |
| 807 | QTVVESGGAVVQPGKSLTLSCEASGFSFSDFAMHW |
| | VRQSPGKGLEWVAVVSYDSRQQYYADSVQGRFRIS |
| | RDNSQYTVTLRMDTLSFEDTGIYFCVASRSSSLDY |
| | WGQGTRVTVSS |
| 808 | QIQLVESGGGVVQPGRSLRLSCAASGFTFTTYGFH |
| | WVRQAPGKGLEWVAVIWYDGSNEAYADSVKGRITI |
| | SRDNSRNTVYLQMNSLRAEDTAIYHCARSRGYGGL |
| | AGVDYWGQGTLVTVSS |
| 809 | DVQLVESGGGLVQPGGSLRLSCLATGFTFRSYSMN |
| | WVRQAPGKGLEWISYLSNDDRTRKYADSVNGRFTI |
| | SRDNDGSSLFLQMDSLRDEDTAIYYCARAYFDDSS |
| | GGFDYWGQGALVIVSS |
| 810 | QVQLQESGPGLVKPAETLSLTCTVSGDSITSYYWS |
| | WIRQPAGKGLEWIGRIYSSGDTNYDPSLKSRVTMS |
| | VDTSKDQFSLRLSSVTAADTAIYYCAGSTYGDYVP |
| | HFYFWGQGTLVTVSS |
| 811 | QVQLQQWGAGLLKPSETLSLTCAVYGGSFSGSYWS |
| | WIRQSPGKGLEWIGEINPSGGSNYNPSLKSRVIIS |
| | LDTSKNQFSLKLNSVTAADTAVYYCARGLSSFTTI |
| | VVVFVGASFYFDSWGQGTLATVAS |
| 812 | QVQLQQWGAGLLKPSETLSLTCAVSGGSFTDHYWT |
| | WIRQPPGKGLEWIGEINHSGRTNYSPSLKSRVTMS |
| | LDTSKNQFSLKLRSVTAADTGIYYCARGTTSTTMI |
| | VIVITAVSTWFDPWGQGTLVTVSS |
| 813 | QLQLQESGPGLVKPSETLSLTCTVSGGSISSSSYY |
| | WGWIRQPPGKGLEWIGSIYYSGSTYYNPSLKSRVT |
| | ISVDTSKNQFSLKLSSVTAADTAVYYCARHPLKVD |
| | TIFGVVIIDPAPFDYWGQGTLVTVSS |
| 814 | QVTLKESGPVLVKPTETLTLTCTVSGFSLSNARMG |
| | VSWIRQPPGKALEWLAHIFSNDEKSYSTSLKSRLT |
| | ISKDTSKSQVVLTMTNMDPVDTATYYCARIASYYY |
| | DSSGYYQTRPIGHAFDIWGQGTMVTVSS |
| 815 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCAKDRAQLLW |
| | FGQSRGMDVWGQGTTVTVSS |
| 816 | EVQLVESGGGLVKPGRSLRLSCTASGFTFGDYAMS |
| | WFRQAPGKGLEWVGFIRSKAYGGTTEYAASVKGRF |
| | TISRDDSKSIAYLQMNSLKTEDTAVYYCTSTSDWW |
| | GQGTLVTVSS |
| 817 | EVQLVESGGGLVQPGGSLKLSCAASGFTFSGSAMH |
| | WVRQASGKGLEWVGRIRSKANSYATAYAASVKGRF |
| | TISRDDSKNTAYLQMNSLKTEDTAVYYCTRLRSGL |
| | VGFDWLPLYGMDVWGQGTTVTVSs |
| 818 | EVQLVQSGAEVKKPGESLRISCKGSGYSFTSYWIS |
| | WVRQMPGKGLEWMGRIDPSDSYTNYSPSFQGHVTI |
| | SADKTISTAYLQWSSLKASDTAMYYCARRGVGILK |
| | DLPVYAMDVWGQGTTVTVSs |
| 819 | QVQLVQSGAEVKKPGASVKVSCKASGYTFTSYYMH |
| | WVRQAPGQGLEWMGIINPSGGSTSYAQKFQGRVTM |
| | TRDTSTSTVYMELSSLRSEDTAVYYCAREARQIFI |
| | TMMTTKTSWFDPWGQGTLVTVSS |
| 820 | QVRLVQSGAEVKKPGSSVKVSCKASGGTFSSYAIS |
| | WVRQAPGQGLEWMGRIIPIFHIANSAQKFQGRVTI |
| | TADKSTSTAYMELSSLRSEDTAVYYCARVSSTAVV |
| | TGLDYYYGMDVWGQGTTVTVSS |
| 821 | EVQLVESGGGLVKPGGSLRLSCAASGFTFSNAWMS |
| | WVRQAPGKGLEWVGRIKSKTDGGTTDYAAPVKGRF |
| | TISRDDSKNTLYLQMNSLKTEDTAVYYCTTISVGL |
| | LWFGLAVRDHYYFDYWGQGTLVTVSS |
| 822 | EVQLVESGGGSVRSGGSLRLSCAASGFTFRSYWMH |
| | WVRQAPGKGLVWVSRIFSDWSTTTYADSVRGRFTI |
| | SRDNAKNTLYLEMNRLKVEDTAVYYCARSYYDSST |
| | GYYPDALDLWGQGTTVTVSS |
| 823 | EVQLLESGGGLVQPGGSLRLSCAASGFTFSTYAMS |
| | WVRQAPGKGLEWVAAISGSGGSTYYADSVKGRFTI |
| | SRDNSKNTLYLQMNSLRAEDTAVYYCAKSGSVWGS |
| | YHKTYYFDYWGQGTLVTVSS |
| 824 | QVQLVESGGGVVQPGRSLRLSCAASGFTFSSYGMH |
| | WVRQAPGKGLEWVAVISYDGSNKNYADSVKGRFTI |
| | SRENSKNTLYLQMNSLRAEDTAVYYCAKEILKGYS |
| | SGWKYYYYGMDVWGQGTTVTVSS |
| 825 | EVQLVESGGGLIQPGGSLRLSCAASGFTVSSNYMN |
| | WVRQAPGKGLEWVSVIYSGSSTYYADSVKGRFTIS |
| | RDNSKNTLYLQMNSLRAEDTAVYYCARATTTMVRG |
| | VIYHYYYYGMDVWGQGTTVTVSS |
| 826 | QVQLQESGPGLVKPSQTLSLTCTVSGGPISSGGYY |
| | WSWIRQHPGKGLEWLGCIYYSGSTYYNPSLKSRVS |
| | ISVDTSKSQFSLKLSSVTAADTAVYYCARERLGRM |
| | VRGVNWFDPWGQGILVTVSS |
| 827 | QLQLQESGPGLVKPSETLSLTCTVSGGSISSSYYY |
| | WGWIRQPPGKGLEWIGSIYYSGSTYYNPSLKSRVT |
| | ISVDTSKNQFSLKLSSVTAADTAVYYCASWTMVRG |
| | VIRWFDPWGQGTLVTVSS |
| 828 | QLQLQESGPGLVKPSETLSLTCTVSGGSISSSSYY |
| | WGWIRQPPGKGLEWIGSIYYSGSTYYNPSLKSRVT |
| | ISVDTSKNQFSLKLSSVTAADTAVYYCARQFHYVG |
| | IVVVVAPHYYYGMDVWGQGTTVTVSS |
| 829 | EVQLVESGGGLVKPGGSLRLSCAASGFTFSSYSMN |
| | WVRQAPGKGLEWVSSISSSSSYIYYADSVKGRFTI |
| | SRDNAKNSLYLQMNSLRAEDTAVYYCASPRGYSYG |
| | PFDYWGQGTLVTVSS |
| 830 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARVLYYDIL |
| | TGYWWYYYGMDVWGQGTTVTVSS |
| 831 | EVQLVESGGGLVQPGGSLRLSCAASGFTFSSYWMH |
| | WVRQAPGKGLVWVSRINSDGSSTSYADSVKGRFTI |
| | SRDNAKNTLYLQMNSLRAEDTAVYYCARGAPITIF |
| | GVVISTWFDPWGQGTLVTVSS |
| 832 | QLQLQESGSGLVKPSQTLSLTCAVSGGSISSGGYS |
| | WSWIRQPPGKGLEWIGYIYHSGSTYYNPSLKSRVT |
| | ISVDRSKNQFSLKLSSVTAADTAVYYCARAHTDSL |
| | ELGIWGQGTMVTVSS |
| 833 | EVQLLQSGGEVRRPGESLKISCKASGYSFPAHWIG |
| | WVRQMPGRGLEWMGSIFPSDSDTEYSPSFEGQVKI |
| | SADKSITTAYLQWSSLKASDTAFYYCVRKYTYDTS |
| | GFFLTSTRSAFDVWGQGTMVTVSS |
| 834 | EVQLEQSGAEEKKPGESLKISCKGSGYSFPAFYIA |
| | WMRQMPGKGLEWMGSIFPGDSETEYNPSFQGQVTI |
| | SADKSITTAYLQWDNLKASDTALYYCARKHVYDTS |
| | GFFLSSSRNAFDVWGQGTKVTVFS |
| 835 | EVQLVQSGAEQRKPGESLRISCKGSGYSFPAHWIA |
| | WVRQMPGRGLEWMGSIFPGDSDTEYNPSFQGHVNI |
| | SADRSINTAYLQWSSLKASDSAIYYCARKYSFDIS |
| | GFFLSSSRYALDVWAQGTTVTVSS |
| 836 | DMQLVESGGGLVQPGGSLKLSCAASGFTFSASAIH |
| | WVRQASGKGLEWVGHIRTRTNRYATAFSESVNGRF |
| | TISRDDSKSTAYLQMNSLKAEDTAVYYCARDEGVT |
| | FHDHWANEIRYGMDVWGRGTTVTVSS |
| 837 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARARTTMI |
| | VVVSQFDYWGQGTLVTVSS |
| 838 | EVQLVESGGGLVQPGGSLRLSCAASGFTFSSYWMH |
| | WVRQAPGKGLVWVSRINSDGSSTSYADSVKGRFTI |
| | SRDNAKNTLYLQMNSLRAEDTAVYYCARDRGGWLL |
| | GSYYYYGMDVWGQGTTVTVSs |
| 839 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARGQISHYG |
| | FGESHWGQGTLVTVSS |
| 840 | QITLKESGPTLVKPTQTLTLTCTFSGFSLSTSGVS |
| | VGWIRQPPGKALEWLALIYWDDDKRYSPSLKSRLT |
| | ITKDTSKKQVVLTLTNMDPVDTASYYCAHSGIAVV |
| | GNQLFHYYAMDVWGQGTTVTVSS |
| 841 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCAKERSSGSQ |
| | WGWTYYYYGMDVWGQGTTVTVSS |
| 842 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARDPYGGNR |
| | RFHGWVYYYYGMDVWGQGTTVTVSS |
| 843 | EVQLVESGGGLVQPGGSLRLSCAASGFTFSSYEMN |
| | WVRQAPGKGLEWVSYISSSGSTIYYADSVKGRFTI |
| | SRDNAKNSLYLQMNSLRAEDTAVYYCARESTPDVR |
| | GVMNYWGQGTLVTVSS |
| 844 | EVQLLESGGGLVLPGGSLRLSCAASGFTFSIYAMS |
| | WVRQAPGKGLEWVSAISGSGGSTYYADSVKGRFTI |
| | SRDNSKNTLYLQMNSLRAEDTAVYYCAKDAVASAG |
| | SPDYWGQGTLVTVSS |
| 845 | QVQLVESGVGVVQPGKSLRLSCAASGFTFTSYGMH |
| | WVRQAPGKGLEWVAVISFDGSNIYYADSVKGRFTI |
| | SRDNFKNTLYLQMNSLRAEDTAVYYCARDKLLWFG |
| | EPVVGYYYYYYMDVWGKGTTVTVSS |
| 846 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCARDGGGDYA |
| | QIYFDYWGQGTLVTVSS |
| 847 | QVQLVESGGGVVHPGRSLRLSCAASGFAFNKYGIH |
| | WVRQAPGKGLEWVALIWNDGNKQYYGDSVKGRFTV |
| | SRDNSKNTVSLQMDTLRDEDTAVYYCARDRLMTTY |
| | NYYSSMDVWGRGATVIVSS |
| 848 | QVQLVESGGGVVQPGRSLRLSCAASGFTFSSYGMH |
| | WVRQAPGKGLEWVAVIWYDGSNKYYADSVKGRFTI |
| | SRDNSKNTLYLQMNSLRAEDTAVYYCAREPGDCSG |
| | GSCYYYGMDVWGQGTTVTVSS |
| 849 | QLQLQESGPGLVKPSETLSLTCTVSGGSISSSSYY |
| | WGWIRQPPGKGLEWIGSIFYSGSTYYNPSLKSRVT |
| | ISVDTSKNQFSLKLSSVTAADTAVYYCARATRGYS |
| | YDDAFDIWGQGTMVTVSS |
| 850 | QVQLQESGPGLVKPSGTLSLTCSVSGGAITTSSYF |
| | WGWIRQPPGRGLEWIGSISYSGDTFYNPSLNDRVT |
| | ISVDSSKNQFFLKLRSVTAADSAVYYCASPSYTDL |
| | LTGYYVPVDYWGQGILVIVSS |
| 851 | QVHLVESGGGVVQPGKSLTLSCAASGFTFSAYGMH |
| | WVRQTPGKGLEWVALISFDGSNKYYRDSVKDRFTI |
| | ARDNSKNTLSLQMNSLRPEDTAIYYCAKDPRVNEL |
| | LWFGSLTQFYFDDWGQGTLVTVSS |
| 852 | QVQLVESGGGVVQPGRSLTLSCAASGFTFNNYGMH |
| | WVRQAPGKGLEWLALISYEGSIRYYGDSVKGRFTI |
| | SRDSSKNTVYLQMISLRAEDTAVYYCAKSGGPFHL |
| | SLYYYMDVWGKGTTVTVSS |
| 853 | QVQLQESGPGLVKPSETLSLTCTVSGGSINSYYWS |
| | WIRQTAGQGLEWIGRIYSGGSTNYNPSLKSRVTMS |
| | VDTSQNQFSLNLNSVTAADTAVYYCARAFYGHAFD |
| | FWGLGVLVIVSS |
| 854 | QVQLVESGGGVVHPGRSLRLSCAASGFTFSRFGMH |
| | WVRQAPGKGLEWVALISYEGSTEQYSDSVKGRFAI |
| | SRDNSKNTLYLQMNSLRPEDTAVYYCAKGLTIPFD |
| | KWGHGTLVTVSS |
| 855 | QVQLVESGGGVVHPGRSLRLSCAASGFTFSRFGMH |
| | WVRQAPGKGLEWVALISYEGSTEQYSDSVKGRFAI |
| | SRDNSKNTLYLQMNSLRAEDTAVYYCAKGLTIPFD |
| | KWGHGTLVTVSS |
| 856 | EVQLVESGGGLVQPGGSLRLSCAASGFTFSSFWMT |
| | WFRQTPGKGLEWVANIKEDGSEKQYVDSVKGRFNI |
| | SRDNAHNSLYLEMNSLRSEDAAVYYCARRGKYCSG |
| | GRCYSWWFDPWGQGTQVTVSS |
| 857 | QVQLVQSGGEMRKPGSSVKVSCKASGGTFSSYTIS |
| | WVRQAPGQGLEWMGRIIPMLNKTYYAQKFQGRVTF |
| | AADESTSTVYMELSSLRSEDTAMYYCARVASLIGD |
| | DYWGQGSLVTVSS |
| 858 | QVQLVQSGGEMRKPGSSVKVSCKASGGSFSSYTIS |
| | WVRQAPGHGLEWMGRIIPMLNKTYYAQKFQGRVTV |
| | AADESTSTVYMELSSLSSEDTAIYYCARVASLIGD |
| | DYWGQGSLVTVSS |
| 859 | QITLKESGPTLVKPTQTLTLTCTFSEFSLDSRGVG |
| | VGWIRQPPGRALEWLALIYWNDNKRYNPSLRSRLT |
| | ITKDTSKNQVVLTMSNMDPVDTATYYCAHKPSGWS |
| | LRFDSWGQGTLVTVSS |
| 860 | QVQLQEAGPGLVKPSETLSLTCSVFGGSISSYYWS |
| | WIRQPPGKGLEWIGYIYYRGSTNYNPSLKSRVTMS |
| | VDTSKNQFSLNLTSVTAADTAVYFCARESLFNWFD |
| | SWGHGTLVTVSS |
| 861 | QVQLVESGGGVVQPGRSLRLSCAASGFTFSRYGMH |
| | WVRQAPGKGLEWVALISYEGSTEQYSDSVKGRFAI |
| | SRDNSKNTLYLQMNSLRHEDTAVYYCAKGLTIPFD |
| | NWGQGTLVTVSS |
| 862 | EVQLVESGGGLVQTGGSLRLSCAASGFPFSGYALN |
| | WVRQAPGKGLEWVSYISSSSSTVYYADSVKGRFTI |
| | SRDNAKNSLYLQMNSLRDEDTAVYYCARVDYDSSR |
| | NYWGQGTLVTVSS |
| 863 | EVQLVESGGGLVQPGGSLRLSCAASGFTFINYDMT |
| | WVRQAPGKGLEWISYISSSSSTTHYSDSVKGRFTI |
| | SRDNARNSLYLEMNSLRAEDTAVYYCARVERWLVL |
| | GYYYYGMDVWGQGTTVTVSS |
| 864 | EVQLVESGGGLVQPGESLRLSCVASGFAFDKFWMA |
| | WLRQAPGKGLEWVALLNKDESEKYYVDSVKGRFTI |
| | SRDNAIDSVFLQMNSLRTEDTAVYYCGSIDYWGQG |
| | ALVTVSS |
| 865 | QVQLQESGPGLVKPSQTLSVTCTVSGGSINRDGHY |
| | WIWIRQHPEKGLEWLGYIYSGRNTFYNPSLRSRLS |
| | ISADTSKSQFSLNLHSVTAADTAVYYCAKMYSDYD |
| | DNYYGLDVWGRGTTVTVSS |
| 866 | QVQLQESGPGLVKPSETLSLTCTVSGGSISSYYWS |
| | WIRQPPGKGLEWIGYIYYSGSTNYNPSLKSRVTIS |
| | VDTSKNQFSLKLSSVTAADTAVYYCARDRYCSSTS |
| | CGGYYYYMDVWGKGTTVTVSS |
| 867 | QVQLQESGPGLVKPSETLSLTCTVSGGSVSSGSYF |
| | WSWIRQPPGKGLEWIGYIYYSGSTNYNPSLKSRVT |
| | ISVDTSKNQFSLKLRSVTAADTAVYYCARAPNDFW |
| | SGYPYYFDYWGQGTLVTVSS |
| 868 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCTRDGSTAAI |
| | FGNIDYWGQGTLVTVSS |
| 869 | QVQLVQSGAEVKKPGASVKVSCKASGYTFTGYYMH |
| | WVRQAPGQGLEWMGWINPNSGGTNYAQKFQGRVTM |
| | TRDTSISTAYMELSRLRSDDTAVYYCARGVVRNDY |
| | GDPGFDYWGQGTLVTVSS |
| 870 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATAPAYCSG |
| | GSCPENNWFDPWGQGTLVTVSS |
| 871 | QVLLVQSGAEVKKPGASVKVSCKASGYRFTSYGIH |
| | WVRQAPGQSLEWMGCINTDNEKTEYSQKFQGRVTI |
| | TRDTSASTAYMELSTLRFEDTAVYYCAILWFGEFY |
| | FYDLFYNAVDVWGQGTTVTVSS |
| 872 | QVLLVQSEAEVKKPGASVKVSCKASGYRFTSYGIH |
| | WVRQAPGQGLEWMGSINTDNGKTEYSQKFQGRVTI |
| | TRDTSAGTAYMELSTLRSEDTAVYYCAILWFGEFY |
| | FYDLFYNAVDVWGQGTTVTVSS |
| 873 | QVQLVQSGAEVKKPGASVKVSCKASGYTFTSYAMH |
| | WVRQAPGQRLEWMGWINAGNGNTRYSQKFQGRVTI |
| | TRDTSASTAYMELSSLRSEDTAVYYCASRREQWLG |
| | DLGYYYYGMDVWGQGTTVTVSS |
| 874 | QVQLVQSGAEVKKPGASVKVSCKASGYTFTSYYMH |
| | WVRQAPEQGLEWMGIINPSGGSTSYAQKFQGRVTM |
| | TRDTSTSTVYMELSSLRSEDTAVYYCARGGAHSED |
| | YWGQGTLVTVSS |
| 875 | QVQMVQSGAEVKKPGASVKVSCKASGYTFTNYYVH |
| | WVRQAPGQGLEWMGRINPSDGSTSYTQKFQGRVTM |
| | TRDTSTSTVYMQLSSLRSEDTALYYCARHQDPLDI |
| | VATVDWGGLDYWGQATLVTVSS |
| 876 | QVQLVQSGGELRKPGSSVKVSCKASGGTFSSYTIS |
| | WVRQAPGQGLEWMGRIIPMLNKTYYAQKFQGRVTF |
| | AADESTNTVYMELSSLRSEDTAMYYCARVASLIGD |
| | DYWGQGSLVTVSS |
| 877 | QVQLVQSGAEVKKPGSAVKVSCKASGGTFNSYAFN |
| | WVRQAPGQGLEWMGGIIPIFGPPNYAQNFQGRVTI |
| | TADESTSTAYMELSSLTSEDTAVYYCATTGTDNYY |
| | YYMDVWGKGTTVTVSS |
| 878 | QVQLVQSGAEVKKPGASVKVSCKASGYTFTSSDIN |
| | WVRQATGQGLEWMGWMNPNTGTTGYAQKFQDRVTM |
| | TRDTSINTAYMELSSLRSEDTAVYYCARKNCSGGI |
| | CYFHDYWGQGTRVTVSS |
| 879 | QITLKESGPTLVKPTQTLTLTCTFSEFSLDARGVG |
| | VGWIRQPPGRALEWLALIYWNDYKRYSPSLQSRLT |
| | ITKDTSKNQVVLTMTNMDPVDTATYYCAHKPSGWS |
| | LRFDSWGQGTLVTVSS |
| 880 | EVQLLESGGGLVQPGGSLRLSCAASGFTFISYATS |
| | WVRQAPGKGLEWVSAISGSGGSTYYADSVKGRFTI |
| | SRDNSKNTLYLQMNSLRAEDTAVYYCAKGQTIQLW |
| | LFGALWGQGTLVTVSS |
| 881 | EVQLLESGGGLVQPGGSLRLSCAASGFTFSSYAMS |
| | WVRQAPGKGLEWVSAISGSGGTTYYADSVKGRFTI |
| | SRDNSKNTLYLQMDSLRGDDTAVYSCALTVSSWYP |
| | GIFENWGQGTLVTVSS |
| 882 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCAKAFSGSYW |
| | DAFDIWGQGTMVTVSs |
| 883 | QVQLVESGGGVVQPGRSLRLSCAASGFTFSSHGMH |
| | WVRQAPGKGLEWVAVISYDGINKYYADSVKGRFTI |
| | SRDNSKNTLFLQLNSLRAEDTAVYYCAKAASGARG |
| | YYGMDVWGQGTTVTVSS |
| 884 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARSSSGHYV |
| | SDLGYWGQGTLVTVSS |
| 885 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARALNGYRY |
| | NDYWGQGTLVTVSS |
| 886 | QVQLVESGGGVVQPGRSLRLSCAASGFTFSTYGMH |
| | WVRQAPGKGLEWVAVMWFDGVDKYYADSVKGRFTI |
| | SRDNSKNTLYLQMNSLRDEDTAVYYCAREEGGGSS |
| | THFDCWGQGTLVTVSS |
| 887 | QVQLVESGGGVVQPGRSLRLSCAASGFTFSTFAMH |
| | WVRQAPGKGLEWVAIISYDEINKYYADSVKGRFTI |
| | SRDNSKNMLYLQMNSLRAEDTAVYYCARTREGSYY |
| | YGMDVWGQGTTVTVSS |
| 888 | EVKLVESGGHLVQPGRSLRLSCTASGFIFGDYAMG |
| | WVRQAPGKGLEWVSFIRGRLVGATVEYAASVKGRF |
| | TMSRDDSERVAYLQMNSLKIEDTGVYYCVRGGLQF |
| | VVAVGPYGVDVWGQGTTVTVSS |
| 889 | EVKLVESGGHLVQPGGSLRLSCTASGFIFGDYAMG |
| | WVRQAPGKGLEWVSFIRGRLVGATVEYAASVKGRF |
| | TMSRDDSERVAYLQMSSLKIDDTGVYYCVRGGLQF |
| | VVAVGPYGVDVWGQGTTVTVSS |
| 890 | EVQLVESGGGLVQPGGSLRLSCAASGFTFSSYWMS |
| | WVRQAPGKGLEWVANIKQDGSEKYYVDSVKGRFTI |
| | SRDNAKNSLYLQMNSLRVEDTAVYYCARDIGGGAP |
| | DYWGQGTLVTVSS |
| 891 | QVLLQESGPGLVRPSQTLSLTCSVSGASISSGDYY |
| | WTWVRQTPGKGLEWLGFIYYSGSTYYNPSLQRRVL |
| | ISMDTAMNQFSLRLTSVTAADTAVYYCAIKPSIPG |
| | YFDPWGQGTLVTVSS |
| 892 | QVQLQQWGAGLLKPSETLSLTCALNGGVLSDYYWS |
| | WIRQPPGQGLEWIGAIHRSGSTSYTPSLKSRVTMS |
| | VDTSKNQFSLRLSSVTAADTAVYYCARVGGWQRSP |
| | RPNWGQGTRVTVSS |
| 893 | QVQLQQWGAGLLKPSETLSLTCALNGGVLSDYYWS |
| | WIRQPPGQGLEWIGAIHRSGSTSYTPSLKGRVTMS |
| | VDTSKNQFSLRLSSVTAADTAVYYCARVGGWQRSP |
| | RPNWGQGTRVTVSS |
| 894 | QVQLQQWGAGLLKPSETLSLTCAVYGGSFSDYYWN |
| | WIRRPPGKGLEWIGEITHSGSTNYNPSLKSRVTIS |
| | VDTSKNQFSLKLSSVTAADTAVYYCARGQGYGRVL |
| | LWFGEWGQGTLVTVSS |
| 895 | QVQLQQWGAGLLKPSETLSLTCAVYGGSFSDYFWY |
| | WIRQPPGKGLEWIGEINHSGSTNYNPSLKSRVSIS |
| | VDTSKNQFSLKLSSVTAADTAVYYCARGQGYGRVL |
| | LWFGEWGQGTLVTVSS |
| 896 | QVQLQESGPGLVKPSGTLSLTCDVSGDSISSNNWW |
| | TWVRQPPGKGLEWIGDIYHSGTTNYNPSLKSRLTM |
| | SVDKSKNHFSLKLTSVTAADTAVYYCARPSSGSRF |
| | DYWGQGTLVTVSS |
| 897 | QVQLQESGPGLVKPSETLSLTCTVSGGSISSYYWS |
| | WIRQPAGKGLEWIGHIYTSGSTNYNPSLKSRVTMS |
| | VDTSKNQFSLKLSSVTAADTAVYYCARGFDYWGQG |
| | TLVTVSS |
| 898 | QVQLQESGPGLVKPSETLSLTCTVSGDSISSYYWS |
| | WIRQSPGKGLEWIGYIYHSGSADYNPSLKSRVSMS |
| | LDASKNQFSLKMSSVTAADTALYYCAKARGVVLFD |
| | YWGQGTLVTVSS |
| 899 | QVQLRESGPGLVKPSETLSLTCTVSGGSISGYYWS |
| | WIRQPPGKGLEWIGYLHYSGRSNSSPSLNSRVSIS |
| | VDTSQNRFSLKVTSLTAADTAVYYCARHSYGSGTY |
| | LDPFDYWGQGTLVTVSS |
| 900 | QVQLQESGPGLVKPSQTLSLTCTVSGGSISSGSYY |
| | WSWIRQPAGKGLEWIGRIYTSGSTNYNPSLKSRVT |
| | ISVDTSKNQFSLKLSSVTAADTAVYYCARQPHLAY |
| | YYDSSGYNDAFDIWGQGTMVTVSS |
| 901 | QVQLQESGPGLVKPSQTLSLICTVSDDSISSGSYY |
| | WSWIRQPAGKGLEWIGRIYAGESTNYNPSLKSRVI |
| | ISVDTSKKQFSLRLSSVTAADTAVYYCARGAVVTP |
| | FGLDSWGQGTLVTVSS |
| 902 | EVQLVQSGAEVKKPGESLKISCKGSGYSFTSYWIG |
| | WVRQMPGKGLEWMGIIYPGDSDTRYSPSFQGQVTI |
| | SADKSISTAYLQWSSLKASDTAMYYCASEDYYDSS |
| | GYYWYWGQGTLVTVSS |
| 903 | EVQLVQSGAEVKKPGESLKISCKGSGYSFTSYWIG |
| | WVRQMPGKGLEWMGIIYPGDSDTRYSPSFQGQVTI |
| | SADKSISTAYLQWGSLKASDTAMYYCARLSAIAVV |
| | GYYYYAMDVWGQGTTVTVSs |
| 904 | QVQLVQSGSELKKPGASVKVSCKASGYTFTSYAMN |
| | WVRQAPGQGLEWMGWINTNTGNPTYAQGFTGRFVF |
| | SLDTSVSTAYLQISSLKAEDTAVYYCARDFIAASP |
| | FYYYYYMDVWGKGTTVTVSS |
| 905 | EVQLVQSGAEVKKPGESLKIFCKGSGYTFSFYWIG |
| | WVRQTPGKGLEWMGIIYPGDFDTRYSPSFQGQVTI |
| | SADKSINTAYLQWSSLKASDTAMYYCATSPGGYGV |
| | RRTVLEDFRHWGQGTLVTVAS |
| 906 | QLQLQESGPGLVKPSETLSLTCTVSGGAFSSGRHY |
| | WGWIRQPPGKGLEWIGSIYSGVITHYNAPLKSRVT |
| | IAVDTSKNQFSLKLSSVTAADTAVYYCWTMEYDDY |
| | SFVYDYWGQGTLVTVSS |
| 907 | QVHLQQWGAGLLKPSQTLSLTCAVYGGSFSSYYWS |
| | WIRQTPGKGLEWIGEVTHSGSTNYKPSLKSRVTMS |
| | VDTSRNQFSLNLTSVTAADTAVYYCARGGKQQLVR |
| | NYYLDSWGQGTLVTVSS |
| 908 | QVQLVQSGAEVKKPGASVKVSCKVSGYTLTELSMD |
| | WVRQAPGKGLEWMGGFDPEDGETIDAQKFQGRVTM |
| | TEDTSTDTAYMELSSLRSEDTAVYYCATGFGGVIV |
| | RGFDYWGQGTLVTVSS |
| 909 | QVQLVESGGGLVKPGGSLRLSCAASGFTFSDYYMS |
| | WIRQAPGKGLEWVSYISSSGSTIYYADSVKGRFTI |
| | SRDNAKNSLYLQMNSLRAEDTAVYYCARVYGDYSY |
| | YMDVWGKGTTVTVSS |
| 910 | EVQLVESGGGLIQPGGSLRLSCAASGITVSSNYMS |
| | WVRQAPGKGLEWVSVIYSGGSTDYADSVKGRFTIS |
| | RDKSKNTLYLQMNSLRAEDTAVYYCARDLGEAGGM |
| | DVWGQGTTVTVSS |
| 911 | EVQLVESGGGLVQPGGSLRLSCAASGFTFSRFWMT |
| | WVRQAPGKGLEWVANIKEDGSVMFYVDSVKGRFSI |
| | SRDNSKNSLYLEMNSLRAEDTAVYFCVREIESGVD |
| | FWSGHYYWGQGTLVTVSS |
| 912 | EVQLVESGGGLVQPGGSQRLSCVASGFTFSNYWMS |
| | WVRQAPGKGLHWVANIKSDGSETYYVDSLRGRFTI |
| | SRDNAKNSLYLQLTSLTVEDTAVYYCARDSAYYDT |
| | IGYYSGDYWGRGTLVTVSS |
| 913 | QVQLVESGGGAVQPGRSLRLSCEASAFSFHLHGMH |
| | WVRQAPGKGLEWVALIWFDGSKKFYADAVKGRFTI |
| | SRDNSKNTLYLQMNSLRVEDTAIYYCGRSFRGSCF |
| | DYLGQGTLVTVSS |
| 914 | EVQLLESGGGLVQPGGSLRLSCAASGFTFSSYAMS |
| | WVRQAPGKGLEWVSAISSSGGGTYYADSVKGRFTI |
| | SRDNSKNTLYVQMNSLRAEDTAVYYCALGTGSYYG |
| | VNYWGQGTLVTVSS |
| 915 | EVQLVESGGGLVQPGRSLRLSCAAFGFIFDDYGMH |
| | WVRQVPGKGLEWVSGITWNSGSIGYADSVKGRFTI |
| | SRDNAKNSLYLQMNSLRAEDTALYFCAKDMGGRYS |
| | SGLYYYYYGMDVWGQGTTVTVSS |
| 916 | EVQLVESGGGLVQPGGSLRLSCAASGFTVSSNYMS |
| | WVRQAPGKGLEWVSVIYSGGSTYYADSVKGRFTIS |
| | RHNSKNTLYLQMNSLRAEDTAVYYCARELRGYFDY |
| | WGQGTLVTVSS |
| 917 | QMRLQESGPGLVKPSETLSLTCTVSGGSIGSSSYF |
| | WGWIRQPPGKGLEWIGNIYYGGSTYYKPSLKSRVT |
| | ISLDTSKNQLTLRLSSVTAADTAVYYCARDPNDFW |
| | SGFPRGAFDIWGQGTMVTVSS |
| 918 | QLQLQESGPGLVKPSETLSLTCTVSGGSISSSSYY |
| | WGWIRQPPGKGLEWIGSIYYSGSTYYNPSLKSRVT |
| | ISVDTSKNQFSLKLSSVTAADTAVYYCASHARYEE |
| | ETFDYWGQGTLVTVSS |
| 919 | EVQLVESGGGLAQPGGSLRLSCAASGFTFSSYDMH |
| | WVRQAAGKGLEWVSTIGTAGDTYYPGSVKGRFTIS |
| | RENDKNSLYLQMNSLRAGDTAVYYCVRDSYTSAWT |
| | PAGYFDLWGRGTLVTVSS |
| 920 | QVQLVESGGGVVQPGRSLRLSCAASGFTFSRSAMH |
| | WVRQGPGKGLEWVAMMSYDGSDIQYADSVKGRFTI |
| | SRGNSKNTLFLQMNSLRLADTAMYYCAKDHYGSID |
| | YWGQGTLVTVSS |
| 921 | QVQLVESGGGVVQPGRSLRLSCVASGFTFSSQSMH |
| | WVRQAPGKGLEWVSIISYDGNNKQYADSVKGRFTI |
| | SRDNSKSTLFLQINSLRPQDTAVYYCARPYTSRWF |
| | WSNWGQGTLVTVSS |
| 922 | EVQLVESGGGLVQPGRSLRLSCAASGFTFEEYSIH |
| | WVRQAPGKGLEWVSGVSWNSGTIAYADSVRGRFTI |
| | SRDNAKNSLYLQMSRLRADDTALYYCALLPPNAYD |
| | YGDGLLDHWGQGTLVTVSS |
| 923 | EVQVVQSGAEVKKPGESLKISCKGSGYTFGRYWIA |
| | WVRQMPGKGLEWMGIINPADSDTRYSPTFQGQVTI |
| | SVDQAISTAYLQWSSLKASDTAMYHCARHRAAGGN |
| | YYYGMDVWGQGTTVTVSS |
| 924 | QVQLVQSGAEVKKPGASVKVSCKASGYTFSTYYMH |
| | WVRQAPGQGLEWMGIINPSGDSTRYAQKFQGRVTM |
| | TRDTSTSTVYMEVSSLRFEDTAVYYCARERVGPAA |
| | GYMDVWGKGTTVTVSS |
| 925 | QVQLVQSGAEVKKPGSSVKVSCKASGGTFSSYAIS |
| | WVRQAPGQGLEWMGRIIPIFGTANYAQKFQGRVTI |
| | TADESTSTAYMELSSLRSEDTAVYYCARAAYYYDS |
| | SGYGWFDPWGQGTLVTVSS |
| 926 | QVQLVQSGAEVKNPGSSVKVSCKTSGATFTTYAIN |
| | WVRQAPGQGLEWIGGIFPIFTAAVYAQKFQGRVTI |
| | TADESTTTAYLELSSLRSEDTAVYYCARGDYTEYS |
| | YYYMDVWGKGTTVTVSS |
| 927 | EVQLLESGGGLVQPGGSLRLSCAASGFTLSSYAMS |
| | WVRQAPGRGLEWVSAVSGSGGSTYYADSVKGRFTI |
| | SRDNSKNMLYLQMNSLRAEDTAIYYCALPTGASSS |
| | YSGPNYWGQGTLVTVSS |
| 928 | QVQLVESGGGVVQPGRSLRLSCVASGFTFSNYDMH |
| | WVRQAPGKGLEWVTVISSDGNNRRYADSVKGRFTI |
| | SRDNSKNMLYLQMNSLKAEDTAVYYCARDEVIAVA |
| | TGEGMDVWGQGTTVTVSP |
| 929 | EVQLVESGGGLVQPGRSLRLSCAASGFTFDDYAMH |
| | WVRQAPGKGLEWVSGISWNSGSIGYADSVKGRFTI |
| | SRDNAKNSLYLQMNSLRAEDTALYYCAKDMGYDIL |
| | TGSGLGDYWGQGTLVTVSS |
| 930 | EVQLVESGGGLVQPGRSLRLSCAASGFTFDDYAMH |
| | WVRQAPGKGLEWVSGISWNSGTIGYEDSVKGRFII |
| | SRDNAKNSLYLQMNSLRAEDTALYYCAKEPLFGET |
| | YGMDVWGQGTTVTVSS |
| 931 | EAQLVESGGGLVQPGRSLTVSCAVSGFTFDDYAMH |
| | WVRQAPGKGLEWVSSISWNSEKIAYADSVKGRFTV |
| | SRDNAKNSLYLQMTSLRPEDTALYYCARDKGSGSY |
| | YSGAYYYYMDVWGKGTTVTVSS |
| 932 | EVQLVESGGGLVPPGGSLRLSCAASGFTFSSYTIN |
| | WVRQAPGKGLEWVSYINSGSSIIYYADSVKGRFTI |
| | SRDNAKNSLYLQMNSLRAEDTAVYYCATFNSGNDN |
| | AYEYWGQGTLVTVSS |
| 933 | EVRLVESGGGWVQPGGSLRLSCEASTFIFSNSEMN |
| | WVRQAPGKGLEWVSYISSSDNSVHYADSVKGRFTI |
| | SKDSAKKTLYLQMNSLRAEDTGVYYCAREYPDFWS |
| | GHYYYYMDVWGKGTTVTVSS |
| 934 | EVQLVESGGGLVQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARLPYGMDVW |
| | GQGTTVTVSS |
| 935 | QVKLQQWGAGLVKPSETLSRTCAVYGGSFSGYFWS |
| | WIRQSPGKGLEWIGEINHSGKTNYSPSLKSRVSIS |
| | VDTSKNQFSLKLTSVTAADTAVYYCARGLYDKSGY |
| | RSDGFDSWGQGAVVTVYS |
| 936 | QVQLQQWGAGLLKPSETLSRTCAVYGGSFSGYYWT |
| | WIRQPPGKGLEWIGEINHSGSTNYNPSLKSRITMS |
| | VDTSKNQFSLELRSVSAADTAVYYCARGFEGYCSG |
| | GRCYSYFDYWGQGTLVTVSS |
| 937 | QVQLQESGPGLVKPSETLSLTCTVSGGSLSSYYWN |
| | WIRQPPGKGLEWIGYMYNSGSTNYNPSLKSRVTIS |
| | VDTSKNQFSLKLSSVTAADTAVYYCARVKNWDYGL |
| | YWGQGTLVIVSS |
| 938 | QVQLQESGPGLVKPSETLSLTCTVSGGSISTFYWN |
| | WVRQPPGKGLEWIGFIYYSGRTNYNPSLKSRVTIS |
| | VDTSKNQFSLKVSSVTAADTAVYYCARDGQSDWHF |
| | DLWGRGTLVTVSS |
| 939 | QVQLQESGPGLVKPSETLSLTCTVSGGSVSSYFWS |
| | WLRQPPGKGLEWIAYIFYTGTSNYNPSLKSRVTIS |
| | LDTSKNQMSLNLSSVTTADTAVYYCARVYGDYLDH |
| | WGQGTVVTVSS |
| 940 | QITLKESGPTLVKPTQTLTLTCTFSGFSFNTPGVG |
| | VGWIRQPPGKAPECLALIYWDDEKLYNPSLKTRLT |
| | ITKDPSKNQVVLTMTTMDPVDTATYYCAHRSFLYN |
| | IFNGYSYAPFDYWGQGSMVTVSS |
| 941 | QVQLVESGGGVVQPGRSLRLSCAASGFSFSNHGMH |
| | WVRQAPGKGLEWVAVIWYDGDNRFYADSVRGRFTI |
| | SRDNSKNTLFLQMDSLRAEDTGIYYCAKDLFSGDR |
| | DFWGQGTLVTVSS |
| 942 | QVQLVESGGGVVQPGRSLRLSCVASGFTFSNSAMH |
| | WVRQAPGMGLEWVAVIYYDGSNEYYADSVKGRFTI |
| | SRDNSKNTLYLQMNSLRADDTAVYYCAKDSGAVLL |
| | WFGADFWGQGTLVTVSS |
| 943 | DVQLVESGGSLVQPGGSLRLSCAASEFTFSSYEMN |
| | WVRQAPGKGLEWVSYIDSSSTTIYYADSVKGRFTI |
| | SRDNAKNSLYLQMNSLRAEDTAVYYCAREGAYDIW |
| | RGSYMRAYDHWGQGTLVTVSS |
| 944 | QVQLVQSGSELKKPGASVKVSCKASGYTFTNFAIN |
| | WVRQAPGQGLEWMGWINTKTGIPTYAQGFTGRFVF |
| | SLDTSVSTAYLQISGLKAEDTAVYYCARYIEMFDP |
| | WGQGTLVTVSS |
| 945 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARQAYGDYG |
| | WDYYYGMDVWGQGTTVTVSS |
| 946 | QVQLAEAGGGVVQPGTSLRLSCVVSGFSFSRYGMH |
| | WVRQAPGKGLEWVAVISHDDSQKYYGDSVKGRFTI |
| | SRDNSKDTLYLEMTSLRLEDTAVYYCLKDWDWEYE |
| | DSRPTLRGSVYWGQGTLVIVSA |
| 947 | QVQLVESGGGAVQPGRSLRLSCVTSGFNFNSYTMH |
| | WIRQAPGKGLEWVAVISYEGSKKYYADSLKGRFTI |
| | SKDNSKNTVYLEMNSLTTEDTAVYYCARGSVFWFG |
| | EGKNWFDPWGQGTLVTVSS |
| 948 | QVQLVESGGGAVQPGRSLRLSCVTSGFNFNSYTMH |
| | WIRQAPGKGLEWVAVISYEGSKKYYADSLKGRFTI |
| | SKDNSKNTVYLEMNSLTTEDTAVYYCARGSVFWFG |
| | EGKNWFDPWGQGTLVIVSS |
| 949 | EVQLVESGGGLVQPGGSLRLSCAASGFTFSSYEMN |
| | WVRQAPGKGLEWVSYISSSGSTIYYADSVKGRFTI |
| | SRDNAKNSLYLQMNSLRAEDTAVYYCAREDSSGWS |
| | RGDYWGQGTLVTVSS |
| 950 | QVQLVQSGSELKKPGASVKVSCKASGYIFTSYGMN |
| | WVRQAPGQGLEWMGWINTNTGSPMYAQGFTGRFVF |
| | SLDTSVSTAYLQISSLKAEDTAVYYCARRFVVREV |
| | EYNWFDPWGQGTLVTVSS |
| 951 | QAQLVQSGSEVRKPGASVKVSCKASGYSFNDYGIT |
| | WVRQAPGQGLEWMGWISAYNGETNYAQKFQDTVTM |
| | TTDTSTNTAYLELRSLRFADTALYYCARDGYCNSM |
| | RCYRYYHGMDVWGQGTTVTVSS |
| 952 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATGPTAKPN |
| | KQWGYWFDPWGQGTLVTVSS |
| 953 | QVQLVQSGAEVKKPGASVKVSCKASGNTFSTYYIH |
| | WVRQAPGQGLEWMGIISPSGDDANYTQKFQDRVTM |
| | TRDTPTNTVYLELSSLRSEDTAVYYCASPVSVEQD |
| | FDIWGQGTMVTVSA |
| 954 | EVQLVESGGGSVKPGGSLRLSCAASGFTFSDVWMS |
| | WVRQAPGKGLEWVGRIRSKSDGGTTDYAAPMKERF |
| | SISRDDAKNTMYLQMNSLKTEDTGVYYCTTPVGDF |
| | WGQGTMVTVSS |
| 955 | EVQLMESGGGLVKPGGSLRLSCAGSGLTFDNAWMS |
| | WVRQAPGKGLEWVGRVKSKTDGGTTDYAAPVKGRF |
| | TISRDDSKNTLFLQMNSLKTEDTAVYYCSTSHPPF |
| | FDYWGQGTLVTVSS |
| 956 | QVQLVESGGGVVQPGRSLRLSCAASRFTFSSYAMH |
| | WVRQAPGKGLEWVALISYDGSNKYYADSVKGRFTI |
| | SRDNSKNTLYLQMDSLRPEDTAVYYCARGLWQLVS |
| | PVFDYWGQGTLVTVSS |
| 957 | EVQLVESGGVVVQPGGSLRLSCAASGFTFDDYAMH |
| | WVRQAPGKGLEWVSLISWDGGSTYYADSVEGRFTI |
| | SRDNSKNSLYLQMNSLRAEDSALYYCAKVTNRGVR |
| | GLYFDYWGQGTLVTVSS |
| 958 | QVQLVQSGAEVKKPGASVKVSCKASGNTFTTYYIH |
| | WVRQAPGQGLEWMGIISPSGDDANYTQKFQDRVTM |
| | TRDTPTNTVYLELSSLRSEDTAVYYCASPVSVEQD |
| | FDIWGQGTMVTVSA |
| 959 | QVQLVQSGAEVKKPGSSVNVSCKASGGTFNSYTLS |
| | WVRQAPGQGLEWMGRIVPMLGITNYAQKFQDRVTI |
| | TADESTATAYMDLSSLTSEDTAVYFCAINTLLVTA |
| | WGQGTLVTVSS |
| 960 | QITLKESGPTLVKPTQTVTLTCTFSGFSLNTPGAG |
| | VGWIRQPPGQALECLALIYWDDDKRYSPSLRSRLS |
| | IAKDTAKNQVVLTVTNLDPVDTATYYCVHRSFLYD |
| | IFSGYSYAPFDYWGQGMLVTVSS |
| 961 | QITLKESGPTLVKPTQTLTLTCTFSGFSFNTPGVG |
| | VGWIRQPPGKAPECLGLIYWDDEKRYSPSLKSRLT |
| | ITKDPSKNQVVLTMTTMDPVDTATYYCAHRSFLYN |
| | IFDGYSYAPFDYWGQGSMVTVSS |
| 962 | QVQLVESGGGLVKPGGSLRLSCAASGFTFTFSDYY |
| | MNWIRQAPGGGLEYIAYISSGGDAIYYADSVKGRF |
| | IISRDNSESSVSLQMTSLRADDTAVYYCAGGADCR |
| | RTSCHYLVSNREEYMGVWGKGTTVTVSS |
| 963 | EVQLVESGGGLVKPGGSLRLSCAASGFTFSDYYMN |
| | WVRQAPGKGLEWVSSMSSDSDYIFYADSVKGRFTI |
| | SRDNAKNSLYLQMNSLRAEDTAVYYCARGLVLSGT |
| | RYSYFYGMDVWGQGTTVTVSS |
| 964 | QVHLAEAGGGVVQPGRSLRLSCVVSGFSFSRYGMH |
| | WVRQAPGKGLEWVAVISHDESQKYYGESVKGRFTI |
| | SRDNSKDTVYLQMDSLRVEDTAVYYCVKDWDWEYE |
| | DNRPTLRGSVYWGQGTLVIVSA |
| 965 | QVQLAEAGGGVVPPGRSLRLSCVVSGLSFSRYGMH |
| | WVRQAPGKGLEWVAVISHDESQKYYGESVKGRFTI |
| | SRDNSKDTLYLQMDGLRVEDTAMYYCVKDWDWEYE |
| | ESRPTLRGSVYWGQGALVIVSA |
| 966 | QVQLVESGGGVVQPGRSLRLSCATSGFSFNNFGMH |
| | WVRQAPGKGLEWLAVISYEGSKKYYADSLKGRFTI |
| | SRDGSKDTLYLQLSSLGVEDTAVYHCAKGGPIFWL |
| | GEGKNWFDAWGPGTPVIVSS |
| 967 | QVQLVESGGGVVQPGRSLRLSCAASGFTLSSYAMH |
| | WVRQAPGKGLEWVAVISYDGSNKYYRDSVKGRFTI |
| | SRDNSKNTLYLQINSLRVDDTAVYYCARDKGGILM |
| | LRGADFWGQGTLATVSS |
| 968 | EVQLVESGGGLVQPGGSLRLSCAASGFTFSSYEMN |
| | WVRQAPGKGLEWVSYITSSGNTILYADSVKGRFTI |
| | SRDNAKNSLYLRMNSLRAEDTALYYCARTLIAAAG |
| | SAFDIWGQGTMVTVSS |
| 969 | EVQLVESGGGLGLPGGSLRLSCAASGFTFSSYAMN |
| | WVRQAPGKGLEWISYISSSSGTIYYADSVKGRFTI |
| | SRDNAKNSLFLQMNSLRDEDTAVYYCARGPTSITM |
| | IVVVDDAFDIWGQGTMVTVSS |
| 970 | QVQLQESGPGLVKPSETLSLTCSVSGGSISPYSWS |
| | WIRQPPGKGLEWIGYIYYTGKTNYNSSLKTRVTIS |
| | LDTSKNQFSLRLTSVTTADTAIYYCARVMNSSWYT |
| | RYYYNYMDVWGKGTSVTVSS |
| 971 | QLQLQESGPRLVKPSATLSLTCTVSGDSIRSSSFY |
| | WGWIRQPPEKGLEWLGSVYNSGTAYYNPSLKSRVS |
| | VSVDTSKNQFSLKVNSVTAADTAVYYCARRGGGCS |
| | EGVCYNFDRWGQGTLVTVSS |
| 972 | QVQLVQSGSELKKPGASVKISCKAFGYSFTTYAMN |
| | WVRQAPGRGLEWMGWIDTNTGKPTYARGFTGRFVF |
| | SLDTSVRTSYMQINTLKAEDTAVYYCARGDPRDYW |
| | GQGTLVTVSS |
| 973 | QVQLVQSGAEVKKPGSSVKVSCKASGGTFSSYAIS |
| | WVRQAPGQGLEWMGRIIPIFGTANYAQKFQGRVTI |
| | TADESTSTAYMELSSLRSEDTAVYYCARGSYYYDS |
| | SGYYLDYWGQGTLVTVSS |
| 974 | QVQLVQSGAEVKKPGSSVKVSCKASGGTFSSYAIS |
| | WVRQAPGQGLEWMGRIIPIFGTANYAQKFQGRVTI |
| | TADESTSTAYMELSSLRSEDTAVYYCARAAYYYDS |
| | SGYGWFDPWGQGTLVTVSS |
| 975 | EVQLVESGGGLVKPGGSLRLSCAASGFTFSHAWMC |
| | WVRQAPGKGLEWVGRIKSNTDGGTTDYAAPVKGRF |
| | TISRHDSKNTLYLQLNSLKTEDTAVYYCTTDLGAT |
| | GIYYYYYMDVWGKGTTVTVSS |
| 976 | EVQLVESGGGLVQPGGSLRLSCAASGFTFSSYEMN |
| | WVRQAPGKGLEWVSYISSSGSTIYYADSVKGRFTI |
| | SRDNAKNSLYLQMNSLRAEDTAVYYCARFPRDYYD |
| | SSGYLIQEGNFDYWGQGTLVTVSS |
| 977 | EVQLVESGGGLIQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARVTRAGAAG |
| | DGGAFDIWGQGTMVTVSS |
| 978 | QVQLQESGPGLVKPSETLSLTCTVSGGSISSYYWS |
| | WIRQPPGKGLEWIGYIYYSGSTKYNPSLKSRVTIS |
| | VDTSKNQFSLKLSSVTAADTAVYYCARSVVPVAGT |
| | DYWGQGTLVTVSS |
| 979 | QVQLVQSGAEVKKPGASVKVSCKASGYTFTTYGIS |
| | WVRQAPGQGLEWMGWISAYNGNTNYAQKLQGRVTM |
| | TTDTSTSTAYMELRSLRSDDTAVYYCARDQHPGYP |
| | ALVYYYYYMDVWGKGTTVTVSS |
| 980 | QVQLVQSGAEVKKPGASVKVSCKASGYTFTNYGIT |
| | WVRQAPGQGLEWMGWISTYSGNTNYAQKLQGRVTM |
| | TTDTSTSTAYMELRSLRSDDTAVYYCARDNIQTFD |
| | YWGQGTLVTVSS |
| 981 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATSSPVAGY |
| | NSWFDPWGQGTLVTVSS |
| 982 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATGPAVIPL |
| | RWFDPWGQGTLVTVSS |
| 983 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCATAPAAAGP |
| | TDWFDPWGQGTLVTVSS |
| 984 | QVQLVQSGAEVKKPGASVKVSCKVSGYTLTELSMH |
| | WVRQAPGKGLEWMGGFDPEDGETIYAQKFQGRVTM |
| | TEDTSTDTAYMELSSLRSEDTAVYYCAISPSVHSL |
| | WWFDPWGQGTLVTVSS |
| 985 | QVQLVQSGAEVKKPGASVKVSCKASGYTFTSYAMH |
| | WVRQAPGQRLEWMGWINAGNGNTKYSQKFQGRVTI |
| | TRDTSASTSYMELSSLRSGDTAVYYCARDEIHYDI |
| | LTGYYNRFWFHPWGQGTLVTVSS |
| 986 | QVQLVQSGAEVKKPGSSVKVSCKASGGTFSSYAIS |
| | WVRQAPGQGLEWMGGIIPIFGTANYAQKFQGRVTI |
| | TADESTSTAYMELSSLRSEDTAVYYCARDAETGYY |
| | DSSGYPINWFDPWGQGTLVTVSS |
| 987 | QVTLKESGPVLVKPTETLTLTCTVSGFSLSNARMG |
| | VSWIRQPPGKALEWLAHIFSNDKKSYSTSLKSRLT |
| | ISKDTSKSQVVLTMTNMDPVDTATYYCARHYYDTG |
| | AYYVPFDHWGQGTLVTVSS |
| 988 | QITLKESGPTLVKPTQTLTLTCTFSGFSLSTTGVG |
| | LAWIRQPPGKALEWLAFIYWDDDKRYSPSLQTRLT |
| | ITKDTSKNQVVLTLTNMDPMDTATYYCAHFQGFGE |
| | SEYFQHWGQGTLVTVSS |
| 989 | QITLKESGPTLVKPTQTLTLTCTFSGFSLSTSGVG |
| | VGWIRQPPGKALEWLALIFWDDDKRYSPSLKSRLT |
| | ITKDTSKNQVVLTMTNMDPVDTATYYCAHRHPLTG |
| | FDSWGQGTLVTVSS |
| 990 | EVQLVESGGGLVKPGGSLRLSCAASGFTFSSYSMN |
| | WVRQAPGKGLEWVSSISSSSSYIYYADSVKGRFTI |
| | SRDNAKNSLYLQMNSLRAEDTAVYYCATPRGYSYG |
| | PLDYWGQGTLVTVSS |
| 991 | EVQLVESGGGLVKPGGSLRLSCAASGFTFSSYSMN |
| | WVRQAPGKGLEWVSSISSSSSYIYYADSVKGRFTI |
| | SRDNAKNSLYLQMNSLRAEDTAVYYCASPRGYSYG |
| | PFDYWGQGTLVTVSS |
| 992 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARDRVDKGY |
| | DFWSSWYFDLWGRGTLVTVSS |
| 993 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCASGGGSYFD |
| | AFDIWGQGTMVTVSS |
| 994 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARDRSGSYY |
| | GGFDYWGQGTLVTVSS |
| 995 | QVQLVESGGGVVQPGWSLRLSCAASGFTFGSYGMH |
| | WVRQAPGKGLEWVALIWNDGSNKYYADSVKGRFTI |
| | SRDKSKNTLYLQMNSLRAEDTAVYYCAKAVYGGNS |
| | VYFDYWGQGTLVTVSS |
| 996 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARIYGGNYE |
| | NYFDYWGQGTLVTVSS |
| 997 | QVQLVESGGGVVQPGRSLRLSCAASGFTFSIYAMH |
| | WVRQAPGKGLEWVAVISYDGSNKYYADSVKGRFTI |
| | SRDNSKNTLYLQMNSLRAEDTAVYYCARESEAGTT |
| | PSFDYWGQGTLVTVSS |
| 998 | EVQLVESGGGLVQPGGSLRLSCAASGFTVSSNYMS |
| | WVRQAPGKGLEWVSVIYSGGSTNYADSVKGRFTIS |
| | RDNSKNTLYLQMNSLRAEDTAVYYCARSLVRGVIT |
| | YFDYWGQGTLVTVSS |
| 999 | EVQLVESGGGLVQPGGSLRLSCAASGFTFSNYWMN |
| | WVRQAPGKGLEWVANIKEDGSETYYVDSVKGRFTI |
| | SRDNAKNSLYLQMNSLRAEDTAVYYCARGLSMEVW |
| | GQGTTVTVSS |
| 1000 | QVQLQQWGAGLLKPSETLSLTCAVYGGSFSGYYWS |
| | WIRQPPGKGLEWIGEIDHSGSTNDNPSLKSRVTIS |
| | VDTSKNQFSLKLSSVTAADAAVYYCARGGYSSSWY |
| | GTKYYFDYWGQGTLVTVSS |
| 1001 | QVQLQQWGAGLLKPSETLSLTCAVYDGSFSGHYWS |
| | WIRQPPGKGLEWIGEINHSGSTNYNPSLKSRVTIS |
| | VDTSKNQFSLKLSSVTAADTAVYYCARGPTVTTFF |
| | RRNAWFDPWGQGTLLTVSS |
| 1002 | QVQLQQWGAGLLKPSETLSLTCAVYGGSFSGYYWS |
| | WIRQPPGKGLEWIGEINHSGSTNYNPSLKSRVTIS |
| | VDTSKNQFSLKLSSVTAADTAVYYCARGRYSSGWY |
| | GSRNWFDPWGQGTLVTVSS |
| 1003 | QVQLQESGPGLVKPSETLSLTCTVSGGSISSYYWS |
| | WIRQPPGKGLEWIGYIYYSGSTNYNPSLKSRVTIS |
| | VDTSKNQFSLKLSSVTAADTAVYYCARLSMGAARQ |
| | SGFDPWGQGTLVTVSS |
| 1004 | QVQLQESGPGLVKPSETLSLTCTVSGGSISSYYWS |
| | WIRQPPGKGLEWIGYIYYSGSTNYNPSLKSRVTIS |
| | VDTSKNQFSLKLSSVTAADTAVYYCARDGGRDGYN |
| | ELGARVYYYYGMDVWGQGTTVTVSS |
| 1005 | EVQLVQSGAEVKKPGESLRISCKGSGYNFTSYWIS |
| | WVRQMPGKGLEWMGTIDPSDSYTNYRPSFQGHVTI |
| | SADKSINTAYLQWSSLKASDTAMYYCARIGSYGIW |
| | GQGTLVTVSS |
| 1006 | QVQLVQSGSELKKPGASVKVSCKASGYTFTSYAMN |
| | WVRQAPGQGLEWMGWINTNTGNPTYAQGFTGRFVF |
| | SLDTSVSTAYLQISSLKAEDTAVYYCAKLGCSGGS |
| | CYYYYGMDVWGQGTTVTVSS |
| 1007 | EVQLVESGGGLIQPGGSLRLSCAASGFTVSSNYVS |
| | WVRQAPGTGLEWVSVVYSGGHAYYADSVKGRFTMS |
| | RDNSENAVYLQMNSLRAEDTAVYYCARGDHYYDRS |
| | GPHKFDYWGQGTLVTVSs |
| 1008 | QVQLQESGPGLVKPSQTLSLTCTVSGGSISSGLYH |
| | WSWIRQPAGKGLEWIGRIFSSGSTAYSPSLKSRVI |
| | ISADTSKNQFSLKLSSVTAADTAVYYCARDSPLKF |
| | DSFGYPLYGMDVWGQGTTVTVSS |
| 1009 | QVQLVQSGAEVKKPGSSVKVSCKASGGTFSSYTIS |
| | WVRQAPGQGLEWMGRIIPILGIANYAQKFQGRVTI |
| | TADKSTSTAYMELSSLRSEDTAVYYCARGIVGATP |
| | GYFDYWGQGTLVTVSS |
| 1010 | QVQLVQSGAEVKKPGASVKVSCKASGFTFGRHGIT |
| | WVRQAPGQGLEWMGWISTYSGNTNYAQNLQGRVTM |
| | TTDTSTNTAYMELRSLFFDDTAVYYCAKAVSGWPI |
| | YFDAWGQGTLVTVSS |
| 1011 | QIQLVQSGAEVKKPGASVRVSCKASGFTFGRYGIT |
| | WVRQVPGQGLEWMGWISTYSGNTNYAQNLQGRVTM |
| | TTDTSTNTAYMELRSLFFDDTAMYYCAKAVSGWPI |
| | YFDAWGQGTLVTVSS |
| 1012 | QITLEESGPTLVKPTQTLTLTCTFSGFSLTTRGEG |
| | VAWIRQPPGKALEWLALIYWDDDQRYTPSLDSRLT |
| | ITKDISKNHVVLTLTDVEPVDTATYFCAHTIHSGY |
| | DRTFDSWGQGTLVIVSS |
| 1013 | QVQLVQSGSELKKPGASVKVSCKASGYTFTFYTIY |
| | WVRQAPGQGLEWMGWINTNTGTPTYAQGFTGRFVF |
| | SLDTSVSTAYLQISSLKAEDTAIYYCAREESYSSS |
| | SPLDYWGPGTLVAVSS |
| 1014 | QMQLVQSGPEVKKPGTSVKVSCKASGFTFTSSAVQ |
| | WVRQARGQRLEWIGWIVVGSGNTNYAQKFQERVTI |
| | TRDMSTSTAYMELSSLRSEDTAVYYCAAGSDFWSG |
| | YYVNYYMDVWGKGTTVTVSS |
| 1015 | QVTLRESGPALVKPTQTLTLTCTFSGFSLSTSGMC |
| | VSWIRQPPGKALEWLARIDWDDDKYYSTSLKTRLT |
| | ISKDTSKNQVVLTMTNMDPVDTATYYCARLTAAGV |
| | YFDYWGQGTLVTVSS |
| 1016 | EVQLLESGGGVVQPGGSLRLSCAASGFTFTTYAMN |
| | WVRQAPGRGLEWVSAISDSGGSAYYADSVKGRFTI |
| | SRDNSKNTLYLQMNSLRAEDTAVYYCAKTRGRGLY |
| | DYVWGSKDYWGQGTLVTVSS |
| 1017 | EVQLLESGGGVVQPGGSLRLSCAASGFAFTTYAMN |
| | WVRQAPGRGLEWVSAISDGGGSAYYADSVKGRFTI |
| | SRDNSKNTLYLQMNSLRAEDTAVYYCAKTRGRGLY |
| | DYVWGSKDYWGQGTLVTVSS |
| 1018 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARDESGSYY |
| | GDQAFDIWGQGTMVTVSS |
| 1019 | QAQLVQSGPEVKKPGASVKVSCEASGYTFSRYGIS |
| | WVRQAPGQGLEWMGWISGYNGNTTSEQKVQGRVTM |
| | TTDTSTNKVFLELRSLRSDDTAMYYCARDRRARAY |
| | EIPFGSDHYYFGMDVWGQGTTVTVSS |
| 1020 | QVQLVQSGAEVKKPGASVKVSCKASGYTFTGYYMH |
| | WVRQAPGQGLEWMGWINPNSGGTNYAQKFQGRVTM |
| | TRDTSISTAYMELSRLRSDDTAVYYCARDYYGSGS |
| | YPIGYMDVWGKGTTVTVSS |
| 1021 | EVQLVESGGGLAKPGGSLRVSCVVSGSGFTFRNAW |
| | MSWVRQAPGKGLEWVGRIKSKNDGGTTDYAASVKG |
| | RFTISRDDSKNSLDLQMQSLKTEDTAVYYCTTSYC |
| | STKVCFDYWFDPWGQGTLVTVSS |
| 1022 | QVQLVESGGDVVQPGNSLRLSCAASGFTFNFYGMH |
| | WVRQAPGKGLEWVAFISYDGNKRYYVDSVRGRFTA |
| | SRDNSKNTLFLQMNGLRNDDSAVYYCASNLYATSP |
| | YGGVKNWGRGTLVAVAS |
| 1023 | EVQLVESGGGLVQPGRSLRLSCAASGFTFDDYAMH |
| | WVRQAPGKGLEWVSGISWNSGSIGYADSVKGRFTI |
| | SRDNAKNSLYLQMNSLRAEDTALYYCAKDIGSGSP |
| | DAFDIWGQGTMVTVSS |
| 1024 | GVQLVESGGGLVQPGRSLRLSCAASGFIFDDYTMH |
| | WVRQAPTKGLEWVSGITWNYATVGYADSVRGRFTI |
| | SRDNVKNSLFLQIHSLRPDDTAFYYCVKDLEFRGG |
| | TGGFDLWGQGTLVTVSS |
| 1025 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARDGHSAWG |
| | AFDIWGQGTMVTVSS |
| 1026 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARDHPTLRR |
| | AFDYWGQGTLVTVSS |
| 1027 | QVELVQSGAQVRKPGASVKVSCKASGDTFNDYHMH |
| | WVRQAPGQGLEWMGWINPNSGETRYSQRFQGTVTM |
| | TRDTSISTVYMELRSLPSDDTAVYFCARDRGSSSW |
| | WGWLDPWGQGTLVTVSS |
| 1028 | QVQLVQSGAEVKKPGSSVKVSCKASGGTFSSYAIS |
| | WVRQAPGQGLEWMGGIIPIFGTANYAHKFQGRVTI |
| | TADESTSTAYMELSSLRSEDTAVYYCATRRGYSGY |
| | GAAYYFDYWGQGTLVTVSS |
| 1029 | QVQLVQSGAEVKKPGSSVKVSCKASGGTFSSYAIS |
| | WVRQAPGQGLEWMGRIIPILGIANYAQKFQGRVTI |
| | TADKSTSTAYMELSSLRSEDTAVYYCAREVYVGGE |
| | DDYSYYYGLDVWGQGTTVTVSS |
| 1030 | EVQLVESGGGLVKPGGSLRLSCAASGFTFSNAWMS |
| | WVRQAPGKGLEWVGRIKSKTDGGTTDYAAPVKGRF |
| | TISRDDSKNTLYLQMNSLKTEDTAVYYCTTDLGEA |
| | GPTEWLRSSLFDYWGQGTLVTVSS |
| 1031 | DIHMAESGGGLVKPGGSLRLSCAVSGLTFTKAWMS |
| | WVRQAPGKGPEWVGRIKSRSDGGKIDYAAPVKGRF |
| | IISRDDSKNTLYLQMHSLKTEDTALYYCTTSYCNP |
| | KVCFDYWFDPWGQGTLVTVSS |
| 1032 | EVQLLESGGGLVQPGGSLRLSCAASGFTFSTYAMS |
| | WVRQAPGKGLEWVSVISGSGGSTYYADSVKGRFTI |
| | SRDNSKNTLYLQMNSLRAEDTAVYYCAKEYYYDSS |
| | GYYYREDAFDIWGQGTMVTVSS |
| 1033 | EVQLLESGGGLVQPGGSLRLSCAASGFTFSNYAMT |
| | WVRQAPGKGLEWVSGISANGRSPYYADSVKGRFTI |
| | SRDNSKNTMYVQMNSLRVEDTAVYYCAKDGGLTAY |
| | LEYWGLGTLVTVSA |
| 1034 | EVQLLESGGGLVQPGGSLRLSCAASGFTFSSYAMS |
| | WVRQAPGKGLEWVSAISGSGGSTYYADSVKGRFTI |
| | SRDNSKNTLYLQMNSLRAEDTAVYYCATEKWEVVD |
| | VCFDYWGQGTLVTVSS |
| 1035 | QVQLVESGGGVVQPGRSLRLSCAASGFTFSSYGMH |
| | WVRQAPGKGLEWVAVISYDGSNKYYADSVKGRFTI |
| | SRDNSKNTLYLQMNSLRAEDTAVYYCAKDIGWDVV |
| | VVAATHGVFDYWGQGTLVTVSS |
| 1036 | QVQLVESGGGVVQPGRSLRLSCAASGFTFSSYGMH |
| | WVRQAPGKGLEWVAVISYDGSNKYYVDSVKGRFTI |
| | SRDNSKNTLYLQMNSLRAEDTAVYYCAKDPYYYGS |
| | GSSNFFDYWGQGTLVTVSS |
| 1037 | QVQLVESGGGVVQPGWSLRLSCAASGFTFSSFAMY |
| | WVRQAPGKGLEWVAVISYDGANKYYADSVKGRFTI |
| | SRDNSKNTLYLQVNSLRVEDTAVYYCARGPDYYDT |
| | GGYFDLWGRGTLVTVSs |
| 1038 | QVQLVESGGGVVQPGRSLRLSCAASGFTFSNYGMH |
| | WVRQAPGKGLEWVAVMWHDGSNKYHSDSVKGRFTI |
| | SRDNSKNTLYLQMKTLRADDTAVYYCARDGYKQIY |
| | WYLDLWGRGTLVTVSS |
| 1039 | QVQLVESGGGVVQPGRSLRLSCAASGFTFSSYGMH |
| | WVRQAPGKGLEWVAVIWYDGSNKYYADSVKGRFTI |
| | SRDNSKNTLYLQMNSLRAEDTAVYYCAKGEGVYGS |
| | GSRYFLDYWGQGTLVTVSS |
| 1040 | QVQLVESGGGVVQPGRSLRLSCAASGFTFSSYATH |
| | WVRQAPGKGLEWVAVISYDGSNKYHADSVKGRFTI |
| | SRDNSKNTLYLQMNSLRAEDTAVYYCAREWSRGAV |
| | AGTGYFDYWGQGTLVTVSS |
| 1041 | EVQLVESGGGLVQPGRSLRLSCAASGFTFHDYAMH |
| | WVRQAPGKGLEWVSGISWNSGSIGYADSVKGRFTI |
| | SRDNAKNSLYLQMNSLRAEDTALYYCAKVAKLPGD |
| | YYGMDVWGQGTTVTVSS |
| 1042 | EVQLVESGGGLIQPGGSLRLSCAASGVIVSRNYMN |
| | WVRQAPGKGLEWVSVIYSGGSTFYADSVKGRFTIS |
| | RDNSKNTLYLQMNSLRAEDTAVYYCARELRGAFDI |
| | WGQGTMVTVSS |
| 1043 | EVQLVESGGGLVQPGGSLRLSCAASGFTVSSNYMS |
| | WVRQAPGKGLEWVSVIYSGGTTYYADSVKGRFTIS |
| | RHNSKNTLYLQMNSLRAEDTAVYYCARDWGEYYFD |
| | YWGQGTLVTVSS |
| 1044 | EVQLVESGGGLIQPGGSLRLSCAASEFTVSSNYMS |
| | WVRQAPGKGLEWVSVIYSGGSTYYADSVKGRFTIS |
| | RDNSKNTLYLQMNSLRAEDTAVYYCARDYGDLYFD |
| | YWGQGTLVTVSS |
| 1045 | EVQLVESGGGLIQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARDRRVGSPY |
| | YYYYMDVWGKGTTVTVSS |
| 1046 | EVQLVESGGGLVQPGGSLRLSCAASGFTVSSNYMS |
| | WVRQAPGKGLEWVSILYSGGTTYYADSVKGRFTIS |
| | RDNSKNTLYLQMNSLRAEDTAVYYCARDLGDNAFD |
| | IWGQGTMVTVSS |
| 1047 | EVQLVESGGGLVQPGGSLRLSCAASGITVSSNYMS |
| | WVRQAPGKGLEWVSVIYSGGSTYYADSVKGRITIS |
| | RDNSKNTLYLQMNSLRAEDTAVYYCARDRYSGYDF |
| | WGQGTLVTVSS |
| 1048 | EVQLVESGGGLVQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARLSGTGYGG |
| | DGGWFDPWGQGTLVTVSS |
| 1049 | EVQLVESGGGLVQPGGSLRLSCAASGFTFSSYWMH |
| | WVRQAPGKGLVWVSRIKSDGSSTSYADSVKGRFTI |
| | SRDNAKNTLYLQMNSLRAEDTAVYYCAGKKIYYGS |
| | SFDPWGQGTLVTVSS |
| 1050 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARGGSGSG |
| | WYGGRFDYWGQGTLVTVSS |
| 1051 | QVQLQESGPGLVKPSQTLSLTCTVSGGSISSGGYY |
| | WSWIRQHPGKGLEWIGYIYYSGSTYYNPSLKSRVT |
| | ISVDTSKNQFSLKLSSVTAADTAVYYCARVWRETY |
| | YYDSSGDSFDYWGQGTLVTVSS |
| 1052 | QVQLQQWGAGLLKPSETLSRTCAVYGGSFSGYYWT |
| | WIRQSPGKRLEWIGEISHGGKTNYNIFFEGRVTLS |
| | VDSSKSQFSLTLASVTAADTAIYYCARGRSITGIR |
| | DVDFWGQGALVTVSS |
| 1053 | QVQLHQWGAGLLKPSETLSLTCAVSGGSFSDDFWN |
| | WIRQPPGKGLEWIGEINHSGTTNYNPSLKSRITMS |
| | VDTSKSQFSLKLNSVTAADSAMYFCARGRGNYMFR |
| | WFDPWGQGTLVTVSS |
| 1054 | QVQLQESGPGLVKPSETLSLTCTVSGGSISSYYWS |
| | WIRQPPGKGLEWIGYIYYSGSTNYNPSLKSRVTIS |
| | VDTSKNQFSLKLSSVTAADTAVYYCARGGLWYDSI |
| | NYYGMDVWGQGTTVTVSS |
| 1055 | EVQLVQSGAEVKKPGESLKISCKGSGYSFTSYWIG |
| | WVRQMPGKGLEWMGIIYPGDSDTRYSPSFQGQVTI |
| | SADKSISTAYLQWSSLKASDTAMYYCARLILRWPT |
| | TWDYFDYWGQGTLVTVSS |
| 1056 | QVQLVQSGTEVKEPGSSVKVSCKASGDTFSNYPIA |
| | WVREAPGQGLEWMGRIIPIVGFANYAQKFQGRVTI |
| | TADKSTSTAYMELSSLRFEDTAVYYCARVDGPFDY |
| | WGQGTLVTVSS |
| 1057 | QVQLVESGGGVVQPGRSLRLSCAASGFTFSTSAMH |
| | WVRQAPGKGLEWVAGISYDGSNEHLDSVKGRFTIS |
| | RDNSKNTLYLQMSSLRPEDTAVYYCARCPFWNYGH |
| | CYLDNWGQGTLVTVSS |
| 1058 | QVQLVESGGGVVQPGGSLRLSCAASGFTFSTYAMH |
| | WLRQAPGRGLEWVAVISYDGSNKYNADSVKGRFTI |
| | SRDNSKNTLSLHMNSLRPEDTAVYYCARPSVRWYY |
| | HAMDVWGQGTTVTVTS |
| 1059 | EMQLLESGGGLVQPGGSLRLSCAASGFTFFSYALS |
| | WVRQAPGKGLEWVSGISGISDSGGNTYYADSVKGR |
| | FTISRDNSQNMLYLQMNSLRVEDTAVYYCAKERRP |
| | VLRYFDWLPIEAPDYWGPGTLVTVSS |
| 1060 | EVQLVESGGGLVQPGGSLRLSCAASGFTVSSNYMN |
| | WVRQAPGKGLEWVSVIYSGGSTYYADSVKGRFTLS |
| | RDNSKNTLYLQMNSLRAEDTAVYYCARGQYDILTG |
| | YQYGAFDIWGQGTMVTVSS |
| 1061 | QVQLQESGPGLVKPSQTLSLTCTVSAGSISSDTYY |
| | WSWIRQPAGKGLEWIGRIYTTGSTIYNPSLNSRVL |
| | ISADTSNNQFSLKLTSVTASDTAVYYCAAHYYSRT |
| | DAFHIWGQGTMVTVSS |
| 1062 | QVQLVQSGAEVKKPGASVKVSCKASGYTFTSYGIS |
| | WVRQAPGQGLEWMGWISAYNGNTNYAQKLQGRVTM |
| | TTDTSTSTAYMELRSLRSDDTAVYYCARDSVSGSG |
| | SYYKGLWFDPWGQGTLVTVSS |
| 1063 | QVQLVQSGAEVKKPGASVKVSCKASGYTFTNYYMH |
| | WMRQAPGQGLEWMGIINPSGGSTSYAHQFQGRVTM |
| | TRDTSTSTVYMEMSSLRSEDTAVYFCVVGIGYCSS |
| | PSCPPLRWFDYWGQGTLVTVSS |
| 1064 | QVQLVQSGAEVKKPGSSVKVSCKASGGTFSSYTIS |
| | WVRQAPGQGLEWMGRIIPILGIANYAQKFQGRVTI |
| | TADKSTSTAYMELSSLRSEDTAVYYCARERGYSGS |
| | GSLYYFDYWGQGTLVTVSS |
| 1065 | QITLKESGPTLVKPTQTLTLTCTFSGFSLSTSGVG |
| | VGWIRQPPGKALEWLALIYWDDDKRYSPSLKSRLT |
| | ITKDTSKNQVVLTMTNMDPVDTATYYCAHYSSSRP |
| | PLFDYWGQGTLVTVSS |
| 1066 | EAQLLESGGGLVQPGGSLRLSCAVSGFTVSSYDMS |
| | WVRQAPGKRLEWVSFISARGSVTYYADSVRGRFTI |
| | SRDNFKNTLYVEMNNLRVEDTAVYYCAKGHWSTWG |
| | QGTLVTVSS |
| 1067 | QVQLVESGGGVVQPGRSLRLSCAASGFTFRNYGMH |
| | WVRQAPGKGLEWVAVISYDGSNKYYADSVKGRFTI |
| | SRDNSKSTLYLQMNSLRAEDTAVYYCANGAYYYGS |
| | GSYYNGAAYWGQGTLVTVSS |
| 1068 | QVQLVESGGGVVQPGKSLRLSCAASGFTFSSYGMH |
| | WVRQAPGKGLEWVAVISNYGSNKYHADSVKGRITI |
| | SRDNSKNTLYLQMNSLRAEDTAVYYCAKGGYYDIL |
| | TGYFPFDYWGQGTLVTVSS |
| 1069 | EVQLVESGGGLIQPGGSLRLSCAASGFTVSRNYMS |
| | WVRQAPGKGLEWVSLIYSGGSTFYADSVKGRFTIS |
| | RDNSKNTLYLQMNTLRSEDTAVYYCARDLVVYGMD |
| | VWGQGTTVTVSS |
| 1070 | EVQLVESGGGLVQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARDPIRNGMD |
| | VWGQGTTVTVSS |
| 1071 | EVQLVESGGGLVQPGGSLRLSCAASGFTVSRNYMS |
| | WVRQAPGKGLEWVSVIYSGGTTHYADSVKGRFTIS |
| | RHNSKNTLYLQMNSLRAEDTAVYYCARDLVVYGMD |
| | VWGQGTTVTVSS |
| 1072 | EVQLVESGGGLIQPGGSLRLSCAASGLTVSSNYMT |
| | WVRQAPGKGLEWVSVIYSGGSTFYADSVKGRFTIS |
| | RHNSKNTLYLQMNSLRAEDTAVYYCARDAMSYGMD |
| | VWGQGTTVTVSS |
| 1073 | EVQLVESGGGLIQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARDRVVYGMD |
| | VWGQGTTVTVSS |
| 1074 | EVQLVESGGGLIQPGGSLRLSCAASGLIVSSNYMS |
| | WVRQAPGKGLEWVSVLYAGGSTDYAGSVKGRFTIS |
| | RDNSKNTLYLQMNSLRAEDTAVYYCARDAAVYGID |
| | VWGQGTTVTVSS |
| 1075 | EVQLVESGGGLVQPGGSLRLSCAASGITVRSNYMS |
| | WVRQAPGKGLEWVSVIYSGGSTFYADSVKGRFTIS |
| | RDNSKNTLYLQMNSLRAEDTAVYYCARDLISRGMD |
| | VWGQGTTVTVSS |
| 1076 | EVQLVESGGGLIQPGGSLRLSCAASGFTVSSNYMN |
| | WVRQAPGKGLEWVSVIYSGGSTFYADSVKGRFTIS |
| | RDNSKNTLYLQMNSLRAEDTAVYYCARDRVVYGMD |
| | VWGQGTTVTVSS |
| 1077 | EVQLVESGGGLVQPGGSLRLSCAASGVTVSSNYMS |
| | WVRQAPGKGLEWVSVIYSGGSTNYADSVKGRFTIS |
| | RDNSKNTLYLQMNSLRAEDTAVYYCARDLVSYGMD |
| | VWGQGTTVTVSS |
| 1078 | EVQLVESGGGLIQPGGSLRLSCAASGLTVSSNYMN |
| | WVRQAPGKGLEWVSVIYSGGSTYYADSVKGRFTIS |
| | RDNSKNMVYLQMNSLRAEDTAVYYCARDLVVYGMD |
| | VWGQGTTVTVSS |
| 1079 | EVQLVESGGGLVQPGGSLRLSCAASGFIVSSNYMT |
| | WVRQAPGKGLEWVSVIYSGGSTFYADSVKGRFTIS |
| | RHNSKNTLFLQMNSLRAEDTAVYYCARDAQNYGMD |
| | VWGQGTTVTVSS |
| 1080 | EVQLVESGGGLVQPGGSLRLSCAASEFIVSRNYMS |
| | WVRQAPGKGLEWVSVIYSGGSTFYADSVKGRFTIS |
| | RDNSKNTLYLQMNSLRAEDTGVYYCARDRGLVSDY |
| | WGQGTLVTVSS |
| 1081 | DIEMTQSPSSLSASVGDRVTITCRASQSIASYAYW |
| | YQQKPGKAPKLLISAASILQSGVPSRFSGSGSGGH |
| | FTLTINSLQPEDVATYYCQQTYIIPYSFGQGTKLE |
| | IK |
| 1082 | AIRMTQSPSSFSASTGDRVTITCRASQGISSYLAW |
| | YQQKPGKAPKLLIYAASTLQSGVPSRFSGSGSGTD |
| | FTLTISCLQSEDFATYYCQQYYSYPYTFGQGTKLE |
| | IK |
| 1083 | QSALTQPPSASGSPGQSVTISCTGTSSDVGGYNYV |
| | SWYQQHPGKAPKLMIYEVSKRPSGVPDRFSGSKSG |
| | NTASLTVSGLQAEDEADYYCSSYAGSNNLVFGGGT |
| | KLTVL |
| 1084 | GIQMTQSPSTLSASVGDRVTITCRASQSISDWLAW |
| | YQQKPGKIPKLLIYKASTLESGVPSRFSGSGSGTE |
| | FTLTISSLQPDDFGTYYCQRYDSYRTFGQGTKVEI |
| | K |
| 1085 | DIQMTQSPSSLSASVGDRVTITCRASQSISSYLNW |
| | YQQKPGKSPKLLIYAASTLQSGVPSRFSGSGSGTD |
| | FTLTISSLQPEDFATYYCQQSYSTPYTFGQGTKLE |
| | IK |
| 1086 | DIQMTQSPSSLSASVGDRVTITCQASQDISNYLNW |
| | YQQKPGKAPKVLIYDASNLKTGVPSRFSGSGSGTD |
| | FTFTISSLQPEDIATYYCQQYDNLPLTFGQGTRLE |
| | IK |
| 1087 | EIVLTQSPGTLSLSPGERATLSCRASQSVSSSYLA |
| | WYQQKPGQAPRLLIYGASHRASGIPDRFSGSGSGT |
| | DFTLTISRLEPGDFAMYYCQQYATSPWTFGQGTTV |
| | EIK |
| 1088 | QSVLAQPPSASGTPGQSVTISCSGNNSNIGINNVY |
| | WYQQFPGTAPKLLIHRSNQRPSGVPDRFSGSRSGT |
| | SASLVISGLRSEDEAEYHCAAWDDSLSSWGFGGGT |
| | KLTVL |
| 1089 | QLVLTQSPSASASLGASVKLTCTLSSGHSSYAIAW |
| | HQQQPEKGPRYLMKLSSDGSHRKGDGIPDRFSGSS |
| | SGAERYLTISSLQSEDEADYYCQTWGTGTVVFGGG |
| | TKLTVL |
| 1090 | SYELTQPPSVSVSPGQTARITCSGDALPKQYAYWY |
| | QQKPGQAPVLVIYKDSERPSGIPERFSGSSSGTTV |
| | TLTISGVQAEDEADYYCQSADSSGTWVFGGGTKLT |
| | VL |
| 1091 | EIVLTQSPATLSLSPGERATLSCRASQSISSYLAW |
| | YQQKPGQAPRLLIYEAANRATGIADRFSGSGSGTD |
| | FTLTISSLEPEDFAIYYCQQRSDWTPTFGQGTKVE |
| | IK |
| 1092 | AIQLTQSPSSLSASVGDRVTITCRASQGISSALAW |
| | YQQKPGKAPKLLIYDASSLESGVPSRFSGSGSGTD |
| | FTLTISSLQPEDFATYYCQQFNSYPRTFGGGTKVE |
| | IK |
| 1093 | QSALTQPASVSGSPGQSITISCTATSSDFGTFHLV |
| | SWYQQHPGKAPQLMIYEVNKRPSGVSDRFSASKSG |
| | NTASLTISGLQPEDEADYYCCSYAGNTTFFGGGTK |
| | LTVL |
| 1094 | QAVLTQPPSVSAAPGQRVSISCSGSAFNIGTNFVS |
| | WYQHLPGAAPKLLIYGDQWRISGTPDRFSGSKSGT |
| | SATLAITGLQSGDEAHYYCSTWDASLKEVLFGGGT |
| | RLDVL |
| 1095 | DVVMTQSPLSLPVTLGQPASISCRSSQSLVYYDGN |
| | TYLNWFQQRPGQSPRRLIYKVSNRDSGVPDRFSGS |
| | GSGTDFTLKISRVEAEDVGVYYCMQGTHWPLTFGP |
| | GTKVDIK |
| 1096 | DIQMTQSPSTLSASVGDSVTITCRPSQSISRWLAW |
| | YQQKPGKAPKLLIYKASTLESGVPSRFSGSGSGTE |
| | FTLTISSLQPDDFATYYCQQYDSYPWTFGQGTKVE |
| | IK |
| 1097 | DIQLTQSPSFLPASVGDRVTITCRASQHISNYVAW |
| | YQQKPGKAPKLLIYAASTLESGVPSRFGGSGSGTE |
| | FTLTINSLQPEDFATYYCQQLTTYPRTFGQGTKLE |
| | IK |
| 1098 | SYELTQPPSVSVSPGQTARITCSGDALPKQYAYWY |
| | QQKPGQAPVLVIYKDSERPSGIPERFSGSSSGTTV |
| | TLTISGVQAEDEADYYCQSADSSGTWVFGGGTKLT |
| | VL |
| 1099 | DIVMTQSPDSLAVSLGERATINCKSSQSVLYSSNN |
| | NKNYLAWYQQKPGQPPKLLIYWASTRESGVPDRFS |
| | GSGSGTDFTLTISSLQAEDVAVYFCQQFYSTPVTF |
| | GPGTKVDIK |
| 1100 | NFMLTQPHSLSESPGKTVTISCTGSGASIASNYVQ |
| | WYQQRPGSAPVTVIFEDTQRPSGVPDRFSGSIDRS |
| | SNSASLTISGLRTEDEADYYCQSYDGSNVVFGGGT |
| | KLTVL |
| 1101 | DIVMTQSPDSLAVSLGERATINCKSSQSVLYSSNN |
| | KNYLAWYQQKPGQPPKLLIYWASTRESGVPDRFSG |
| | SGSGTDFTLTISSLQAEDVAVYYCQQYYSTPLTFG |
| | GGTKVEIK |
| 1102 | DIVMIQSPDSLAVSLGERATINCKSSHSVFFSKVN |
| | KDYLAWYQQKPGLPPKLLIYWASTRQTGVPDRFSG |
| | SGSGTDFSLTISNLQAEDVAVYYCQQYYDTPMYTF |
| | GQGTKLEIK |
| 1103 | DIQMTQSPSTLSASVGDRVTITCRASQSISSWLAW |
| | YQQKPGKAPKLLIYKASSLESGVPSRFSGSGSGTE |
| | FTLTISSLQPDDFATYYCQQYNSYPYTFGQGTKLE |
| | IK |
| 1104 | QSALTQPASVSGSPGQSITISCTGTSSDVGGYNYV |
| | SWYQQHPGKAPKLMIYDVSKRPSGVSNRFSGSKSG |
| | NTASLTISGLQAEDEADYYCSSYTSSSTFVFGTGT |
| | KVTVL |
| 1105 | SYELTQPPSVSVSPGQTARITCSGDALPKQYAYWY |
| | QQKPGQAPVLVIYKDSERPSGIPERFSGSSSGTTV |
| | TLTISGVQAEDEADYYCQSADSSGTYSNWVFGGGT |
| | KLTVL |
| 1106 | QSALTQPASVSGSPGQSITISCTGTSSDVGGYNYV |
| | SWYQQHPGKAPKLMIYDVSNRPSGVSNRFSGSKSG |
| | NTASLTISGLQAEDEADYYCSSYTSSSTVVFGGGT |
| | KLTVL |
| 1107 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPLTFGGGTKV |
| | EIK |
| 1108 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPLTFGGGTKV |
| | EIK |
| 1109 | DFQMTQSPSSLSASVGDRVTISCQASEDIDNHLNW |
| | YQQKPGKAPRLLIYDASNLETGVPSRFSGSGSGTD |
| | FLFTITSLQPEDFATYYCQQYGAFGQGTKVEIK |
| 1110 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPWTFGQGTKV |
| | EIK |
| 1111 | QSVLTQPPSASGTPGQRVTISCSGSRSNIGSKNVH |
| | WYQQLPGTAPKFLIYSNNQRPSGVPDRFSGSKSGT |
| | SASLAISGLQSEDEADYYCAVWDDSLNGVVFGGGT |
| | KLTVL |
| 1112 | QSALTQPPSASGSPGQSVTISCTGTSSDVGSYHYV |
| | SWYQQHPGKAPKLIIYEVSKRPSGVPDRFSGSKSG |
| | NTASLTVSGLQTDDEADYYCSSFAGSNNPYVFGTG |
| | TKVTVL |
| 1113 | DIVMTQSPDSLAVSLGERATINCRSSQSVLYSANN |
| | KYYLAWYQHKPGQPPKLLIHWASTRESGVPDRFSG |
| | SGSGTDFTLTISSLQAEDVAIYYCQQYYSTPYTFG |
| | QGTKLEIK |
| 1114 | EIVMTQSPATLSVSPGERATLSCRASQSVKSYLAW |
| | YQQKAGQAPRLLIYGASSRATGIPARFSGSRSGTE |
| | FTLTISSLQSEDFAVYFCHQYDSWPPTFGGGTKVE |
| | IK |
| 1115 | DVVLTQSPATLSLSPGERATLSCRASKDINSYLAW |
| | YQQKPGQAPRLLIYDASKRATGVPVRFSGSGSGTD |
| | FTLTISSLEPEDSAIYFCQNRDDWPPLFTFGPGTK |
| | VDFK |
| 1116 | DIVMTQSPDSLAVSLGERATINCKSSQSVLYSSNN |
| | KNYLAWYQQKPGQPPKLLIYWASTRESGVPDRFSG |
| | SGSGTDFTLTISSLQAEDVAVYYCQQYYSTPRTFG |
| | QGTKLEIK |
| 1117 | DMQMTQSPSSVSASVGDRVTITCRASQDISSSLAW |
| | YQQKPGKPPKLLIYAASSLQRGVPSRFSGSGSGTD |
| | FTLTISSLQPEDFATYYCQQAHSFLSLTFGGGTKV |
| | EIK |
| 1118 | SCELTQPPSVSVSPGQTARITCSGDALSNQYTYWY |
| | QQRPGQAPLLVIYKGTKRPSAIPERFSGSRSGTTV |
| | TLTISGVQAEDEADYYCQSADTSGTYLWVFGGGTK |
| | LTVL |
| 1119 | DIQMTQSPSSLSASVGDRVTITCQASQDISNFLNW |
| | YQQKPGKAPELLIYDASNLETGVPSRISGSGSGTD |
| | FTFTISSLQPEDIATYYCQQYDSLPITFGQGTRLE |
| | IK |
| 1120 | DIQMTQSPSSLSAVLGDRVTITCRASQAISNSLAW |
| | YQQKPGKAPKLLLYAASRLESGVPSRFSGSGSGTD |
| | YTLTISSLRPEDFATYYCQQYYGIPTFGQGTRLEN |
| | K |
| 1121 | DVQMTQSPSSLSASVGDRVTITCQASRDIHNLLNW |
| | YQQKPGKAPKLLIYDASNLETGVPSRFSGSGSGTD |
| | FTFTITGLQPEDVATYYCQKCDNFPWTFGQGTKVE |
| | IK |
| 1122 | QSVLTQSPSASGTPGQRVTISCSGSNSNIGSNYVF |
| | WYHQLPGTTPKLLIYKNNQRPSGVPDRFSGSKSGT |
| | SASLAISGLRSEDEADYYCAAWDDSLSVVVFGGGT |
| | KLTVL |
| 1123 | DIHMTQSPSSLSASEGDRVTISCRASQGISTNYLN |
| | WYQQKSGKAPRLLIYATSTLQSGVSSRFSGSGSGT |
| | DFTLTINSVQPEDFATYYCQQSYSSPPTFGGGTKL |
| | DIK |
| 1124 | QSVLTQPPSVSGAPGQRVTISCTGIGARYNVHWYQ |
| | QVPGTAPKLLIYRNTNRPSGVPDRFSGSKSDTSAS |
| | LAITGLQAEDEADYYCQSYDDTLTIFGGGTKLTVL |
| 1125 | DIQMTQSPSSLSASVGDRVTITCRASQSISNHFNW |
| | YQHRPQKAPKLLIYSASNLQSGVPSRFSGSGSGRN |
| | FTLTISSLQPEDFATYYCQQSYGAPPTFGGGTKVE |
| | IK |
| 1126 | DIQMTQSPSSLSASEGDRVTITCRANQSISTNYLN |
| | WYQQQSGKAPKLLIYASSTLQSGVPTRFSGSGSGT |
| | DFALTINSLQPEDFAAYYCQQSYSTPPTFGGGTRV |
| | DLR |
| 1127 | DIQMTQSPSSLSASEGDRVTILCRASQSISTNYLN |
| | WYQQKSGKAPKLLIYSTSNLQSGVPSRFSGSGSGT |
| | DFTLTIDSLQGEDFATYYCQQSFSTPPTFGGGTKV |
| | DIK |
| 1128 | DIQMTQSPSSLSASEGDRVTITCRANQSISTNYLN |
| | WYQQKSGKAPNLLIYATSSLERGVPSRFSGSGSGT |
| | EFSLTINSLQPEDFVTYYCQQSYSSPPTFGGGTKV |
| | EIKRMEIK |
| 1129 | SYELTQPPSVSVSPGQTARITCSGDALPKKYAYWY |
| | QQKSGQAPVLVIYEDSKRPSGIPERFSGSSSGTMA |
| | TLTISGAQVEDEADYYCYSTDSSGNHWVFGGGTKL |
| | TVL |
| 1130 | QAVVTQEPSLTVSPGGTVTLTCGSSTGAVTSGHYP |
| | YWFQQKPGQGPRTLIYDINNKYSWTPARFSGSLLG |
| | GKAALTLFGAQPEDEADYYCLLSYSGVRIFGGGTK |
| | LTVL |
| 1131 | QSVLTQPPSVSGAPGQRVTISCTGSSSNIGAGYDV |
| | HWYQQLPGTAPKLLIYGNSNRPSGVPDRFSGSKSG |
| | TSASLAITGLQAEDEADYYCQSYDSSLSKVFGGGT |
| | KLTVL |
| 1132 | SYELTQPPSVSVSPGQTASITCSGDKLGDKYACWY |
| | QQKPGQSPVLVIYQDSKRPSGIPERFSGSNSGNTA |
| | TLTISGTQAMDEADYYCQAWDSSTFYVFGTGTKVT |
| | VL |
| 1133 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAVVFGGGT |
| | KLTVL |
| 1134 | EIVMTQSPATLSVSPGERATLSCRASQSVSSNLAW |
| | YQQKPGQAPRLLIYGASTRATGIPARFSGSGSGTE |
| | FTLTISSLQSEDFAVYYCQQYNNWPWTFGQGTKVE |
| | IK |
| 1135 | QAVVTQEPSLTVSPGGTVTLTCGSSTGAVTSGHYP |
| | YWFQQKPGQAPRTLIYDTSNKHSWTPARFSGSLLG |
| | GKAALTLSGAQPEDEAEYYCLLSYSGARPVFGGGT |
| | KLTVL |
| 1136 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPPYTFGQGTKL |
| | EIK |
| 1137 | QSALTQPASVSGSPGQSITISCTGTSSDVGGYNYV |
| | SWYQQHPGKAPKLMIYDVSKRPSGVSNRFSGSKSG |
| | NTASLTISGLQAEDEADYYCSSYTSSSTRVVFGGG |
| | TKLTVL |
| 1138 | DIVMTQSPDSLAVSLGERATINCKSSQSVLYSSNN |
| | KNYLAWYQQKPGQPPKLLIYWASTRESGVPDRFSG |
| | SGSGTDFTLTISSLQAEDVAVYYCQQYYSTPITFG |
| | QGTRLEIK |
| 1139 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPLTFGGGTKV |
| | EIK |
| 1140 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSVVVFGGGT |
| | KLTVL |
| 1141 | QSALTQPASVSGSPGQSITISCTGTSSDVGGYNYV |
| | SWYQQHPGKAPKLMIYDVSKRPSGVSNRFSGSKSG |
| | NTASLTISGLQAEDEADYYCSSYTSSSTFAVFGGG |
| | TQLTVL |
| 1142 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTPLTFGG |
| | GTKVEIK |
| 1143 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTVFTFGP |
| | GTKVDIK |
| 1144 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTVFTFGP |
| | GTKVDIK |
| 1145 | DIEMTQSPSSLSASVGDRVTITCRASQSIASYAYW |
| | YQQKPGKAPKLLISAASILQSGVPSRFSGSGSGGH |
| | FTLTINSLQPEDVATYYCQQTYIIPYSFGQGTKLE |
| | IK |
| 1146 | AIRMTQSPSSFSASTGDRVTITCRASQGISSYLAW |
| | YQQKPGKAPKLLIYAASTLQSGVPSRFSGSGSGTD |
| | FTLTISCLQSEDFATYYCQQYYSYPYTFGQGTKLE |
| | IK |
| 1147 | SYVLTQPPSVSVAPGKTARITCGGNNIGSKSVHWY |
| | QQKPGQAPVLVIYYDSDRPSGIPERFSGSNSGNTA |
| | TLTISRVEAGDEADYYCQVWDSSSDHVVFGGGTKL |
| | TVL |
| 1148 | SYELTQPPSVSVSPGQTASITCSRDKLGDEYACWY |
| | QQKPGQSPILVIYQNNKRPAGIPERFSGSNSGNTA |
| | TLTISGTQAMDEADYYCQAWDSSTSYVVFGGGTKL |
| | TVL |
| 1149 | QSVLTQPPSVSAAPGQKVTISCSGSSSNIGNNYVS |
| | WYQQLPGTAPKLLIYDNNKRLSGIPDRFSGSKSGT |
| | SATLDITGLQTGDEADYYCGTWDSSLSVGVFGGGT |
| | KLTVL |
| 1150 | QSALTQPASVSGSPGQSITISCTGTSSDVGGYNYV |
| | SWYQQHPGKAPKLMIYDVSKRPSGVSNRFSGSKSG |
| | NTASLTISGLQAEDEADYYCNSYTSNSTAVFGGGT |
| | KLTVL |
| 1151 | EVVLTQSPATLSASPGERATLSCRASLSINTDLAW |
| | YQQRPGQPPRLLIYGASTRATGIPARFSGSGSGTE |
| | FTLTVSSLQSEDFALYYCQQSYNWPRTFGQGTRVE |
| | IK |
| 1152 | DIQMTQSPSAMSASVGDRVTITCRASQGMSNYLAW |
| | FQQKPGKVPKRLIYAASSLASGVPSRFSGSGSGTE |
| | FTLTISSLQPEDFATYYCLQHNSYPYTFGQGTKLE |
| | IK |
| 1153 | DIQMTQSPSTLSAPVGDRVTITCRASQSINSWLAW |
| | YQQKPGKAPKLLIYKASNLESGVSSRFSGSGSGTE |
| | FTLTISSLQPDDFATYYCQQYNGYPHTFGQGTKLE |
| | IK |
| 1154 | DIQMTQSPSTLSASVGDRVTITCRASQSISSWLAW |
| | YQQKPGKAPKLLIYKASNLESGVPSRFSGSGSGTD |
| | FTLTISSLQPDDFATYYCQQYSYYSAFGQGTQVEF |
| | K |
| 1155 | EIVLTQSPGTLSLSPGERASLSCRASQTVSSTYLA |
| | WYQQKPGQAPRLLIYGASSRATGIPDRFSGSGSGT |
| | DFTLTITRLEPEDFAVYYCQQYGTFGQGTKLEIK |
| 1156 | QAGLTQPPSVSKGLRQTATLTCTGTSSNVGNQGAA |
| | WLQQHQGHPPKLLSYRNDNRPSGISERLSASRSGN |
| | TASLTITGLQPEDEADYYCSAWDSSLSAWVFGGGT |
| | KLTVL |
| 1157 | DIVMTQSPDSLAVSLGERATINCKSSQSVLYNSNN |
| | KDYLAWYQQKPGQPPKLLFSWASTRQSGVPARFSG |
| | GGSGTDFTLTISSLQAEDVAVYYCQQYYSTPITFG |
| | GGTKVEIK |
| 1158 | DFVLTQPHSVSESPGKTVTISCTRSSGSIASYFVH |
| | WYQQRPGSAPTTVIYEDNQRPSGVPDRFSGSIDSS |
| | SNSASLIISGLKTEDEADYYCQSFDDNDQVFGGGT |
| | KLTVL |
| 1159 | QTVVTQEPSFSVSPGGTVTLTCGLTSGSVSTTYYP |
| | SWYQQTPGQPPRTLIYSTNIRSSGVPDRFSGSILG |
| | NKAALTITGAQADDESNYYCLLYVGGGIWVFGGGT |
| | KLTVL |
| 1160 | EIVMTQSPATLAVSPGERATLSCRASQSVSDNLAW |
| | YQQRPGQPPRLLIYAASTRATGIPPRFSGSGSGTE |
| | FTLTIASLQSEDFALYYCQQYNIWLTFGGGTKVEI |
| | K |
| 1161 | AVVMTQSPLSLPVTLGQPASISCRSSQSLVHSDGN |
| | TYLNWFQQRPGQSPRRLIYKVSDRDSGVPDRFSGS |
| | GSGTDFTLKINRVEAEDVGVYYCMQGTLLLTFGGG |
| | TKVEIK |
| 1162 | QSVLTQPPSVSAAPGQKVTISCSGSSSNIGNNYVS |
| | WYQQLPGTAPKLLIYDNNKRPSGVPDRFSGSKSGT |
| | SATLGITGLQTGDEADYYCETWDSSLDAVIFGGGT |
| | KLTVL |
| 1163 | QSVLTQPPSASGTPGQRVTISCSGSSSNIGSNYVY |
| | WYQQLPGTAPKVLIYRNNQRPSGVPDRFSGSKSGT |
| | SASLAISGLRSEDEADYYCAAWDDSLSGRVFGGGT |
| | KLTVL |
| 1164 | DVVVTQSPLSLSVTLGQPASISCRSSQSLVHSDGN |
| | TYLNWFQQRPGQSPRRLIYKVSNRDSGVPDRFSGS |
| | GSGTDFTLKISRVEADDVGVYYCMQGTHWPHPTFG |
| | QGTRVEIK |
| 1165 | AVVVTQSPLSLPVTLGQPASISCRSSQSLVYSDGN |
| | TYLNWFQQRPGQSPRRLIYKVSNRDSGVPDRFSGS |
| | GSGTDFTLQISKVEAEDVGVYYCMQGTPWPTFGQG |
| | TKVEIK |
| 1166 | EIVLTQSPGTLSLSPGERATLSCRASQSVRSNYLA |
| | WYQLKPGQAPRLLIYGASSRATGIPDRFSGSGSGT |
| | DFTLTISRLEPEDFAVYYCQQSGSSYTFGQGTKLE |
| | IK |
| 1167 | AIVMTQSPLSLPVTPGEPASISCRSSQSLRQSQRF |
| | SYLDWYVQKPGQSPQLLIYLNSRRAPGVPDRFSAS |
| | GSGTDFTLKISRVEAEDVGVYYCMQSLPSGFTFGP |
| | GTNVHIK |
| 1168 | DIVMTQAPLSLSVTPGQPASISCKSSQSLLHSIGK |
| | THLYWYLQKPGQPPQLLIYEVSNRFSGVPERVSGS |
| | GSGTDFTLTISRVEAEDVGVYYCMQSLDLPPTFGQ |
| | GTKVDIK |
| 1169 | DIQMTQSPSFVSASVGDRVTITCRASHDIRTWLSW |
| | YQQKPGKAPKLLIYTAFRLQSGVPSRFSGSGSGTD |
| | FTLTISSLQPEDFATYFCQQGSSFPLTFGGGTTVD |
| | IR |
| 1170 | DIQMTQSPSSLSASVGDSVTVTCRASQDIGNWLAW |
| | YQLKPEKAPRSLIFAASILRSGVPSRFSGSGSGTE |
| | FTLTISSLQPEDFGVFYCQQYDSSPITFGQGTRLE |
| | IK |
| 1171 | SSQLTQDPAVSVALGQTVRITCQGDSLETYYATWY |
| | QQKPGQAPLLVIYGKNSRPSGIPDRFSGSSSGNTA |
| | SLTITGAQAEDEADYYCNSRDSSGQLHVVVFGGGT |
| | KLTVL |
| 1172 | SSELTQDPAVSVALGQTVRITCQGDSLRTSYASWY |
| | QQKPGQAPMLVIYEKNNRPSGVPDRFSGSTSFNTA |
| | SLTITGAQAEDEAEYYCNSRDNNDDLPLFGGGTRL |
| | TVL |
| 1173 | QSALTQPPSASGSPGQSVTISCTGTSSDVGGYNYV |
| | SWYQQHPGKAPKLMIYEVSKRPSGVPDRFSGSKSG |
| | NTASLTVSGLQAEDEADYYCSSYAGSNNLGVFGTG |
| | TKVTVL |
| 1174 | QSVLTQPPSVSGAPGQRVTISCTGSSSNIGAGYDV |
| | HWYQQLPGTAPKLLIYGNSNRPSGVPDRFSGSKSG |
| | TSASLAITGLQAEDEADYYCQSYDSSLSGVVFGGG |
| | TKLTVL |
| 1175 | DIVMTQSPDSLAVSLGERATINCKSSQSVLYSSNN |
| | KNYLAWYQQKPGQPPKLLIYWASTRESGVPDRFSG |
| | SGSGTDFTLTISSLQAEDVAVYYCQQYYSTPFTFG |
| | PGTKVDIK |
| 1176 | DVVMTQSPLSLPVTLGQPASISCRSSQSLVHSDGN |
| | TYLNWFQQRPGQSPRRLIYKVSNRDSGVPDRFSGS |
| | GSGTDFTLKISRVEAEDVGVYYCMQGTHWPITFGQ |
| | GTRLEIK |
| 1177 | QSALTQPASVSGSPGQSITISCTGTSSDVGGYNYV |
| | SWYQQHPGKAPKLMIYDVSKRPSGVSNRFSGSKSG |
| | NTASLTISGLQAEDEADYYCSSYTSSSTLVVFGGG |
| | TKLTVL |
| 1178 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPYTFGQGTKLE |
| | IK |
| 1179 | QSALTQPRSVSGSPGQSVTISCTGTSSDVGGYNYV |
| | SWYQQHPGKAPKLMIYDVSKRPSGVPDRFSGSKSG |
| | NTASLTISGLQAEDEADYYCCSYAGSYVVFGGGTK |
| | LTVL |
| 1180 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTLHTFGQGTKVE |
| | IK |
| 1181 | SSELTQDPAVSVALGQTVRITCQGDSLRSYYASWY |
| | QQKPGQAPVLVIYGKNNRPSGIPDRFSGSSSGNTA |
| | SLTITGAQAEDEADYYCNSRDSSGNHLVFGGGTKL |
| | TVL |
| 1182 | SYELTQPPSVSVSPGQTATITCSGDELGDTDIAWY |
| | QQKPGQSPVLVILQDTKRPSGIPERFSGSNSGTTA |
| | TLTIGGTQAMDEAEYYCQAWDTITHEEVFGGGTKL |
| | TVL |
| 1183 | DIQMTQSPSTLSASVGDRVTITCRASQSISSWLAW |
| | YQQKPGKAPKLLIYKASSLESGVPSRFSGSGSGTE |
| | FTLTISSLQPDDFATYYCQQYNYYPVAFGQGTKVE |
| | IK |
| 1184 | DIVMTQTPLSSPVTLGQPASISCRSSQSLVHSDGN |
| | TYLSWLQQRPGQPPRLLIYKISNRFSGVPDRFSGS |
| | GAGTDFTLKISRVEAEDVGVYYCTQATQFPLTFGG |
| | GTKVEIK |
| 1185 | DIQMTQSPSSLSASVGDRVTITCRASQSISSYLNW |
| | YQQKPGKAPKLLIYAASSLQSGVPSRFSGSGSGTD |
| | FTLTISSLQPEDLATYYCQQSYSTPPYTFGQGTKL |
| | EIK |
| 1186 | QSVLTQPPSVSGAPGQRVTISCTGSSSNIGAGYDV |
| | HWYQQLPGIAPKLLIYGNNNRPSGVPDRFSGSKSG |
| | TSASLAITGLQAEDEADYYCQSYDSSLSSPVVFGG |
| | GTKLTVL |
| 1187 | QSVLTQPPSASGTPGQRVTISCSGSSSNIGSNYVY |
| | WYQQLPGTAPKLLIYRNNQRPSGVPDRFSGSKSGT |
| | SASLAISGLRSEDEADYYCAAWDDSLSGPVFGGGT |
| | KLTVL |
| 1188 | SSELTQDPAVSVALGQTVRITCQGDSLRSYYASWY |
| | QQKPGQAPVLVIYGKNNRPSGIPDRFSGSSSGNTA |
| | SLTITGAQAEDEADYYCNSRDSSGNHLVFGGGTKL |
| | TVL |
| 1189 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPYTFGQGTKLE |
| | IK |
| 1190 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAGVFGGGT |
| | KLTVL |
| 1191 | EIVMTQSPATLSVSPGERATLSCRASQSVSSNLAW |
| | YQQKPGQAPRLLIYGASTRATGIPARFSGSGSGTE |
| | FTLTISSLQSEDFAVYYCQQYNNWPPWTFGQGTKV |
| | EIK |
| 1192 | SYELTQPPSVSVSPGQTASITCSGDKLGDKYACWY |
| | QQKPGQSPVLVIYQDSKRPSGIPERFSGSNSGNTA |
| | TLTISGTQAMDEADYYCQAWDSSTYVVFGGGTKLT |
| | VL |
| 1193 | DIQMTQSPSSLSASEGDRVTITCRASQSISTNYLN |
| | WYQQKSGRAPTLLIYATSTLQSGVPSRFSGSGSGT |
| | DFTLTISSLQPEDFATYYCQQSYSSPPTFGGGTTV |
| | DVK |
| 1194 | DIQMTQSPSSVYASEGDRVTITCRASHSISTNYLN |
| | WYQQNSGKAPKLLIYATSSLQSGVPFRFSGSGSGT |
| | DFTLTISSLQPEDFATYYCQQSYSSPPTFGGGTKV |
| | EIK |
| 1195 | DIQMTQSPSSLSASEGDRVTISCRASQTISTNYLN |
| | WYQQKSGKAPRLLIYATSTLESGVPSRFSGSGSGT |
| | DFTLTINTLQPDDFATYYCQQSYSSPPTFGGGTKV |
| | DIK |
| 1196 | EIVLTQSPATLSLSPGERAALSCRASQTINSGYLA |
| | WYQQKPGQAPRLLIYAASHRATGIPNRFSGSGSAT |
| | DFTLTITRLEPEDVAVYYCHHYGTSPPFTFGPGTK |
| | VDIK |
| 1197 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPLTFGGGTKV |
| | EIK |
| 1198 | SYELTQPPSVSVSPGQTARITCSGDALPKQYAYWY |
| | QQKPGQAPVLVIYKDSERPSGIPERFSGSSSGTTV |
| | TLTISGVQAEDEADYYCQSADSSGTYYVFGTGTKV |
| | TVL |
| 1199 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPRTFGQGTKVE |
| | IK |
| 1200 | SYELTQPPSVSVSPGQTASISCSGDKLGDTYASWY |
| | QQKPGQSPVLVMYQDNKRPSGIPERFSGSNSGNTA |
| | TLTISGTQAMDEADYYCQAWDSSTVVFGGGTKLTV |
| | L |
| 1201 | DIVMTQTPLSLSVTPGQPASISCKSSQSLLHSDGK |
| | TYLYWYLQKPGQSPQLLIYEVSNRFSGVPDRFSGS |
| | GSGTDFTLKISRVEAEDVGVYYCMQSIQLPLTFGG |
| | GTKVEIK |
| 1202 | DIVMTQTPLSLSVTPGQPASISCKSSQSLLHSDGK |
| | TYLYWYLQKPGQSPQLLIYEVSNRFSGVPDRFSGS |
| | GSGTDFTLKISRVEAEDVGVYYCMQSIQLPFTFGQ |
| | GTRLEIK |
| 1203 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTYTFGQG |
| | TKLEIK |
| 1204 | SYELTQPPSVSVSPGQTARITCSGDALPKKYAYWY |
| | QQKSGQAPVLVIYEDSKRPSGIPERFSGSSSGTMA |
| | TLTISGAQVEDEADYYCYSTDSSGNHRRVFGGGTK |
| | LTVL |
| 1205 | QSALTQPASVSGSPGQSITISCTGTSSDVGGYNYV |
| | SWYQQHPGKAPKLMIYDVSKWPSGVSNRFSGSKSG |
| | NTASLTISGLQAEDEADYYCSSYTSSSTLVFGGGT |
| | KLTVL |
| 1206 | SYELTQPPSVSVSPGQTARITCSGDALPKKYAYWY |
| | QQKSGQAPVLVIYEDSKRPSGIPERFSGSSSGTMA |
| | TLTISGAQVEDEADYYCYSTDSSGNHRGVFGGGTK |
| | LTVL |
| 1207 | DIQMTQSPDTLSASVGDRVTITCRASESISNWLAW |
| | YQKKVGQAPNLLIDKASNLHRGVPSRFSGSGSGTE |
| | FTLTITSLQPDDSASYYCQQYNSFPYTFGQGTTLE |
| | IK |
| 1208 | EIVLTQSPATLSLSPGERATLSCRASQSVSSYLAW |
| | YQQKPGQAPRLLIYDASNRATGIPARFSGSGSGTD |
| | FTLTISSLEPEDFAVYYCQQRSNWPVTFGQGTKVE |
| | IK |
| 1209 | DIQMTQSPSSLSASVGDRVTITCRASQGIRNDLGW |
| | YQQKPGKAPKRLIYAASSLQSGVPSRFSGSGSGTE |
| | FTLTISSLQPEDFATYYCLQHNSYPLTFGGGTKVE |
| | IK |
| 1210 | DIQMTQSPSSLSASVGDRVTITCRASQGIGNDLGW |
| | FQQKPGKAPKRLIYGASNLQSGVPSRFSGSGSGTE |
| | FTLTISSLQPEDFATYYCLQHNSYPFTFGGGTKVE |
| | IK |
| 1211 | ETVLTQSPGTLSLSPGERATLSCRASQSVSGSYLA |
| | WYQQKPGQAPRLLIYGASRRATGIADRFSGSGSGT |
| | DFTLTISRLEPEDFAVYYCQQYGTSAGTFGQGTKV |
| | EIK |
| 1212 | EIVLTQSPGTLSLSPGERGTLSCRASQSVSSSYLA |
| | WYQQKPGQAPRLLIYGASTRATGIPDRFSGSGSGT |
| | DFTLTISRLEPEDFAVYYCQQYGNLPPFTFGPGTK |
| | VDIK |
| 1213 | DIVVTQSPDSLAVSLGERATINCKSSQSLLYNFNN |
| | ENYLGWYQQKPGQPPKLLIYWASTRESGVPDRFNG |
| | SGSGTDFTLTISSLQAEDVAVYYCQQYYSTPLTFG |
| | GGTKVEIK |
| 1214 | GIVMTQSPLSLSVTPGQPASISCKSSQSLLDSDGK |
| | TYMCWYLQKPGQPPQLLIYEVSNRFSGVPERFSGS |
| | GSGTDFTLKISRVETEDVGVYYCMQNRHLYTFGQG |
| | TKLEIK |
| 1215 | GIVMTQSPLSLSVTPGQPASISCKSSQSLLDSDGK |
| | TYMCWYLQKPGQPPQLLIYEVSNRFSGVPERFSGS |
| | GSGTDFTLKISRVEAEDVGVYYCMQNRHLYTFGQG |
| | TKLEIK |
| 1216 | NIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQTLQTSITFGQ |
| | GTRLEIK |
| 1217 | EIVLTQSPGTLSLSPGERATLSCRASQSVSTYLAW |
| | YQQRPGQAPRLLIYGSSSRAAGIPDRFSGSGSGTD |
| | FTLTISRLEPEDFAVYYCQQYGSSQYSFGQGTKLE |
| | IK |
| 1218 | EIVLTQSPGTLSLSPGERATLSCRASQSVSSYLAW |
| | YQQRPGQAPRLLIYGASSRAAGIPDRFSGSGSGTD |
| | FTLTISRLEPEDFAVYYCQQYGSSQYTFGQGTKLE |
| | IK |
| 1219 | DIQMTQSPSSLSASVGDRVTITCQASQDSSKYLNW |
| | YQQKPGKAPKLLIYDASTLETGVPSRFSGSGSGTD |
| | FTFTISGLQPEDVATYYCQHYDTLLTFGPGTKVEI |
| | K |
| 1220 | DIVMTQSPDSLAVSLGERATINCKSSQSVSFTSNN |
| | KNYLAWYQQKPGQPPKLLIYWASTRESGVPDRFSG |
| | SGSGTDFTLTISSLQAEDVALYLCQQYFDTPWTFG |
| | QGTKVEIK |
| 1221 | GIVMTQSPLSLSVTPGQPASISCKSSQSLLDSDGK |
| | TYLCWYLQKPGQPPQLLIYEVSNRFSGVPERFSGS |
| | GSGTDFTLKISRVEAEDVGVYYCMQNRQLYTFGQG |
| | TKLEIK |
| 1222 | DIQMTQSPSSLSASVGDRVTITCQASQDISTYLNW |
| | YQQKPGKAPKLLIYDASNLETGVPSRFSGSGSGTD |
| | FTFTISSLQPEDIATYYCQQFDNLPPFTFGPGTRV |
| | HIT |
| 1223 | DIQMTQSPSSLSASVGDRVTITCRASQSISNYLNW |
| | YQQKPGRAPKVLIYGASTLQSGVPSRFSGSGSGTD |
| | FTLTISSLQPEDFATYYCQQSYSARMSTFGQGTKL |
| | EIK |
| 1224 | DVVMTQSPLSLPVTLGQPASISCRSSQSVVHSDGK |
| | TYLNWYHQRPGQSPRRLIYEVSNRDSGVPDRFSGS |
| | GSGTDFTLKISRVEAEDVGVYYCMQGTQWPWTFGQ |
| | GTKVEIK |
| 1225 | EIVLTQSPGTLSLSPGERATLSCRASHTISSSYLA |
| | WYQQKAGQAPRLLIYAASSRATGIPARFSGSGSGT |
| | DFTLTISRLEPEDFAVYYCQQFDNSPPWTFGRGTK |
| | VEMR |
| 1226 | SYELTQPPSVSVSPGQTARITCSGDALPKQYAYWY |
| | QQKPGQAPVLVIYKDSERPSGIPERFSGSSSGTTV |
| | TLTISGVQAEDEADYYCQSADSSGTYVVFGGGTKL |
| | TVL |
| 1227 | QSALTQPRSVSGSPGQSVTISCTGTSSDVGGYNYV |
| | SWYQQHPGKAPKLMIYDVSKRPSGVPDRFSGSKSG |
| | NTASLTISGLQAEDEADYYCCSYAGSYTLVFGGGT |
| | KLTVL |
| 1228 | DIQMTQSPSSLSASVGDRVTITCRASQSISNYLNW |
| | YQQKPGKAPKLLIYAASGLQSGVPSRFSGSGSGTD |
| | FTLTISSLQPEDFATYYCQQSYSTPFTFGPGTKVD |
| | IK |
| 1229 | DVVMTQSPLSLPVTLGQPASISCRSSQSLVYSDGN |
| | TYLNWFQQRPGQSPRRLIYKVSNRDSGVPDRFSGS |
| | GSGTDFTLKISRVEAEDVGVYYCMQGTHSYTFGQG |
| | TKLEIK |
| 1230 | SYELTQPPSVSVSPGQTASITCSGDKLGDKYACWY |
| | QQKPGQSPVLVIYQDSKRPSGIPERFSGSNSGNTA |
| | TLTISGTQAMDEADYYCQAWDSSTASYVFGTGTKV |
| | TVL |
| 1231 | QSALTQPASVSGSPGQAITISCTGTSSDVGGHDYV |
| | SWYQQHPGKVPKLVVYDVTNRPSGVSNRFSGSKSG |
| | NTASLTISGLQAEDEADYYCSSYTSASTVVFGGGT |
| | KLTVL |
| 1232 | QSALTQPASVSGSPGQAITISCTGTSSDVGGHDYV |
| | SWYQQHPGKVPKLVVYDVTNRPSGISNRFSGSKSG |
| | NTASLTISGLQAEDEADYYCSSYTSASTVVFGGGT |
| | KLTVL |
| 1233 | DVVMTQSPLSLPVTLGQPASISCRSSQSLVYSDGN |
| | TYLNWFQQRPGQSPRRLIYKVSNRDSGVPDRFSGS |
| | GSGTDFTLKISRVEAEDVGVYYCMQGTHSPWTFGQ |
| | GTKVEIK |
| 1234 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYHCGTWDSSLSAWVFGGGT |
| | KLTVL |
| 1235 | SYELTQPPSVSVSPGQTASITCSGDALPKQYGYWY |
| | QQKPGQAPVMVIYKDNERPSGIPERFSGSSSGTTV |
| | TLTISGAQAEDEADYYCQSADGRGDWVFGGGTKLT |
| | VL |
| 1236 | EIVLTQSPGTLSLSPGERATLSCRASQSVSTYLAW |
| | YQQRPGQAPRLLIYGSSSRAAGIPDRFSGSGSGTD |
| | FTLTISRLEPEDFAVYFCQQYGSSQYSFGQGTKLD |
| | IK |
| 1237 | DIQMTQSPSTLSASIGDRVTITCRASQSISSWLAW |
| | YQQKPGKAPKLLIYETSSLEPGVPSRFSGSGSGTE |
| | FTLTISSLQPDDFATYYCQQYDSYSGTFGQGTKVE |
| | IK |
| 1238 | QPVLTQSSSASASLGSSVKLTCTLSVGHDYFTIAW |
| | HQQQPGKAPRFLMKLEGSGSYYKGSGVPDRFSGSS |
| | SGADRYLIISNLQSEDEADYFCETWDSPYVVFGGG |
| | TKLTVL |
| 1239 | DIQMTQYPSSLSASVGDTVTITCQASQDSNTYLNW |
| | YQQKPGKAPKLLIYDASNLETGVPSRFSGSGSGTD |
| | FTFTISGLQPEDIATYYCQHYDSLLTFGPGTKVDI |
| | K |
| 1240 | QSALTQPASVSGSPGQSITISCNGTNSDVGGYNYV |
| | SWYQQHPGKAPKLMIYDVSKRPSGVSNRFSGSKSG |
| | NTASLTISGLQAEDDADYYCSSYTSSSTVVFGGGT |
| | KLTVL |
| 1241 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTLTFGPG |
| | TKVDIK |
| 1242 | DIQMTQSPSTLSASVGDRVTITCRASQSISSWLAW |
| | YQQKPGKAPKLLIYKASSLESGVPSRFSGSGSGTE |
| | FTLTISSLQPDDFATYYCQQYNSYPLFTFGPGTKV |
| | DIK |
| 1243 | DVVLTQSPLSLPVTLGQPASISCRSSQSLIYSDGN |
| | TYLNWFQQRPGQSPRRLIYKVSNRDSGVPDRFSGS |
| | GSGTDFTLRISRVEAEDVGVYYCMQGTHWPMTFGQ |
| | GTKVEIK |
| 1244 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPMYTFGQGTK |
| | LEIK |
| 1245 | DIQMTQSPSSVSASVGDRVTITCRASQGISSWLAW |
| | YQQKPGKAPKLLIYAASSLQSGVPSRFSGSGSGTD |
| | FTLTISSLQPEDFATYYCQQANSFPAFGGGTKVEI |
| | K |
| 1246 | SYEVTQSPSVSVSPGQTASITCSGDKLGDKYACWY |
| | QQRPGQSPVLVIYQDSKRPSGIPERFSGSNSGNTA |
| | TLTISGTQAMDEADYYCQAWDSHTVVFGGGTKLTV |
| | L |
| 1247 | DIQMTQSPSTLSASVGDRVTITCRASQSISTWLAW |
| | YQQRPGKAPKLLIYKASSLESGVPSRFSGSGSGTE |
| | FTLTISSLQPDDFATYYCQQYNSYSWTFGQGTKVE |
| | IK |
| 1248 | ETMMTQSPVALSVSPGDRATLSCEASQYVGDNLAW |
| | YQQKPGQAPRLLIYGAFTRATGVPARFSASGSGAG |
| | FTLTISSLQSEDFAVYYCQQYTSWPLTFGGGTKVE |
| | IK |
| 1249 | ETMMTQSPVALSVSPGDRATLSCKASQYIGDNLAW |
| | YQQKPGQTPRLLIYGASTRATGVPARFSASGSGAG |
| | FTLTISSLQSEDFAVYYCQQYTSWPLTFGGGTKVE |
| | IK |
| 1250 | SYELTQPPSVSVSPGQTARITCSGDALPKKYAYWY |
| | QQKSGQAPVLVIYEDSKRPSGIPERFSGSSSGTMA |
| | TLTISGAQVEDEADYYCYSPKVFGGGTKLTVL |
| 1251 | DIQMTQSPSSLSASVGDRVTVACQASQDVSIYLNW |
| | YQQKPGRAPKLLIYDAYNLQTGVPSRFSGSGSGTH |
| | FTLTISSLQPEDVATYHCQQYNILPHTFGGGTKVE |
| | LT |
| 1252 | DIVMTQSPDSLAVSLGERATIKCKSSQSVYDSSNS |
| | KNYLAWFQQKPGQPPQLLIFWASTRESGVPDRFSG |
| | SGSGTDFTLTISSLQAEDVAVYYCQQYYNAPLSFG |
| | GGTKVEIK |
| 1253 | DIVMTQSPDSLPVSLGERATIKCKSSQSVYDTSNS |
| | KNYLAWFQQKPGQPPQLLIFWASTRESGVPDRFSG |
| | SGSGTDFTLTISSLQAEDVAVYYCQQYYNAPLSFG |
| | GGTKVEIK |
| 1254 | EIVLTQSPATLSLSPGERATLSCRASQSVSTYLAW |
| | YQQKPGQAPRLLIYDASNRATGIPARFSGSGSGTD |
| | FTLTISSLEPEDFAVYYCQQRSNWITFGQGTRLEI |
| | K |
| 1255 | EIVLTQSPATLSLSPGERATLSCRASQSVSSYLAW |
| | YQQKPGQAPRLLIYDASNRATGIPARFSGSGSGTD |
| | FTLTISSLEPEDFAVYYCQQRSNWITFGQGTRLEI |
| | K |
| 1256 | QSVLTQPPSASGTPGQGVTISCSGGSSNIGAYTVS |
| | WYQQLPGTAPKLLIYSTDQRPSGVPDRFSGSKSGT |
| | SASLAVTGLQSEDEADYYCAAWDDSLNGPVFGGGT |
| | KLTVL |
| 1257 | EIVLTQSPGTLSLSPGERATLSCRASQSVSSIYLA |
| | WYQQKPGQAPRLLIYGASSRATGIPDRFSGSGSGT |
| | DFTLTISRLEPEDFAVYYCQQYGSSPQTFGQGTKL |
| | EIK |
| 1258 | EIVMTQSPATLSVSPGERATLSCRASQSVTSYLAW |
| | YQQKPGQAPRLLIYGASTRATGIPARFSGSGSGTE |
| | FTLTISSLQSEDFAVYYCQQYNNWPPLTFGGGTKV |
| | EIK |
| 1259 | DIQMTQSPSTLSASVGDRVTITCRASQSITNWLAW |
| | YQQRPGKAPKLLLSKASSLESGVPSRFSGSGSGTD |
| | FTLTISSLQPDDFATYYCQQYYSYSLTFGGGTKVE |
| | SK |
| 1260 | QSALTQPASVSGSPGQSITISCTGTSSDVGSYNLV |
| | SWYQQHPGKAPKLMIYEVSKRPSGVSNRFSGSKSG |
| | NTASLTISGLQAEDEADYYCCSYAGSSTFYVFGTG |
| | TKVTVL |
| 1261 | SYELTQPPSVSVSPGQTARITCSGDALPKQYAYWY |
| | QQKTGQAPVLVIYKDSERPSGIPERVSGSSSGTTV |
| | TLTISGVQAEDEADYYCQSADSSGTWVFGGGTKLT |
| | VL |
| 1262 | EIVLTQSPGTLSLSPGERATLSCRASQSVSSRYLA |
| | WYQQKPGQAPRLLIYGASRRATGIPDRFSGSGSGT |
| | DFTLTISRLEPEDFAVYYCQQYGSSPEMYTFGQGT |
| | KLEIK |
| 1263 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCHQYGSGLGTFGPGTKV |
| | DIK |
| 1264 | DIVMTQTPLSLSVTPGQPASISCKSSQSLLDSDGK |
| | TYLYWYLQKPGQTPQLLIYEVSDRFSGVPDRFSGS |
| | GSGTDFTLKISRVEAEDVGVYYCMQSIQLRTFGQG |
| | TKVEIK |
| 1265 | EIVLTQSPATLSLSPGERATLSCRASQRVGSSLAW |
| | YQQKPGQAPRLLIYGASNRATGIPARFSGSGSGTD |
| | FTLTITRLEPEDFAVYYCQQCSSWPLSLTFGGGTK |
| | VEIR |
| 1266 | EIVMTQSPATLSVSPGERATLSCRASQSVSSNLAW |
| | YQQKPGQAPRLLIYGASTRATGIPARFSGSGSGTE |
| | FTLTISSLQSEDFAVYYCQQYNNWPPITFGQGTRL |
| | DIK |
| 1267 | DIQMTQSPSSVSASVGDRVTITCRASQGIRFWLAW |
| | YQQKPGKAPKLLIYAASTLQSGVPSRFSGSGSGTD |
| | FTLTISSLQPEDFATYYCQQSNSFPPTFGGGTKVE |
| | IK |
| 1268 | QSVLTQPPSVSGAPGQRVTISCTGSSSNIGAGYDV |
| | HWYQQLPGTAPKLLIYGNTNRPSGVPDRFSGSKSG |
| | TSPSLAITGLQAEDEAGYYCQSYDISLSAYVFGGG |
| | TKLTVL |
| 1269 | DIQMTQSPSTLSASVGDRVTITCRASQSISSWLAW |
| | YQQKPGKAPKLLIYDASSLESGVPSRFSGSGSGTE |
| | FTLTISSLLPADFATYYCQQYNTYSLTFGQGTRLE |
| | IK |
| 1270 | AIQLTQSPSSLSASVGDRVTITCRASQGISSYLAW |
| | YQQKPGKAPKLLIYAASTLQSGVPSRFSGSGSGTD |
| | FTLTISSLQPEDFATYYCQQLNSYPPAFGGGTKVE |
| | IK |
| 1271 | EIVMTQSPDSLAVSLGERATINCKSSQSVLYSASN |
| | KNYLAWYQQKQGQSPKLLIYWASTRESGVPDRFSG |
| | SGSGTDFTLTINGLQAEDVAVYYCQQYYRTPLTFG |
| | GGTKVEIK |
| 1272 | DIQMTQSPSAMSASVGDRVTITCRASQDISNYLAW |
| | FQQRPGKVPKRLIYAASSLQSGVPSRFSGTGSGTE |
| | FTLTISSLQPEDFATYYCLQHHTYPLTFGGGTKVE |
| | IR |
| 1273 | EIVLTQTPLSLSVTPGQPASISCKSSHSLLHSDGK |
| | TYVYWYLQRPGQPPQLLIYELFNRFSGVPDRFSGS |
| | GSGTDFTLKISRVEAEDVGTYYCMQSIQLWSFGQG |
| | TKVEIK |
| 1274 | QAVVTQEPSLTVSPGGTVTLTCGSSTGAVTSGHYP |
| | YWFQQKPGQAPRTLIYDTNNKHSWTPARFSGSLLG |
| | GKAALTLSGAQPEDEAEYYCLLSYSGPWVFGGGTK |
| | LTVL |
| 1275 | QSALTQPPSASGSPGQSVTISCTGTSSDVGGYNYV |
| | SWYQQHPGKAPKLMIYEVSKRPSGVPDRFSGSKSG |
| | NTASLTVSGLQAEDEADYYCSSYAGSNNYVFGTGT |
| | KVTVL |
| 1276 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPSFTFGPGTKV |
| | DIK |
| 1277 | QSALTQPRSVSGSPGQSVTISCTGTSSDVGGYNYV |
| | SWYQQHPGKAPKLMIYDVNKRPSGVPDRFSGSKSG |
| | NTASLTISGLQAEDEADYYCCSYAGSYTLVFGGGT |
| | KLSVL |
| 1278 | QSALTQPASVSGSPGQSITISCTGTSSDVGSYNLV |
| | SWYQQHPGKAPKLMIYEVSKRPSGVSNRFSGSKSG |
| | NTASLTISGLQAEDEADYYCCSYAGSSTVVFGGGT |
| | KLTVL |
| 1279 | DIQMTQSPSSLSASVGDRVTISCRASQSIGKYLNW |
| | YQQKPGKAPKLLIYAASSLQSGVPSRFSGSGSGTD |
| | FTLTINNLQPEDFATYYCQQSYNVPPWTFGQGTKV |
| | EIK |
| 1280 | DVVMTQSPVSLTVTLGQPASISCRPSQSIEHSDGN |
| | IYLNWFQQRPGQSPRRLIYKISNRDSGVPDRISGS |
| | GSGTDFTLKISRVEAEDVGVYYCMQGTHWPWTFGQ |
| | GTKVEIK |
| 1281 | QSVLTQPPSVSGAPGQRVIIPCTGSSSNTGAGYDV |
| | HWYQQLPGTAPKLVIYDNSHRPSGVPDRFSGSKSG |
| | TSASLAITGLQAEDEADYYCQSYDINLSAVFGGGT |
| | KLTVL |
| 1282 | EIVLTQSPGTLSLSPGERATLSCRATQSLTSSSLA |
| | WYQQKPGQAPRLLIYGASSRATGIPDRFSGSGSGT |
| | DFTLTISRLKPEDFAVYYCHQYHNSPWTFGQGTKV |
| | EIK |
| 1283 | DFVMTQSPLSLPVTPGEPASISCRSSQSLLHGNGY |
| | TYLDWYLQKPGQSPQLLIYLGSTRASGVPDRFSGS |
| | GSGTDFTLKISRVEAEDVGVYYCMQALQTPYTFGQ |
| | GTKLEIK |
| 1284 | SYVLTQPPSVSVAPGKTARITCGGNNIGSKSVHWY |
| | QQKPGQAPVLVVYDDSDRPSGIPERFSGSNSGNTA |
| | TLTISRVEAGDEADYYCQVWDSSSDHYVFGTGTKV |
| | TVL |
| 1285 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPRTFGQGTKV |
| | EIK |
| 1286 | QIVMTQSPATLSVSPGGGATLSCRASQSVSSKVAW |
| | YQQKPGQAPRLLIYGASTRATGIPARFSGSGSGTE |
| | FTLTISSLQSEDSAVYYCQQYDNWLPYTFGQGTKL |
| | EIK |
| 1287 | QAVVTQEPSLTVSPGGTVTLTCGSSTGAVTSGHYP |
| | YWFQQKPGQAPRTLIYDTSNKHSWTPARFSGSLLG |
| | GKAALTLSGAQPEDEAEYYCLLSYSGAYVLFGGGT |
| | KLTVL |
| 1288 | EIVMTQSPAILSVSPGERATLSCRASQSVTRNLAW |
| | YQQKPGQAPRLLIYGASTRATNIPARFSGSGFGTE |
| | FTLIISSLQSEDFAVYYCQQYSNWPLYTFGQGTKL |
| | EIK |
| 1289 | QSVLTQPPSASGTPGQRVTISCSGSSSNIGSNTVN |
| | WYQQLPGTAPKLLIYSNNQRPSGVPDRFSGSKSGT |
| | SASLAISGLQSEDEADYYCAAWDDSLNGPYVFGTG |
| | TKVTVL |
| 1290 | QSALTQPASVSGSPGQSITISCTGTSSDVGGYNYV |
| | SWYQHHPGKAPKLMIYEVSNRPSGVSNRFSGSKSA |
| | NTASLTISGLQTEDEADYYCSSYTSISTVLFGGGT |
| | KLTVL |
| 1291 | SDALTQPPSVSVAPGKTAAITCGGDNIGSKNVHWY |
| | QQKPGQAPLLVVFDDGDRPSGIPERFSGSNSGNTA |
| | TLTISRVEAGDEADYYCQVWDGGSDDRGYVFGTGT |
| | KVTVL |
| 1292 | QSALTQPASVSGSPGQSITISCTGTSSDVGAYNYV |
| | SWYQQHPGKAPKLMIYDVTKRPSGVSNRFSGSKSG |
| | NTASLTISGLQAEDEADYYCSSFTSNGAWVFGGGT |
| | KVTVL |
| 1293 | DIQMTQSPSSLSASVGDRVTITCRASQSISNYLNW |
| | YQQKPGKAPKFLIYAASTLHTGVPSRFSGSGSGTD |
| | FTLTISSLQPEDFATYYCQQNYIRPYTFGQGTKLE |
| | IK |
| 1294 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPITFGGGTKVE |
| | IK |
| 1295 | QSVLTQPPSTSGAPGQRVTISCSGSSSNVALNAVS |
| | WYQQLPRMAPKLLIYRDNQRPSGVPERFSGSRSGT |
| | SASLAITGLQSDDEADYYCATWDDSLNGVFGGGTK |
| | LTVL |
| 1296 | EIVMTQSPATLSVSPGERATLSCRASQSVSSNLAW |
| | YQQKPGQAPRLLIYGASSRATGIPARFSGSGSGTE |
| | FTLTISSLQSEDFGVYYCQQYNNWPYTFGQGTKLE |
| | IK |
| 1297 | QPVLTQPPSASASLGASVTLTCTLSSGYSNYKVDW |
| | YQQRPGKGPRFVMRVGTGGIVGSKGDGIPDRFSVL |
| | GSGLNRYLTIKNIQEEDESDYHCGADHGSGSNFVY |
| | VFGTGTKVTVL |
| 1298 | QSALTQPASVSGSPGQSITISCTGTSSDVGSYNLV |
| | SWYQQHPGKAPKLMIYEVSKRPSGVSNRFSGSKSG |
| | NTASLTISGLQAEDEADYYCCSYAGSSTLVFGGGT |
| | KVTVL |
| 1299 | DIKMTQSPSTLSASVGDRVTITCRASQHINRWLAW |
| | YQQKPGKAPKLLIYEASSLKSGVPSRFSGSGSGTE |
| | FTLTITSLQLDDFATYSCQQHDSAPYTFGQGTKLE |
| | IK |
| 1300 | DIQMTQSPSTLSASLGDRVMITCRASQNISRWLAW |
| | YQQKPGKAPKFLIYKASALETGVPSRFSGSGSGTE |
| | FTLTITGLQPDDFATYYCQQYNSYVTFGGGTKVEM |
| | K |
| 1301 | DIVMTQTPLSLSVTPGQPASISCKSSQSLLHSDGK |
| | TYLYWFLLKPGQSPQFLIYEVSSRFSGVPDRFRGS |
| | GSGTDFTLKISRVEAEDVGVYYCMQGKHLRWTFGQ |
| | GTKVEIK |
| 1302 | SYELAQPPSVSVSPGQTARITCSGDALPIKYAYWY |
| | QQKSGQAPVLVISEDSKRPSGIPERFSGSSSGTMA |
| | TLTISGAQVEDEADYYCYSTDYSGNHGVFGGGTKL |
| | TVV |
| 1303 | EIVLTQSPATLSLSPGERATLSCRASQSVSTYLAW |
| | YQQKPGQAPRLLIYDASNRATGIPARFSGSGSGTD |
| | FTLTISSLEPEDFAVYYCQQCSNWPNTFGQGTKLE |
| | IK |
| 1304 | SYELTQPPSVSVSPGQTARITCSGDELPKQYSYWF |
| | QQRPGQAPVLVIYKDRERPSGIPERFSGSHSGTTV |
| | TLTISGVQAEDEADYYCQSADSNDSWVFGGGTKLT |
| | VL |
| 1305 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAGVFGGGT |
| | KLTVL |
| 1306 | GIQLTQSPSSVSASLGDTVTITCRASQNINVFLAW |
| | YQQRPGSAPSLLIYAASNLQSGVPSRFVGSGSGTD |
| | FTLTISGLQPEDFATYYCQQGHNFPWTFGRGTKVE |
| | VK |
| 1307 | EIVLTQSPGTLSLSPGDRATLSCRASQSLNNNQLA |
| | WYQQKLGQAPRLLIYGASSRATGIPDKISGSGSGT |
| | VFTLTISRLEPEDFAVYYCQQYGSLPLTFGGGTKV |
| | EIK |
| 1308 | EIVLTQSPGTLSLSPGDRATLSCRASQSLNNNQLA |
| | WYQQKLGQAPRLLIYGASSRATGIPDKISGSGSGT |
| | VFTLTISRLEPEDFAVYYCQQYGSLPLTFGGGTKV |
| | EIK |
| 1309 | QSVLTQPPSVSGAPGQRVTISCTGSSSNIGAGYDV |
| | HWYQQLPGTAPKLLIYGNSNRPSGVPDRFSGSKSG |
| | TSASLAITGLQAEDEADYYCQSYDSSLSGWVFGGG |
| | TKLTVL |
| 1310 | EIVLTQSPATLSLSPGERATLSCRASQSISSHLGW |
| | YQQKPGQAPRLLIYDASNRAPGIPARFSGSGSGTD |
| | FTLTISSLEPEDFAVYYCQQRRNWPLTFGGGTKVE |
| | IK |
| 1311 | EIVLTQSPATLSLSPGEGATLSCRASQSVASYLAW |
| | YQQKPGQAPRLLIYDASNRATGIPARFSGSGSGTD |
| | FTLTISSLEPEDFAVYYCQHRSNWPYTFGQGTKLE |
| | IK |
| 1312 | QSVLTQPPSASGTPGQRVTISCSGSSSNIGSNTVN |
| | WYQQLPGTAPKLLIYSNNQRPSGVPDRFSGSKSGT |
| | SASLAISGLQSEDEADYYCAAWDDSLNGVVFGGGT |
| | KLTVL |
| 1313 | DIVMTQTPLSSPVILGQSASISCRSSHSLLHNNGN |
| | TYLSWLHQRPGQPPRLLIYEISNRFSGVPDRFSGS |
| | GAGTDFTLKISRVEAEDVGIYYCMQTTQFPRTFGQ |
| | GTKVEIR |
| 1314 | SYELTQPPSVSVSPGQTAKITCSGDALPKEFAYWY |
| | QQKPGQAPVLIIYKDKERPSGIPERFSGSSSGTTV |
| | TLTISGVQAEDEADYYCQSQDSSATYVVFGGGTKL |
| | TVL |
| 1315 | SSDLTQPPSVSVSPGQTASIACSGDKLGDKYVSWY |
| | QQKPRQSPVLVIYQDNKRPSGIPERFAGSNSGNTA |
| | TLTISGTQTMDEADYYCQAWDSSIEVFGTGTKVTV |
| | L |
| 1316 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPPWTFGQGTK |
| | VEIK |
| 1317 | SYELTQPPSVSVSPGQTARITCSADALSKQYAYWY |
| | QQKPGQAPVLVIYKDSERPSGIPERFSGSNSGTTV |
| | TLTISGVQAEDEAEYYCQSGDSSGTYVVFGGGTKL |
| | TVL |
| 1318 | DIVMTQTPLSSPVILGQSASISCRSSQSLLHNNGN |
| | TYLSWLHQRPGQPPRLLIYEISNRFSGVPDRFSGS |
| | GAGTDFTLKISRVEAEDVGIYYCMQTTQFPRTFGQ |
| | GTKVEIR |
| 1319 | DIQMTQSPSAMSASVGDRVTITCRASQGIRNSLAW |
| | FQQKPGKVPKRLIYDASNLQSGVPSRFSGSGSGTE |
| | FTLTISSLQPEDFATYYCLQYNTYSYSFGQGTKLE |
| | IK |
| 1320 | DIQMTQSPSILSASVGDRVTITCRASQNISRWLAW |
| | YQQKPGKAPKFLIYKASGLESGVPSRFSGSGSGTE |
| | FTLTISSLQPDDFATYYCQQYNSYITFGGGTKIEI |
| | K |
| 1321 | DIQMTQSPSTLSASVGDRVIITCRASQNISRWLAW |
| | YQQKPGTAPKFLIYKASALESGVPSRFSGSGSGTE |
| | FTLTITSLQPDDFATYYCQQYNSYVTFGGGTKVEM |
| | K |
| 1322 | SYELTQPPSVSVSPGQTARITCSGDALPQRYAYWY |
| | QQKSGQAPVLVIYEDTKRPSGIPERFSGFSLGTLA |
| | TLTISGAQVEDEADYYCYSTDSSDNQRVFGGGTKL |
| | TVL |
| 1323 | AIQLTQSPSSLSASVGDRVTITCRASQGVASYLAW |
| | YQQKPGKAPNLLIYAASTLQGGVPSRFSGSGSGTD |
| | FTLTISNLQPEDFATYYCQHLKSYPLTFGGGTKVE |
| | IK |
| 1324 | DIQMTQSPPSVSASIGDTVTITCRATQNINVFLAW |
| | YQQKPGSAPTLLIYGASSLQSGVPSRFVGSGSGTD |
| | FTLTISGLQPEDFATYYCQQGHNFPWTFGRGTKVE |
| | VK |
| 1325 | DIQMTQSPSSVSASIGDTVTITCRATQNINVFLAW |
| | YQQKPGSAPTLLIYGASSLQSGVPSRFVGSGSGTD |
| | FTLTISGLQPEDFATYYCQQGHNFPWTFGRGTKVE |
| | VK |
| 1326 | EIVMTQSPATLSVSPGERATLSCRASQSLNSNLAW |
| | YQQKPGQAPRLVIYGASTRAAGFPARFSGSGSETE |
| | FTLTISSLQSEDFAIYFCQQYHNFPLTFGQGTEVE |
| | VR |
| 1327 | QSVLTQPPSVSGAPGQRVTISCTGSSSNIGAGYDV |
| | HWYQLLPGTAPKLLIYGNNNRPSGVPDRFSGSKSG |
| | TSASLAIIGLQAEDEATYYCQSYDSSLSVVFGGGT |
| | KVTVL |
| 1328 | SYELTQPPSVSVSPGQTAIITCSGDKLGEKYASWY |
| | QQRPGQSPMLVIYQDTKRPSGIPERFSGSNSGNTA |
| | TLTISGTQAVDEADYFCQAWDSNTGVFGTGTKVTV |
| | L |
| 1329 | QSVLTQPPSLSGAPGQRVTISCTGSSSNIGAGYDV |
| | HWYQQLPGTAPKLLIYGDSNRPSGVPDRFSGSKSG |
| | TSASLAITGLQAEDEADYYCQSYDSSLSGSVFGGG |
| | TKLTVL |
| 1330 | SYELTQPPSVSVSPGQTARISCSADALPKQNAYWY |
| | QCKPGQAPILLIYKDTERPSGIPERFSGSSSGTTV |
| | TLTISGVQPEDDADYYCQSVDNTGASPHVVFGGGT |
| | KLTVL |
| 1331 | DIQMTQSPSTLSASVGDSVTITCRANETIASWVAW |
| | YQQKPGKAPKLLIYKASSLESGVPSRFSGSESGTE |
| | FTLTISSLQPDDFATYYCQQYHTYWTFGQGTKVEV |
| | K |
| 1332 | SYELTQPPSVSVSPGQTASIACSGDKLGDKYTCWY |
| | QQKPGQSPVLVMYQDSKRPSGIPERFSGSNSGNTA |
| | TLTISGTQVMDEADYYCQAWDSGTVVFGGGTKLTV |
| | L |
| 1333 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPRTFGQGTKV |
| | EIK |
| 1334 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPRTFGQGTKV |
| | EIK |
| 1335 | DIVLTQSPGTLSLSPGERATLSCRASQSISSSYLA |
| | WYQQKPGQAPRLVIHGASSRATGIPDRFSGSGSGT |
| | DFTLTISRLEPEDFAVYYCQHYGTSPYTFGQGTKL |
| | EIK |
| 1336 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSTLVTFGQGTK |
| | VEIK |
| 1337 | QSALTQPASVSGSPGQSITISCTGTSSDVGSYNLV |
| | SWYQQHPGKAPKLMIYEVSKRPSGVSNRFSGSKSG |
| | NTASLTISGLQAEDEADYYCCSYAGSSLWVFGGGT |
| | KLTVL |
| 1338 | NFMLTQPHSVSESPGKTVTISCTGSSGSIASNYVQ |
| | WYQQRPGSAPTTVIYEDNQRPSGVPDRFSGSIDSS |
| | SNSASLTISGLKTEDEADYYCQSYDSSFWVFGGGT |
| | KLTVL |
| 1339 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAVVFGGGT |
| | KLTVL |
| 1340 | SYELTQPPSVSVSPGQTARITCSGDALPEKYAYWF |
| | QQKSGQAPVLVIYEDNKRPSGIPERFSGSSSGTMA |
| | TLTISGAQVEDEADYYCYSTDRSGNHRGVFGTGTK |
| | VTVL |
| 1341 | SSELTQDPAVSVALGQTVRITCQGDSLRSYYASWY |
| | QQKPGQAPVLVIYGKNNRPSGIPDRFSGSSSGNTA |
| | SLTITGAQAEDEADYYCNSRDSSGNHLYWVFGGGT |
| | KLTVL |
| 1342 | QSVLTQPPSVSGAPGQRVTISCTGSSSNIGAGYDV |
| | HWYQQLPGTAPKLLIYGNSNRPSGVPDRFSGSKSG |
| | TSASLAITGLQAEDEADYYCQSYDSSLSGHVVFGG |
| | GTKLTVL |
| 1343 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAGGVFGTG |
| | TKVTVL |
| 1344 | QSALTQPASVSGSPGQSITISCTGTSSDVGSYNLV |
| | SWYQQHPGKAPKLMIYEGSKRPSGVSNRFSGSKSG |
| | NTASLTISGLQAEDEADYYCCSYAGSSTFVVFGGG |
| | TKLTVL |
| 1345 | QSVLTQPPSVSAAPGQKVTISCSGSSSNIGNNYVS |
| | WYQQFPGTAPKLLIYDNNKRPSGIPDRFSGSKSGT |
| | SATLGITGLQTGDEADYYCGTWDSSLSAVVFGGGT |
| | KLTVL |
| 1346 | AIQLTQSPSSLSASVGDRVTITCRASQGISSYLAW |
| | YQQKPGKAPKLLIYAASTLQSGVPSRFSGSGSGTD |
| | FTLTISSLQPEDFATYYCQQLNSYPPTFGQGTKVE |
| | IK |
| 1347 | DIQMTQSPSSLSASVGDRVTITCRASQSIRFYLNW |
| | YQQKPGKAPKLLIYAASSLQSGVPSRFSGSGSGTD |
| | FTLTISSLQPEDFATYYCQQSYSTLWTFGQGTKVE |
| | IK |
| 1348 | EIVLTQSPGTLSLSPGERATLSCRASQSVSSTYLA |
| | WYQQKPGQAPRLLIYDASSRATGIPDRFSGGGSGT |
| | DFTLTISRLEPEDFAVYYCQQYGDSPETFGQGTKV |
| | EIK |
| 1349 | SYELTQPPSVSVSPGQTASITCSGDKLGDNYASWY |
| | QQKSGQSPVLVIYQDTKRPSGIPERFSGSNSGNTA |
| | TLTISGTQAMDEADYYCQAWDSSTVVFGGGTKLTV |
| | L |
| 1350 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPYTFGQGTKLE |
| | IK |
| 1351 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPYTFGQGTKLE |
| | IK |
| 1352 | EIVLTQSPATLSLSPGERATLSCRASQSVSSYLAW |
| | YQQKPGQAPRLLIYDASNRATGIPARFSGSGSGTD |
| | FTLTISSLEPEDFAVYYCQQRSNWPSITFGQGTRL |
| | EIK |
| 1353 | DIQMTQSPSSVSASVGDRVTITCRASQGISNWLAW |
| | YQQKPGKAPKLLIYAASSLQSGVPSRFSGSGSGTD |
| | FTLTISSLQPEDFATYYCQQANSFPLAFGGGTKVE |
| | IK |
| 1354 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPFGFGPGTKVD |
| | IK |
| 1355 | QSVLTQPPSVSGAPGQRVTISCTGSRSNIGAGFDV |
| | HWYQQLPGTAPKLLIYGNSNRPSGVPDRFSGSKSG |
| | TSASLAITGLQAEDEAVYYCLSYDSSLSGSVFGGG |
| | TKLTVL |
| 1356 | AIQLTQSPSSLSASVGDRVTITCRASQGISSALAW |
| | YQQKPGKAPKVLIYDASSLESGVPPRFSGSGSGTD |
| | FTLTISSLQPEDFATYYCQQFNNYPLTFGGGTKVE |
| | IK |
| 1357 | DIQMTQSPSSLSASVGDRVTITCQASQDMSNYLNW |
| | YQQKPGKAPKLLIYDASNLETGVPSRFSGSGSGTD |
| | FTFTISSLQPEDIATYYCQQYDNLPFTFGPGTKVD |
| | IK |
| 1358 | QSALTQPASVSGSPGQSITISCTGTSSDVGGYNYV |
| | SWYQQHPGKAPKLMIYDVSNRPSGVSNRFSGSKSG |
| | NTASLTISGLQAEDEADYYCSSYTSSSAYVFGTGT |
| | KVTVL |
| 1359 | SSELTQDPAVSVALGQTVRITCQGDSLRSYSASWY |
| | QQKPGQAPVLVIYVKNNRPSGIPDRFSGSSSGNTA |
| | SLTITGAQAEDEADYYCNSRDSSGNHVVFGGGTRL |
| | TVL |
| 1360 | QSALTQPRSVSGSPGQSVTISCTGTSSDVGDYDYV |
| | SWYQHHPGKAPKLMIYDVSKRPSGVPDRFSGSKSG |
| | NTASLTISGLQAEDDADYYCCSYAGSYPVVFGGGT |
| | KLTVL |
| 1361 | QSALTQPPSASGSPGQSVTISCTGTSSDVGGYNYV |
| | SWYQQHPGKAPKLMIYEVSKRPSGVPDRFSGSKSG |
| | NTASLTVSGLQAEDEADYYCSSYAGSNKVFGGGTK |
| | LTVL |
| 1362 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSGGYTFGQGTK |
| | LEIK |
| 1363 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPYTFGQGTKLE |
| | IK |
| 1364 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSSWTFGQGTKV |
| | EIK |
| 1365 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPYTFGQGTKLE |
| | IK |
| 1366 | SYELTQPPSVSVSPGQTASITCSGDKLGNKYACWY |
| | QQKPGQSPVLVIYQDSKRPSGIPERFSGSNSGNTA |
| | TLTISGTQAMDEADYYCQAWDSSTANWVFGGGTKL |
| | TVL |
| 1367 | QSALTQPASVSGSPGQSITISCTATSGDVGGYNYV |
| | SWYQQHPGKAPKLMIFDVYNRPSGVSNRFSGSKSG |
| | NTASLTISGLQAEDEADYYCSSFTDSSTLVVFGGG |
| | TKLTVL |
| 1368 | DIQMTQSPSSLSASVRDKVTITCRASQSISSCLNW |
| | YQQKPGKAPKVLIYAASSLQSGVPSRFSGSGSGTD |
| | FTLTISSVQPEDFATYYCQQSYSVPHTFGQGTKVE |
| | IK |
| 1369 | EIVMTQSPATLSVSPGERATLSCRASQSVSSNLAW |
| | YQQKPGQAPRLLIYGASTRATGIPARFSGSGSGTE |
| | FTLTISSLQSEDFAVYYCQQYNNWLTFGGGTKVEI |
| | K |
| 1370 | EIVMTQSPATLSVSPGERVTLSCRASQSINRNLAW |
| | YQQKPGQAPRLLVYDASTRAPGIPTRVSGSGSGTD |
| | FTLTISSLQSEDFAVYYCQQYNNWPPITFGQGTRL |
| | EIQ |
| 1371 | EIVMTQSPATLSVSPGERVTLSCRASQSVNRNLAW |
| | YQQKPGQAPRLLVYDASTRAPGIPTRVRGSGSGTD |
| | FTLTISSLQSEDFAVYYCQQYNNWPPITFGQGTRL |
| | EIQ |
| 1372 | QTALTQPPSASGSPGQSVTISCTGSSGDVGGYNYV |
| | SWYQQYPGKAPKLILSEVSQRPSGVPDRFFGSKSG |
| | NTASLTVFGLQAEDEADYYCSSYAGTNKILFGGGT |
| | KLTVL |
| 1373 | DIQMTQSPSSLSASVGDRVTITCRASQSISSFLNW |
| | FQQKPGKAPKLLIYAASSLQGGVPSRFSGSGSGTD |
| | FTLTINSLQPEDFATYYCQQSYSTPLTFGGGTKVE |
| | IK |
| 1374 | QSALTQPASVSGSPGQSITISCTGTSSDVGGYNYV |
| | SWYQQHPGKAPKLMIYDVSKRPSGVSNRFSGSKSG |
| | NTASLTISGLQAEDEADYYCSSYTSSSTWVFGGGT |
| | KLTVL |
| 1375 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPYTFGQGTKLE |
| | IK |
| 1376 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTPGTFGQ |
| | GTRLEIK |
| 1377 | DIVMTQSPLSLPVTPGEPASISCRSSQSLLHSNGY |
| | NYLDWYLQKPGQSPQLLIYLGSNRASGVPDRFSGS |
| | GSGTDFTLKISRVEAEDVGVYYCMQALQTPGTFGQ |
| | GTRLEIK |
| 1378 | DIQMTQSPSTLSASVGDRVTITCRASQSISSWLAW |
| | YQQKPGKAPKLLIYKASSLESGVPSRFSGSGSGTE |
| | FTLTISSLQPDDFATYYCQQYNSYSAFGQGTKLEI |
| | K |
| 1379 | PYDLTQPPSVSVSPGQTATITCSGDKLGKKYACWY |
| | QQKPGQSPVLLIYQDVKRPSGIPERFSGSNSGTTA |
| | TLTISETQTMDEADYYCQAWDRTTATFGGGTRLTV |
| | L |
| 1380 | QSVLTQPPSVSGAPGQRVTISCTGSSSNIGAGYDV |
| | HWYQQLPGTAPKLLIYGNSNRPSGVPDRFSGSKSG |
| | TSASLAITGLQAEDEADYYCQSYDSSLSGWVFGGG |
| | TKLTVL |
| 1381 | QSALTQPASVSGSPGQSITISCTGTTFDVGVYDFV |
| | SWYQQLPGKAPKLIIHDDTHRPSGVSDRFSGSRSG |
| | TTASLTISGLQADDEADYYCSSYTSLNTLEVVFGG |
| | GTKLTVL |
| 1382 | DIVMTQSPLSLPVTPGEPASMSCKSTQSLLHSNGN |
| | YYVTWYLQKPGQSPHLLIYLASNRASGVPDRFSGS |
| | GSGTDFTLKISSVEAEDVGVYYCMQALQTPYSFGQ |
| | GTKLEIK |
| 1383 | SYVLTQPPSVSVAPGKTARITCGGNNIGSKSVHWY |
| | QQKPGQAPVLVIYYDSDRPSGIPERFSGSNSGNTA |
| | TLTISRVEAGDEADYYCQVWDSSSDRTVVFGGGTK |
| | LTVL |
| 1384 | QSVLTQPPSASGTPGQRVTISCSGSSSNIGNNYVY |
| | WYQHLPGTAPKLLIYRNNQRPSGVPDRFSGSKSGT |
| | SASLAISGLRSENEADYYCASWDDKVRGWVFGGGT |
| | KLTVL |
| 1385 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPWTFGQGTKV |
| | EIK |
| 1386 | DIQMTQSPSTLSASVGDRVTITCRASQSISSWLAW |
| | YQQKPGKAPKLLIYKASSLESGVPSRFSGSGSGTE |
| | FTLTISSLQPDDFATYYCQQYNSYSRTFGQGTKVE |
| | IK |
| 1387 | DIQMTQSPSTLSASVGDRVTITCRASQSISDWLAW |
| | YQQKPGKAPKLLIYKASTLEGGVPSRFSGSESGTE |
| | FTLTISSLQPDDFATYYCQQYNTSPLTFGGGTKVE |
| | IK |
| 1388 | QSVLTQPPSVSGAPGQRVTISCTGSSSNIGAGYDV |
| | HWYQQLPGTAPKLLIYGNSNRPSGVPDRFSGSKSG |
| | TSASLAITGLQAEDEADYYCQSYDSSLSGSLFGGG |
| | TKLTVL |
| 1389 | SYELTQPPSVSVSPGQTARITCSGDALPKQYAYWY |
| | QQKPGQAPVLVIYKDSERPSGIPERFSGSSSGTTV |
| | TLTISGVQAEDEADYYCQSADSSGTYRVFGGGTKL |
| | TVL |
| 1390 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGRTFGQGTKVEIK |
| 1391 | QSALTQPASVAGSPGQTITISCTGPNSDINSYDYV |
| | SWYQQRPGKAPKLIIHDVDHRPSGVSDRFSGFMSD |
| | NTASLTISGLQAEDEAHYYCSSYTNIDTLEIVFGA |
| | GTKLTVL |
| 1392 | DIQMTQSPSAMSASVGDRVTITCRASQGISNYLAW |
| | FQQKPGKVPKRLIYAASSLQSGVPSRFSGSGSGTE |
| | FTLTISSLQPEDFATYYCLQHNSYPRTFGQGTKVE |
| | IK |
| 1393 | SYELTQPPSVSVAPGKAASITCGGINIGSKSVHWY |
| | QQKPGQAPVLVVYDDSDRPSGIPERFSGSNSGNTA |
| | TLTISRVESGDEADYYCQVWHSSFDPWVFGGGTKL |
| | TVL |
| 1394 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYSTPPTTFGPGTKV |
| | DIK |
| 1395 | DIQMTQSPSTLSASVGDRVTITCRASQSISSWLAW |
| | YQQKPGKAPKLLIYKASSLESGVPSRFSGSGSGTE |
| | FTLTISSLQPDDFATYYCQQYNSYFPTFGQGTKVE |
| | IK |
| 1396 | SYVLTQPPSVSVAPGKTARITCGGNNIGSKSVHWY |
| | QQKPGQAPVLVVYDDNDRPSGIPDRFSGSNSGNTA |
| | TLTISRVEAGDEADYYCQVWDSSSDHYWVFGGGTK |
| | LTVL |
| 1397 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAGVFGGGT |
| | KLTVL |
| 1398 | QLVVTQSPSASASLGASVKLTCTLSSGHSSYVIAW |
| | HQQQPEKGPRFLMKLNSDGSHNKGDGIPDRFSGSS |
| | SGAERYLTISNLQSEDEADYYCQTWGTGPQVLFGG |
| | GTKLTVL |
| 1399 | DIQMTQSPSSLSASVGDRVTITCQASQDISNYLNW |
| | YQQKPGRAPKLLIYDASNLETGVPSRFSGSGSGTD |
| | FTFTISSLQPEDIATYYCQQYDNLLTFGGGTKVEI |
| | K |
| 1400 | SYVLTQPPSVSVAPGKTARITCGGNNIGSKSVHWY |
| | QQKPGQAPVLVVYDDSDRPSGIPERFSGSNSGNTA |
| | TLTISRVEAGDEADYYCQVWDSSGDHWVFGGGTKL |
| | TVL |
| 1401 | EIVLTQSPATLSLSPGERATLSCRASQSVSNYLAW |
| | YQQKPGQVPRLLIYDASNRATGIPARFSGSGSGTD |
| | FTLTISSLEPEDFAVYYCQQRSNWLTFGGGTKVEI |
| | K |
| 1402 | DIQMTQSPSSLSASVGDRVTITCQASQDISNYLNW |
| | YQQKPGKAPKLLISDASLLETGVPSRFSGSGSGTD |
| | FTFTISSLQPEDIATYYCQQHDNLPSFTFGPGTKV |
| | DIK |
| 1403 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPRTFGQGTKV |
| | EIK |
| 1404 | EIVLTQSPGTLSLSPGERATLSCRASQSVSSSYLA |
| | WYQQTPGQAPRLLIYGASSRATGIPDRFSGSGSGT |
| | DFTLTISRLEPEDFAVYYCQQYGSSPRTFGQGTKV |
| | EIK |
| 1405 | QTVVTQEPSLTVSPGGTVTLTCASSTGAVTSGYYP |
| | NWFQQKPGQAPRALIYSTSNKHSWTPARFSGSLLG |
| | GKAALTLSGVQPEDEAEYYCLLYYGGAPVFGGGTK |
| | LTVL |
| 1406 | DIQLTQSPSFLSASVGDRVTITCRASQGISSYLAW |
| | YQQKPGKAPKLLIYAASTLQSGVPSRFSGSGSGTE |
| | FTLTISSLQPEDFATYYCQQLNSYPPAFGQGTKVE |
| | IK |
| 1407 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPQTFGPGTKVD |
| | IK |
| 1408 | QSALTQPASVSGSPGQSITISCTGTSSDVGSYNLV |
| | SWYQQHPGKAPKLMIYEVSKRPSGVSNRFSGSKSG |
| | NTASLTISGLQAEDEADYYCCSYAGSSLWVFGGGT |
| | KLTVL |
| 1409 | NFMLTQPHSVSESPGKTVTISCTRSSGSIASNYVQ |
| | WYQQRPGSAPTTVIYEDNQRPSGVPDRFSGSIDSS |
| | SNSASLTISGLKTEDEADYYCQSYDSSNQVFGGGT |
| | KLTVL |
| 1410 | EIVLTQSPATLSLSPGERATLSCRASQSVSSYLAW |
| | YQQKPGQAPRLLIYDASNRATGIPARFSGSGSGTD |
| | FTLTISSLEPEDFAVYYCQQRSNWLFTFGPGTKVD |
| | IK |
| 1411 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAGVFGGGT |
| | KLTVL |
| 1412 | VIVLTQTPLSSPVTLGQPASISCRSRRSLVHTNGN |
| | TYLSWLHQRPGQTPRLLIHNVSNRFSGVPDRFSGS |
| | GAGTDFTLNISRVEADDVGIYYCMQASQFPLTFGG |
| | GTKLEIK |
| 1413 | QSALTQPASVSGSPGQSITISCTGTFSDIGNYDLV |
| | SWYQQHPGKAPKVIIYEGYKRPSGVSDRFSGSKSG |
| | NTASLTISGLQAEDEADYFCCSFAGSNREFGGGTK |
| | LTVL |
| 1414 | EIVLTQSPGTLSLSPGERATLSCRASQSVSSSYLA |
| | WYQQKPGQAPRLLIYGASSRATGIPDRFSGSGSGT |
| | DFTLTISRLEPEDFAVYYCQQYGSSPWTFGQGTKV |
| | EIK |
| 1415 | QSVLTQPPSVSAAPGQKVTISCSGSSSNIGNNYVS |
| | WYQQLPGTAPKLLIYDNNKRPSGIPDRFSGSKSGT |
| | SATLGITGLQTGDEADYYCGTWDSSLSAWVFGGGT |
| | KLTVL |
| 1416 | EIVLTQSPGTLSLSPGERATLSCRASQSVSNYLAW |
| | YQHKPGQAPRLLIYGASNGATGIPDRFSGSGSGTD |
| | FTLTISRLEPEDFAVYYCQHYSSSAPITFGQGTRL |
| | EIK |
| 1417 | EIVLTQSPATLSLSPGERATLSCRASQSVSSYLAW |
| | YQQKPGQAPRLLIFDASNRATGIPARFSGSGSGTD |
| | FTLTISSLEPEDFAVYYCQQRNKWPGTFGQGTKVE |
| | IK |
| 1418 | ETVLTQSPGTLSLSPGERATLSCRASQSVNSNYLA |
| | WYQQKPGQAPRLLIYGASSRATGIPDNFSGSGSGT |
| | DFTLTISRLEPEDFAVYYCQQYGDSPYTFGQGTNL |
| | EIK |
| 1419 | AIQLTQSPSSLSASVGDRVTITCRASQGISSSLAW |
| | YQQKPGKAPKLLIYSASTLQSGVPSRFSGSGSGTD |
| | FTLTITSLQPEDFATYYCQQLNSYPLTFGGGTKVE |
| | IK |
| 1420 | QSALTQPASVSGSPGQSITISCTGTSSDVGTYNLV |
| | SWYQQHPGKAPKLMIYEVSKRPSGVSNRFSGSKSG |
| | NTASLTISGLQAEDEADYYCCTYAGSSTWVFGGGT |
| | KLTVL |
| 1421 | DIQMTQSPSSLSASVGDRVTITCRASQSIAKFLNW |
| | YQKKPGKAPNLLISTASSFQSGVPSRFSGSGSGTD |
| | YTLTISGLQPEDFATYYCQQSYSSPYTFGQGTNLE |
| | IK |
| 1422 | DIQMTQSPSSVSASVGDRVTITCRASQGISSWLAW |
| | YQQKPGKAPKLLIYAASSLQSGIPSRFSGSGSGTD |
| | FTLTISSLQPEDFATYYCQQANSFPRTFGQGTKVE |
| | IK |
| 1423 | AIQLTQSPSSLSASVGDRVTITCRASQGISSALAW |
| | YQQKPGKAPKVLIYDASGLQSGVPSRFSGGGSGTD |
| | FTLTISSLQPEDFATYYCQQFNDYPLTFGGGTKVE |
| | IK |
| 1424 | QSVLTQPPSVSGAPGQRVTISCTGSNSNIGAGYDV |
| | HWYQQLPGTAPKLLIYVNTNRPSGVPDRFSGSKSG |
| | TSASLAITGLQAEDEAHYYCQSYDSSLSGSVFGGG |
| | TKLTVL |
| 1425 | DIQMTQSPSTLSASVGDRVTITCRASQIISSWLAW |
| | YQQKPGKAPKLLIYKASSLESGVPSRFSGSGSGTE |
| | FTLTISSLQPDDFATYYCQQYSTYYTFGQGTKLEI |
| | K |
| 1426 | DVVLTQSPLSLPVTLGQPASISCRSSHSLVYSDGY |
| | THLHWIQERPGQSPRRLIYSVSHRDSGVPDRFSGS |
| | GSATDFTLQISRVEAEDVGVYYCMQGSHWPWTFGQ |
| | GTKVEIK |
| 1427 | QSVLTQPPSASGTPGQRVTISCSGSSSNIGSNTVN |
| | WYQQLPGTAPKLLIYSNNQRPSGVPDRFSGSKSGT |
| | SASLAISGLQSEDEADYYCAAWDDSLNGPWVFGGG |
| | TKLTVL |
| 1428 | QSALTQPRSVSGSPGQSVTISCTGTSSDVGGYNYV |
| | SWYQQYPGKAPKLMIYEVSKRPSGVPDRFSGSKSG |
| | NTASLTISGLQAEDEADYYCCSYAGSYTWVFGTGT |
| | KVTVL |
| 1429 | DIQLTQSPSFLSASVGDRVTITCRASQGISSYLAW |
| | YQQKPGKAPKLLIYAASTLQVGVPSRFSGSGSGTE |
| | FTLTISSLQPEDFATYFCQQLNSYPFTFGPGTKVD |
| | IK |
| 1430 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPRTFGQGTKVE |
| | IK |
| 1431 | DIQLTQSPSFLSASVGDRVTITCRASQGISSYLAW |
| | YQQKPGKAPKLLIYAASTLQSGVPSSFSGSGSGTE |
| | FTLTISSLQPEDFATYYCQQLNSYPLTFGGGTKVE |
| | IK |
| 1432 | DIQMTQSPSSLSASVGDRVTITCRASQSISNFLNW |
| | YQQKPGKAPKLLIYAASSLQSGVPSRFSGSGSGTG |
| | FTLTISSLQPEDFATYYCQQSYSTPPDTFGQGTRL |
| | EIK |
| 1433 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPPTFGGGTKVE |
| | IK |
| 1434 | DIQMTQSPSSLSASVGDRVTITCRASQSISSYLNW |
| | YQQKPGKAPKLLIYAASSLQSGVPSRFSGSGSGTD |
| | FTLTISSLQPEDFATYYCQQSYTTPLFTFGPGTKV |
| | DIK |
| 1435 | DIQLTQSPSSLSASVGDRVTITCRASQGISSYLAW |
| | YQQKPGKAPKLLIYAASTLQSGVPSRFSGSGSGTD |
| | FTLTISSLQPEDFATYYCQQLNGYPHSAFGPGTKV |
| | DIK |
| 1436 | DIQMTQSPSSLSASVGDRVTITCQASQDIINYLNW |
| | YQQKPGKAPKLLIYGASNLETGVPSRFSGGGSGTD |
| | FTFTISSLQPEDIATYYCHQYDNLPPTFGQGTRLE |
| | IK |
| 1437 | DIQLTQSPSFLSASVGDRVTITCRASQGISSYLAW |
| | YQQKPGKAPKLLIYAASTLQSGVPSRFSGSGSGTE |
| | FTLTISSLQPEDFATYYCQQLNSYPLTFGGGTKVE |
| | IK |
| 1438 | DIQLTQSPSSLSASVGDRVTITCRASQGISSYLAW |
| | YQQKPGKAPKLLIYAASTLQSGVPSRFSGSGSGTD |
| | FTLTISSLQPEDVATYYCQQLNSNPPITFGPGTKV |
| | DIK |
| 1439 | DIQMTQSPSSLSASVGDRVTITCRASQSISSYLNW |
| | YQQKPGKAPKLLIFAASSLQTGAPSRFSGSGSGTD |
| | FTLTISSLQPEDFATYYCQQSYSTPPYTFGQGTKL |
| | EIK |
| 1440 | DIQMTQSPSSLSASVGDRVTITCQASQDINKYLNW |
| | YQQKPGKAPKLLIFDASHLETGVPSRFSASGSGTD |
| | FTFTISSLQPEDIATYYCHQYDNLPRTFGQGTRLE |
| | IK |
| 1441 | GGSFSDYY |
| 1442 | GGSFSDYF |
| 1443 | GITVSSNY |
| 1444 | GYTFTSYA |
| 1445 | ITHSGST |
| 1446 | INHSGST |
| 1447 | IYSGGST |
| 1448 | INTNTGNP |
| 1449 | QSVSTY |
| 1450 | QSVSSY |
| 1451 | QGISSY |
| 1452 | QSISSW |
| 1453 | DAS |
| 1454 | DAS |
| 1455 | AAS |
| 1456 | KAS |
| 1457 | ASTKGPSVFPLAPSSKSTSGGTAALGCLVKDYFPE |
| | PVTVSWNSGALTSGVHTFPAVLQSSGLYSLSSVVT |
| | VPSSSLGTQTYICNVNHKPSNTKVDKKVEPKSCDK |
| | THTCPPCPAPELLGGPSVFLFPPKPKDTLMISRTP |
| | EVTCVVVDVSHEDPEVKFNWYVDGVEVHNAKTKPR |
| | EEQYNSTYRVVSVLTVLHQDWLNGKEYKCKVSNKA |
| | LPAPIEKTISKAKGQPREPQVYTLPPSRDELTKNQ |
| | VSLTCLVKGFYPSDIAVEWESNGQPENNYKTTPPV |
| | LDSDGSFFLYSKLTVDKSRWQQGNVFSCSVMHEAL |
| | HNHYTQKSLSLSPGK |
| 1458 | RTVAAPSVFIFPPSDEQLKSGTASVVCLLNNFYPR |
| | EAKVQWKVDNALQSGNSQESVTEQDSKDSTYSLSS |
| | TLTLSKADYEKHKVYACEVTHQGLSSPVTKSFNRG |
| | EC |
| 1459 | RVQPTESIVRFPNITNLCPFGEVFNATRFASVYAW |
| | NRKRISNCVADYSVLYNSASFSTFKCYGVSPTKLN |
| | DLCFTNVYADSFVIRGDEVRQIAPGQTGKIADYNY |
| | KLPDDFTGCVIAWNSNNLDSKVGGNYNYLYRLFRK |
| | SNLKPFERDISTEIYQAGSTPCNGVEGFNCYFPLQ |
| | SYGFQPTNGVGYQPYRVVVLSFELLHAPATVCGPK |
| | KSTNLVKNKCVNF |
| 1460 | MFVFLVLLPLVSSQCVNLTTRTQLPPAYTNSFTRG |
| | VYYPDKVFRSSVLHSTQDLFLPFFSNVTWFHAIHV |
| | SGTNGTKRFDNPVLPFNDGVYFASTEKSNIIRGWI |
| | FGTTLDSKTQSLLIVNNATNVVIKVCEFQFCNDPF |
| | LGVYYHKNNKSWMESEFRVYSSANNCTFEYVSQPF |
| | LMDLEGKQGNFKNLREFVFKNIDGYFKIYSKHTPI |
| | NLVRDLPQGFSALEPLVDLPIGINITRFQTLLALH |
| | RSYLTPGDSSSGWTAGAAAYYVGYLQPRTFLLKYN |
| | ENGTITDAVDCALDPLSETKCTLKSFTVEKGIYQT |
| | SNFRVQPTESIVRFPNITNLCPFGEVFNATRFASV |
| | YAWNRKRISNCVADYSVLYNSASFSTFKCYGVSPT |
| | KLNDLCFTNVYADSFVIRGDEVRQIAPGQTGKIAD |
| | YNYKLPDDFTGCVIAWNSNNLDSKVGGNYNYLYRL |
| | FRKSNLKPFERDISTEIYQAGSTPCNGVEGFNCYF |
| | PLQSYGFQPTNGVGYQPYRVVVLSFELLHAPATVC |
| | GPKKSTNLVKNKCVNFNFNGLTGTGVLTESNKKFL |
| | PFQQFGRDIADTTDAVRDPQTLEILDITPCSFGGV |
| | SVITPGTNTSNQVAVLYQDVNCTEVPVAIHADQLT |
| | PTWRVYSTGSNVFQTRAGCLIGAEHVNNSYECDIP |
| | IGAGICASYQTQTNSPRRARSVASQSIIAYTMSLG |
| | AENSVAYSNNSIAIPTNFTISVTTEILPVSMTKTS |
| | VDCTMYICGDSTECSNLLLQYGSFCTQLNRALTGI |
| | AVEQDKNTQEVFAQVKQIYKTPPIKDFGGFNFSQI |
| | LPDPSKPSKRSFIEDLLFNKVTLADAGFIKQYGDC |
| | LGDIAARDLICAQKFNGLTVLPPLLTDEMIAQYTS |
| | ALLAGTITSGWTFGAGAALQIPFAMQMAYRFNGIG |
| | VTQNVLYENQKLIANQFNSAIGKIQDSLSSTASAL |
| | GKLQDVVNQNAQALNTLVKQLSSNFGAISSVLNDI |
| | LSRLDKVEAEVQIDRLITGRLQSLQTYVTQQLIRA |
| | AEIRASANLAATKMSECVLGQSKRVDFCGKGYHLM |
| | SFPQSAPHGVVFLHVTYVPAQEKNFTTAPAICHDG |
| | KAHFPREGVFVSNGTHWFVTQRNFYEPQIITTDNT |
| | FVSGNCDVVIGIVNNTVYDPLQPELDSFKEELDKY |
| | FKNHTSPDVDLGDISGINASVVNIQKEIDRLNEVA |
| | KNLNESLIDLQELGKYEQYIKWPWYIWLGFIAGLI |
| | AIVMVTIMLCCMTSCCSCLKGCCSCGSCCKFDEDD |
| | SEPVLKGVKLHYT |
| 1461 | GFTFSSYG |
| 1462 | GFTFSSYG |
| 1463 | GYSFTSYW |
| 1464 | GYSFTSYW |
| 1465 | GYSFTSYW |
| 1466 | GYSFTSYW |
| 1467 | GYSFTSYW |
| 1468 | GGSINRNHF |
| 1469 | GGSINRNHF |
| 1470 | GYTFTSYG |
| 1471 | GFTFSYFE |
| 1472 | GFTFSYFE |
| 1473 | GYKFSNYY |
| 1474 | GYIFTNFY |
| 1475 | GFNFSSYA |
| 1476 | GIIVSRNE |
| 1477 | GGTFSTYA |
| 1478 | GFTFSSYG |
| 1479 | GFTFNNYA |
| 1480 | GFVFSNYW |
| 1481 | GGSISSGGYY |
| 1482 | GYNFNNYW |
| 1483 | GYTFTSYA |
| 1484 | GYTLTELS |
| 1485 | GFTFSSYG |
| 1486 | GFTFSSYP |
| 1487 | GGSISSGGYY |
| 1488 | GGSISSGGYY |
| 1489 | GFTVSSNY |
| 1490 | GGSISSYY |
| 1491 | gytftgyf |
| 1492 | GFTASSNY |
| 1493 | GGTFSSYG |
| 1494 | GGRFGSFA |
| 1495 | GFTFTDYA |
| 1496 | GGSISSYY |
| 1497 | gytftdyy |
| 1498 | GYSFTGHY |
| 1499 | GFTFSNYG |
| 1500 | GGSISSDVYS |
| 1501 | GDTFNSYA |
| 1502 | GFTFSHYG |
| 1503 | GYSFPAHW |
| 1504 | GYNFDTYW |
| 1505 | GYSFSGYW |
| 1506 | GYYFAAHW |
| 1507 | GYSFPAFW |
| 1508 | GYSFPAYW |
| 1509 | GYTLTELS |
| 1510 | gytftryw |
| 1511 | GFTFSSYS |
| 1512 | GFTFSSYS |
| 1513 | GGSISSSSYY |
| 1514 | GFSLSTSGVG |
| 1515 | GFTFSNAW |
| 1516 | GFTFSSYE |
| 1517 | GFTFSSYE |
| 1518 | GGSISSSSYY |
| 1519 | GGSISSGGYY |
| 1520 | GGSISSRSYY |
| 1521 | GFSLSNARMG |
| 1522 | GFSLSTSGVG |
| 1523 | GFTISPYG |
| 1524 | GFTISPYG |
| 1525 | GYTFGDYG |
| 1526 | GYTFGDYG |
| 1527 | GFSLSTSGVG |
| 1528 | GGSISTYR |
| 1529 | GFTFSNAW |
| 1530 | GYIFTNYA |
| 1531 | GYAFTSYQ |
| 1532 | GFTFGDYA |
| 1533 | GASFSSYY |
| 1534 | GYSFTKYW |
| 1535 | GFTFSSYA |
| 1536 | GDSVSSNTVA |
| 1537 | GFTFDDYG |
| 1538 | GFAFDDFA |
| 1539 | GFTVSSTF |
| 1540 | GGSIKRRGYY |
| 1541 | GGSFSAYY |
| 1542 | GGSISSSDYY |
| 1543 | GFTFSNAW |
| 1544 | GGTFSTYA |
| 1545 | GLRFTDAW |
| 1546 | GFSFSSYA |
| 1547 | GFSFSDFA |
| 1548 | GFTFTTYG |
| 1549 | GFTFRSYS |
| 1550 | GDSITSYY |
| 1551 | GGSFSGSY |
| 1552 | GGSFTDHY |
| 1553 | GGSISSSSYY |
| 1554 | GFSLSNARMG |
| 1555 | GFTFSSYG |
| 1556 | GFTFGDYA |
| 1557 | GFTFSGSA |
| 1558 | GYSFTSYW |
| 1559 | gytftsyy |
| 1560 | GGTFSSYA |
| 1561 | GFTFSNAW |
| 1562 | GFTFRSYW |
| 1563 | GFTFSTYA |
| 1564 | GFTFSSYG |
| 1565 | GFTVSSNY |
| 1566 | GGPISSGGYY |
| 1567 | GGSISSSYYY |
| 1568 | GGSISSSSYY |
| 1569 | GFTFSSYS |
| 1570 | GYTFTSYG |
| 1571 | GFTFSSYW |
| 1572 | GGSISSGGYS |
| 1573 | GYSFPAHW |
| 1574 | GYSFPAFY |
| 1575 | GYSFPAHW |
| 1576 | GFTFSASA |
| 1577 | GGSISSGGYY |
| 1578 | GFTFSSYW |
| 1579 | GYTFTSYG |
| 1580 | GFSLSTSGVS |
| 1581 | GFTFSSYG |
| 1582 | GFTFSSYA |
| 1583 | GFTFSSYE |
| 1584 | GFTFSIYA |
| 1585 | GFTFTSYG |
| 1586 | GFTFSSYG |
| 1587 | GFAFNKYG |
| 1588 | GFTFSSYG |
| 1589 | GGSISSSSYY |
| 1590 | GGAITTSSYF |
| 1591 | GFTFSAYG |
| 1592 | GFTFNNYG |
| 1593 | GGSINSYY |
| 1594 | GFTFSRFG |
| 1595 | GFTFSRFG |
| 1596 | GFTFSSFW |
| 1597 | GGTFSSYT |
| 1598 | GGSFSSYT |
| 1599 | EFSLDSRGVG |
| 1600 | GGSISSYY |
| 1601 | GFTFSRYG |
| 1602 | GFPFSGYA |
| 1603 | GFTFINYD |
| 1604 | GFAFDKFW |
| 1605 | GGSINRDGHY |
| 1606 | GGSISSYY |
| 1607 | GGSVSSGSYF |
| 1608 | GYTFTSYG |
| 1609 | GYTFTGYY |
| 1610 | GYTLTELS |
| 1611 | GYRFTSYG |
| 1612 | GYRFTSYG |
| 1613 | GYTFTSYA |
| 1614 | gytftsyy |
| 1615 | GYTFTNYY |
| 1616 | GGTFSSYT |
| 1617 | GGTFNSYA |
| 1618 | GYTFTSSD |
| 1619 | EFSLDARGVG |
| 1620 | GFTFISYA |
| 1621 | GFTFSSYA |
| 1622 | GFTFSSYG |
| 1623 | GFTFSSHG |
| 1624 | GFTFSSYA |
| 1625 | GFTFSSYA |
| 1626 | GFTFSTYG |
| 1627 | GFTFSTFA |
| 1628 | GFIFGDYA |
| 1629 | GFIFGDYA |
| 1630 | GFTFSSYW |
| 1631 | GASISSGDYY |
| 1632 | GGVLSDYY |
| 1633 | GGVLSDYY |
| 1634 | GGSFSDYY |
| 1635 | GGSFSDYF |
| 1636 | GDSISSNNW |
| 1637 | GGSISSYY |
| 1638 | GDSISSYY |
| 1639 | GGSISGYY |
| 1640 | GGSISSGSYY |
| 1641 | DDSISSGSYY |
| 1642 | GYSFTSYW |
| 1643 | GYSFTSYW |
| 1644 | GYTFTSYA |
| 1645 | GYTFSFYW |
| 1646 | GGAFSSGRHY |
| 1647 | GGSFSSYY |
| 1648 | GYTLTELS |
| 1649 | GFTFSDYY |
| 1650 | GITVSSNY |
| 1651 | GFTFSRFW |
| 1652 | GFTFSNYW |
| 1653 | AFSFHLHG |
| 1654 | GFTFSSYA |
| 1655 | GFIFDDYG |
| 1656 | GFTVSSNY |
| 1657 | GGSIGSSSYF |
| 1658 | GGSISSSSYY |
| 1659 | GFTFSSYD |
| 1660 | GFTFSRSA |
| 1661 | GFTFSSQS |
| 1662 | GFTFEEYS |
| 1663 | GYTFGRYW |
| 1664 | gytfstyy |
| 1665 | GGTFSSYA |
| 1666 | GATFTTYA |
| 1667 | GFTLSSYA |
| 1668 | GFTFSNYD |
| 1669 | GFTFDDYA |
| 1670 | GFTFDDYA |
| 1671 | GFTFDDYA |
| 1672 | GFTFSSYT |
| 1673 | TFIFSNSE |
| 1674 | GFTVSSNY |
| 1675 | GGSFSGYF |
| 1676 | GGSFSGYY |
| 1677 | GGSLSSYY |
| 1678 | GGSISTFY |
| 1679 | GGSVSSYF |
| 1680 | GFSFNTPGVG |
| 1681 | GFSFSNHG |
| 1682 | GFTFSNSA |
| 1683 | EFTFSSYE |
| 1684 | gytftnfa |
| 1685 | GYTFTSYG |
| 1686 | GFSFSRYG |
| 1687 | GFNFNSYT |
| 1688 | GFNFNSYT |
| 1689 | GFTFSSYE |
| 1690 | GYIFTSYG |
| 1691 | GYSFNDYG |
| 1692 | GYTLTELS |
| 1693 | GNTFSTYY |
| 1694 | GFTFSDVW |
| 1695 | GLTFDNAW |
| 1696 | RFTFSSYA |
| 1697 | GFTFDDYA |
| 1698 | GNTFTTYY |
| 1699 | GGTFNSYT |
| 1700 | GFSLNTPGAG |
| 1701 | GFSFNTPGVG |
| 1702 | GFTFTFSDYY |
| 1703 | GFTFSDYY |
| 1704 | GFSFSRYG |
| 1705 | GLSFSRYG |
| 1706 | GFSFNNFG |
| 1707 | GFTLSSYA |
| 1708 | GFTFSSYE |
| 1709 | GFTFSSYA |
| 1710 | GGSISPYS |
| 1711 | GDSIRSSSFY |
| 1712 | GYSFTTYA |
| 1713 | GGTFSSYA |
| 1714 | GGTFSSYA |
| 1715 | GFTFSHAW |
| 1716 | GFTFSSYE |
| 1717 | GFTVSSNY |
| 1718 | GGSISSYY |
| 1719 | GYTFTTYG |
| 1720 | GYTFTNYG |
| 1721 | GYTLTELS |
| 1722 | GYTLTELS |
| 1723 | GYTLTELS |
| 1724 | GYTLTELS |
| 1725 | GYTFTSYA |
| 1726 | GGTFSSYA |
| 1727 | GFSLSNARMG |
| 1728 | GFSLSTTGVG |
| 1729 | GFSLSTSGVG |
| 1730 | GFTFSSYS |
| 1731 | GFTFSSYS |
| 1732 | GFTFSSYA |
| 1733 | GFTFSSYA |
| 1734 | GFTFSSYA |
| 1735 | GFTFGSYG |
| 1736 | GFTFSSYA |
| 1737 | GFTFSIYA |
| 1738 | GFTVSSNY |
| 1739 | GFTFSNYW |
| 1740 | GGSFSGYY |
| 1741 | DGSFSGHY |
| 1742 | GGSFSGYY |
| 1743 | GGSISSYY |
| 1744 | GGSISSYY |
| 1745 | GYNFTSYW |
| 1746 | GYTFTSYA |
| 1747 | GFTVSSNY |
| 1748 | GGSISSGLYH |
| 1749 | GGTFSSYT |
| 1750 | GFTFGRHG |
| 1751 | GFTFGRYG |
| 1752 | GFSLTTRGEG |
| 1753 | GYTFTFYT |
| 1754 | GFTFTSSA |
| 1755 | GFSLSTSGMC |
| 1756 | GFTFTTYA |
| 1757 | GFAFTTYA |
| 1758 | GYTFTSYG |
| 1759 | GYTFSRYG |
| 1760 | GYTFTGYY |
| 1761 | GSGFTFRNAW |
| 1762 | GFTFNFYG |
| 1763 | GFTFDDYA |
| 1764 | GFIFDDYT |
| 1765 | GYTFTSYG |
| 1766 | GYTFTSYG |
| 1767 | GDTFNDYH |
| 1768 | GGTFSSYA |
| 1769 | GGTFSSYA |
| 1770 | GFTFSNAW |
| 1771 | GLTFTKAW |
| 1772 | GFTFSTYA |
| 1773 | GFTFSNYA |
| 1774 | GFTFSSYA |
| 1775 | GFTFSSYG |
| 1776 | GFTFSSYG |
| 1777 | GFTFSSFA |
| 1778 | GFTFSNYG |
| 1779 | GFTFSSYG |
| 1780 | GFTFSSYA |
| 1781 | GFTFHDYA |
| 1782 | GVIVSRNY |
| 1783 | GFTVSSNY |
| 1784 | EFTVSSNY |
| 1785 | GFTVSSNY |
| 1786 | GFTVSSNY |
| 1787 | GITVSSNY |
| 1788 | GFTVSSNY |
| 1789 | GFTFSSYW |
| 1790 | GGSISSGGYY |
| 1791 | GGSISSGGYY |
| 1792 | GGSFSGYY |
| 1793 | GGSFSDDF |
| 1794 | GGSISSYY |
| 1795 | GYSFTSYW |
| 1796 | GDTFSNYP |
| 1797 | GFTFSTSA |
| 1798 | GFTFSTYA |
| 1799 | GFTFFSYA |
| 1800 | GFTVSSNY |
| 1801 | AGSISSDTYY |
| 1802 | GYTFTSYG |
| 1803 | GYTFTNYY |
| 1804 | GGTFSSYT |
| 1805 | GFSLSTSGVG |
| 1806 | GFTVSSYD |
| 1807 | GFTFRNYG |
| 1808 | GFTFSSYG |
| 1809 | GFTVSRNY |
| 1810 | GFTVSSNY |
| 1811 | GFTVSRNY |
| 1812 | GLTVSSNY |
| 1813 | GFTVSSNY |
| 1814 | GLIVSSNY |
| 1815 | GITVRSNY |
| 1816 | GFTVSSNY |
| 1817 | GVTVSSNY |
| 1818 | GLTVSSNY |
| 1819 | GFIVSSNY |
| 1820 | EFIVSRNY |
| 1821 | IWYDGSNK |
| 1822 | IWYDGSNK |
| 1823 | IDPSDSYT |
| 1824 | IDPSDSYT |
| 1825 | IDPSDSYT |
| 1826 | IDPSDSYT |
| 1827 | IDPSDSYT |
| 1828 | ASYTGTT |
| 1829 | ASYTGTT |
| 1830 | ISAYNGNT |
| 1831 | ISSSGTNI |
| 1832 | ISSSGTNI |
| 1833 | INPYSGET |
| 1834 | VNPNDGSS |
| 1835 | ISATGGTT |
| 1836 | ISSSGTGV |
| 1837 | IIPIFGTP |
| 1838 | ISYDGSNK |
| 1839 | ISSYGDNT |
| 1840 | IKQDESEE |
| 1841 | IYYSGST |
| 1842 | IYGGDSDT |
| 1843 | INTNTGNP |
| 1844 | FDPEDGET |
| 1845 | ISYDGSNK |
| 1846 | ISYDGSNK |
| 1847 | IYYSGST |
| 1848 | IYYSGST |
| 1849 | LYSGGNE |
| 1850 | IYYSGST |
| 1851 | INPSSGVA |
| 1852 | IYAGGGT |
| 1853 | ILPVLDTT |
| 1854 | VTPIVGVP |
| 1855 | ISYDGNDK |
| 1856 | IYYSGST |
| 1857 | VNPNRGGT |
| 1858 | INPDSGGT |
| 1859 | ITGSGGST |
| 1860 | VFHTGSA |
| 1861 | IIPILRLA |
| 1862 | IWYDGSKK |
| 1863 | IFPGDSDT |
| 1864 | IYPGDSDS |
| 1865 | IFPSDSDT |
| 1866 | IFPSDSDT |
| 1867 | VFPGDSDT |
| 1868 | IFPGDSDT |
| 1869 | FDPEDGET |
| 1870 | MKPGDGKT |
| 1871 | ISSSSSYI |
| 1872 | ISSSSSTI |
| 1873 | IYYSGST |
| 1874 | IYWDDDK |
| 1875 | IKSKTDGGTT |
| 1876 | ISSSGSTI |
| 1877 | ISSSGSTI |
| 1878 | IYYSGST |
| 1879 | IYYSGST |
| 1880 | IYYSGST |
| 1881 | IFSNDEK |
| 1882 | IYWDDDK |
| 1883 | IWYDGSNK |
| 1884 | IWYDGSNK |
| 1885 | ISGYNGDP |
| 1886 | ISGYNGDP |
| 1887 | IYWDDDK |
| 1888 | IYYSGRT |
| 1889 | IKRIIDGGTI |
| 1890 | TNTNTGNP |
| 1891 | INPSGSAT |
| 1892 | ITWNSGNI |
| 1893 | ISQSAST |
| 1894 | IYPDDSET |
| 1895 | ISGSGDKT |
| 1896 | TYYRSNWYN |
| 1897 | ISWNSNSV |
| 1898 | INWNSDNI |
| 1899 | IYTVGDT |
| 1900 | IYYSGTT |
| 1901 | INRRGNT |
| 1902 | IYYSGNT |
| 1903 | IKSKTDGGTT |
| 1904 | IIPSLRTA |
| 1905 | IKSRGSGGTI |
| 1906 | ISYDGRNK |
| 1907 | VSYDSRQQ |
| 1908 | IWYDGSNE |
| 1909 | LSNDDRTR |
| 1910 | IYSSGDT |
| 1911 | INPSGGS |
| 1912 | INHSGRT |
| 1913 | IYYSGST |
| 1914 | IFSNDEK |
| 1915 | ISYDGSNK |
| 1916 | IRSKAYGGTT |
| 1917 | IRSKANSYAT |
| 1918 | IDPSDSYT |
| 1919 | INPSGGST |
| 1920 | IIPIFHIA |
| 1921 | IKSKTDGGTT |
| 1922 | IFSDWSTT |
| 1923 | ISGSGGST |
| 1924 | ISYDGSNK |
| 1925 | IYSGSST |
| 1926 | IYYSGST |
| 1927 | IYYSGST |
| 1928 | IYYSGST |
| 1929 | ISSSSSYI |
| 1930 | ISAYNGNT |
| 1931 | INSDGSST |
| 1932 | IYHSGST |
| 1933 | IFPSDSDT |
| 1934 | IFPGDSET |
| 1935 | IFPGDSDT |
| 1936 | IRTRTNRYAT |
| 1937 | IYYSGST |
| 1938 | INSDGSST |
| 1939 | ISAYNGNT |
| 1940 | IYWDDDK |
| 1941 | ISYDGSNK |
| 1942 | ISYDGSNK |
| 1943 | ISSSGSTI |
| 1944 | ISGSGGST |
| 1945 | ISFDGSNI |
| 1946 | ISYDGSNK |
| 1947 | IWNDGNKQ |
| 1948 | IWYDGSNK |
| 1949 | IFYSGST |
| 1950 | ISYSGDT |
| 1951 | ISFDGSNK |
| 1952 | ISYEGSIR |
| 1953 | IYSGGST |
| 1954 | ISYEGSTE |
| 1955 | ISYEGSTE |
| 1956 | IKEDGSEK |
| 1957 | IIPMLNKT |
| 1958 | IIPMLNKT |
| 1959 | IYWNDNK |
| 1960 | IYYRGST |
| 1961 | ISYEGSTE |
| 1962 | ISSSSSTV |
| 1963 | ISSSSSTT |
| 1964 | LNKDESEK |
| 1965 | IYSGRNT |
| 1966 | IYYSGST |
| 1967 | IYYSGST |
| 1968 | ISAYNGNT |
| 1969 | INPNSGGT |
| 1970 | FDPEDGET |
| 1971 | INTDNEKT |
| 1972 | INTDNGKT |
| 1973 | INAGNGNT |
| 1974 | INPSGGST |
| 1975 | INPSDGST |
| 1976 | IIPMLNKT |
| 1977 | IIPIFGPP |
| 1978 | MNPNTGTT |
| 1979 | IYWNDYK |
| 1980 | ISGSGGST |
| 1981 | ISGSGGTT |
| 1982 | ISYDGSNK |
| 1983 | ISYDGINK |
| 1984 | ISYDGSNK |
| 1985 | ISYDGSNK |
| 1986 | MWFDGVDK |
| 1987 | ISYDEINK |
| 1988 | IRGRLVGATV |
| 1989 | IRGRLVGATV |
| 1990 | IKQDGSEK |
| 1991 | IYYSGST |
| 1992 | IHRSGST |
| 1993 | IHRSGST |
| 1994 | ITHSGST |
| 1995 | INHSGST |
| 1996 | IYHSGTT |
| 1997 | IYTSGST |
| 1998 | IYHSGSA |
| 1999 | LHYSGRS |
| 2000 | IYTSGST |
| 2001 | IYAGEST |
| 2002 | IYPGDSDT |
| 2003 | IYPGDSDT |
| 2004 | INTNTGNP |
| 2005 | IYPGDFDT |
| 2006 | IYSGVIT |
| 2007 | VTHSGST |
| 2008 | FDPEDGET |
| 2009 | ISSSGSTI |
| 2010 | IYSGGST |
| 2011 | IKEDGSVM |
| 2012 | IKSDGSET |
| 2013 | IWFDGSKK |
| 2014 | ISSSGGGT |
| 2015 | ITWNSGSI |
| 2016 | IYSGGST |
| 2017 | IYYGGST |
| 2018 | IYYSGST |
| 2019 | IGTAGDT |
| 2020 | MSYDGSDI |
| 2021 | ISYDGNNK |
| 2022 | VSWNSGTI |
| 2023 | INPADSDT |
| 2024 | INPSGDST |
| 2025 | IIPIFGTA |
| 2026 | IFPIFTAA |
| 2027 | VSGSGGST |
| 2028 | ISSDGNNR |
| 2029 | ISWNSGSI |
| 2030 | ISWNSGTI |
| 2031 | ISWNSEKI |
| 2032 | INSGSSII |
| 2033 | ISSSDNSV |
| 2034 | IYSGGST |
| 2035 | INHSGKT |
| 2036 | INHSGST |
| 2037 | MYNSGST |
| 2038 | IYYSGRT |
| 2039 | IFYTGTS |
| 2040 | IYWDDEK |
| 2041 | IWYDGDNR |
| 2042 | IYYDGSNE |
| 2043 | IDSSSTTI |
| 2044 | INTKTGIP |
| 2045 | ISAYNGNT |
| 2046 | ISHDDSQK |
| 2047 | ISYEGSKK |
| 2048 | ISYEGSKK |
| 2049 | ISSSGSTI |
| 2050 | INTNTGSP |
| 2051 | ISAYNGET |
| 2052 | FDPEDGET |
| 2053 | ISPSGDDA |
| 2054 | IRSKSDGGTT |
| 2055 | VKSKTDGGTT |
| 2056 | ISYDGSNK |
| 2057 | ISWDGGST |
| 2058 | ISPSGDDA |
| 2059 | IVPMLGIT |
| 2060 | IYWDDDK |
| 2061 | IYWDDEK |
| 2062 | ISSGGDAI |
| 2063 | MSSDSDYI |
| 2064 | ISHDESQK |
| 2065 | ISHDESQK |
| 2066 | ISYEGSKK |
| 2067 | ISYDGSNK |
| 2068 | ITSSGNTI |
| 2069 | ISSSSGTI |
| 2070 | IYYTGKT |
| 2071 | VYNSGTA |
| 2072 | IDTNTGKP |
| 2073 | IIPIFGTA |
| 2074 | IIPIFGTA |
| 2075 | IKSNTDGGTT |
| 2076 | ISSSGSTI |
| 2077 | IYSGGST |
| 2078 | IYYSGST |
| 2079 | ISAYNGNT |
| 2080 | ISTYSGNT |
| 2081 | FDPEDGET |
| 2082 | FDPEDGET |
| 2083 | FDPEDGET |
| 2084 | FDPEDGET |
| 2085 | INAGNGNT |
| 2086 | IIPIFGTA |
| 2087 | IFSNDKK |
| 2088 | IYWDDDK |
| 2089 | IFWDDDK |
| 2090 | ISSSSSYI |
| 2091 | ISSSSSYI |
| 2092 | ISYDGSNK |
| 2093 | ISYDGSNK |
| 2094 | ISYDGSNK |
| 2095 | IWNDGSNK |
| 2096 | ISYDGSNK |
| 2097 | ISYDGSNK |
| 2098 | IYSGGST |
| 2099 | IKEDGSET |
| 2100 | IDHSGST |
| 2101 | INHSGST |
| 2102 | INHSGST |
| 2103 | IYYSGST |
| 2104 | IYYSGST |
| 2105 | IDPSDSYT |
| 2106 | INTNTGNP |
| 2107 | VYSGGHA |
| 2108 | IFSSGST |
| 2109 | IIPILGIA |
| 2110 | ISTYSGNT |
| 2111 | ISTYSGNT |
| 2112 | IYWDDDQ |
| 2113 | INTNTGTP |
| 2114 | IWGSGNT |
| 2115 | IDWDDDK |
| 2116 | ISDSGGSA |
| 2117 | ISDGGGSA |
| 2118 | ISAYNGNT |
| 2119 | ISGYNGNT |
| 2120 | INPNSGGT |
| 2121 | IKSKNDGGTT |
| 2122 | ISYDGNKR |
| 2123 | ISWNSGSI |
| 2124 | ITWNYATV |
| 2125 | ISAYNGNT |
| 2126 | ISAYNGNT |
| 2127 | INPNSGET |
| 2128 | IIPIFGTA |
| 2129 | IIPILGIA |
| 2130 | IKSKTDGGTT |
| 2131 | IKSRSDGGKI |
| 2132 | ISGSGGST |
| 2133 | ISANGRSP |
| 2134 | ISGSGGST |
| 2135 | ISYDGSNK |
| 2136 | ISYDGSNK |
| 2137 | ISYDGANK |
| 2138 | MWHDGSNK |
| 2139 | IWYDGSNK |
| 2140 | ISYDGSNK |
| 2141 | ISWNSGSI |
| 2142 | IYSGGST |
| 2143 | IYSGGTT |
| 2144 | IYSGGST |
| 2145 | IYSGGST |
| 2146 | LYSGGTT |
| 2147 | IYSGGST |
| 2148 | IYSGGST |
| 2149 | IKSDGSST |
| 2150 | IYYSGST |
| 2151 | IYYSGST |
| 2152 | ISHGGKT |
| 2153 | INHSGTT |
| 2154 | IYYSGST |
| 2155 | IYPGDSDT |
| 2156 | IIPIVGFA |
| 2157 | ISYDGSN |
| 2158 | ISYDGSNK |
| 2159 | ISGISDSGGNT |
| 2160 | IYSGGST |
| 2161 | IYTTGST |
| 2162 | ISAYNGNT |
| 2163 | INPSGGST |
| 2164 | IIPILGIA |
| 2165 | IYWDDDK |
| 2166 | ISARGSVT |
| 2167 | ISYDGSNK |
| 2168 | ISNYGSNK |
| 2169 | IYSGGST |
| 2170 | IYSGGST |
| 2171 | IYSGGTT |
| 2172 | IYSGGST |
| 2173 | IYSGGST |
| 2174 | LYAGGST |
| 2175 | IYSGGST |
| 2176 | IYSGGST |
| 2177 | IYSGGST |
| 2178 | IYSGGST |
| 2179 | IYSGGST |
| 2180 | IYSGGST |
| 2181 | QSIASY |
| 2182 | QGISSY |
| 2183 | SSDVGGYNY |
| 2184 | QSISDW |
| 2185 | QSISSY |
| 2186 | QDISNY |
| 2187 | QSVSSSY |
| 2188 | NSNIGINN |
| 2189 | SGHSSYA |
| 2190 | ALPKQY |
| 2191 | QSISSY |
| 2192 | QGISSA |
| 2193 | SSDFGTFHL |
| 2194 | AFNIGTNF |
| 2195 | QSLVYYDGNTY |
| 2196 | QSISRW |
| 2197 | QHISNY |
| 2198 | ALPKQY |
| 2199 | QSVLYSSNNNKNY |
| 2200 | GASIASNY |
| 2201 | QSVLYSSNNKNY |
| 2202 | HSVFFSKVNKDY |
| 2203 | QSISSW |
| 2204 | SSDVGGYNY |
| 2205 | ALPKQY |
| 2206 | SSDVGGYNY |
| 2207 | QSVSSSY |
| 2208 | QSVSSSY |
| 2209 | EDIDNH |
| 2210 | QSVSSSY |
| 2211 | RSNIGSKN |
| 2212 | SSDVGSYHY |
| 2213 | QSVLYSANNKYY |
| 2214 | QSVKSY |
| 2215 | KDINSY |
| 2216 | QSVLYSSNNKNY |
| 2217 | QDISSS |
| 2218 | ALSNQY |
| 2219 | QDISNF |
| 2220 | QAISNS |
| 2221 | RDIHNL |
| 2222 | NSNIGSNY |
| 2223 | QGISTNY |
| 2224 | GARYN |
| 2225 | QSISNH |
| 2226 | QSISTNY |
| 2227 | QSISTNY |
| 2228 | QSISTNY |
| 2229 | ALPKKY |
| 2230 | TGAVTSGHY |
| 2231 | SSNIGAGYD |
| 2232 | KLGDKY |
| 2233 | SSNIGNNY |
| 2234 | QSVSSN |
| 2235 | TGAVTSGHY |
| 2236 | QSISSY |
| 2237 | SSDVGGYNY |
| 2238 | QSVLYSSNNKNY |
| 2239 | QSVSSSY |
| 2240 | SSNIGNNY |
| 2241 | SSDVGGYNY |
| 2242 | QSLLHSNGYNY |
| 2243 | QSLLHSNGYNY |
| 2244 | QSLLHSNGYNY |
| 2245 | QSIASY |
| 2246 | QGISSY |
| 2247 | NIGSKS |
| 2248 | KLGDEY |
| 2249 | SSNIGNNY |
| 2250 | SSDVGGYNY |
| 2251 | LSINTD |
| 2252 | QGMSNY |
| 2253 | QSINSW |
| 2254 | QSISSW |
| 2255 | QTVSSTY |
| 2256 | SSNVGNQG |
| 2257 | QSVLYNSNNKDY |
| 2258 | SGSIASYF |
| 2259 | SGSVSTTYY |
| 2260 | QSVSDN |
| 2261 | QSLVHSDGNTY |
| 2262 | SSNIGNNY |
| 2263 | SSNIGSNY |
| 2264 | QSLVHSDGNTY |
| 2265 | QSLVYSDGNTY |
| 2266 | QSVRSNY |
| 2267 | QSLRQSQRFSY |
| 2268 | QSLLHSIGKTH |
| 2269 | HDIRTW |
| 2270 | QDIGNW |
| 2271 | SLETYY |
| 2272 | SLRTSY |
| 2273 | SSDVGGYNY |
| 2274 | SSNIGAGYD |
| 2275 | QSVLYSSNNKNY |
| 2276 | QSLVHSDGNTY |
| 2277 | SSDVGGYNY |
| 2278 | QSISSY |
| 2279 | SSDVGGYNY |
| 2280 | QSISSY |
| 2281 | SLRSYY |
| 2282 | ELGDTD |
| 2283 | QSISSW |
| 2284 | QSLVHSDGNTY |
| 2285 | QSISSY |
| 2286 | SSNIGAGYD |
| 2287 | SSNIGSNY |
| 2288 | SLRSYY |
| 2289 | QDISNY |
| 2290 | SSNIGNNY |
| 2291 | QSVSSN |
| 2292 | KLGDKY |
| 2293 | QSISTNY |
| 2294 | HSISTNY |
| 2295 | QTISTNY |
| 2296 | QTINSGY |
| 2297 | QSVSSSY |
| 2298 | ALPKQY |
| 2299 | QSISSY |
| 2300 | KLGDTY |
| 2301 | QSLLHSDGKTY |
| 2302 | QSLLHSDGKTY |
| 2303 | QSLLHSNGYNY |
| 2304 | ALPKKY |
| 2305 | SSDVGGYNY |
| 2306 | ALPKKY |
| 2307 | ESISNW |
| 2308 | QSVSSY |
| 2309 | QGIRND |
| 2310 | QGIGND |
| 2311 | QSVSGSY |
| 2312 | QSVSSSY |
| 2313 | QSLLYNFNNENY |
| 2314 | QSLLDSDGKTY |
| 2315 | QSLLDSDGKTY |
| 2316 | QSLLHSNGYNY |
| 2317 | QSVSTY |
| 2318 | QSVSSY |
| 2319 | QDSSKY |
| 2320 | QSVSFTSNNKNY |
| 2321 | QSLLDSDGKTY |
| 2322 | QDISTY |
| 2323 | QSISNY |
| 2324 | QSVVHSDGKTY |
| 2325 | HTISSSY |
| 2326 | ALPKQY |
| 2327 | SSDVGGYNY |
| 2328 | QSISNY |
| 2329 | QSLVYSDGNTY |
| 2330 | KLGDKY |
| 2331 | SSDVGGHDY |
| 2332 | SSDVGGHDY |
| 2333 | QSLVYSDGNTY |
| 2334 | SSNIGNNY |
| 2335 | ALPKQY |
| 2336 | QSVSTY |
| 2337 | QSISSW |
| 2338 | VGHDYFT |
| 2339 | QDSNTY |
| 2340 | NSDVGGYNY |
| 2341 | QSLLHSNGYNY |
| 2342 | QSISSW |
| 2343 | QSLIYSDGNTY |
| 2344 | QSVSSSY |
| 2345 | QGISSW |
| 2346 | KLGDKY |
| 2347 | QSISTW |
| 2348 | QYVGDN |
| 2349 | QYIGDN |
| 2350 | ALPKKY |
| 2351 | QDVSIY |
| 2352 | QSVYDSSNSKNY |
| 2353 | QSVYDTSNSKNY |
| 2354 | QSVSTY |
| 2355 | QSVSSY |
| 2356 | SSNIGAYT |
| 2357 | QSVSSIY |
| 2358 | QSVTSY |
| 2359 | QSITNW |
| 2360 | SSDVGSYNL |
| 2361 | ALPKQY |
| 2362 | QSVSSRY |
| 2363 | QSVSSSY |
| 2364 | QSLLDSDGKTY |
| 2365 | QRVGSS |
| 2366 | QSVSSN |
| 2367 | QGIRFW |
| 2368 | SSNIGAGYD |
| 2369 | QSISSW |
| 2370 | QGISSY |
| 2371 | QSVLYSASNKNY |
| 2372 | QDISNY |
| 2373 | HSLLHSDGKTY |
| 2374 | TGAVTSGHY |
| 2375 | SSDVGGYNY |
| 2376 | QDISNY |
| 2377 | SSDVGGYNY |
| 2378 | SSDVGSYNL |
| 2379 | QSIGKY |
| 2380 | QSIEHSDGNIY |
| 2381 | SSNTGAGYD |
| 2382 | QSLTSSS |
| 2383 | QSLLHGNGYTY |
| 2384 | NIGSKS |
| 2385 | QSVSSSY |
| 2386 | QSVSSK |
| 2387 | TGAVTSGHY |
| 2388 | QSVTRN |
| 2389 | SSNIGSNT |
| 2390 | SSDVGGYNY |
| 2391 | NIGSKN |
| 2392 | SSDVGAYNY |
| 2393 | QSISNY |
| 2394 | QDISNY |
| 2395 | SSNVALNA |
| 2396 | QSVSSN |
| 2397 | SGYSNYK |
| 2398 | SSDVGSYNL |
| 2399 | QHINRW |
| 2400 | QNISRW |
| 2401 | QSLLHSDGKTY |
| 2402 | ALPIKY |
| 2403 | QSVSTY |
| 2404 | ELPKQY |
| 2405 | SSNIGNNY |
| 2406 | QNINVF |
| 2407 | QSLNNNQ |
| 2408 | QSLNNNQ |
| 2409 | SSNIGAGYD |
| 2410 | QSISSH |
| 2411 | QSVASY |
| 2412 | SSNIGSNT |
| 2413 | HSLLHNNGNTY |
| 2414 | ALPKEF |
| 2415 | KLGDKY |
| 2416 | QSVSSSY |
| 2417 | ALSKQY |
| 2418 | QSLLHNNGNTY |
| 2419 | QGIRNS |
| 2420 | QNISRW |
| 2421 | QNISRW |
| 2422 | ALPQRY |
| 2423 | QGVASY |
| 2424 | QNINVF |
| 2425 | QNINVF |
| 2426 | QSLNSN |
| 2427 | SSNIGAGYD |
| 2428 | KLGEKY |
| 2429 | SSNIGAGYD |
| 2430 | ALPKQN |
| 2431 | ETIASW |
| 2432 | KLGDKY |
| 2433 | QSVSSSY |
| 2434 | QSVSSSY |
| 2435 | QSISSSY |
| 2436 | QSVSSSY |
| 2437 | SSDVGSYNL |
| 2438 | SGSIASNY |
| 2439 | SSNIGNNY |
| 2440 | ALPEKY |
| 2441 | SLRSYY |
| 2442 | SSNIGAGYD |
| 2443 | SSNIGNNY |
| 2444 | SSDVGSYNL |
| 2445 | SSNIGNNY |
| 2446 | QGISSY |
| 2447 | QSIRFY |
| 2448 | QSVSSTY |
| 2449 | KLGDNY |
| 2450 | QDISNY |
| 2451 | QDISNY |
| 2452 | QSVSSY |
| 2453 | QGISNW |
| 2454 | QSISSY |
| 2455 | RSNIGAGFD |
| 2456 | QGISSA |
| 2457 | QDMSNY |
| 2458 | SSDVGGYNY |
| 2459 | SLRSYS |
| 2460 | SSDVGDYDY |
| 2461 | SSDVGGYNY |
| 2462 | QSVSSSY |
| 2463 | QSISSY |
| 2464 | QSVSSSY |
| 2465 | QSISSY |
| 2466 | KLGNKY |
| 2467 | SGDVGGYNY |
| 2468 | QSISSC |
| 2469 | QSVSSN |
| 2470 | QSINRN |
| 2471 | QSVNRN |
| 2472 | SGDVGGYNY |
| 2473 | QSISSF |
| 2474 | SSDVGGYNY |
| 2475 | QSISSY |
| 2476 | QSLLHSNGYNY |
| 2477 | QSLLHSNGYNY |
| 2478 | QSISSW |
| 2479 | KLGKKY |
| 2480 | SSNIGAGYD |
| 2481 | TFDVGVYDF |
| 2482 | QSLLHSNGNYY |
| 2483 | NIGSKS |
| 2484 | SSNIGNNY |
| 2485 | QSVSSSY |
| 2486 | QSISSW |
| 2487 | QSISDW |
| 2488 | SSNIGAGYD |
| 2489 | ALPKQY |
| 2490 | QSVSSSY |
| 2491 | NSDINSYDY |
| 2492 | QGISNY |
| 2493 | NIGSKS |
| 2494 | QSISSY |
| 2495 | QSISSW |
| 2496 | NIGSKS |
| 2497 | SSNIGNNY |
| 2498 | SGHSSYV |
| 2499 | QDISNY |
| 2500 | NIGSKS |
| 2501 | QSVSNY |
| 2502 | QDISNY |
| 2503 | QSVSSSY |
| 2504 | QSVSSSY |
| 2505 | TGAVTSGYY |
| 2506 | QGISSY |
| 2507 | QDISNY |
| 2508 | SSDVGSYNL |
| 2509 | SGSIASNY |
| 2510 | QSVSSY |
| 2511 | SSNIGNNY |
| 2512 | RSLVHTNGNTY |
| 2513 | FSDIGNYDL |
| 2514 | QSVSSSY |
| 2515 | SSNIGNNY |
| 2516 | QSVSNY |
| 2517 | QSVSSY |
| 2518 | QSVNSNY |
| 2519 | QGISSS |
| 2520 | SSDVGTYNL |
| 2521 | QSIAKF |
| 2522 | QGISSW |
| 2523 | QGISSA |
| 2524 | NSNIGAGYD |
| 2525 | QIISSW |
| 2526 | HSLVYSDGYTH |
| 2527 | SSNIGSNT |
| 2528 | SSDVGGYNY |
| 2529 | QGISSY |
| 2530 | QDISNY |
| 2531 | QGISSY |
| 2532 | QSISNF |
| 2533 | QDISNY |
| 2534 | QSISSY |
| 2535 | QGISSY |
| 2536 | QDIINY |
| 2537 | QGISSY |
| 2538 | QGISSY |
| 2539 | QSISSY |
| 2540 | QDINKY |
| 2541 | AAS |
| 2542 | AAS |
| 2543 | EVS |
| 2544 | KAS |
| 2545 | AAS |
| 2546 | DAS |
| 2547 | GAS |
| 2548 | RSN |
| 2549 | LSSDGSH |
| 2550 | KDS |
| 2551 | EAA |
| 2552 | DAS |
| 2553 | EVN |
| 2554 | GDQ |
| 2555 | KVS |
| 2556 | KAS |
| 2557 | AAS |
| 2558 | KDS |
| 2559 | WAS |
| 2560 | EDT |
| 2561 | WAS |
| 2562 | WAS |
| 2563 | KAS |
| 2564 | DVS |
| 2565 | KDS |
| 2566 | DVS |
| 2567 | GAS |
| 2568 | GAS |
| 2569 | DAS |
| 2570 | GAS |
| 2571 | SNN |
| 2572 | EVS |
| 2573 | WAS |
| 2574 | GAS |
| 2575 | DAS |
| 2576 | WAS |
| 2577 | AAS |
| 2578 | KGT |
| 2579 | DAS |
| 2580 | AAS |
| 2581 | DAS |
| 2582 | KNN |
| 2583 | ATS |
| 2584 | RNT |
| 2585 | SAS |
| 2586 | ASS |
| 2587 | STS |
| 2588 | ATS |
| 2589 | EDS |
| 2590 | DIN |
| 2591 | GNS |
| 2592 | QDS |
| 2593 | DNN |
| 2594 | GAS |
| 2595 | DTS |
| 2596 | AAS |
| 2597 | DVS |
| 2598 | WAS |
| 2599 | GAS |
| 2600 | DNN |
| 2601 | DVS |
| 2602 | LGS |
| 2603 | LGS |
| 2604 | LGS |
| 2605 | AAS |
| 2606 | AAS |
| 2607 | YDS |
| 2608 | QNN |
| 2609 | DNN |
| 2610 | DVS |
| 2611 | GAS |
| 2612 | AAS |
| 2613 | KAS |
| 2614 | KAS |
| 2615 | GAS |
| 2616 | RND |
| 2617 | WAS |
| 2618 | EDN |
| 2619 | STN |
| 2620 | AAS |
| 2621 | KVS |
| 2622 | DNN |
| 2623 | RNN |
| 2624 | KVS |
| 2625 | KVS |
| 2626 | GAS |
| 2627 | LNS |
| 2628 | EVS |
| 2629 | TAF |
| 2630 | AAS |
| 2631 | GKN |
| 2632 | EKN |
| 2633 | EVS |
| 2634 | GNS |
| 2635 | WAS |
| 2636 | KVS |
| 2637 | DVS |
| 2638 | AAS |
| 2639 | DVS |
| 2640 | AAS |
| 2641 | GKN |
| 2642 | QDT |
| 2643 | KAS |
| 2644 | KIS |
| 2645 | AAS |
| 2646 | GNN |
| 2647 | RNN |
| 2648 | GKN |
| 2649 | DAS |
| 2650 | DNN |
| 2651 | GAS |
| 2652 | QDS |
| 2653 | ATS |
| 2654 | ATS |
| 2655 | ATS |
| 2656 | AAS |
| 2657 | GAS |
| 2658 | KDS |
| 2659 | AAS |
| 2660 | QDN |
| 2661 | EVS |
| 2662 | EVS |
| 2663 | LGS |
| 2664 | EDS |
| 2665 | DVS |
| 2666 | EDS |
| 2667 | KAS |
| 2668 | DAS |
| 2669 | AAS |
| 2670 | GAS |
| 2671 | GAS |
| 2672 | GAS |
| 2673 | WAS |
| 2674 | EVS |
| 2675 | EVS |
| 2676 | LGS |
| 2677 | GSS |
| 2678 | GAS |
| 2679 | DAS |
| 2680 | WAS |
| 2681 | EVS |
| 2682 | DAS |
| 2683 | GAS |
| 2684 | EVS |
| 2685 | AAS |
| 2686 | KDS |
| 2687 | DVS |
| 2688 | AAS |
| 2689 | KVS |
| 2690 | QDS |
| 2691 | DVT |
| 2692 | DVT |
| 2693 | KVS |
| 2694 | DNN |
| 2695 | KDN |
| 2696 | GSS |
| 2697 | ETS |
| 2698 | LEGSGSY |
| 2699 | DAS |
| 2700 | DVS |
| 2701 | LGS |
| 2702 | KAS |
| 2703 | KVS |
| 2704 | GAS |
| 2705 | AAS |
| 2706 | QDS |
| 2707 | KAS |
| 2708 | GAF |
| 2709 | GAS |
| 2710 | EDS |
| 2711 | DAY |
| 2712 | WAS |
| 2713 | WAS |
| 2714 | DAS |
| 2715 | DAS |
| 2716 | STD |
| 2717 | GAS |
| 2718 | GAS |
| 2719 | KAS |
| 2720 | EVS |
| 2721 | KDS |
| 2722 | GAS |
| 2723 | GAS |
| 2724 | EVS |
| 2725 | GAS |
| 2726 | GAS |
| 2727 | AAS |
| 2728 | GNT |
| 2729 | DAS |
| 2730 | AAS |
| 2731 | WAS |
| 2732 | AAS |
| 2733 | ELF |
| 2734 | DTN |
| 2735 | EVS |
| 2736 | DAS |
| 2737 | DVN |
| 2738 | EVS |
| 2739 | AAS |
| 2740 | KIS |
| 2741 | DNS |
| 2742 | GAS |
| 2743 | LGS |
| 2744 | DDS |
| 2745 | GAS |
| 2746 | GAS |
| 2747 | DTS |
| 2748 | GAS |
| 2749 | SNN |
| 2750 | EVS |
| 2751 | DDG |
| 2752 | DVT |
| 2753 | AAS |
| 2754 | DAS |
| 2755 | RDN |
| 2756 | GAS |
| 2757 | VGTGGIVG |
| 2758 | EVS |
| 2759 | EAS |
| 2760 | KAS |
| 2761 | EVS |
| 2762 | EDS |
| 2763 | DAS |
| 2764 | KDR |
| 2765 | DNN |
| 2766 | AAS |
| 2767 | GAS |
| 2768 | GAS |
| 2769 | GNS |
| 2770 | DAS |
| 2771 | DAS |
| 2772 | SNN |
| 2773 | EIS |
| 2774 | KDK |
| 2775 | QDN |
| 2776 | GAS |
| 2777 | KDS |
| 2778 | EIS |
| 2779 | DAS |
| 2780 | KAS |
| 2781 | KAS |
| 2782 | EDT |
| 2783 | AAS |
| 2784 | GAS |
| 2785 | GAS |
| 2786 | GAS |
| 2787 | GNN |
| 2788 | QDT |
| 2789 | GDS |
| 2790 | KDT |
| 2791 | KAS |
| 2792 | QDS |
| 2793 | GAS |
| 2794 | GAS |
| 2795 | GAS |
| 2796 | GAS |
| 2797 | EVS |
| 2798 | EDN |
| 2799 | DNN |
| 2800 | EDN |
| 2801 | GKN |
| 2802 | GNS |
| 2803 | DNN |
| 2804 | EGS |
| 2805 | DNN |
| 2806 | AAS |
| 2807 | AAS |
| 2808 | DAS |
| 2809 | QDT |
| 2810 | DAS |
| 2811 | DAS |
| 2812 | DAS |
| 2813 | AAS |
| 2814 | AAS |
| 2815 | GNS |
| 2816 | DAS |
| 2817 | DAS |
| 2818 | DVS |
| 2819 | VKN |
| 2820 | DVS |
| 2821 | EVS |
| 2822 | GAS |
| 2823 | AAS |
| 2824 | GAS |
| 2825 | AAS |
| 2826 | QDS |
| 2827 | DVY |
| 2828 | AAS |
| 2829 | GAS |
| 2830 | DAS |
| 2831 | DAS |
| 2832 | EVS |
| 2833 | AAS |
| 2834 | DVS |
| 2835 | AAS |
| 2836 | LGS |
| 2837 | LGS |
| 2838 | KAS |
| 2839 | QDV |
| 2840 | GNS |
| 2841 | DDT |
| 2842 | LAS |
| 2843 | YDS |
| 2844 | RNN |
| 2845 | GAS |
| 2846 | KAS |
| 2847 | KAS |
| 2848 | GNS |
| 2849 | KDS |
| 2850 | GAS |
| 2851 | DVD |
| 2852 | AAS |
| 2853 | DDS |
| 2854 | AAS |
| 2855 | KAS |
| 2856 | DDN |
| 2857 | DNN |
| 2858 | LNSDGSH |
| 2859 | DAS |
| 2860 | DDS |
| 2861 | DAS |
| 2862 | DAS |
| 2863 | GAS |
| 2864 | GAS |
| 2865 | STS |
| 2866 | AAS |
| 2867 | DAS |
| 2868 | EVS |
| 2869 | EDN |
| 2870 | DAS |
| 2871 | DNN |
| 2872 | NVS |
| 2873 | EGY |
| 2874 | GAS |
| 2875 | DNN |
| 2876 | GAS |
| 2877 | DAS |
| 2878 | GAS |
| 2879 | SAS |
| 2880 | EVS |
| 2881 | TAS |
| 2882 | AAS |
| 2883 | DAS |
| 2884 | VNT |
| 2885 | KAS |
| 2886 | SVS |
| 2887 | SNN |
| 2888 | EVS |
| 2889 | AAS |
| 2890 | DAS |
| 2891 | AAS |
| 2892 | AAS |
| 2893 | DAS |
| 2894 | AAS |
| 2895 | AAS |
| 2896 | GAS |
| 2897 | AAS |
| 2898 | AAS |
| 2899 | AAS |
| 2900 | DAS |
| 2901 | GLTVSSNY |
| 2902 | GFTVSRNY |
| 2903 | GVIVSSNY |
| 2904 | GFTVSSNY |
| 2905 | GVTVSSNY |
| 2906 | GIIVSSNY |
| 2907 | GIIVSSNY |
| 2908 | GFTVSSNY |
| 2909 | GLTVSSNY |
| 2910 | GLTVSSNY |
| 2911 | GIIVSSNY |
| 2912 | GVTVSRNY |
| 2913 | GITVSSNY |
| 2914 | GFTVSSNY |
| 2915 | GLTVSSNY |
| 2916 | GLTVSSNY |
| 2917 | GLIVSSNY |
| 2918 | GFTVSSNY |
| 2919 | GFIVSSNY |
| 2920 | GFTVSSNY |
| 2921 | GFIVSRNY |
| 2922 | GITVSSNY |
| 2923 | GFTVSSNY |
| 2924 | GFTVSSNY |
| 2925 | GFTVSSNY |
| 2926 | GVTVSSNY |
| 2927 | GFTVSSNY |
| 2928 | GYTFSSYG |
| 2929 | GYSFTYYG |
| 2930 | GFTFSSYD |
| 2931 | GFIVSSNY |
| 2932 | EFIVSRNY |
| 2933 | GFTVSSNY |
| 2934 | GFTVSSNY |
| 2935 | GFTVSFNY |
| 2936 | IYSGGST |
| 2937 | IYSGGTT |
| 2938 | IYSGGTT |
| 2939 | IYSGGST |
| 2940 | IYSGGST |
| 2941 | IYSGGST |
| 2942 | IYSGGST |
| 2943 | IYSGGST |
| 2944 | IYSGGST |
| 2945 | IYSGGST |
| 2946 | IYSGGST |
| 2947 | IYSGGST |
| 2948 | IYSGGST |
| 2949 | IYSGGST |
| 2950 | IYSGGST |
| 2951 | IYSGGST |
| 2952 | IYSGGST |
| 2953 | IYRGGST |
| 2954 | IYSGGST |
| 2955 | IYPGGST |
| 2956 | IYSGGST |
| 2957 | IYSGGST |
| 2958 | IYSGGST |
| 2959 | IYSGGST |
| 2960 | IYSGGST |
| 2961 | VYSGGST |
| 2962 | IYSGGST |
| 2963 | ISGYNGHT |
| 2964 | ISPYNGDT |
| 2965 | IGTAGDT |
| 2966 | IYSGGST |
| 2967 | IYSGGST |
| 2968 | IYSGGST |
| 2969 | IYSGGST |
| 2970 | IYPGGST |
| 2971 | ARDLDYYGMDV |
| 2972 | ARDLVVYGMDV |
| 2973 | ARDLDYYGMDV |
| 2974 | ARDLDYGGGMDV |
| 2975 | ARPIVGARSGMDV |
| 2976 | ARDLGTYGMDV |
| 2977 | ARDLGPYGMDV |
| 2978 | ARDLGAYGMDV |
| 2979 | ARDLYYYGMDV |
| 2980 | ARDLDYYGMDV |
| 2981 | ARDLDYYGMDV |
| 2982 | ARDGYGMDV |
| 2983 | ARGGAYYYGMDV |
| 2984 | ARDLDYMDV |
| 2985 | ARLPYGMDV |
| 2986 | ARLPYGMDV |
| 2987 | ARARIYTYGPDY |
| 2988 | ARVGDSRSWPFEY |
| 2989 | ARAPYSSRSET |
| 2990 | AREIRVITPVEV |
| 2991 | ARGPYPRFDY |
| 2992 | ARERGGRFDY |
| 2993 | ARDRPAAAIRF |
| 2994 | ARDYAGRV |
| 2995 | ARELSYSSSSGVGPKY |
| 2996 | ARLINHYYDSSGDGGAFDI |
| 2997 | ARIGGVAAAGTADGAFDI |
| 2998 | ARERFGISHDY |
| 2999 | AKGVVALTGTLLRLDP |
| 3000 | ARDRDNGSGSYLGWAFDI |
| 3001 | ARDYGDYYFDY |
| 3002 | ARDYGDYYFDY |
| 3003 | ARDYGDYWFDP |
| 3004 | ARSYGDYYFDY |
| 3005 | ARDYGDFYFDY |
| 3006 | QGISSY |
| 3007 | QGISSY |
| 3008 | QGISSY |
| 3009 | QGISSY |
| 3010 | QDINNY |
| 3011 | QGISSY |
| 3012 | QGISSD |
| 3013 | QGISSY |
| 3014 | QGISSY |
| 3015 | QGISSY |
| 3016 | QGISSY |
| 3017 | QGISSY |
| 3018 | QGISSY |
| 3019 | QGISSY |
| 3020 | QDVSKY |
| 3021 | QDIRNY |
| 3022 | QDINNY |
| 3023 | QDISNY |
| 3024 | QDIRNY |
| 3025 | QDINKY |
| 3026 | QDIRNY |
| 3027 | QDISNY |
| 3028 | QDISNY |
| 3029 | QDIRSY |
| 3030 | QDISNY |
| 3031 | SSDVGSYNL |
| 3032 | SSDVGSYNL |
| 3033 | QSVGSN |
| 3034 | QSVRTN |
| 3035 | QSISSY |
| 3036 | QSVSSSY |
| 3037 | QGVSSF |
| 3038 | QSVSSSY |
| 3039 | QGISSY |
| 3040 | QSVSSSY |
| 3041 | AAS |
| 3042 | AAS |
| 3043 | AAS |
| 3044 | AAS |
| 3045 | DAS |
| 3046 | AAS |
| 3047 | AAS |
| 3048 | AAS |
| 3049 | AAS |
| 3050 | AAS |
| 3051 | AAS |
| 3052 | AAS |
| 3053 | AAS |
| 3054 | AAS |
| 3055 | DAS |
| 3056 | DAS |
| 3057 | DAS |
| 3058 | DAS |
| 3059 | DAS |
| 3060 | DAS |
| 3061 | DAS |
| 3062 | DAS |
| 3063 | DAS |
| 3064 | DAS |
| 3065 | DAS |
| 3066 | EVT |
| 3067 | EGS |
| 3068 | GAF |
| 3069 | EAS |
| 3070 | AAS |
| 3071 | GAS |
| 3072 | GAS |
| 3073 | GTS |
| 3074 | AAS |
| 3075 | GAS |
| 3076 | QHLNSYPPIT |
| 3077 | QQLNSYPLT |
| 3078 | QQLNSYGLT |
| 3079 | QQLNSYPHRFT |
| 3080 | QQHDNLPVT |
| 3081 | QQLNSYLYT |
| 3082 | QQLNSDLYT |
| 3083 | QQLNSDLYT |
| 3084 | QQLDSYPL |
| 3085 | QQLNSYLAIT |
| 3086 | QQLNSYPPFT |
| 3087 | QQLNSYPPA |
| 3088 | QQLNTYPPFG |
| 3089 | QQLNSYPPMYT |
| 3090 | QQYDNLPVT |
| 3091 | QQYDNLPIT |
| 3092 | QQYDNLPPV |
| 3093 | QQYDNLPLFT |
| 3094 | QQYDNLPIT |
| 3095 | HQYDNLPRT |
| 3096 | QQYDNLPVT |
| 3097 | QQHDNLPSFT |
| 3098 | QQYDNLPPA |
| 3099 | QQYDNLPQT |
| 3100 | QQYDNLPPT |
| 3101 | CSYAGSSTWV |
| 3102 | CSYAGSSTWV |
| 3103 | QQYNNWYT |
| 3104 | QQYNNWPPIT |
| 3105 | QQSYSMPPVT |
| 3106 | QQYGSTPRT |
| 3107 | QQYGSSPRT |
| 3108 | QQYGSSPRT |
| 3109 | QQLNS |
| 3110 | QQYDSSPRT |
| 3111 | EVQLVESGGGLIQPGGSLRLSCAASGLTVSSNYMS |
| | WVRQAPGKGLEWVSVIYSGGSTYYADSVKGRFTIS |
| | RDNSTNTLYLQMNSLRAEDTAVYYCARDLDYYGMD |
| | VWGQGTTVTVSS |
| 3112 | EVQLVESGGGLVQPGGSLRLSCAASGFTVSRNYMS |
| | WVRQAPGKGLEWVSVIYSGGTTHYADSVKGRFTIS |
| | RHNSKNTLYLQMNSLRAEDTAVYYCARDLVVYGMD |
| | VWGQGTTVTVSS |
| 3113 | EVQLVESGGGLVQPGGSLRLSCAASGVIVSSNYMR |
| | WVRQAPGKGLEWVSVIYSGGTTYYADSVKGRFTIS |
| | RHNSKNTLYLQMNSLRTEDTAVYYCARDLDYYGMD |
| | VWGQGTTVTVSS |
| 3114 | EVQLVESGGGLVQPGGSLRLSCAASGFTVSSNYMS |
| | WVRQAPGKGLEWVSVIYSGGSTYYADSVKGRFTIS |
| | RHNSKNTLYLQMNSLRAEDTAVYYCARDLDYGGGM |
| | DVWGQGTTVTVSS |
| 3115 | EVQLVESGGGLIQPGGSLRLSCAASGVTVSSNYMS |
| | WVRQAPGKGLEWVSLIYSGGSTYYADSVKGRFTIS |
| | RDNSKNTLYLQMNSLRAEDTAVYYCARPIVGARSG |
| | MDVWGQGTTVTVSS |
| 3116 | EVQLVESGGGLIQPGGSLRLSCAASGIIVSSNYMS |
| | WVRQAPGKGLEWVSVIYSGGSTFYADSVKGRFTIS |
| | RDNSKNTLYLQMNTMRAEDTAVYYCARDLGTYGMD |
| | VWGQGTTVTVS |
| 3117 | EVQLVESGGGLIQPGGSLRLSCAASGIIVSSNYMT |
| | WVRQAPGKGLEWVSVIYSGGSTFYADSVKGRFTIS |
| | RDNSKNTLYLQMSSLRAEDTAVYYCARDLGPYGMD |
| | VWGQGTTVTVSS |
| 3118 | EVQLVESGGGLIQPGGSLRLSCAASGFTVSSNYMS |
| | WVRQAPGKGLEWVSVIYSGGSTFYADSVKGRFTIS |
| | RDNSKNTLYLQMNSLRAEDTAVYYCARDLGAYGMD |
| | VWGQGTTVTVSS |
| 3119 | EVQLVESGGGLIQPGGSLRLSCAASGLTVSSNYMS |
| | WVRQAPGKGLEWVSVIYSGGSTFYADSVKGRFTIS |
| | RDNSKNTLYLQMNSLRAEDTAVYYCARDLYYYGMD |
| | VWGQGTTVTVSS |
| 3120 | EVQLVESGGGLIQPGGSLRLSCAASGLTVSSNYMS |
| | WVRQAPGKGLEWVSVIYSGGSTYYADSVKGRFTIS |
| | RDNSKNTLYLQMNSLRAEDTAVYYCARDLDYYGMD |
| | VWGQGTTVTVSS |
| 3121 | EVQLVESGGGLVQPGGSLRLSCAASGIIVSSNYMS |
| | WVRQAPGKGLEWVSVIYSGGSTYYADSVKGRFTIS |
| | RDNSKNTLYLQMNSLRAEDTAVYYCARDLDYYGMD |
| | VWGQGTTVTVSS |
| 3122 | EVQLVESGGGLVQPGGSLRLSCAASGVTVSRNYMS |
| | WVRQAPGKGLEWVSVIYSGGSTDYADSVKGRFTIS |
| | RHNSKNTLYLQMNSLRVEDTAVYYCARDGYGMDVW |
| | GQGTTVTVSS |
| 3123 | EVQLVESGGGLIQPGGSLRLSCAASGITVSSNYMS |
| | WVRQAPGKGLEWVSVIYSGGSTYYADSVKGRFTIS |
| | RDNSKNTLYLQMNSLRAEDTAVYYCARGGAYYYGM |
| | DVWGQGTTVTVSS |
| 3124 | EVQLVESGGGLVQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARDLDYMDVW |
| | GKGTTVTVSS |
| 3125 | EVQLVESGGGLVQPGGSLRLSCAASGLTVSSNYMS |
| | WVRQAPGKGLEWVSVIYSGGSTFYADSVKGRFTIS |
| | RDNSKNTLYLQMNSVRAEDTAVYYCARLPYGMDVW |
| | GQGTTVTVSS |
| 3126 | EVQLVESGGGLVQPGGSLRLSCAASGLTVSSNYMS |
| | WVRQAPGKGLNWVSVIYSGGSTYYADSVKGRFTIS |
| | RDNSKNTLYLEMNSLKPEDTAVYYCARLPYGMDVW |
| | GQGTTVTVSS |
| 3127 | QVQLVESGGGLVQPGGSLRLSCAASGLIVSSNYMS |
| | WVRQAPGEGLEWVSVIYSGGSTYYADSVKGRFTIS |
| | RDTSKNTLYLQMNSLRAEDTAVYYCARARIYTYGP |
| | DYWGQGTLVTVSS |
| 3128 | EVQLVESGGGLVQPGGSLRLSCAASGFTVSSNYMS |
| | WVRQAPGRGLEWVSVIYRGGSTYYADSVKGRFSIS |
| | RDNSKNTLYLQMNSLRVEDTAVYYCARVGDSRSWP |
| | FEYWGQGTLVTVSS |
| 3129 | EVQLVESGGGLVQPGGSLRLSCAASGFIVSSNYMS |
| | WVRQAPGKGLEWVSVIYSGGSTYYADSVKGRFTIS |
| | RDNSKNTLYLRMNSLRAEDTAVYYCARAPYCSSRS |
| | CETWGQGTLVTVSS |
| 3130 | EVQLVESGGGLVQPGGSLRLSCAASGFTVSSNYMS |
| | WVRQAPGKGLEWVSLIYPGGSTYYADSVEGRFTIS |
| | RDNSKNTLYLQMNSLRAEDTAVYYCAREIRVITPV |
| | EVWGQGTLVTVSS |
| 3131 | EVQLVESGGGLVQPGGSLRLSCAVSGFIVSRNYMT |
| | WVRQAPGKGLEWVSLIYSGGSTFYTNSVKGRFTIS |
| | RDNSKNTLYLQMDSLRAEDTAVYYCARGPYPRFDY |
| | WGQGTLVTVSS |
| 3132 | EVQLVESGGGLIQPGGSLRLSCAASGITVSSNYMS |
| | WVRQAPGKGLEWVSVIYSGGSTFYSDSVKGRFTIS |
| | RDNSKNTLYLQMNSLRAEDTAVYYCARERGGRFDY |
| | WGQGTLVTVSS |
| 3133 | EVQLVESGGGLVQPGGSLRLSCAASGFTVSSNYMS |
| | WVRQAPGKGLEWVSLIYSGGSTYYADSVKGRFTIS |
| | RDNSKNTLYLQMNSLRAEDTAVYYCARDRPAAAIR |
| | FGQGTLVTVSS |
| 3134 | EVQLVESGGGLVQPGGSLRLSCAASGFTVSSNYMS |
| | WVRQAPGKGLEWVSIIYSGGSTYYADSVKGRFTIS |
| | RDNSKNTLYLQMNSLRAEDTAVYYCARDYAGRVWG |
| | QGTLVTVSS |
| 3135 | EVQLVESGGGLVQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARELSYSSSS |
| | GVGPKYWGQGTLVTVSS |
| 3136 | EVQLVESGGGLVQPGGSLRLSCAASGVTVSSNYMS |
| | WVRQAPGKGLEWVSAVYSGGSTYYADSVKGRFTIS |
| | RHNSKNTLYLQMKSLRPEDTAIYYCARLINHYYDS |
| | SGDGGAFDIWGQGTMVTVSS |
| 3137 | EVQLVESGGGLVQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARIGGVAAAG |
| | TADGAFDIWGQGTMVTVSS |
| 3138 | QVQLVQSGAEVKKPGASVKVSCKTSGYTFSSYGLS |
| | WVRQAPGQGLEWMGWISGYNGHTVNAQNFQDRVTM |
| | TTDTSTDTAYMELRSLRSDDTALYFCARERFGISH |
| | DYWGQGTLVIVSS |
| 3139 | QIQLVQSGPEVKRPGASVKVSCKASGYSFTYYGIS |
| | WVRQAPGQGLEWMGWISPYNGDTKFAQKFQDRVIL |
| | TTDTSTSTAYMELKSLRSDDTAVYYCAKGVVALTG |
| | TLLRLDPWGQGTLVTVSS |
| 3140 | EVQLVESGGGLVQPGGSLRLSCAASGFTFSSYDMH |
| | WVRQATGKGLEWVSVIGTAGDTYYPGSVKGRFTIS |
| | RENAKNSLYLQMNSLRAGDTAVYYCARDRDNGSGS |
| | YLGWAFDIWGQGTMVTVSS |
| 3141 | EVQLVESGGGLIQPGGSLRLSCAASGFIVSSNYMS |
| | WVRQAPGKGLEWVSVIYSGGSTYYADSVKGRFTIS |
| | RDNSKNTLYLQMNSLRAEDTAVYYCARDYGDYYFD |
| | YWGQGTLVTVSS |
| 3142 | EVQLVESGGGLIQPGGSLRLSCAASEFIVSRNYMS |
| | WVRQAPGKGLEWVSVIYSGGSTYYADSVKGRFTIS |
| | RDNSKNTLNLQMNSLRAEDTAVYYCARDYGDYYFD |
| | YWGQGTLVTVSS |
| 3143 | EVQLVESGGGLIQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARDYGDYWFD |
| | PWGQGTLVTVSS |
| 3144 | EVQLVESGGGLIQPGGSLRLSCAASGFTVSSNYMS |
| | WVRQAPGKGLEWVSVIYSGGSTYYADSVKGRFTIS |
| | RDNSKNTLYLQMNSLRAEDTAVYYCARSYGDYYFD |
| | YWGQGTLVTVSS |
| 3145 | EVQVVESGGGLVQPGGSLRLSCAASGFTVSFNYMS |
| | WVRQAPGKGLEWVSVIYPGGSTYYADSVKGRFTIS |
| | RHNSKNTVYLQMNSLRAEDTAVYYCARDYGDFYFD |
| | YWGQGTLVTVSS |
| 3146 | DIQLTQSPSSLSASVGDRVTITCRASQGISSYLAW |
| | YQQKPGKAPNLLIYAASTLQSGVPSRFSGSGSGTD |
| | FTLTISSLQPEDFATYYCQHLNSYPPITFGQGTRL |
| | EIK |
| 3147 | DIQLTQSPSFLSASVGDRVTITCRASQGISSYLAW |
| | YQQKPGKAPKLLIYAASTLQSGVPSSFSGSGSGTE |
| | FTLTISSLQPEDFATYYCQQLNSYPLTFGGGTKVE |
| | IK |
| 3148 | DIQLTQSPSSLSASVGDRVTITCRASQGISSYLAW |
| | YQQKPGKAPKLLIYAASTLQRGVPSRFSGSGSGTD |
| | FNLTISSLQPEDFGTYYCQQLNSYGLTFGGGTKVE |
| | IK |
| 3149 | DIQLTQSPSFLSASVGDRVTITCRASQGISSYLAW |
| | YQQKPGKAPKLLIYAASTLQSGVPSRFSGSGSGTE |
| | FTLTISSLQPEDFATYYCQQLNSYPHRFTFGPGTK |
| | VDIK |
| 3150 | DIQMTQSPSSLSASVGDRVTITCQASQDINNYLNW |
| | YQQKPGKAPKLLIYDASNLETGVPSRFSGSGSGTD |
| | FTFIISSLQPEDIATYYCQQHDNLPVTFGGGTKVE |
| | IK |
| 3151 | DIQLTQSPSFLSASVGDRVTITCRASQGISSYLAW |
| | YEQKPGKAPKLLIYAASTLQSGVPSRFSGSGSGTE |
| | FTLTISTLQPGDFATYYCQQLNSYLYTFGQGTKLE |
| | IK |
| 3152 | DIQLTQSPSFLSASVGDRVTITCRASQGISSDLAW |
| | YQQKPGKAPNLLIYAASTLQSGVPSRFSGSGSGTE |
| | FTLTISSLQPEDFATYYCQQLNSDLYTFGQGTKLE |
| | IK |
| 3153 | DIQLTQSPSFLSASVGDRVTITCRASQGISSYLAW |
| | YQQKPGKAPKLLIYAASTLQSGVPSRFSGSGSGTE |
| | FTLTISSLQPEDFATYYCQQLNSDLYTFGQGTKLE |
| | IK |
| 3154 | AIQLTQSPSSLSASVGDRVTITCRASQGISSYLAW |
| | YQQKPGKAPKLLIFAASTLQSGVPSRFSGSGSGTD |
| | FTLTISSLQPEDFATYYCQQLDSYPLFGGGTKVEI |
| | K |
| 3155 | AIQLTQSPSSLSASVGDRVTITCRASQGISSYLAW |
| | YQQKPGKAPKLLIYAASTLQSGVPSRFSGSGSGTD |
| | FTLTISSLQPEDFATYYCQQLNSYLAITFGQGTRL |
| | EIK |
| 3156 | DIQLTQSPSFLSASVGDRVTITCRASQGISSYLAW |
| | YQQKPGKAPKLLIYAASTLQSGVPSRFSGSGSGTE |
| | FTLTISSLQPEDFATYYCQQLNSYPPFTFGPGTKV |
| | DIK |
| 3157 | DIQLTQSPSFLSASVGDRVTITCRASQGISSYLAW |
| | YQQKPGKAPNLLIYAASTLQSGVPSRFSGSGSGTE |
| | FTLTISSLQPEDFATYYCQQLNSYPPAFGPGTKVD |
| | IK |
| 3158 | DIQLTQSPSFLSASVGDRVTITCRASQGISSYLAW |
| | YQQKPGKAPKLLIYAASTLQSGVPSRFSGSGSGTE |
| | FTLTISSLQPEDFATYYCQQLNTYPPFGFGPGTKV |
| | DIK |
| 3159 | DIQLTQSPSSLSASVGDRVTITCRASQGISSYLAW |
| | YQQKPGKAPKLLIYAASTLQSGVPSRFSGSGSGTD |
| | FTLTISSLQPEDFATYYCQQLNSYPPMYTFGQGTK |
| | LEIK |
| 3160 | DIQMTQSPSSLSASVGDRVTITCQASQDVSKYLNW |
| | YQQKPGKAPKLLIHDASNLQTGVPSRFSGGGSGTD |
| | FTFTISSLQPEDIATYYCQQYDNLPVTFGGGTKVE |
| | IK |
| 3161 | DIQMTQSPSSLSASVGDRVTITCQASQDIRNYLNW |
| | YQQKPGKAPKLLIHDASNLETGVPSRFIGSGSGTD |
| | FTFTISSLQPEDIATYYCQQYDNLPITFGQGTRLE |
| | IK |
| 3162 | DIQMTQSPSSLSASVGDRVTITCQASQDINNYLNW |
| | YQQKPGKAPKLLIYDASNLETGVPSRFSGSGSGTD |
| | FTFTISSLQPEDIATYYCQQYDNLPPVFGPGTKVD |
| | IK |
| 3163 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPLFTFGPGTKV |
| | DIK |
| 3164 | DIQMTQSPSSLSASVGDRVTITCQASQDIRNYLNW |
| | YQQKPGKAPNLLIYDASNLETGVPSRFSGSGSGTD |
| | FTFTINSLQPEDIATYYCQQYDNLPITFGQGTRLE |
| | IK |
| 3165 | DIQMTQSPSSLSASVGDRVTITCQASQDINKYLNW |
| | YQLKPGKAPNLLIYDASNLETGVPSRFSGSGSGTD |
| | FTFTISSLQPEDIATYYCHQYDNLPRTFGQGTKVE |
| | IK |
| 3166 | DIQMTQSPSSLSASLGDRVTITCQASQDIRNYLNW |
| | YQQKPGKAPKLLIYDASNLETGVPSRFSGSGSGTD |
| | FTFTISSLQPEDIATYYCQQYDNLPVTFGGGTKVE |
| | IK |
| 3167 | DIQMTQSPSSLSASVGDRVTITCQASQDISNYLNW |
| | YQQKPGKAPKLLIYDASTLETGVPSRFSGSGSGTD |
| | FTFTISSLQPEDIATYYCQQHDNLPSFTFGPGTKV |
| | DIK |
| 3168 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPPAFGGGTKVE |
| | IK |
| 3169 | DIQMTQSPSSLSASVGDRVTITCQASQDIRSYLNW |
| | YQQKPGKAPKLLIYDASNLETGVASRFSGSGSGTD |
| | FTFTISSLQPEDIATYYCQQYDNLPQTFGQGTKLE |
| | IK |
| 3170 | DIQMTQSPSSLSASVGDRVTITCQASQDISNYLNW |
| | YQQKPGKAPKLLIYDASNLETGVPSRFSGSGSGTD |
| | FTFTISSLQPEDIATYYCQQYDNLPPTFGGGTKVE |
| | IK |
| 3171 | QSALTQPASVSGSPGQSITISCTGTSSDVGSYNLV |
| | SWYQQRPGKAPKLILYEVTKRPSGVSNRFSGSKSG |
| | NTASLAISGLQAEDEADYYCCSYAGSSTWVFGGGT |
| | KLTVL |
| 3172 | QSALTQPASVSGSPGQSITISCTGTSSDVGSYNLV |
| | SWYQQHPGKAPKLMIYEGSKRPSGVSNRFSGSKSG |
| | NTASLTISGLQAEDEADYYCCSYAGSSTWVFGGGT |
| | KLTVL |
| 3173 | EIVMTQFPATLSVSPGERATLFCRASQSVGSNLAW |
| | YQQKPGQAPRLLIYGAFTRATGVPARFSGSGSGSE |
| | FSLTISSLQSEDFAVYYCQQYNNWYTFGQGTKLEI |
| | K |
| 3174 | EIVMTQSPATLSVSPGERATLSCRASQSVRTNLAW |
| | YQQKRGQAPRLLIYEASTRATGVPDRFSGSGSGTE |
| | FTLTISSLQSEDFAVYYCQQYNNWPPITFGQGTRL |
| | DIK |
| 3175 | DIQMTQSPSSLSASVGDRVTITCRASQSISSYLNW |
| | YQQKPGKAPKLLIYAASSLQSGVPSRFSASGSGTD |
| | FTLTISSLQPEDFATYYCQQSYSMPPVTFGQGTKV |
| | EIK |
| 3176 | EIVLTQSPGTLSLSPGERATLSCRASQSVSSSYLA |
| | WYQQKPGQAPRLLIYGASSRATGIPERFSGSGSGT |
| | DFTLTISRLEPEDFAVYYCQQYGSTPRTFGQGTKV |
| | EIK |
| 3177 | EIVLTQSPGTLSLSPGERATLSCRASQGVSSFLAW |
| | YQQKPGQAPRLLIHGASSRATGIPDRFSGSGSGTD |
| | FTLTITRLEPEDFAVYYCQQYGSSPRTFGQGTKVE |
| | IK |
| 3178 | EIVLTQSPGTLSLSPGERATLSCRASQSVSSSYLA |
| | WYQQKPGQAPRLLIYGTSSRATGIPDRFSGSGSGT |
| | DFTLTISRLEPEDFAVYYCQQYGSSPRTFGQGTKV |
| | EIK |
| 3179 | DIQLTQSPSSLSASVGDRVTITCRASQGISSYLAW |
| | YQQKPGKAPKLLIYAASTLQSGVPSRFSGSGSGTD |
| | FTLTISSLQPEDFATYYCQQLNSFGPGTKVDIK |
| 3180 | EIVLTQSPGTLSLSPGERATLSCRASQSVSSSYLA |
| | WYQQKPGQAPRLLIYGASSRATGIPDRFSGSGSGT |
| | DFTLTISRLEPEDFAMYYCQQYDSSPRTFGQGTKV |
| | EIK |

Although the specific embodiments described herein have been described in detail, a person skilled in the art would understand that: Various modifications and changes in the details can be made according to all the teachings disclosed and these changes are within the scope of the protection of the present invention. All of the present invention are given by the appended claims and any equivalents thereof

What is claimed is:

1. An antigen-binding unit comprising a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3, wherein the VH CDR3 comprises a sequence selected from SEQ ID NOs: 1-360 and 2971-3005 or a sequence comprising one or more amino acid additions, deletions, or substitutions compared with SEQ ID NOs: 1-360 and 2971-3005, and/or wherein the VL CDR3 comprises a sequence selected from SEQ ID NOs: 361-720 and 3076-3110 or a sequence comprising one or more amino acid additions, deletions, or substitutions compared with SEQ ID NOs: 361-720 and 3076-3110.

2. The antigen-binding unit of any one of the preceding claims, wherein the antigen-binding unit binds to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

3. The antigen-binding unit of any one of the preceding claims, wherein the antigen-binding unit neutralizes the novel coronavirus (SARS-CoV-2) with an IC₅₀ of less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, or less than 0.001 μg/ml.

4. The antigen-binding unit of any one of the preceding claims, wherein the VH CDR1 comprises a sequence selected from SEQ ID NOs: 1461-1820 and 2901-2935 or a sequence comprising one or more amino acid additions, deletions, or substitutions compared with SEQ ID NOs: 1461-1820 and 2901-2935.

5. The antigen-binding unit of any one of the preceding claims, wherein the VH CDR1 comprises a sequence selected from SEQ ID NOs: 1461-1820 and 2901-2935.

6. The antigen-binding unit of any one of the preceding claims, wherein the VH CDR1 comprises a sequence comprising 5, 4, 3, 2 or 1 amino acid additions, deletions, or substitutions compared with SEQ ID NOs: 1461-1820 and 2901-2935.

7. The antigen-binding unit of any one of the preceding claims, wherein the VH CDR1 comprises the same sequence as CDR1 contained in SEQ ID NOs: 721-1080 and 3111-3145.

8. The antigen-binding unit of any one of the preceding claims, wherein the VH CDR2 comprises a sequence selected from SEQ ID NOs: 1821-2180 and 2936-2970 or a sequence comprising one or more amino acid additions, deletions, or substitutions compared with SEQ ID NOs: 1821-2180 and 2936-2970.

9. The antigen-binding unit of any one of the preceding claims, wherein the VH CDR2 comprises a sequence selected from SEQ ID NOs: 1821-2180 and 2936-2970.

10. The antigen-binding unit of any one of the preceding claims, wherein the VH CDR2 comprises a sequence comprising 5, 4, 3, 2 or 1 amino acid additions, deletions, or substitutions compared with SEQ ID NOs: 1821-2180 and 2936-2970.

11. The antigen-binding unit of any one of the preceding claims, wherein the VH CDR2 comprises the same sequence as CDR2 contained in SEQ ID NOs: 721-1080 and 3111-3145.

12. The antigen-binding unit of any one of the preceding claims, wherein the VL CDR1 comprises a sequence selected from SEQ ID NOs: 2181-2540 and 3006-3040 or a sequence comprising one or more amino acid additions, deletions, or substitutions compared with SEQ ID NOs: 2181-2540 and 3006-3040.

13. The antigen-binding unit of any one of the preceding claims, wherein the VL CDR1 comprises a sequence selected from SEQ ID NOs: 2181-2540 and 3006-3040.

14. The antigen-binding unit of any one of the preceding claims, wherein the VL CDR1 comprises a sequence comprising 5, 4, 3, 2 or 1 amino acid additions, deletions, or substitutions compared with SEQ ID NOs: 2181-2540 and 3006-3040.

15. The antigen-binding unit of any one of the preceding claims, wherein the VL CDR1 comprises the same sequence as CDR1 contained in SEQ ID NOs: 1081-1440 and 3146-3180.

16. The antigen-binding unit of any one of the preceding claims, wherein the VL CDR2 comprises a sequence selected from SEQ ID NOs: 2541-2900 and 3041-3075 or a sequence comprising one or more amino acid additions, deletions, or substitutions compared with SEQ ID NOs: 2541-2900 and 3041-3075.

17. The antigen-binding unit of any one of the preceding claims, wherein the VL CDR2 comprises a sequence selected from SEQ ID NOs: 2541-2900 and 3041-3075.

18. The antigen-binding unit of any one of the preceding claims, wherein the VL CDR2 comprises a sequence comprising 5, 4, 3, 2 or 1 amino acid additions, deletions, or substitutions compared with SEQ ID NOs: 2541-2900 and 3041-3075.

19. The antigen-binding unit of any one of the preceding claims, wherein the VL CDR2 comprises the same sequence as CDR2 contained in SEQ ID NOs: 1081-1440 and 3146-3180.

20. The antigen-binding unit of any one of the preceding claims, wherein the VH comprises a sequence selected from SEQ ID NOs: 721-1080 and 3111-3145 or a sequence comprising one or more amino acid additions, deletions, or substitutions compared with SEQ ID NOs: 721-1080 and 3111-3145.

21. The antigen-binding unit of any one of the preceding claims, wherein the VH comprises a sequence selected from SEQ ID NOs: 721-1080 and 3111-3145.

22. The antigen-binding unit of any one of the preceding claims, wherein the VH comprises a sequence comprising 10, 9, 8, 7, 6, 5, 4, 3, 2 or 1 amino acid additions, deletions, or substitutions compared with SEQ ID NOs: 721-1080 and 3111-3145.

23. The antigen-binding unit of any one of the preceding claims, wherein the VH comprises a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145.

24. The antigen-binding unit of any one of the preceding claims, wherein the VL comprises a sequence selected from SEQ ID NOs: 1081-1440 and 3146-3180 or a sequence comprising one or more amino acid additions, deletions, or substitutions compared with SEQ ID NOs: 1081-1440 and 3146-3180.

25. The antigen-binding unit of any one of the preceding claims, wherein the VL comprises a sequence selected from SEQ ID NOs: 1081-1440 and 3146-3180.

26. The antigen-binding unit of any one of the preceding claims, wherein the VL comprises a sequence comprising 10, 9, 8, 7, 6, 5, 4, 3, 2 or 1 amino acid additions, deletions, or substitutions compared with SEQ ID NOs: 1081-1440 and 3146-3180.

27. The antigen-binding unit of any one of the preceding claims, wherein the VL comprises a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180.

28. An antigen-binding unit comprising a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3, wherein the VH CDR1 comprises a sequence selected from SEQ ID NOs: 1461-1820 and 2901-2935, a sequence comprising one or more amino acid additions, deletions, or substitutions compared with SEQ ID NOs: 1461-1820 and 2901-2935, or the same sequence as CDR1 contained in SEQ ID NOs: 721-1080 and 3111-3145, wherein the VH CDR2 comprises a sequence selected from SEQ ID NOs: 1821-2180 and 2936-2970, a sequence comprising one or more amino acid additions, deletions, or substitutions compared with SEQ ID NOs: 1821-2180 and 2936-2970, or the same sequence as CDR2 contained in SEQ ID NOs: 721-1080 and 3111-3145, and wherein the VH CDR3 comprises a sequence selected from SEQ ID NOs: 1-360 and 2971-3005, a sequence comprising one or more amino acid additions, deletions, or substitutions compared with SEQ ID NOs: 1-360 and 2971-3005, or the same sequence as CDR3 contained in SEQ ID NOs: 721-1080 and 3111-3145, and/or wherein the VL CDR1 comprises a sequence selected from SEQ ID NOs: 2181-2540 and 3006-3040, a sequence comprising one or more amino acid additions, deletions, or substitutions compared with SEQ ID NOs: 2181-2540 and 3006-3040, or the same sequence as CDR1 contained in SEQ ID NOs: 1081-1440 and 3146-3180, the VL CDR2 comprises a sequence selected from SEQ ID NOs: 2541-2900 and 3041-3075, a sequence comprising one or more amino acid additions, deletions, or substitutions compared with SEQ ID NOs: 2541-2900 and 3041-3075, or the same sequence as CDR2 contained in SEQ ID NOs: 1081-1440 and 3146-3180, and the VL CDR3 comprises a sequence selected from SEQ ID NOs: 361-720 and 3076-3110, a sequence comprising one or more amino acid additions, deletions, or substitutions compared with SEQ ID NOs: 361-720 and 3076-3110, or the same sequence as CDR3 contained in SEQ ID NOs: 1081-1440 and 3146-3180.

29. An antigen-binding unit comprising a heavy chain variable region (VH) and a light chain variable region (VL), wherein the heavy chain variable region comprises VH CDR1, VH CDR2 and VH CDR3, and the light chain variable region comprises VL CDR1, VL CDR2 and VL CDR3, wherein the VH CDR1 comprises a sequence selected from SEQ ID NOs: 1461-1820 and 2901-2935 or a sequence comprising one or more amino acid additions, deletions, or substitutions compared with SEQ ID NOs: 1461-1820 and 2901-2935, wherein the VH CDR2 comprises a sequence selected from SEQ ID NOs: 1821-2180 and 2936-2970 or a sequence comprising one or more amino acid additions, deletions, or substitutions compared with SEQ ID NOs: 1821-2180 and 2936-2970, and wherein the VH CDR3 comprises a sequence selected from SEQ ID NOs: 1-360 and 2971-3005 or a sequence comprising one or more amino acid additions, deletions, or substitutions compared with SEQ ID NOs: 1-360 and 2971-3005, and/or wherein the VL CDR1 comprises a sequence selected from SEQ ID NOs: 2181-2540 and 3006-3040 or a sequence comprising one or more amino acid additions, deletions, or substitutions compared with SEQ ID NOs: 2181-2540 and 3006-3040, the VL CDR2 comprises a sequence selected from SEQ ID NOs: 2541-2900 and 3041-3075 or a sequence comprising one or more amino acid additions, deletions, or substitutions compared with SEQ ID NOs: 2541-2900 and 3041-3075, and the VL CDR3 comprises a sequence selected from SEQ ID NOs: 361-720 and 3076-3110 or a sequence comprising one or more amino acid additions, deletions, or substitutions compared with SEQ ID NOs: 361-720 and 3076-3110.

30. The antigen-binding unit of any one of the preceding claims, wherein the VH comprises a sequence selected from SEQ ID NOs: 721-1080 and 3111-3145 or a sequence comprising one or more amino acid additions, deletions, or substitutions compared with SEQ ID NOs: 721-1080 and 3111-3145.

31. The antigen-binding unit of any one of the preceding claims, wherein the VH comprises a sequence selected from SEQ ID NOs: 721-1080 and 3111-3145.

32. The antigen-binding unit of any one of the preceding claims, wherein the VH comprises a sequence comprising 10, 9, 8, 7, 6, 5, 4, 3, 2 or 1 amino acid additions, deletions, or substitutions compared with SEQ ID NOs: 721-1080 and 3111-3145.

33. The antigen-binding unit of any one of the preceding claims, wherein the VH comprises a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145.

34. The antigen-binding unit of any one of the preceding claims, wherein the VL comprises a sequence selected from SEQ ID NOs: 1081-1440 and 3146-3180 or a sequence comprising one or more amino acid additions, deletions, or substitutions compared with SEQ ID NOs: 1081-1440 and 3146-3180.

35. The antigen-binding unit of any one of the preceding claims, wherein the VL comprises a sequence selected from SEQ ID NOs: 1081-1440 and 3146-3180.

36. The antigen-binding unit of any one of the preceding claims, wherein the VL comprises a sequence comprising 10, 9, 8, 7, 6, 5, 4, 3, 2 or 1 amino acid additions, deletions, or substitutions compared with SEQ ID NOs: 1081-1440 and 3146-3180.

37. The antigen-binding unit of any one of the preceding claims, wherein the VL comprises a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180.

38. The antigen-binding unit of any one of the preceding claims, wherein the antigen-binding unit binds to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

39. The antigen-binding unit of any one of the preceding claims, wherein the antigen-binding unit neutralizes the novel coronavirus (SARS-CoV-2) with an IC₅₀ of less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, or less than 0.001 μg/ml.

40. An antigen-binding unit comprising a heavy chain variable region (VH) and a light chain variable region (VL), wherein the VH comprises a sequence having at least 80%, 85%, 90%, 91%, 92%, 93%, 94%, 95%, 96%, 97%, 98%, 99% or 99% identity to a sequence selected from SEQ ID NOs: 721-1080 and 3111-3145, and/or wherein the VL comprises a sequence having at least 80%, 85%, 90%, 91%, 92%, 93%, 94%, 95%, 96%, 97%, 98%, 99% or 99% identity to a sequence selected from SEQ ID NOs: 1081-1440 and 3146-3180.

41. The antigen-binding unit of any one of the preceding claims, wherein the antigen-binding unit binds to a receptor binding domain (RBD) of an S protein of a novel coronavirus (SARS-CoV-2) with an equilibrium dissociation constant (KD) of less than 100 nM, less than 50 nM, less than 20 nM, less than 15 nM, less than 10 nM, less than 5 nM, less than 4 nM, less than 3 nM, less than 2 nM, less than 1 nM, less than 0.5 nM, less than 0.1 nM, less than 0.05 nM, or less than 0.01 nM.

42. The antigen-binding unit of any one of the preceding claims, wherein the antigen-binding unit neutralizes the novel coronavirus (SARS-CoV-2) with an IC₅₀ of less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, less than 0.5 μg/ml, less than 0.25 μg/ml, less than 0.2 μg/ml, less than 0.1 μg/ml, less than 0.05 μg/ml, or less than 0.001 μg/ml.

43. A pharmaceutical composition comprising the antigen-binding unit of any one of claims 1 to 42 and a pharmaceutically acceptable excipient.

44. An isolated nucleic acid encoding the antigen-binding unit of any one of claims 1 to 42.

45. A vector comprising a nucleic acid sequence encoding the antigen-binding unit of any one of claims 1 to 42.

46. A host cell expressing the antigen-binding unit of any one of claims 1 to 42.

47. A host cell comprising a nucleic acid encoding the antigen-binding unit of any one of claims 1 to 42.

48. A method for producing the antigen-binding unit of any one of claims 1 to 42, comprising: culturing the host cell of claim 46 or 47 under conditions suitable for the expression of the antigen-binding unit; and isolating the antigen-binding unit expressed by the host cell.

49. A conjugate comprising the antigen-binding unit of any one of claims 1 to 42, wherein the antigen-binding unit is conjugated to a chemically functional moiety.

50. The conjugate of claim 49, wherein the chemically functional moiety is selected from a radioisotope, an enzyme, a fluorescent compound, a chemiluminescent compound, a bioluminescent compound, a substrate, a cofactor and an inhibitor.

51. A method for preventing and/or treating SARS-CoV-2 infection in a patient in need thereof, comprising administering the antigen-binding unit of any one of claims 1 to 42 or a nucleic acid encoding the antigen-binding unit to the patient.

52. A kit for detecting SARS-CoV-2, comprising the antigen-binding unit of any one of claims 1 to 42.
